(19) United States
(12) Patent Application Publication
(10) Pub. No.: US 2023/0011571 A1
(43) Pub. Date: Jan. 12, 2023

(54) SMALL MOLECULE FURIN INHIBITORS FOR TREATING INFECTIOUS DISEASES

(71) Applicant: BP Asset V, Inc., Cambridge, MA (US)

(72) Inventors: Keith Wilcoxen, Cambridge, MA (US); Claudine Mapa, Cambridge, MA (US)

(73) Assignee: BP Asset V, Inc., Cambridge, MA (US)

(21) Appl. No.: 17/776,067

(22) PCT Filed: Nov. 12, 2020
(86) PCT No.: PCT/US2020/060108

§ 371 Date: May 11, 2022

Related U.S. Application Data
(60) Provisional application No. 62/934,407, filed on Nov. 12, 2019.

(51) Int. Cl. A61K 31/496; A61P 31/14

(52) U.S. Cl. CPC A61K 31/496; A61P 31/14

(57) ABSTRACT

Provided herein are methods of treating a viral infection in a subject in need thereof, comprising administering to the subject a compound of Formula (I), or a pharmaceutical composition comprising Formula (I). Further provided herein are methods for inhibiting the replication of a virus (e.g., a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburg virus (e.g., Marburg virus, Ravn virus)), human respiratory syncytial virus (i.e., human orthopneumo virus), a flavivirus (e.g., dengue virus, Usutu virus, Japanese encephalitis virus, Powassan virus, yellow fever), a paramyxoviridae family virus (e.g., an orthoparamyxovirinae virus (e.g., a henipavims (e.g., Nipah virus), a morbillivims (e.g., measles morbillivirus))) in a subject in need thereof. Also provided herein are methods for treating and/or preventing a disorder due to a microbial toxin (e.g., due to P. aeruginosa exotoxin A, Clostridium septicum alpha-toxin, diphtheria toxin(s), shiga toxin(s)) in a subject in need thereof, comprising administering to the subject a therapeutically effective amount of a compound of Formula (I), or a pharmaceutical composition comprising a compound of Formula (I) as described herein. Also provided are pharmaceutical compositions and kits including a compound of Formula (I) for use in the treatment and/or prevention of a viral infection in a subject in need thereof.

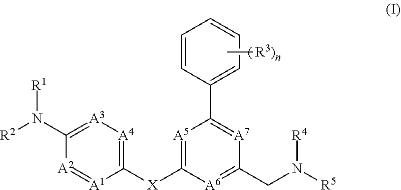

### RELATED APPLICATIONS

The present application claims priority under 35 U.S.C. § 119(e) to U.S. provisional application, U.S. Ser. No. 62/934,407, filed Nov. 12, 2019, which is incorporated herein by reference.

### BACKGROUND OF THE INVENTION

The human genome encodes more than 550 proteases. These molecular scissors play important roles in essentially all physiological processes. Proteolytic cleavage is certainly one of the most important post-translational modifications, generating a plethora of bioactive proteins and peptides with key roles in cell proliferation, immunity, and inflammation. Not surprisingly, mutations in proteases and/or aberrant protease activity are associated with numerous pathological processes including cancer, cardiovascular disorders, and autoimmune diseases (Chakraborti, S., Dhalla N. S.; Pathophysiological Aspects of Proteases. Berlin, Germany: Springer, 2017). Intriguingly, also many viral pathogens exploit cellular proteases for the proteolytic processing and maturation of their own proteins. Similarly, activation of bacterial toxins frequently requires cleavage by proteases of the infected or intoxicated host.

In recent years, modulation of protease activity has therefore emerged as a potential therapeutic approach in a variety of infectious and noninfectious diseases. One particularly promising target for therapeutic intervention is the cellular protease furin. This protease likely cleaves and activates more than 150 mammalian, viral, and bacterial substrates (Tian, S., Huang, Q., Fang, Y. et al. “FurinDB: a database of 20-residue furin cleavage site motifs, substrates and their associated drugs.” Int. J. Mol. Sci. 2011; 12:1060-1065.) Among them are viral envelope glycoproteins and bacterial toxins, as well as cellular factors that promote tumor development and growth if they are hyperactivated.

Furin is a member of the evolutionarily ancient family of proprotein convertases. Their similarity with bacterial subtilisin and yeast kexin proteases has coined the abbreviation PCSK (proprotein convertase subtilisin/kexin type). Humans encode nine members of this protease family (PCSK1-9), with PCSK3 representing furin. PCSKs are well known for their ability to activate other cellular proteins. The proteolytic conversion of inactive precursor proteins into bioactive molecules has already been described in the 1960s (Steiner, D. F., Cunningham, D., Spigelman, L. et al. “Insulin biosynthesis: evidence for a precursor.” Science 1967; 157:697-700). However, it took more than 20 years until furin was identified as the first mammalian proprotein convertase (van de Ven, W. J., Voorberg, J., Fontijn, R. et al. “Furin is a subtilisin-like proprotein processing enzyme in higher eukaryotes.” Mol. Biol. Rep. 1990; 14: 265-275). To date, more than 200 cellular substrates of PCSKs have been described, including hormones, receptors, growth factors, and adhesion molecules.

Known furin inhibitors are peptidic in nature and derived from the natural substrate motif sequence, or are designed peptidomimetic compounds with lysine and arginine sidechains to enable high affinity binding to furin. A potent peptidic furin inhibitor was identified by incorporating a reactive chloromethyl ketone (CMK) moiety (WO 2009/023306 A2; Garten, W., Hallenberger, S., Ortmann, D., Schafer, W., Vey, M., Angliker, H., et al. Biochimie 1994, 76(3-4), 217-225). This non-selective CMK peptide (Decanoyl-Arg-Val-Lys-Arg-CMK) engages the active site of furin at the catalytic Ser368 residue to give a tetrahedral hemiketal that irreversibly alkylates the His194 residue. This well-known irreversible protease inhibition mechanism of a halomethylketone provides very high and durable potency, however also can account for non-selective protease inhibition, particularly against other PCSK family members. Furin inhibitors have been found to protect macrophages from processing of anthrax (WO 2013/138666 A1) and to restore fluid balance in CF cells (Reihill, J. A., Walker, B., Hamilton, R. A., Ferguson, T. E., Elborn, J. S., Stutts, M. J., et al., “Inhibition of Protease-Epithelial Sodium Channel Signaling Improves Mucociliary Function in Cystic Fibrosis Airways.” Am. J. Respir. Crit. Care Med. 2016, 194(6), 701-710).

The reported potent inhibitors of furin are peptide derivatives or peptidomimetics containing polybasic residues in order to achieve high inhibitory potency. As a consequence of the highly basic nature of the inhibitors, reactivity, and peptide structure, their chemical and pharmacokinetics properties limit use as clinical therapeutic agents. Furin plays a diverse biological role in health and diseases with high unmet medical need. Therefore, potent and selective small molecule furin inhibitors with drug-like properties are desirable as an attractive approach to provide therapeutic benefit in many diseases such as infectious diseases.

Infectious diseases may be spread from one person to another and are caused by pathogenic microorganisms such as bacteria, viruses, parasites, or fungi. Pathogenicity is the ability of a microbial agent to cause disease, and virulence is the degree to which an organism is pathogenic. In order for viruses to enter host cells and replicate, the envelope glycoproteins must be proteolytically activated (Nakayama K. Biochem. J. 1997, 327(3), 625-635). The processing of envelope glycoproteins may in some cases impact viral pathogenicity (Nakayama K. Biochem. J. 1997, 327(3), 625-635). The glycoprotein precursors of many virulent viruses, such as human immunodeficiency virus (HIV), avian influenza virus, measles virus, respiratory syncytial virus (RSV), Ebola virus, anthrax, and Zika virus (ZIKV), are cleaved at a site marked by a consensus sequence consistent with furin recognition (Thomas G. Nat. Rev. Mol. Cell. Biol. 2002, 3(10), 753-766; 2, 36-38). The cleavage of HIV glycoprotein160 and infectious virus production are blocked when the furin inhibitor al-PDX is expressed in cells (Nakayama K. Biochem. J. 1997, 327(3), 625-635). It is thus conceivable for the therapeutic use of furin inhibitor in a pandemic situation or biological warfare.

### SUMMARY OF THE INVENTION

Provided herein are methods for treating a viral infection (e.g., resulting from a togaviridae family viruses (e.g., alphaviruses (e.g., Chikungunya virus, Eastern equine encephalitis virus, Mayaro virus, Onyong-nyong virus, Ross River virus, Semliki Forest virus, Sindbis virus, Venezuelan equine encephalitis virus, Western equine encephalitis virus)), flaviviridae family viruses (e.g., flaviviruses (e.g., Dengue virus, Japanese encephalitis virus, Kyasanur Forest disease virus, Murray Valley encephalitis virus, Omsk hemorrhagic fever virus, Powassan virus, Rocio encephalitis virus, Saint Louis encephalitis virus, Tick-borne encephalitis virus, West Nile virus, Yellow fever virus, Usutu virus)), paramyxoviridae family viruses (e.g., orthoparamyxovirinae viruses (e.g., respiroviruses (e.g., human respirovirus 1, human respirovirus 3, murine respirovirus), henipaviruses (e.g., Cedar virus, Kumasi virus, Hendra virus, Mojiang virus, Nipah virus), morbilliviruses (e.g., Canine morbillivirus; Cetacean morbillivirus; Feline morbillivirus; Feline morbillivirus 2; Measles morbillivirus; Phocine morbillivirus; Rinderpest morbillivirus; Small ruminant morbillivirus)), filoviradae family viruses (e.g., Marburgviruses (e.g., Marburg virus, Ravn virus)), human respiratory syncytial virus (i.e., Human orthopneumovirus)), comprising administering to the subject a therapeutically effective amount of a compound of Formula (I), or a pharmaceutical composition comprising a compound of Formula (I) as described herein.

Further provided herein are methods for preventing a viral infection (e.g., resulting from a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus (e.g., a respirovirus (e.g., human respirovirus 1, human respirovirus 3, murine respirovirus), a henipavirus (e.g., Cedar virus, Kumasi virus, Hendra virus, Mojiang virus, Nipah virus), a morbillivirus (e.g., measles morbillivirus))), comprising administering to the subject a prophylactically effective amount of a compound of Formula (I), or a pharmaceutical composition comprising a compound of Formula (I) as described herein.

In another aspect, the present disclosure provides methods of decreasing viral infectivity (e.g., infectivity of a virus (e.g., a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus (e.g., a henipavirus (e.g., Nipah virus), a morbillivirus (e.g., measles morbillivirus))) in a subject, the method comprising administering to the subject an effective amount of a compound of Formula (I), or a pharmaceutical composition comprising Formula (I) as described herein.

In another aspect, the pharmaceutical compositions useful in the present disclosure comprise a compound of Formula (I) as described herein, and optionally a pharmaceutically acceptable excipient.

In yet another aspect, the present invention provides compounds of Formula (I), and pharmaceutical compositions thereof, for use in the treatment of a viral infection (e.g., infection from a virus (e.g., a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus (e.g., a henipavirus (e.g., Nipah virus), a morbillivirus (e.g., measles morbillivirus))) in a subject in need thereof.

In yet another aspect, the present invention provides compounds of Formula (I), and pharmaceutical compositions thereof, for use in the prevention of a viral infection (e.g., infection from a virus (e.g., a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus (e.g., a henipavirus (e.g., Nipah virus), a morbillivirus (e.g., measles morbillivirus))) in a subject in need thereof.

In another aspect, the present disclosure provides uses of compounds of Formula (I), and pharmaceutical compositions thereof, in the manufacture of a medicament for treating a viral infection (e.g., infection from a virus (e.g., a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus (e.g., a henipavirus (e.g., Nipah henipavirus), a morbillivirus (e.g., measles morbillivirus))) in a subject in need thereof.

In another aspect, the present disclosure provides uses of compounds of Formula (I), and pharmaceutical compositions thereof, in the manufacture of a medicament for preventing a viral infection (e.g., infection from a virus (e.g., a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus (e.g., a henipavirus (e.g., Nipah henipavirus), a morbillivirus (e.g., measles morbillivirus))) in a subject in need thereof.

Provided herein are methods for treating and/or preventing a disorder due to a microbial toxin (e.g., due to P. aeruginosa exotoxin A, Clostridium septicum alpha-toxin, diphtheria toxin(s), shiga toxin(s)) in a subject in need thereof, comprising administering to the subject a therapeutically effective amount of a compound of Formula (I), or a pharmaceutical composition comprising a compound of Formula (I) as described herein.

Further provided herein are methods for preventing the activation of a toxin (e.g., P. aeruginosa exotoxin A) in a subject, comprising administering to the subject a prophylactically effective amount of a compound of Formula (I), or a pharmaceutical composition comprising a compound of Formula (I) as described herein.

In yet another aspect, the present invention provides compounds of Formula (I), and pharmaceutical compositions thereof, for use in treating and/or preventing a disorder due to a microbial toxin (e.g., P. aeruginosa exotoxin A, Clostridium septicum alpha-toxin, diphtheria toxin(s), shiga toxin(s)) in a subject in need thereof.

In another aspect, the present disclosure provides uses of compounds of Formula (I), and pharmaceutical compositions thereof, in the manufacture of a medicament for treating and/or preventing a disorder due to a microbial toxin (e.g., P. aeruginosa toxin A, Clostridium septicum alpha-toxin, diphtheria toxin(s), shiga toxin(s)) in a subject in need thereof.

In certain embodiments, the compounds useful in the present disclosure are of the Formula (I):

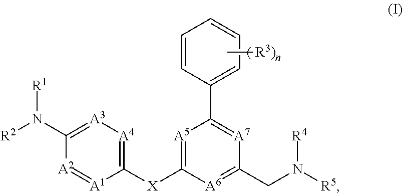

or a pharmaceutically acceptable salt thereof.

In certain embodiments, the compound of Formula (I) useful in the present disclosure is of the formula:

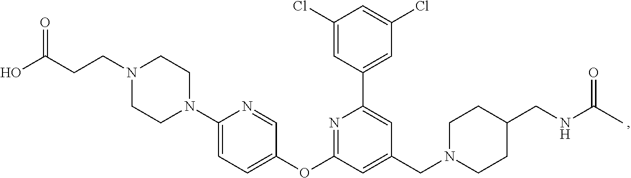 [SMILES: CC(=O)NCC1CCN(Cc2cc(Oc3ccc(N4CCN(CCC(=O)O)CC4)nc3)nc(-c3cc(Cl)cc(Cl)c3)c2)CC1]

or a pharmaceutically acceptable salt thereof.

In certain embodiments, the compound of Formula (I) useful in the present disclosure is of the formula:

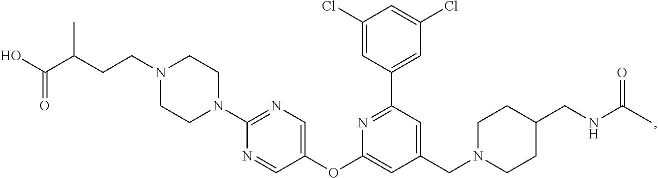 [SMILES: CC(=O)NCC1CCN(Cc2cc(Oc3cnc(N4CCN(CCC(C)C(=O)O)CC4)nc3)nc(-c3cc(Cl)cc(Cl)c3)c2)CC1]

or a pharmaceutically acceptable salt thereof.

Another aspect of the present disclosure relates to kits comprising a container with a compound, or pharmaceutical composition thereof, as described herein. The kits described herein may include a single dose or multiple doses of the compound or pharmaceutical composition. The kits may be useful in a method of the disclosure. In certain embodiments, the kit further includes instructions for using the compound or pharmaceutical composition. A kit described herein may also include information (e.g. prescribing information) as required by a regulatory agency, such as the U.S. Food and Drug Administration (FDA).

The details of certain embodiments of the disclosure are set forth in the Detailed Description of Certain Embodiments, as described below. Other features, objects, and advantages of the disclosure will be apparent from the Definitions, Examples, and Claims.

### Definitions

Terms are used within their ordinary and accepted meanings. The following definitions are meant to clarify, but not limit, the terms defined herein.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind. 1972). This disclosure also encompasses compounds as individual isomers substantially free of other isomers, and alternatively, as mixtures of various isomers.

In a formula, the bond [symbol] is a single bond, the dashed line --- is a single bond or absent, and the bond [symbol] or [symbol] is a single or double bond.

Unless otherwise provided, a formula includes compounds that do not include isotopically enriched atoms and also compounds that include isotopically enriched atoms.

Compounds that include isotopically enriched atoms may be useful, for example, as analytical tools and/or probes in biological assays.

When a range of values (“range”) is listed, it is intended to encompass each value and sub-range within the range. A range is inclusive of the values at the two ends of the range unless otherwise provided. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups. The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋ₛ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

“Alkoxy” refers to a group containing an alkyl radical, attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups include, without limitation, methoxy, ethoxy, n-propoxy, isopropoxy, n-butoxy, s-butoxy, isobutoxy, and t-butoxy.

When the term “alkyl” is used in combination with other substituent groups, such as “halo(C₁-C₆)alkyl”, “(C₃-C₆)cycloalkyl(C₁-C₄)alkyl-”, or “(C₁-C₄)alkoxy(C₂-C₄)alkyl-”, the term “alkyl” is intended to encompass a divalent straight or branched-chain hydrocarbon radical, wherein the point of attachment is through the alkyl moiety. The term “halo(C₁-C₆)alkyl” is intended to mean a radical having one or more halogen atoms, which may be the same or different, at one or more carbon atoms of an alkyl moiety containing from 1 to 6 carbon atoms, which is a straight or branched-chain carbon radical. Examples of “halo(C₁-C₆)alkyl” groups include, but are not limited to, —CH₂F (fluoromethyl), —CHF₂ (difluoromethyl), —CF₃ (trifluoromethyl), —CCl₃ (trichloromethyl), 1,1-difluoroethyl, 2-fluoro-2-methylpropyl, 2,2-difluoropropyl, 2,2,2-trifluoroethyl, and hexafluoroisopropyl. Examples of “(C₃-C₆)cycloalkyl(C₁-C₄)alkyl-” groups include, but are not limited to, cyclobutylmethyl, cyclopentylmethyl, cyclohexylmethyl, cyclobutylethyl, cyclopentylethyl, and cyclohexylethyl. Examples of “(C₁-C₄)alkoxy(C₂-C₄)alkyl-” groups include, but are not limited to, methoxyethyl, methoxyisopropyl, ethoxyethyl, ethoxyisopropyl, isopropoxyethyl, isopropoxyisopropyl, t-butoxyethyl, and t-butoxyisopropyl.

The term “haloalkyl” is a substituted alkyl group, wherein one or more of the —H atoms are independently replaced by a halogen, e.g., fluoro, bromo, chloro, or iodo. “Perhaloalkyl” is a subset of haloalkyl, and refers to an alkyl group wherein all of the —H atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl —H atoms are independently replaced with fluoro to provide a “perfluoroalkyl” group. In some embodiments, all of the haloalkyl —H atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms), such as oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 2 to 20 carbon atoms and one or more carbon-carbon double bonds (e.g., 1, 2, 3, or 4 double bonds). In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). The one or more carbon-carbon double bonds can be internal (such as in 2-butenyl) or terminal (such as in 1-butenyl).

Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₅), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

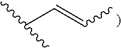

may be in the (E)- or (Z)-configuration.

The term “heteroalkenyl” refers to an alkenyl group, which further includes at least one heteroatom (e.g., 1, 2, 3, or 4 heteroatoms) such as oxygen, nitrogen, or sulfur within (e.g., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 11 carbon atoms, at least one double bond, and 1 or more heteroatoms within the parent chain (“heteroC₂₋₁₁ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 1 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₂₀ alkenyl. In certain embodiments, the heteroalkenyl group is a substituted heteroC₂₋₂₀ alkenyl.

The term “alkynyl” refers to a radical of a straight-chain or branched hydrocarbon group having from 2 to 20 carbon atoms and one or more carbon-carbon triple bonds (e.g., 1, 2, 3, or 4 triple bonds) (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). The one or more carbon-carbon triple bonds can be internal (such as in 2-butynyl) or terminal (such as in 1-butynyl). Examples of C₂₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₂₀ alkynyl. In certain embodiments, the alkynyl group is a substituted C₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a non-aromatic, monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C=_₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits. Exemplary “(C₃-C₆)cycloalkyl” groups include cyclopropyl, cyclobutyl, cyclopentyl, and cyclohexyl.

The term “heterocyclyl” or “heterocyclic” refers to a radical of a 3- to 14-membered non-aromatic ring system having ring carbon atoms and 1 to 4 ring heteroatoms, wherein each heteroatom is independently nitrogen, oxygen, or sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings.

“Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 4-11 membered heterocyclyl. In certain embodiments, the heterocyclyl group is a substituted 4-11 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently nitrogen, oxygen, or sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently nitrogen, oxygen, or sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently nitrogen, oxygen, or sulfur (“5-6 membered heterocyclyl”). In some embodiments, the 5-6 membered heterocyclyl group has 1-3 ring heteroatoms, such as nitrogen, oxygen, or sulfur. In some embodiments, the 5-6 membered heterocyclyl group has 1-2 ring heteroatoms such as nitrogen, oxygen, or sulfur. In some embodiments, the 5-6 membered heterocyclyl group has 1 ring heteroatom such as nitrogen, oxygen, or sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 n electrons shared in a cyclic array) having ring carbon atoms and 1-4 ring heteroatoms provided in the aromatic ring system, wherein each heteroatom is independently nitrogen, oxygen, or sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently nitrogen, oxygen, or sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently nitrogen, oxygen, or sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently nitrogen, oxygen, or sulfur (“5-6 membered heteroaryl”). In some embodiments, the 5-6 membered heteroaryl has 1-3 ring heteroatoms nitrogen, oxygen, or sulfur. In some embodiments, the 5-6 membered heteroaryl has 1-2 ring heteroatoms nitrogen, oxygen, or sulfur. In some embodiments, the 5-6 membered heteroaryl has 1 ring heteroatom nitrogen, oxygen, or sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one —H present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. Heteroatoms such as nitrogen may have —H substituents and/or any suitable substituent as described herein which satisfy the valencies of the heteroatoms and results in the formation of a stable moiety. This disclosure is not intended to be limited in any manner by the exemplary substituents described herein.

Exemplary carbon atom substituents include halogen, —CN, —NO₂, —N₃, —SO₂H, —SO₃H, —OH, —OR^{aa}—, —ON(R^{bb})₂, —N(R^{bb})₂, —N(R^{bb})₃⁺X⁻, —N(OR^{cc})R^{bb}, —SH, —SR^{aa}—, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R—, —NR^{bb}CO₂R^{aa}, —NR^{bb}C(═O)N(R^{bb})₂, —C(═NR^{bb})R^{aa}, —C(═NR^{bb})OR^{aa}—, —OC(═NR^{bb})R^{aa}—, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{aa})₃⁺X⁻, —P(OR^{cc})₃⁺X⁻, —P(R^{cc})₄, —P(OR^{cc})₄, —OP(R^{cc})₂, —OP(R^{aa})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, or 5-14 membered heteroaryl; or optionally, two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is independently —H, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, or 5-14 membered heteroaryl; or optionally two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{cc} is, independently, —H, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, or 5-14 membered heteroaryl; or optionally two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is independently halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, or 5-10 membered heteroaryl, wherein each alkyl, alkenyl, alkynyl, heteroalkyl, heteroalkenyl, heteroalkynyl, carbocyclyl, heterocyclyl, aryl, or heteroaryl is independently substituted with 0, 1, 2, 3, 4, or 5 R^{gg} groups, or two geminal R^{dd} substituents can be joined to form ═O or ═S; wherein X⁻ is a counterion; each instance of R^{ee} is, independently, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 3-10 membered heteroaryl, wherein each alkyl, alkenyl, alkynyl, heteroalkyl, heteroalkenyl, heteroalkynyl, carbocyclyl, heterocyclyl, aryl, or heteroaryl is independently substituted with 0, 1, 2, 3, 4, or 5 R^{gg} groups; each instance of R^{ff} is independently —H, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl or 5-10 membered heteroaryl; or optionally two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{gg} is independently halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —COH, —C₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and each X⁻ is a counterion.

In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is —H, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently —H, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, the carbon atom substituents are independently halogen, substituted (e.g., substituted with one or more halogen) or unsubstituted C₁₋₆ alkyl, —OR^{aa}, —SR—, —N(R^{bb})₂, —CN, —SCN, or —N₀₂. In certain embodiments, the carbon atom substituents are independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is —H, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently —H, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, —H, fluorine, chlorine, bromine, iodine, oxygen, sulfur, nitrogen, and/or silicon atoms. In certain embodiments, a carbon atom substituent consists of carbon, —H, fluorine, chlorine, bromine, iodine, oxygen, sulfur, and/or nitrogen atoms. In certain embodiments, a carbon atom substituent consists of carbon, —H, fluorine, chlorine, bromine, and/or iodine atoms. In certain embodiments, a carbon atom substituent consists of carbon, —H, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than —H, and includes groups —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, or —OP(═O)(N(R^{bb}))₂, wherein X⁻, R^{aa}, R^{bb}, and R^{cc} are as defined herein.

“Oxo” represents a double-bonded oxygen moiety; for example, if attached directly to a carbon atom forms a carbonyl moiety (C═O).

The term “amino” refers to the group —NH₂. The term “substituted amino,” by extension, refers to a monosubstituted amino, a disubstituted amino, or a trisubstituted amino.

In certain embodiments, the “substituted amino” is a monosubstituted amino or a disubstituted amino group.

The term “monosubstituted amino” refers to an amino group wherein the nitrogen atom directly attached to the parent molecule is substituted with one —H and one group other than —H, and includes —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, or —NHP(═O)(N(R^{bb})₂)₂, wherein R^{aa}, R^{bb} and R^{cc} are as defined herein, and wherein R^{bb} of the group —NH(R^{bb}) is not —H.

The term “disubstituted amino” refers to an amino group wherein the nitrogen atom directly attached to the parent molecule is substituted with two groups other than —H, and includes groups —N(R^{bb})₂, —NR^{bb} C(═O)R—, —NR^{bb}CO₂R—, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, or —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with —H.

The term “trisubstituted amino” refers to an amino group wherein the nitrogen atom directly attached to the parent molecule is substituted with three groups, and includes —N(R^{bb})₃ or —N(R^{bb})₃⁺X⁻, wherein R^{bb} and X⁻ are as defined herein.

The term “sulfonyl” refers to —SO₂N(R^{bb})₂, —SO₂R^{aa}, or —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, or —C(═NR^{X1})N(R^{X1})₂, wherein R^{X1} is —H; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), or imines (—C(═NR^{bb})R^{aa}, —C(═NR^{bb})OR^{aa}), —C(═NR^{bb})N(R^{bb})₂), wherein R^{aa} and R^{bb} are as defined herein.

As used herein, the term “optionally” means that the subsequently described event(s) may or may not occur, and includes both event(s) that occur and event(s) that do not occur.

The terms “treatment,” “treat,” and “treating” refer to reversing, alleviating, delaying the onset of, or inhibiting the progress of a “pathological condition” (e.g., an infectious disease, or one or more signs or symptoms thereof) as described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The term “prevent,” “preventing,” or “prevention” refers to a prophylactic treatment of a subject who is not and/or was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. For use in therapy, therapeutically effective amounts of a compound of Formula (I-a), as well as salts thereof, may be administered as the raw chemical. Additionally, the active ingredient may be presented as a pharmaceutical composition.

The term “inhibition,” “inhibiting,” “inhibit,” or “inhibitor” refer to the ability of a compound to reduce, slow, halt, or prevent activity of a particular biological process (e.g., furin activity, viral infectivity, viral replication, toxin activation and/or activity) in a subject relative to vehicle.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)). In certain embodiments, the animal is a mammal. The animal may be a male or female and at any stage of development.

The terms “administer,” “administering,” or “administration,” refers to implanting, absorbing, ingesting, injecting, inhaling, or otherwise introducing a compound, or a pharmaceutical composition thereof to a subject.

The term “microbial toxin” refers to any toxin generated by a micro-organism (e.g., a bacteria). In certain embodiments, a microbial toxin is P. aeruginosa toxin A. In certain embodiments, the microbial toxin is Clostridium septicum alpha-toxin. In certain embodiments, the microbial toxin is a diphtheria toxin. In certain embodiments, the microbial toxin is a shiga toxin (e.g., Stx1 or Stx2).

### DETAILED DESCRIPTION OF CERTAIN EMBODIMENTS OF THE INVENTION

The present disclosure provides methods for the treatment and/or prevention of a range of viral infections including, but not limited to, infections caused by togaviridae family viruses (e.g., alphaviruses (e.g., Chikungunya virus, Eastern equine encephalitis virus, Mayaro virus, Onyong-nyong virus, Ross River virus, Semliki Forest virus, Sindbis virus, Venezuelan equine encephalitis virus, Western equine encephalitis virus)), flaviviridae family viruses (e.g., flaviviruses (e.g., dengue virus, Japanese encephalitis virus, Kyasanur Forest disease virus, Murray Valley encephalitis virus, Omsk hemorrhagic fever virus, Powassan virus, Rocio encephalitis virus, Saint Louis encephalitis virus, Tick-borne encephalitis virus, West Nile virus, Yellow fever virus, Usutu virus)), paramyxoviridae family viruses (e.g., orthoparamyxovirinae viruses (e.g., respiroviruses (e.g., human respirovirus 1, human respirovirus 3, murine respirovirus), henipaviruses (e.g., Cedar virus, Kumasi virus, Hendra virus, Mojiang virus, Nipah virus), morbilliviruses (e.g., Canine morbillivirus; Cetacean morbillivirus; Feline morbillivirus; Feline morbillivirus 2; Measles morbillivirus; Phocine morbillivirus; Rinderpest morbillivirus; Small ruminant morbillivirus)), filoviradae family viruses (e.g., Marburgviruses (e.g., Marburg Virus, Ravn Virus)), human respiratory syncytial virus (i.e., Human orthopneumovirus)), comprising administering to the subject a therapeutically effective amount of a compound of Formula (I), or a pharmaceutical composition comprising a compound of Formula (I) as described herein.

Further provided herein are methods for inhibiting the replication of a virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus (e.g., a henipavirus (e.g., Nipah virus), a morbillivirus (e.g., measles morbillivirus))) in a subject in need thereof, comprising administering to the subject a therapeutically effective amount of a compound of Formula (I), or a pharmaceutical composition comprising a compound of Formula (I) as described herein.

In certain embodiments, the present disclosure provides methods for the treatment and/or prevention of viral infections caused by togaviridae family viruses, comprising administering to the subject a therapeutically effective amount of a compound of Formula (I), or a pharmaceutical composition comprising a compound of Formula (I) as described herein. In certain embodiments, the provided methods are for the treatment and/or prevention of viral infections caused by togaviridae family viruses. In certain embodiments, the provided methods are for the treatment and/or prevention of viral infections caused by alphaviruses. In certain embodiments, the provided methods are for the treatment and/or prevention of viral infections caused by Chikungunya virus, Eastern equine encephalitis virus, Mayaro virus, Onyong-nyong virus, Ross River virus, Semliki Forest virus, sindbis virus, Venezuelan equine encephalitis virus, Western equine encephalitis virus. In certain embodiments, the present disclosure provides methods for the treatment and/or prevention of viral infections caused by Chikungunya virus.

In certain embodiments, the provided methods are for the treatment and/or prevention of flaviviruses. In certain embodiments, the provided methods are for the treatment and/or prevention of dengue virus, Japanese encephalitis virus, Kyasanur Forest disease virus, Murray Valley encephalitis virus, omsk hemorrhagic fever virus, Powassan virus, Rocio encephalitis virus, Saint Louis encephalitis virus, Tick-borne encephalitis virus, West Nile virus, and/or yellow fever virus. In certain embodiments, the provided methods are for the treatment and/or prevention of dengue virus. In certain embodiments, the provided methods are for the treatment and/or prevention of Usutu virus. In certain embodiments, the provided methods are for the treatment and/or prevention of Japanese encephalitis virus. In certain embodiments, the provided methods are for the treatment and/or prevention of Powassan virus. In certain embodiments, the provided methods are for the treatment and/or prevention of yellow fever.

In certain embodiments, the present disclosure provides methods for the treatment and/or prevention of viral infections caused by filoviradae family viruses, comprising administering to the subject a therapeutically effective amount of a compound of Formula (I), or a pharmaceutical composition comprising a compound of Formula (I) as described herein. In certain embodiments, the provided methods are for the treatment and/or prevention of Marburgviruses. In certain embodiments, the provided methods are for the treatment and/or prevention of Marburg Virus and Ravn Virus. In certain embodiments, the provided methods for the treatment and/or prevention of Marburg virus.

In certain embodiments, the present disclosure provides methods for the treatment and/or prevention of viral infections caused by paramyxoviridae family viruses, comprising administering to the subject a therapeutically effective amount of a compound of Formula (I), or a pharmaceutical composition comprising a compound of Formula (I) as described herein. In certain embodiments, the provided methods are for the treatment and/or prevention of an orthoparamyxovirinae virus. In certain embodiments, the provided methods are for the treatment and/or prevention of a respirovirus. In certain embodiments, the provided methods are for the treatment and/or prevention of human respirovirus 1 (i.e., human parainfluenza virus type 1). In certain embodiments, the provided methods are for the treatment and/or prevention of human respirovirus 3 (i.e., human parainfluenza virus type 3). In certain embodiments, the provided methods are for the treatment and/or prevention of murine respirovirus (i.e., murine parainfluenza virus type 1). In certain embodiments, the provided methods are for the treatment and/or prevention of a henipavirus. In certain embodiments, the provided methods are for the treatment and/or prevention of Cedar virus (i.e., Cedar henipavirus). In certain embodiments, the provided methods are for the treatment and/or prevention of Kumasi virus (i.e., Ghanaian bat henipavirus). In certain embodiments, the provided methods are for the treatment and/or prevention of Hendra virus (i.e., Hendra henipavirus). In certain embodiments, the provided methods are for the treatment and/or prevention of Mojiang virus (i.e., Mojiang henipavirus). In certain embodiments, the provided methods are for the treatment and/or prevention of Nipah virus (i.e., Nipah henipavirus). In certain embodiments, the provided methods are for the treatment and/or prevention of morbilliviruses. In certain embodiments, the provided methods are for the treatment and/or prevention of Canine morbillivirus, Cetacean morbillivirus, Feline morbillivirus, Feline morbillivirus 2, Measles morbillivirus, Phocine morbillivirus, Rinderpest morbillivirus, and/or Small ruminant morbillivirus. In certain embodiments, the provided methods are for the treatment and/or prevention of Measles morbillivirus.

In another aspect, the present disclosure provides methods of treating a viral infection (e.g., infections resulting from togaviridae family viruses (e.g., alphaviruses (e.g., Chikungunya virus)), filoviradae family viruses (e.g., Marburgviruses (e.g., Marburg Virus, Ravn Virus)), human respiratory syncytial virus (i.e., Human orthopneumovirus), a flavivirus (e.g., dengue virus)) in a subject in need thereof, the method comprising administering to the subject in need thereof an effective amount (e.g., therapeutically effective amount) of a compound of Formula (I), or a pharmaceutical composition comprising a compound of Formula (I) as described herein. In certain embodiments, provided herein are methods of treating viral infections resulting from Chikungunya virus. In certain embodiments, provided herein are methods of treating viral infections resulting from dengue virus. In certain embodiments, provided herein are methods of treating viral infections resulting from Chikungunya virus. In certain embodiments, provided herein are methods of treating viral infections resulting from Eastern equine encephalitis. In certain embodiments, provided herein are methods of treating viral infections resulting from Mayaro virus. In certain embodiments, provided herein are methods of treating viral infections resulting from Venezuelan equine encephalitis virus. In certain embodiments, provided herein are methods of treating a viral infections resulting from Western equine encephalitis.

In another aspect, the present disclosure provides methods of preventing a viral infection in a subject in need thereof, the method comprising administering to the subject in need thereof an effective amount (e.g., a prophylactically effective amount) of a compound of Formula (I), or a pharmaceutical composition comprising Formula (I) as described herein. In certain embodiments, provided herein are methods of preventing viral infections resulting from Chikungunya virus. In certain embodiments, provided herein are methods of preventing viral infections resulting from dengue virus. In certain embodiments, provided herein are methods of preventing viral infections resulting from Chikungunya virus.

In another aspect, the present disclosure provides methods of inhibiting the replication of a virus (e.g., a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus, (e.g., a henipavirus (e.g., Nipah virus), a morbillivirus (e.g., measles morbillivirus))) in a subject in need thereof, the method comprising administering to the subject in need thereof an effective amount of a compound described herein, or a pharmaceutical composition described herein. In certain embodiments, the provided methods are for inhibiting the replication of Chikungunya virus in a subject in need thereof. In certain embodiments, the provided methods are for inhibiting the replication of Marburg virus in a subject in need thereof. In certain embodiments, the provided methods are for inhibiting the replication of dengue virus in a subject in need thereof.

Provided herein are methods of inhibiting the replication of a virus (e.g., a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus, (e.g., a henipavirus (e.g., Nipah virus), a morbillivirus (e.g., measles morbillivirus))) in a subject in a subject in need thereof, comprising administering to the subject an effective amount of a compound of Formula (I), or a pharmaceutically acceptable salt thereof. In certain embodiments, provided herein are methods of inhibiting the replication of a virus in a subject by at least 1%, at least 3%, at least 10%, at least 20%, at least 30%, at least 40%, at least 50%, at least 60%, at least 70%, at least 80%, or at least 90%. In certain embodiments, the replication of a virus in a subject is inhibited by at least 1%, at least 3%, at least 10%, at least 20%, at least 30%, at least 40%, at least 50%, at least 60%, at least 70%, at least 80%, or at least 90%. In certain embodiments, provided herein are methods of inhibiting the replication of a virus in a subject by at least 30%. In certain embodiments, provided herein are methods of inhibiting the replication of a virus in a subject by at least 50%. In certain embodiments, provided herein are methods of inhibiting the replication of a virus in a subject by at least 75%.

In another aspect, the present disclosure provides methods of decreasing viral infectivity (e.g., infectivity of a virus (e.g., a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus, (e.g., a henipavirus (e.g., Nipah virus), a morbillivirus (e.g., measles morbillivirus))) in a subject, the method comprising administering to the subject an effective amount of a compound of Formula (I), or a pharmaceutical composition comprising Formula (I) as described herein. In certain embodiments, the present disclosure provides methods of decreasing viral infectivity of Chikungunya virus in a subject. In certain embodiments, the present disclosure provides methods of decreasing viral infectivity of dengue virus in a subject. In certain embodiments, the present disclosure provides methods of decreasing viral infectivity of Marburg virus in a subject.

In another aspect, the present disclosure provides compounds of Formula (I) and pharmaceutical compositions described herein for use in treating and/or preventing a viral infection including, but not limited to, infections caused by togaviridae family viruses (e.g., alphaviruses (e.g., Chikungunya virus, Eastern equine encephalitis virus, Mayaro virus, Onyong-nyong virus, Ross River virus, Semliki Forest virus, Sindbis virus, Venezuelan equine encephalitis virus, Western equine encephalitis virus)), flaviviridae family viruses (e.g., flaviviruses (e.g., dengue virus, Japanese encephalitis virus, Kyasanur Forest disease virus, Murray Valley encephalitis virus, Omsk hemorrhagic fever virus, Powassan virus, Rocio encephalitis virus, Saint Louis encephalitis virus, Tick-borne encephalitis virus, West Nile virus, Yellow fever virus, Usutu virus)), filoviradae family viruses (e.g., Marburgviruses (e.g., Marburg Virus, Ravn Virus)) human respiratory syncytial virus (i.e., Human orthopneumovirus)), comprising administering to the subject a therapeutically effective amount of a compound of Formula (I), or a pharmaceutical composition comprising a compound of Formula (I) as described herein.

In another aspect, the present disclosure provides compounds of Formula (I) and pharmaceutical compositions described herein for use in treating and/or preventing a viral infection including, but not limited to, infections caused by a virus (e.g., a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus, (e.g., a henipavirus (e.g., Nipah virus), a morbillivirus (e.g., measles morbillivirus))) in a subject in need thereof. In certain embodiments, the present disclosure provides compounds of Formula (I) and pharmaceutical compositions described herein for use in treating and/or preventing a viral infection caused by Chikungunya virus in a subject in need thereof. In certain embodiments, the present disclosure provides compounds of Formula (I) and pharmaceutical compositions described herein for use in treating and/or preventing a viral infection caused by Marburg virus in a subject in need thereof. In certain embodiments, the present disclosure provides compounds of Formula (I) and pharmaceutical compositions described herein for use in treating and/or preventing a viral infection caused by dengue virus in a subject in need thereof.

In another aspect, the present disclosure provides uses of compounds of Formula (I) and pharmaceutical compositions described herein in the manufacture of a medicament for treating a viral infection (e.g., viral infections resulting from a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus, (e.g., a henipavirus (e.g., Nipah virus), a morbillivirus (e.g., measles morbillivirus))) in a subject in need thereof. In certain embodiments, the present disclosure provides uses of compounds of Formula (I) and pharmaceutical compositions described herein in the manufacture of a medicament for treating viral infections resulting from Chikungunya virus in a subject in need thereof. In certain embodiments, the present disclosure provides uses of compounds of Formula (I) and pharmaceutical compositions described herein in the manufacture of a medicament for treating a viral infection resulting from dengue virus in a subject in need thereof. In certain embodiments, the present disclosure provides uses of compounds of Formula (I) and pharmaceutical compositions described herein in the manufacture of a medicament for treating Marburg virus in a subject in need thereof.

In another aspect, the present disclosure provides uses of compounds of Formula (I) and pharmaceutical compositions described herein in the manufacture of a medicament for preventing a viral infection (e.g., infection resulting from a togaviridae family virus (e.g., an alphavirus (e.g., Chikungunya virus, Eastern equine encephalitis, Mayaro virus, Venezuelan equine encephalitis virus, Western equine encephalitis)), a filoviradae family virus (e.g., a Marburgvirus (e.g., Marburg virus, Ravn virus)), human respiratory syncytial virus (i.e., human orthopneumovirus), a flavivirus (e.g., dengue virus, Usutu virus, Japanese encephalitis virus, Powassan virus, yellow fever), a paramyxoviridae family virus (e.g., an orthoparamyxovirinae virus, (e.g., a henipavirus (e.g., Nipah virus), a morbillivirus (e.g., measles morbillivirus))) in a subject in need thereof. In another aspect, the present disclosure provides uses of compounds of Formula (I) and pharmaceutical compositions described herein in the manufacture of a medicament for preventing Chikungunya virus in a subject in need thereof. In another aspect, the present disclosure provides uses of compounds of Formula (I) and pharmaceutical compositions described herein in the manufacture of a medicament for preventing Marburg virus in a subject in need thereof. In another aspect, the present disclosure provides uses of compounds of Formula (I) and pharmaceutical compositions described herein in the manufacture of a medicament for preventing a viral infection caused by dengue virus in a subject in need thereof.

In certain embodiments, the virus is a togaviridae family virus. In certain embodiments, the togaviridae family virus is an alphavirus. In certain embodiments, the alphavirus is Chikungunya virus, Eastern equine encephalitis virus, Mayaro virus, Onyong-nyong virus, Ross River virus, Semliki Forest virus, Sindbis virus, Venezuelan equine encephalitis virus, or Western equine encephalitis virus. In certain embodiments, the alphavirus is dengue virus.

In certain embodiments, the virus is a paramyxoviridae family virus. In certain embodiments, the paramyxoviridae family virus is an orthoparamyxovirinae virus. In certain embodiments, the orthoparamyxovirinae virus is a henipavirus. In certain embodiments, the henipavirus is Nipah virus (i.e., Nipah henipavirus). In certain embodiments, the henipavirus is Cedar virus (i.e., Cedar henipavirus). In certain embodiments, the henipavirus is Kumasi virus (i.e., Ghanaian bat henipavirus. In certain embodiments, the henipavirus Hendra virus (i.e., Hendra henipavirus). In certain embodiments, the henipavirus is Mojiang virus (i.e., Mojiang henipavirus). In certain embodiments, the orthoparamyxovirinae virus is a respirovirus. In certain embodiments, the respirovirus is human respirovirus 1 (i.e., human parainfluenza virus type 1). In certain embodiments, the respirovirus is human respirovirus 3 (i.e., Human parainfluenza virus type 3). In certain embodiments, the respirovirus is murine respirovirus (i.e, Murine parainfluenza virus type 1). In certain embodiments, the paramyxoviridae family virus is a morbillivirus. In certain embodiments, the morbillivirus is a Canine morbillivirus, Cetacean morbillivirus, Feline morbillivirus, Feline morbillivirus 2, Measles morbillivirus, Phocine morbillivirus, Rinderpest morbillivirus, or Small ruminant morbillivirus. In certain embodiments, the morbillivirus is Measles morbillivirus (i.e., measles).

In certain embodiments, the virus is a flaviviridae family virus. In certain embodiments, the flaviviridae family virus is a flavivirus. In certain embodiments, the flavivirus is dengue virus, Japanese encephalitis virus, Kyasanur Forest disease virus, Murray Valley encephalitis virus, Omsk hemorrhagic fever virus, Powassan virus, Rocio encephalitis virus, Saint Louis encephalitis virus, Tick-borne encephalitis virus, West Nile virus, or Yellow fever virus. In certain embodiments, the flavivirus is dengue virus.

In certain embodiments, the virus us a filoviradae family virus. In certain embodiments, the filoviradae family virus is a Marburgvirus. In certain embodiments, the Marburgvirus is Marburg Virus, or Ravn Virus. In certain embodiments, the Marburgvirus is Marburg virus. Virus. In certain embodiments, the Marburgvirus is Ravn virus.

Without wishing to be bound by any theory, in certain embodiments the compounds of Formula (I) useful in the methods and uses of this disclosure prevents or inhibits the furin-mediated processing of viral precursor protein E3E2, which prevents or inhibits viral fusion and infection.

Without wishing to be bound by any theory, in certain embodiments the compounds of Formula (I) useful in the methods and uses of this disclosure prevents or inhibits the furin-mediated cleavage of the virion premembrane (prM). Cleavage of prM is the defining event in flavivirus maturation and is a required step in the virus infection cycle. Thus, inhibition of prM cleavage prevents or inhibits viral infectivity.

Without wishing to be bound by any theory, in certain embodiments the compounds of Formula (I) useful in the methods and uses of this disclosure inhibits viral fusion by cleaving the glycoproteins of a virus.

Also provided herein are methods for treating and/or preventing a disorder due to a microbial toxin (e.g., P. aeruginosa toxin A, Clostridium septicum alpha-toxin, diphtheria toxin(s), shiga toxin(s)) in a subject in need thereof, comprising administering to the subject a therapeutically effective amount of a compound of Formula (I).

Further provided herein are methods for preventing the activation of a toxin (e.g., P. aeruginosa toxin A, Clostridium septicum alpha-toxin, diphtheria toxin(s), shiga toxin(s)) in a subject in need thereof, comprising administering to the subject a therapeutically effective amount of a compound of Formula (I).

In certain embodiments, provided are methods of decreasing the activity of a toxin (e.g., P. aeruginosa toxin A, Clostridium septicum alpha-toxin, diphtheria toxin(s), shiga toxin(s)) in a subject by at least 1%, at least 3%, at least 10%, at least 20%, at least 30%, at least 40%, at least 50%, at least 60%, at least 70%, at least 80%, or at least 90%. In certain embodiments, the activity of a toxin (e.g., P. aeruginosa toxin A, Clostridium septicum alpha-toxin, diphtheria toxin(s), shiga toxin(s)) in a subject is decreased by at least 1%, at least 3%, at least 10%, at least 20%, at least 30%, at least 40%, at least 50%, at least 60%, at least 70%, at least 80%, or at least 90%. In some embodiments, the activity of a toxin (e.g., P. aeruginosa toxin A, Clostridium septicum alpha-toxin, diphtheria toxin(s), shiga toxin(s)) in a subject is selectively decreased by a compound of Formula (I) or pharmaceutical composition described herein.

In another aspect, the present disclosure provides compounds of Formula (I) and pharmaceutical compositions described herein for use in treating and/or preventing a disorder due to a microbial toxin (e.g., P. aeruginosa toxin A, Clostridium septicum alpha-toxin, diphtheria toxin(s), shiga toxin(s)) in a subject in need thereof.

In another aspect, the present disclosure provides uses of compounds of Formula (I) and pharmaceutical compositions described herein in the manufacture of a medicament for treating and/or preventing a disorder due to a microbial toxin (e.g., P. aeruginosa toxin A, Clostridium septicum alpha-toxin, diphtheria toxin(s), shiga toxin(s)) in a subject in need thereof.

Without wishing to be bound by any theory, in certain embodiments the compounds of Formula (I) useful in the methods and uses of this disclosure prevents or inhibits the processing of Pseudomonas aeruginosa exotoxin A by furin to prevent active forms of Pseudomonas aeruginosa exotoxin A from forming.

In certain embodiments, the compound of Formula (I) useful in the present disclosure is of the formula:

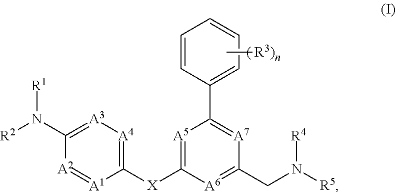

or a pharmaceutically acceptable salt thereof, wherein:

A¹, A², A³, A⁴, A⁵, A⁶, and A⁷ are each independently —N═ or —C(R⁶)═;

X is —O— or —N(R⁸)—.

R¹ and R² are each independently H or optionally substituted (C₁-C₄)alkyl;

optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;

each R³ is independently halogen, —CN, —O(C₁-C₄)alkyl, or optionally substituted (C₁-C₄)alkyl;

R⁴ and R⁵ are each independently H or optionally substituted (C₁-C₄)alkyl;

optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;

each R⁶ is independently H, halogen, optionally substituted (C₁-C₄)alkyl, —OH, or optionally substituted (C₁-C₄)alkoxy;

each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);

each of R⁸ and R⁹ is independently H, optionally substituted (C₁-C₄)alkyl, or optionally substituted (C₃-C₆)cycloalkyl; and

n is 1, 2, 3, or 4.

Formula (I) contains the substituents A¹, A², A³, A⁴, A⁵ A⁶, and A⁷, wherein each of A¹, A², A³, A⁴, A⁵ A⁶, and A⁷ are independently ═N— or CR⁶; wherein each R⁶ is independently —H, halogen, —(C₁-C₄)alkyl, -halo(C₁-C₄)alkyl, —OH, or —(C₁-C₄)alkoxy. In certain embodiments, A¹ is —N═. In certain embodiments, A¹ is —CR⁶═. In certain embodiments, A¹ is —CH═. In certain embodiments, A² is —N═. In certain embodiments, A² is —CR⁶═. In certain embodiments, A² is —CH═. In certain embodiments, A³ is —N═. In certain embodiments, A³ is —CR⁶═. In certain embodiments, A³ is —CH═. In certain embodiments, A⁴ is —N═. In certain embodiments, A⁴ is —CR⁶═. In certain embodiments, A⁴ is —CH═. In certain embodiments, A⁵ is —N═. In certain embodiments, A⁵ is —CR⁶═. In certain embodiments, A⁵ is —CH═. In certain embodiments, A⁶ is —N═. In certain embodiments, A⁶ is —CR⁶═. In certain embodiments, A⁶ is —CH═. In certain embodiments, A⁷ is —N═. In certain embodiments, A⁷ is —CR⁶═. In certain embodiments, A⁷ is —CH═. In one embodiment, A¹, A², A³, A⁴, A⁵, A⁶, and A⁷ are each independently —N═, —CH═, or CR⁶, wherein zero, one, two, or three of A¹, A², A³, A⁴, A⁵, A⁶, and A⁷ are —N═. In another embodiment, A¹, A², A³, A⁴, A⁵, A⁶, and A⁷ are each independently —N═, CH, or CR⁶, wherein two or three of A¹, A², A³, A⁴, A⁵, A⁶, and A⁷ are —N═. In another embodiment, A¹, A², A³, A⁴, and A⁵ are each independently —N═ or CH, wherein two or three of A¹, A², A³, A⁴, and A⁵ are —N═. In another embodiment, A¹, A², A³, A⁴, and A⁵ are each independently —N═ or CH, wherein three of A¹, A², A³, A⁴, and A⁵ are —N═. In another embodiment, A¹, A², A³, A⁴, and A⁵ are each independently —N═ or CH, wherein two of A¹, A², A³, A⁴, and A⁵ are —N═. In another embodiment, A¹, A², and A³ are each independently —N═ or —CH═, wherein two of A¹, A², and A³ are —N═. In another embodiment, A¹, A², and A³ are each independently —N═ or CH, wherein one of A¹, A², and A³ is —N═. In another embodiment, one of A¹ and A² is —N═ and the other is CH. In another embodiment, A¹ and A² are each CH. In another embodiment, A¹ and A² are each —N═. In another embodiment, A¹ and A³ are each —N═. In another embodiment, A² and A³ are each —N═. In another embodiment, A¹ and A⁴ are each —N═. In another embodiment, A³ and A⁵ are each —N═. In another embodiment, A³ and A⁵ are each —N═, and each of A¹, A², A⁴, A⁶, and A⁷ are CH. In another embodiment, A⁴ and A⁶ are each CH. In another embodiment, A², A³ and A⁵ are each —N═. In another embodiment, A², A³ and A⁵ are each —N═, and each of A¹, A⁴, A⁶, and A⁷ are CH.

In certain embodiments, the compound of Formula (I) useful in the present disclosure is of the Formula (I-1):

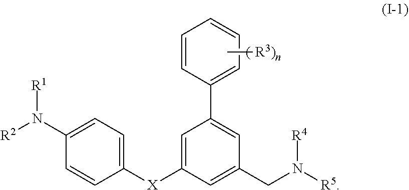

In certain embodiments, the compound of Formula (I) useful in the present disclosure is of the Formula (I-2):

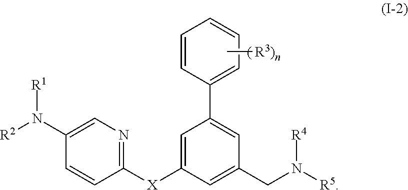

In certain embodiments, the compound of Formula (I) useful in the present disclosure is of the Formula (I-3):

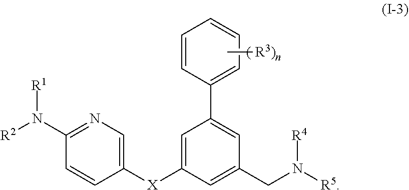

In certain embodiments, the compound of Formula (I) useful in the present disclosure is of the Formula (I-4):

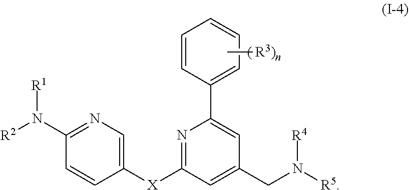

In certain embodiments, the compound of Formula (I) useful in the present disclosure is of the Formula (I-5):

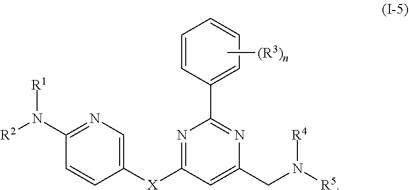

In certain embodiments, the compound of Formula (I) useful in the present disclosure is of the Formula (I-6):

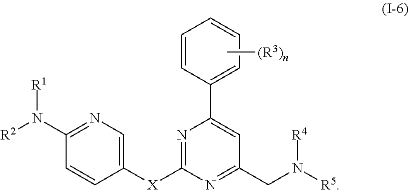

In certain embodiments, the compound of Formula (I) useful in the present disclosure is of the Formula (I-7):

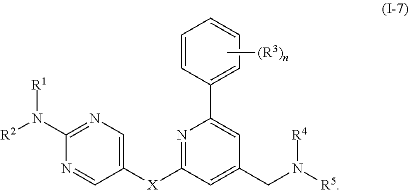

In certain embodiments, the compound of Formula (I) useful in the present disclosure is of the Formula (I-8):

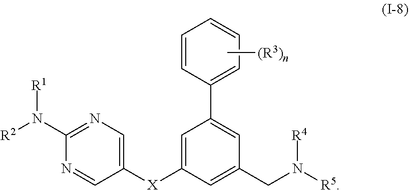

In certain embodiments, the compound of Formula (I) useful in the present disclosure is of the Formula (I-9):

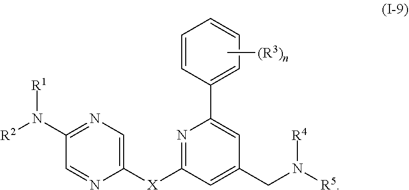

In certain embodiments, the compound of Formula (I) useful in the present disclosure is of the Formula (I-10):

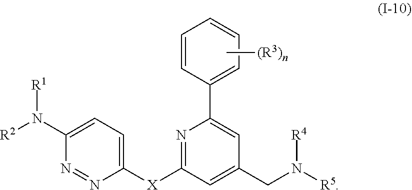

In certain embodiments, the compound of Formula (I) useful in the present disclosure is of the Formula (I-11):

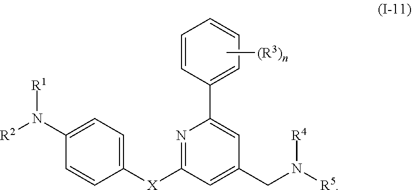

In certain embodiments, the compound of Formula (I) useful in the present disclosure is of the Formula (I-12):

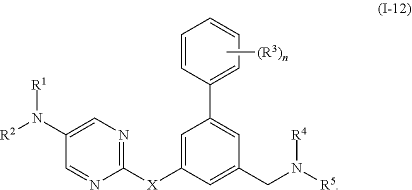

In one embodiment, X is O or NR⁸, wherein R⁸ is (C₁-C₄)alkyl. In another embodiment, X is —NR⁸, wherein R⁸ is (C₁-C₄)alkyl. In certain embodiments, X is O.

In certain embodiments, R³ is optionally substituted —O(C₁-C₄)alkyl. In certain embodiments, R³ is optionally substituted —OCF₃. In certain embodiments, R³ is optionally substituted (C₁-C₄)alkyl. In certain embodiments, R³ is -Me. In certain embodiments, R³ is —CF₃. In certain embodiments, R³ is —CHF₂. In certain embodiments, R³ is —CH₂F. In certain embodiments, R³ is halogen. In certain embodiments, R³ is —F. In certain embodiments, R³ is —Cl. In certain embodiments, R³ is —Br. In certain embodiments, R³ is —I. In certain embodiments, R³ is -Me. In certain embodiments, each R³ is independently halogen, methyl, or difluoromethyl. In another embodiment, each R³ is independently fluoro, chloro, bromo, methyl, or difluoromethyl. In one embodiment, each R³ is independently halogen. In another embodiment, each R³ is independently fluoro, chloro, or bromo. In another embodiment, each R³ is independently fluoro or chloro. In certain embodiments, each R³ is chloro. In certain embodiments, R³ is —CN.

In certain embodiments, R¹ and R² are each independently H, (C₁-C₄)alkyl, or (C₁-C₄)alkylNH₂. In certain embodiments, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷. In one embodiment, R¹ and R² are each independently H, (C₁-C₄)alkyl, or —(C₁-C₄)alkylNH₂. In another embodiment, R¹ and R² are each independently H or —(C₁-C₄)alkylNH₂. R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷. In certain embodiments, R¹ and R² taken together with the nitrogen atom to which they are attached form an optionally substituted pyrrolidine, pyrazolidine, imidazolidine, piperidine, piperazine, or morpholine ring.

In another embodiment, R¹ and R² taken together with the nitrogen atom to which they are attached represent a 6- or 7-membered monocyclic ring, optionally containing one or two additional heteroatoms independently selected from oxygen, nitrogen, and sulfur, wherein said ring is optionally substituted by one, two, or three substituents independently halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷. In another embodiment, R¹ and R² taken together with the nitrogen atom to which they are attached represent a 6- or 7-membered monocyclic ring, optionally containing one or two additional nitrogen heteroatoms, wherein said ring is optionally substituted by one, two, or three substituents independently selected from halogen, hydroxyl, oxo, R⁷, —OR⁷, —NHR⁸, —NR⁷R⁸, and —C(O)R⁷. In another embodiment, R¹ and R² taken together with the nitrogen atom to which they are attached represent a 6- or 7-membered monocyclic ring, optionally containing one additional nitrogen heteroatom, wherein said ring is optionally substituted by one substituent which is R⁷. In certain embodiments, R¹ and R² taken together with the nitrogen atom to which they are attached represent an optionally substituted piperazine ring.

In certain embodiments, R¹ and R² taken together with the nitrogen atom to which they are attached form an optionally substituted piperazine ring. In certain embodiments, R¹ and R² taken together with the nitrogen atom to which they are attached form a piperazine ring of the formula:

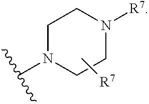

In certain embodiments, R¹ and R² taken together with the nitrogen atom to which they are attached form a piperazine ring of the formula:

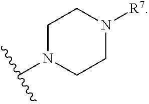

In certain embodiments, R¹ and R² taken together with the nitrogen atom to which they are attached form a piperazine ring of the formula:

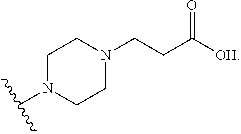 [SMILES: *N1CCN(CCC(=O)O)CC1]

In certain embodiments, R¹ and R² taken together with the nitrogen atom to which they are attached form a piperazine ring of the formula:

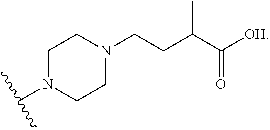 [SMILES: *N1CCN(CCC(C)C(=O)O)CC1]

In certain embodiments, R¹ and R² taken together with the nitrogen atom to which they are attached form a piperazine ring of the formula:

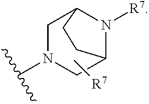

In certain embodiments, R¹ and R² taken together with the nitrogen atom to which they are attached form a piperazine ring of the formula:

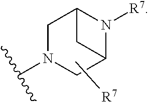

In certain embodiments, R¹ and R² taken together with the nitrogen atom to which they are attached form a piperazine ring of the formula:

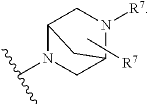

In certain embodiments, R¹ and R² taken together with the nitrogen atom to which they are attached form a piperazine ring of the formula:

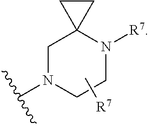

In certain embodiments, R¹ and R² taken together with the nitrogen atom to which they are attached form a ring of the formula:

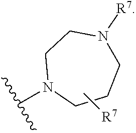

In certain embodiments, R¹ and R² taken together with the nitrogen atom to which they are attached form a ring of the formula:

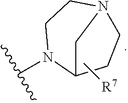

In certain embodiments, R¹ and R² taken together with the nitrogen atom to which they are attached form a piperidine ring of the formula:

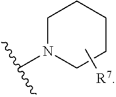

In certain embodiments, R¹ and R² taken together with the nitrogen atom to which they are attached form a piperidine ring of the formula:

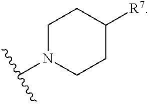

In certain embodiments, R¹ and R² taken together with the nitrogen atom to which they are attached form a ring of the formula:

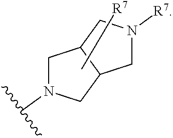

In certain embodiments, R¹ and R² taken together with the nitrogen atom to which they are attached form a ring of the formula:

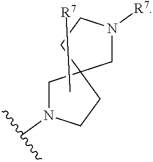

In certain embodiments, R¹ and R² taken together with the nitrogen atom to which they are attached form a pyrrolidine ring of the formula:

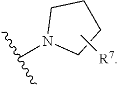

In certain embodiments, R⁴ and R⁵ are each independently H, or optionally substituted (C₁-C₄)alkyl. In certain embodiment, R⁴ and R⁵ are the same. In certain embodiments, R⁴ and R⁵ are different. In certain embodiments, R⁴ is H. In certain embodiments, R⁵ is H. In one embodiment, R⁴ and R⁵ are each independently H, (C₁-C₄)alkyl, or (C₂-C₄)alkyl(C₁-C₄)alkoxy. In certain embodiments, R⁴ is -Me. In certain embodiments, R⁴ is —C(O)R⁷. In certain embodiments, R⁴ is —C(O)Me. In another embodiment, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹. In another embodiment, R⁴ and R⁵ taken together with the nitrogen atom to which they are attached form a piperidine ring of the formula:

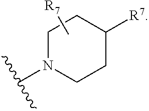

In another embodiment, R⁴ and R⁵ taken together with the nitrogen atom to which they are attached form a piperidine ring of the formula:

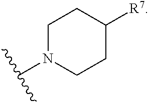

In another embodiment, R⁴ and R⁵ taken together with the nitrogen atom to which they are attached form a piperidine ring of the formula:

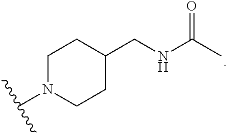 [SMILES: *N1CCC(CNC(C)=O)CC1]

In another embodiment, R⁴ and R⁵ taken together with the nitrogen atom to which they are attached form a ring of the formula:

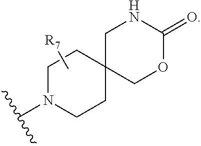

In another embodiment, R⁴ and R⁵ taken together with the nitrogen atom to which they are attached form a ring of the formula:

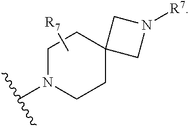

In another embodiment, R⁴ and R⁵ taken together with the nitrogen atom to which they are attached form a piperazine ring of the formula:

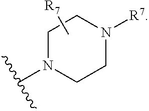

In another embodiment, R⁴ and R⁵ taken together with the nitrogen atom to which they are attached form ring of the formula

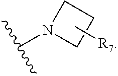

In another embodiment, R⁴ and R⁵ taken together with the nitrogen atom to which they are attached form a pyrrolidine ring of the formula:

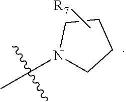

In another embodiment, R⁴ and R⁵ taken together with the nitrogen atom to which they are attached form a pyrrolidine ring of the formula:

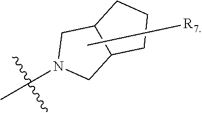

In another embodiment, R⁴ and R⁵ taken together with the nitrogen atom to which they are attached form a pyrrolidine ring of the formula:

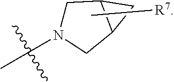

In another embodiment, R⁴ and R⁵ taken together with the nitrogen atom to which they are attached form a ring of the formula:

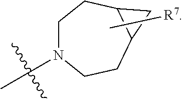

In another embodiment, R⁴ and R⁵ taken together with the nitrogen atom to which they are attached form a ring of the formula:

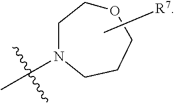

In one embodiment, each R⁶ is independently halogen or (C₁-C₄)alkyl. In another embodiment, each R⁶ is independently halogen. In another embodiment, each R⁶ is independently selected from the group consisting of fluoro, chloro, bromo, and methyl. In another embodiment, each R⁶ is independently selected from the group consisting of fluoro, chloro, and bromo. In another embodiment, each R⁶ is independently fluoro or chloro. In certain embodiments, each R⁶ is fluoro. In another embodiment, each R⁶ is chloro. In another embodiment, each R⁶ is independently (C₁-C₄)alkyl. In another embodiment, each R⁶ is methyl.

In one embodiment, each R⁷ is independently (C₁-C₆)alkyl, halo(C₁-C₆)alkyl, (C₃-C₆)cycloalkyl, or —(C₁-C₄)alkyl(C₃-C₆)cycloalkyl, each of which is optionally substituted by one or two of triazolyl, tetrazolyl, —CO₂R⁸, —CONR⁸R⁹, —CON(R⁸)CO₂(C₁-C₄)alkyl, —OH, (C₁-C₄)alkoxy, —OCONR⁸R⁹, —OCON(R⁸)C(O)R⁹, (C₁-C₄)alkyl, —(C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹). In another embodiment, each R⁷ is independently (C₁-C₄)alkyl, (C₂-C₄)alkenyl, halo(C₁-C₄)alkyl, (C₃-C₆)cycloalkyl, or —(C₁-C₂)alkyl(C₃-C₆)cycloalkyl, each of which is optionally substituted with —CO₂R⁸, —CONR⁸R⁹, —OH, oxo, —(C₁-C₄)alkoxy, —OCONR⁸R⁹, —(C₁-C₄)alkylOH, —NR⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)SO₂R⁹, —SO(C₁-C₄)alkyl, —SO₂(C₁-C₄)alkyl, —SO₃R⁸, —SO₂NR⁸R⁹, or —P(O)(OR⁸)(OR⁹). In another embodiment, each R⁷ is independently (C₁-C₄)alkyl, (C₂-C₄)alkenyl, halo(C₁-C₄)alkyl, (C₃-C₆)cycloalkyl, or —(C₁-C₂)alkyl(C₃-C₆)cycloalkyl, each of which is optionally substituted by one or two substituents —CO₂R⁸, —CONR⁸R⁹, —OH, (C₁-C₄)alkoxy, —OCONR⁸R⁹, —(C₁-C₄)alkylOH, —NR⁸R⁹, —N(R⁸)C(O)R⁹, —N(R⁸)CO₂(C₁-C₄)alkyl, —N(R⁸)CONR⁸R⁹, —N(R⁸)SO₂R⁹, —SO(C₁-C₄)alkyl, —SO₂(C₁-C₄)alkyl, —SO₃R⁸, —SO₂NR⁸R⁹, or —P(O)(OR⁸)(OR⁹). In another embodiment, each R⁷ is (C₁-C₆)alkyl which is optionally substituted by one substituent which is —CO₂H, —OH, —N(R⁸)C(O)R⁹, or —SO(C₁-C₄)alkyl. In another embodiment, each R⁷ is (C₁-C₄)alkyl which is optionally substituted by one substituent which is —CO₂H, —OH, —N(R⁸)C(O)R⁹, or —SO(C₁-C₄)alkyl.

In certain embodiments, each of R⁸ and R⁹ is independently H, optionally substituted (C₁-C₄)alkyl, or optionally substituted (C₃-C₆)cycloalkyl. In one embodiment, each R⁸ and R⁹ is independently H or (C₁-C₄)alkyl. In another embodiment, each R⁸ and R⁹ is independently (C₁-C₄)alkyl. In another embodiment, R⁸ and R⁹ are each methyl. In another embodiment, each R⁸ and R⁹ is H. In another embodiment, R⁸ is H; and R⁹ is (C₁-C₄)alkyl. In another embodiment, R⁸ is H; and R⁹ is -Me. In another embodiment, R⁸ is (C₁-C₄)alkyl. In another embodiment, R⁸ is -Me. In another embodiment, R⁸ is —H. In another embodiment, R⁹ is (C₁-C₄)alkyl. In another embodiment, R⁹ is -Me. In another embodiment, R⁹ is —H.

In one embodiment, n is 1, 2, or 3. In another embodiment, n is 2 or 3. In another embodiment, n is 2.

In certain embodiments, the disclosed methods comprise administering to the subject in need thereof a therapeutically effective amount of any one of the compounds found in Table 1.

In certain embodiments, the compound of Formula (I), or a pharmaceutically acceptable salt thereof, useful in the present disclosure is of the formula:

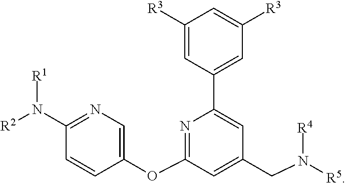

In certain embodiments, the compound of Formula (I), or a pharmaceutically acceptable salt thereof, useful in the present disclosure is of the formula:

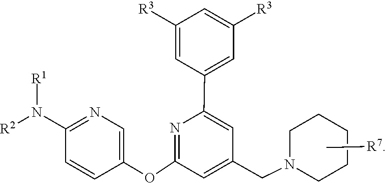

In certain embodiments, the compound of Formula (I), or a pharmaceutically acceptable salt thereof, useful in the present disclosure is of the formula:

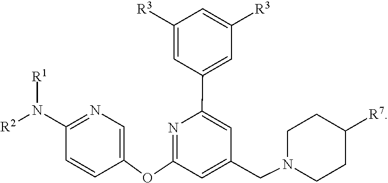

In certain embodiments, the compound of Formula (I), or a pharmaceutically acceptable salt thereof, useful in the present disclosure is of the formula:

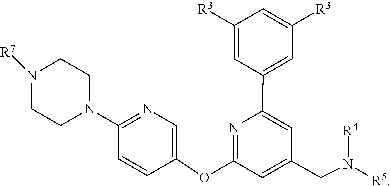

In certain embodiments, the compound of Formula (I), or a pharmaceutically acceptable salt thereof, useful in the present disclosure is of the formula:

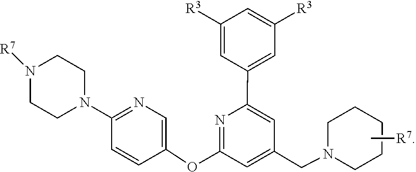

In certain embodiments, the compound of Formula (I) useful in the present disclosure is of the formula thereof, useful in the present disclosure is of the formula:

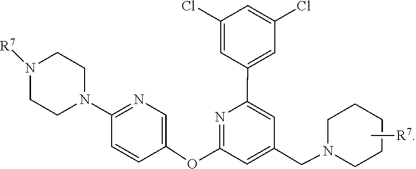

In certain embodiments, the compound of Formula (I) useful in the present disclosure is of the formula:

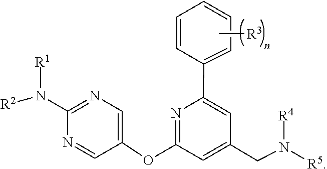

In certain embodiments, the compound of Formula (I) useful in the present disclosure is of the formula:

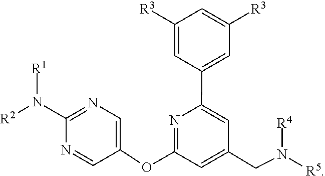

In certain embodiments, the compound of Formula (I) useful in the present disclosure is of the formula:

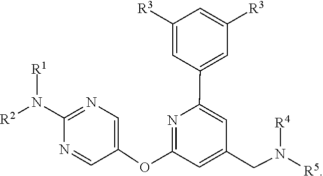

In certain embodiments, the compound of Formula (I) useful in the present disclosure is of the formula:

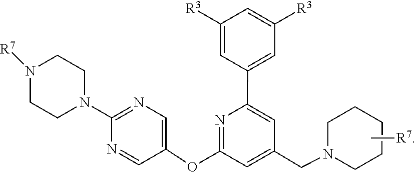

In certain embodiments, the compound of Formula (I) useful in the present disclosure is of the formula:

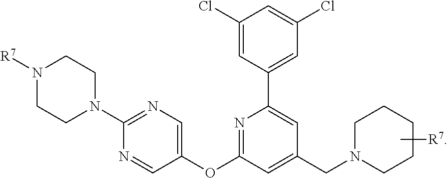

In certain embodiments, the compound of Formula (I) useful in the present disclosure is of the formula:

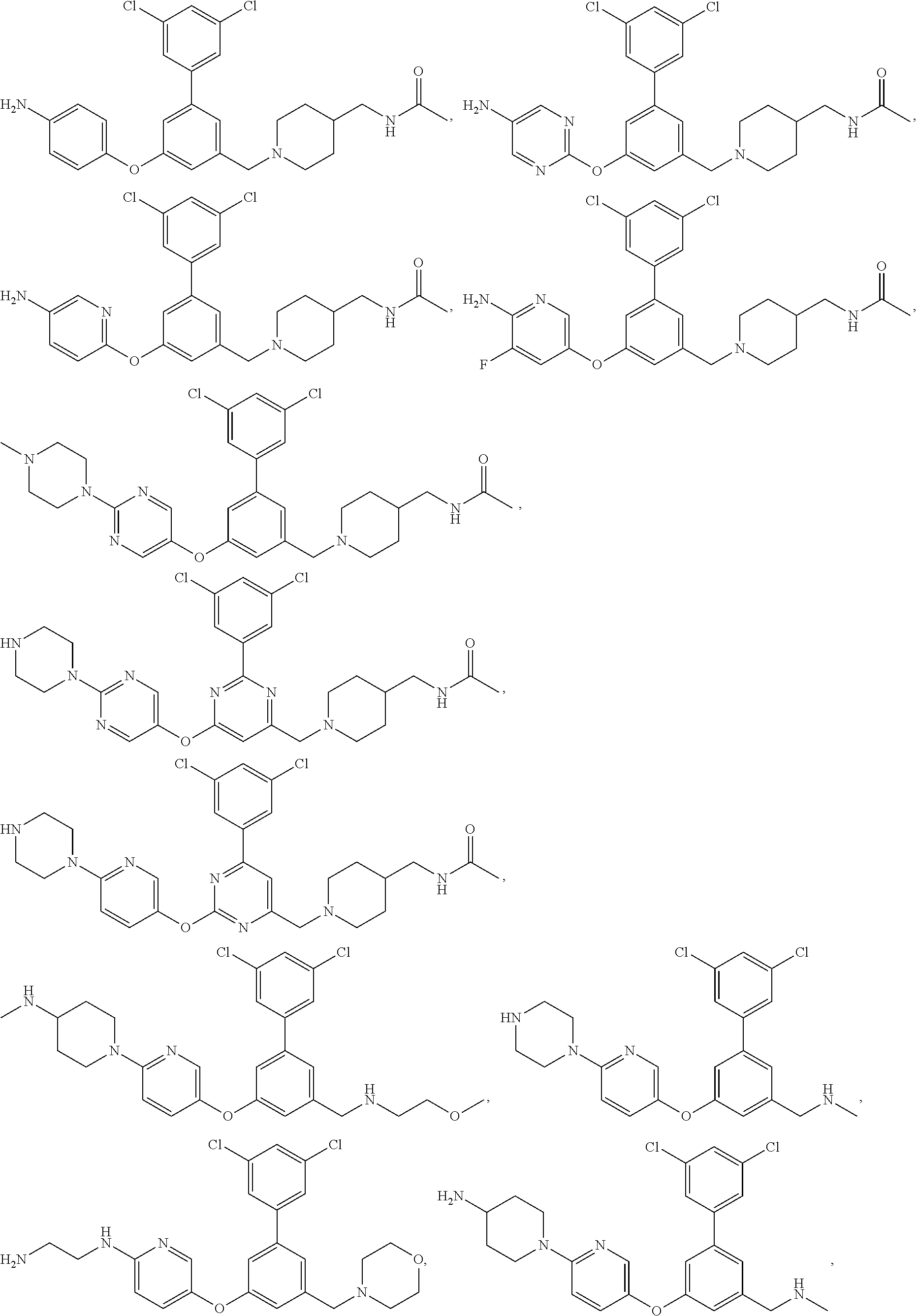

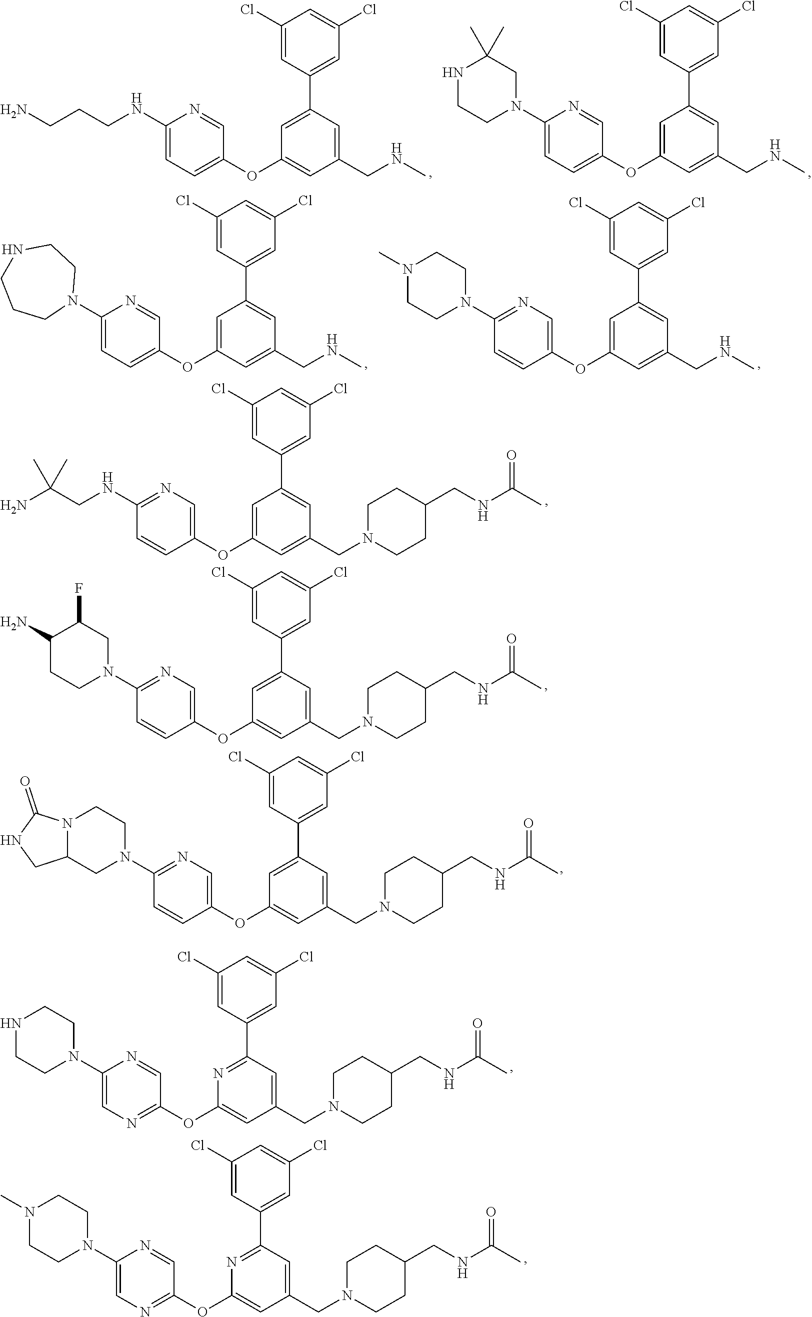

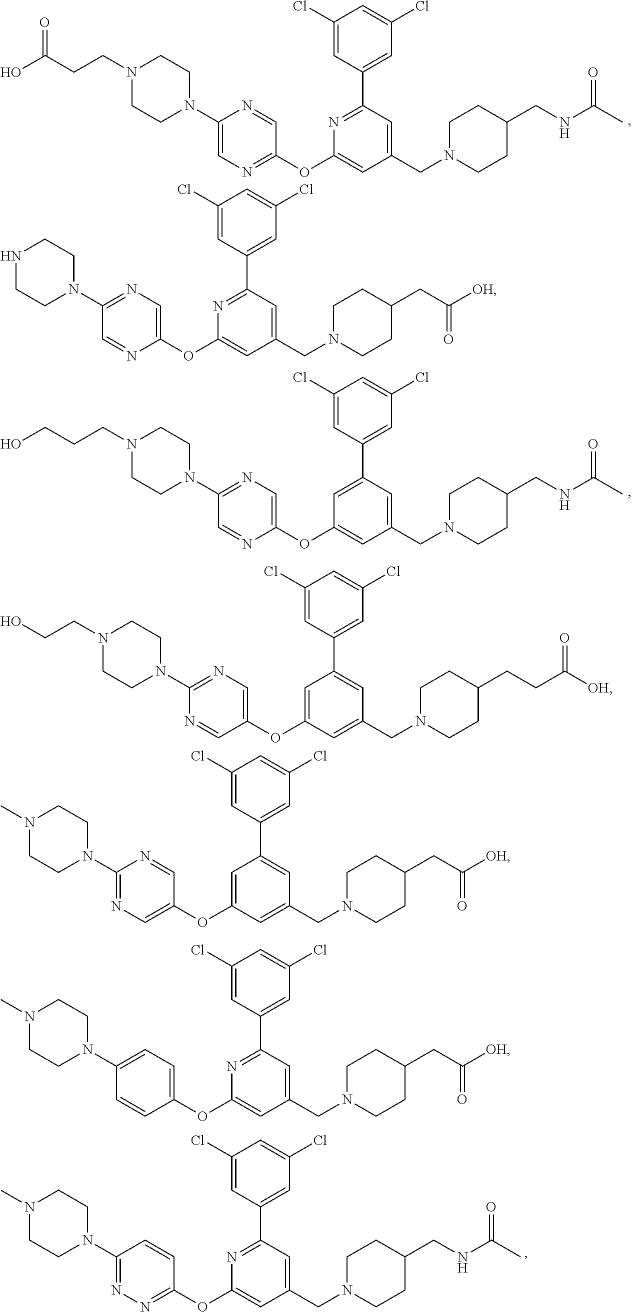

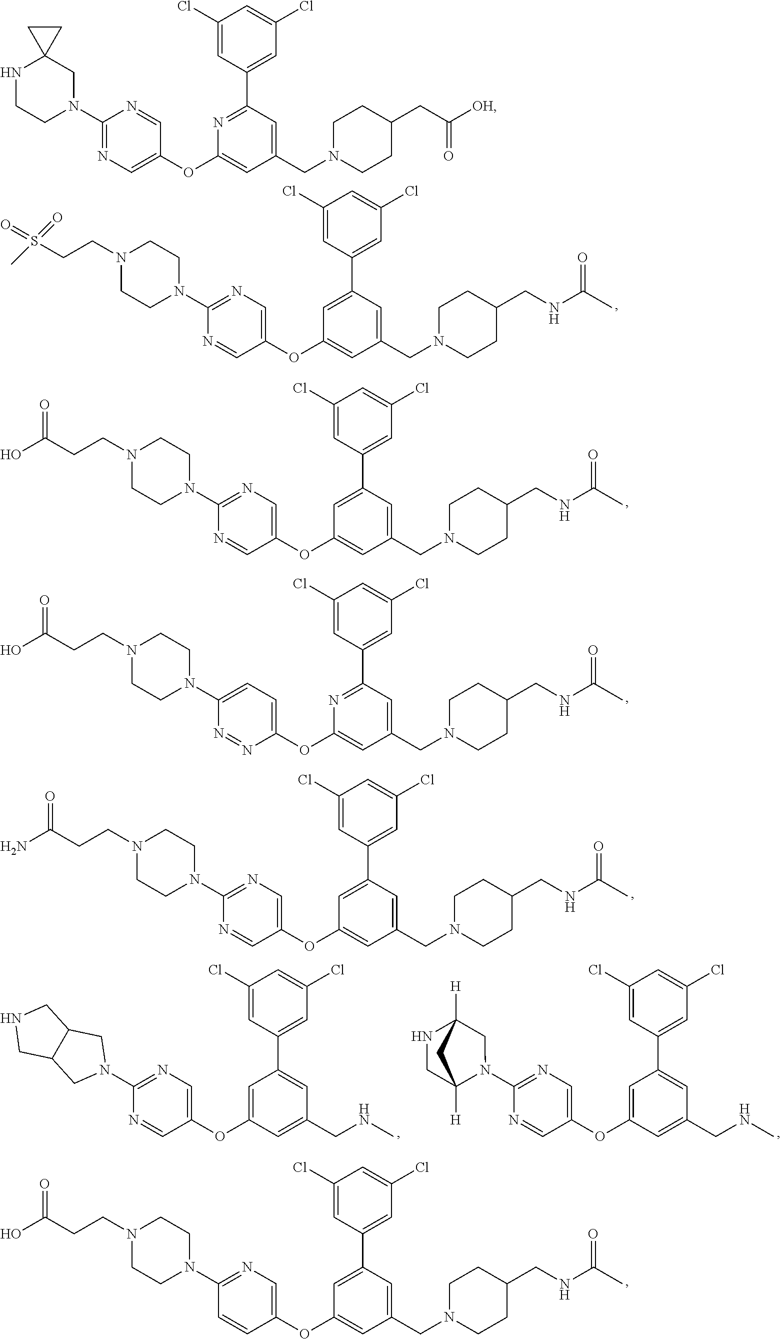

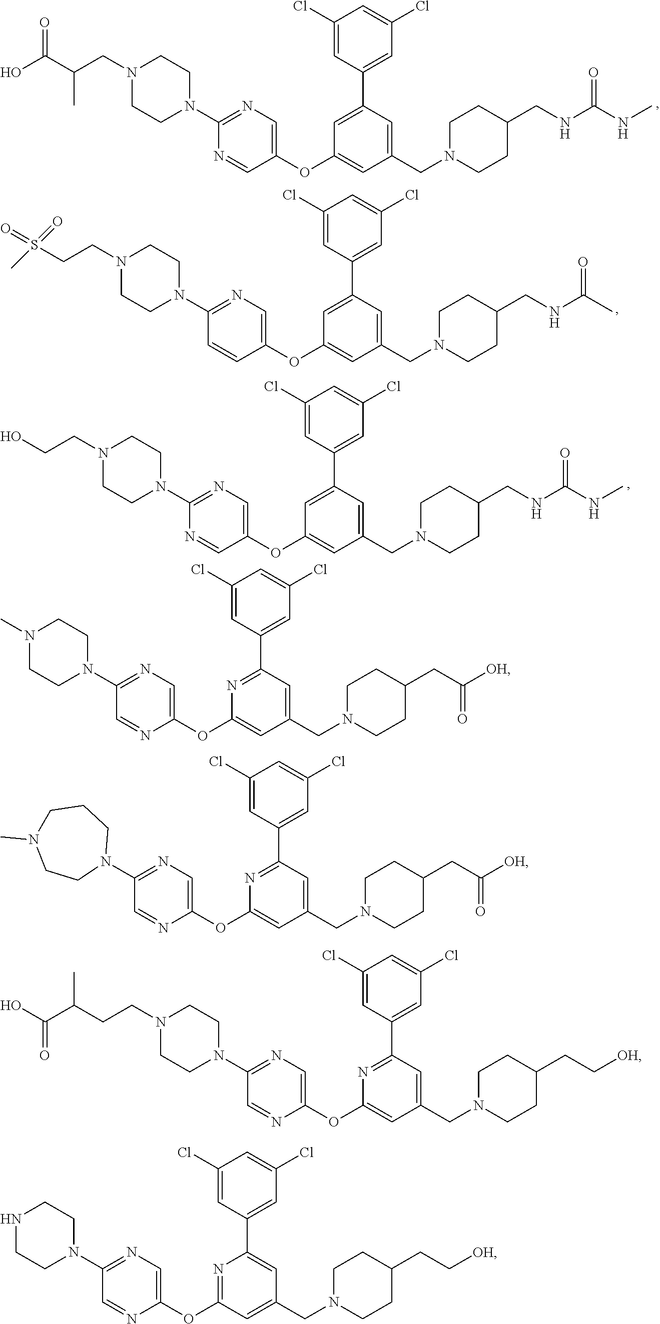

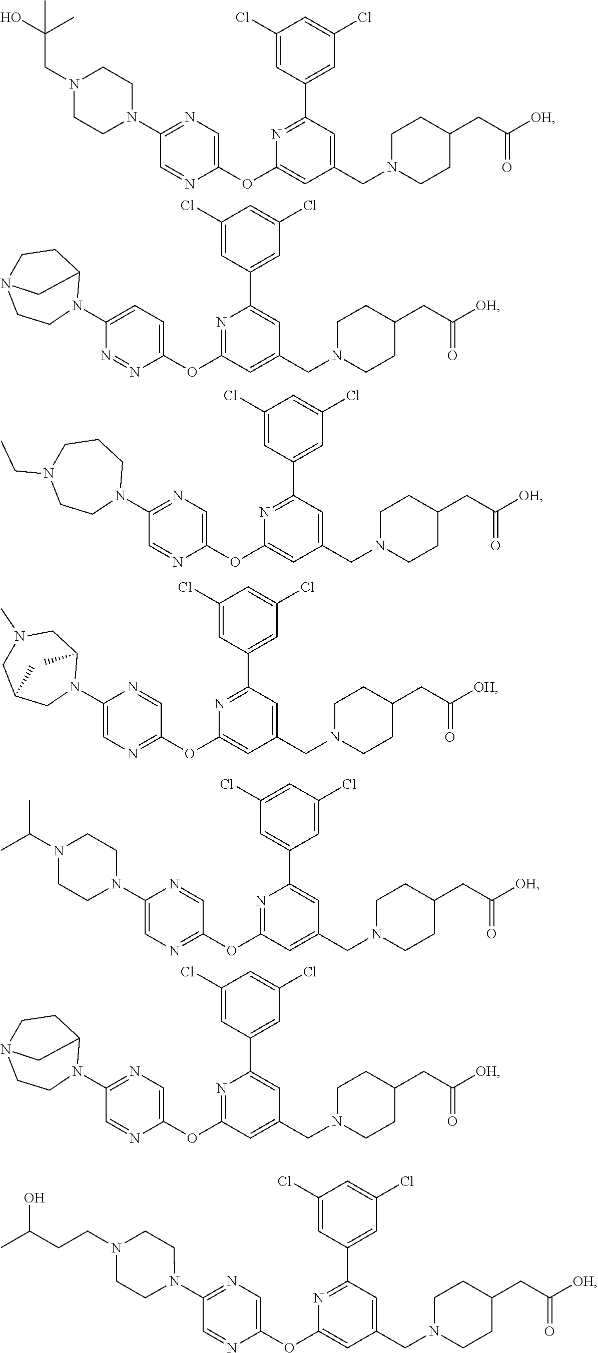

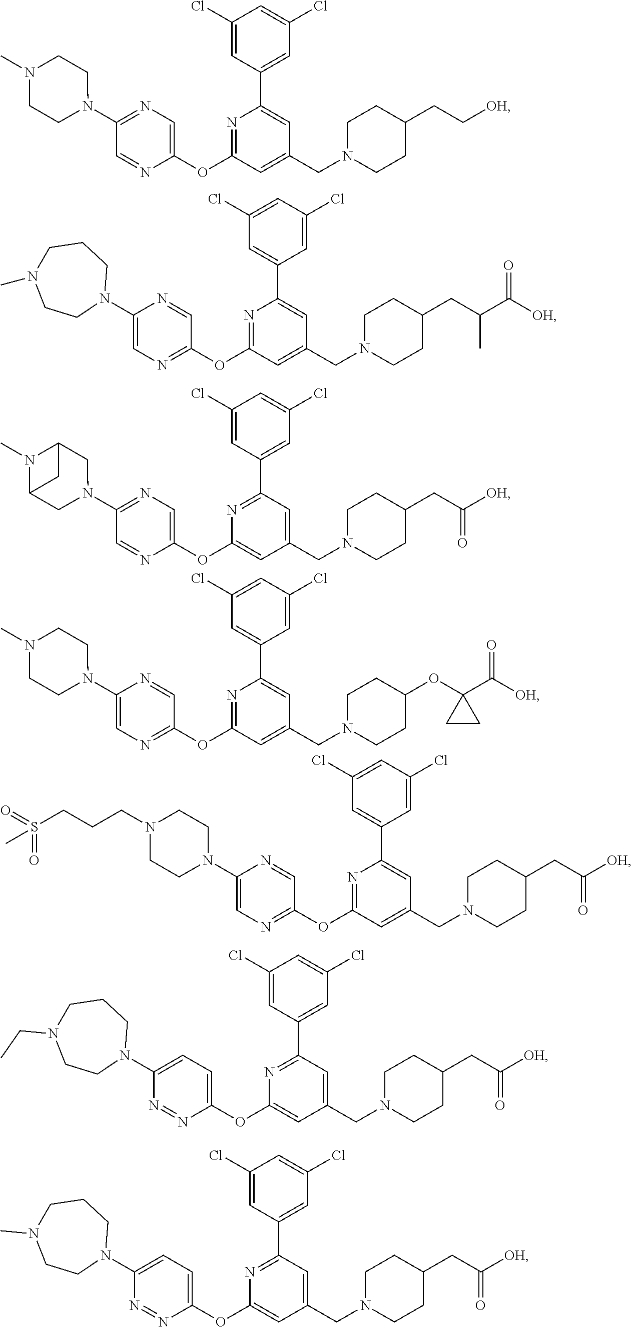

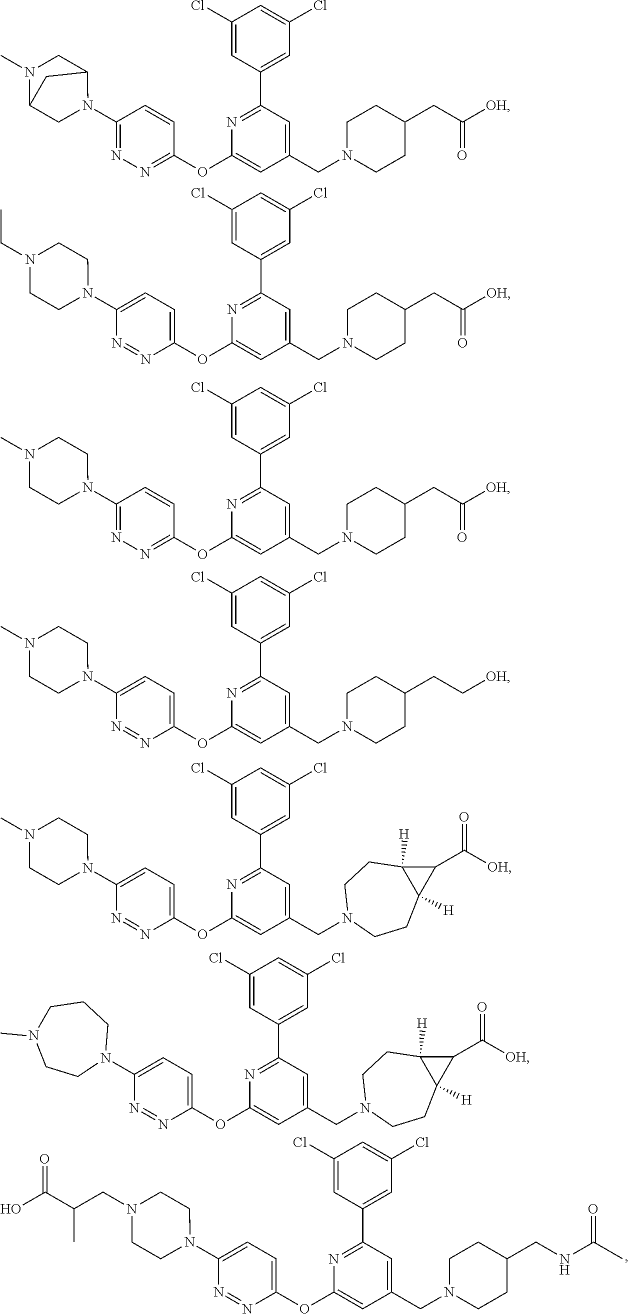

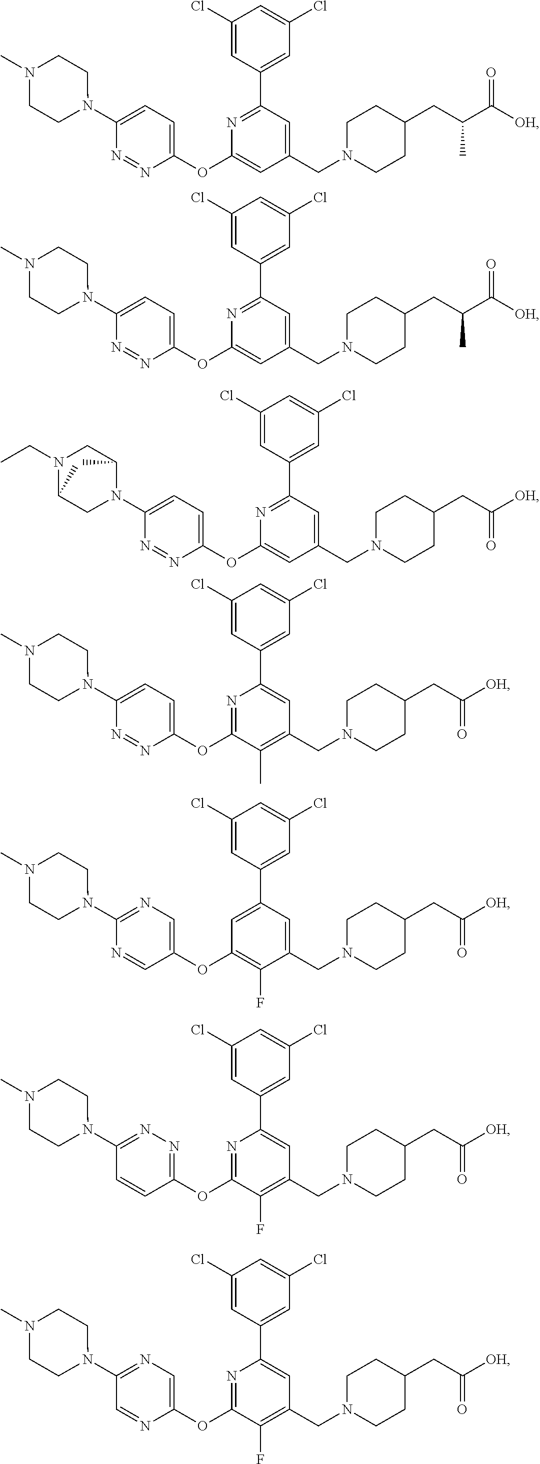

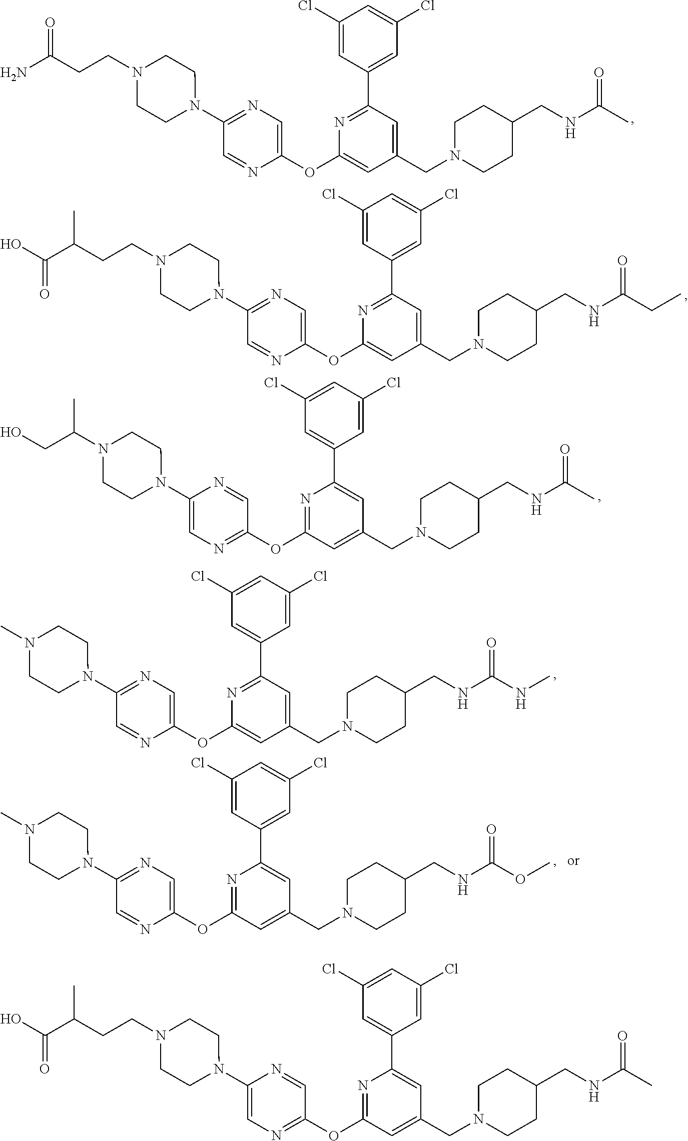

or a pharmaceutically acceptable salt thereof.

In certain embodiments, the compound of Formula (I) useful in the present disclosure is of the formula:

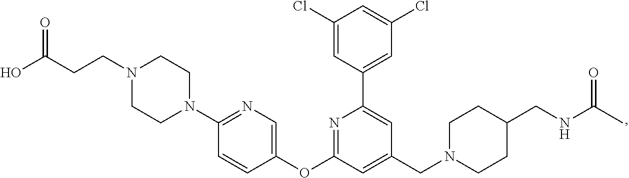 [SMILES: CC(=O)NCC1CCN(Cc2cc(Oc3ccc(N4CCN(CCC(=O)O)CC4)nc3)nc(-c3cc(Cl)cc(Cl)c3)c2)CC1]

or a pharmaceutically acceptable salt thereof.

In certain embodiments, the compound of Formula (I) useful in the present disclosure is of the formula:

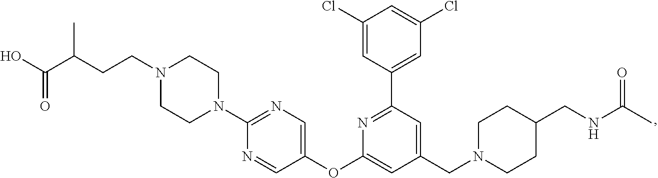 [SMILES: CC(=O)NCC1CCN(Cc2cc(Oc3cnc(N4CCN(CCC(C)C(=O)O)CC4)nc3)nc(-c3cc(Cl)cc(Cl)c3)c2)CC1]

or a pharmaceutically acceptable salt thereof.

The synthesis and characterization of all compounds in Table 1 can be found in U.S. Provisional Application, U.S. Ser. No. 62/670,050, filed on May 11, 2018, and the corresponding international PCT application, Application No.: PCT/EP2019/062098, filed on May 10, 2019, the contents of both are incorporated herein by reference.

TABLE 1
Compounds useful in the disclosure

| # | Structure | Name |
|---|---|---|
| 1 | 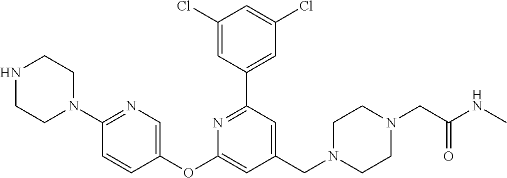 [SMILES: CNC(=O)CN1CCN(Cc2cc(Oc3ccc(N4CCNCC4)nc3)nc(-c3cc(Cl)cc(Cl)c3)c2)CC1] | 2-(4-((2-(3,5-dichlorophenyl)-6-((6- (piperazin-1-yl)pyridin-3-yl)oxy) pyridin-4-yl)methyl)piperazin- 1-yl)-N-methylacetamide |
| 2 | 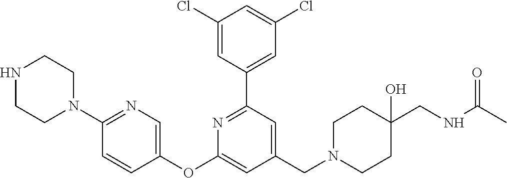 [SMILES: CC(=O)NCC1(O)CCN(Cc2cc(Oc3ccc(N4CCNCC4)nc3)nc(-c3cc(Cl)cc(Cl)c3)c2)CC1] | N-((1-((2-(3,5-dichlorophenyl)-6-((6- (piperazin-1-yl)pyridin-3-yl)oxy) pyridin-4-yl)methyl)-4-hydroxy- piperidin-4-yl)methyl)acetamide |
| 3 | 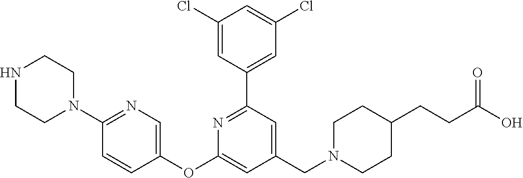 [SMILES: O=C(O)CCC1CCN(Cc2cc(Oc3ccc(N4CCNCC4)nc3)nc(-c3cc(Cl)cc(Cl)c3)c2)CC1] | 3-(1-((2-(3,5-dichlorophenyl)-6-((6- (piperazin-1-yl)pyridin-3-yl)oxy) pyridin-4-yl)methyl)piperidin-4-yl) propanoic acid |
| 4 | 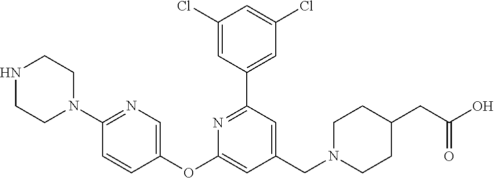 [SMILES: O=C(O)CC1CCN(Cc2cc(Oc3ccc(N4CCNCC4)nc3)nc(-c3cc(Cl)cc(Cl)c3)c2)CC1] | 2-(1-((2-(3,5-dichlorophenyl)-6-((6- (piperazin-1-yl)pyridin-3-yl)oxy) pyridin-4-yl)methyl)piperidin-4-yl) acetic acid |
| 5 | 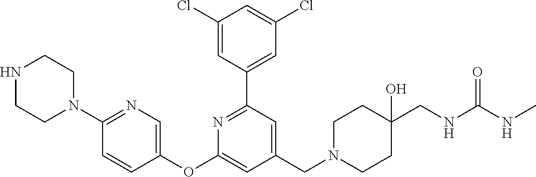 [SMILES: CNC(=O)NCC1(O)CCN(Cc2cc(Oc3ccc(N4CCNCC4)nc3)nc(-c3cc(Cl)cc(Cl)c3)c2)CC1] | 1-((1-((2-(3,5-dichlorophenyl)-6-((6- (piperazin-1-yl)pyridin-3-yl)oxy) pyridin-4-yl)methyl)-4-hydroxy- piperidin-4- yl)methyl)-3-methylurea |
| 6 | 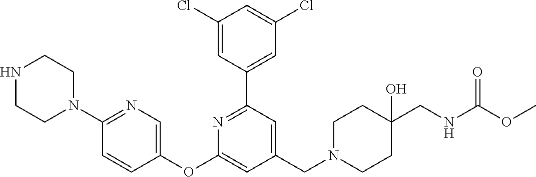 [SMILES: COC(=O)NCC1(O)CCN(Cc2cc(Oc3ccc(N4CCNCC4)nc3)nc(-c3cc(Cl)cc(Cl)c3)c2)CC1] | methyl ((1-((2-(3,5-dichlorophenyl)-6- ((6-(piperazin-1-yl)pyridin-3-yl)oxy) pyridin-4-yl)methyl)-4- hydroxypiperidin- 4-yl)methyl)carbamate |
| 7 | 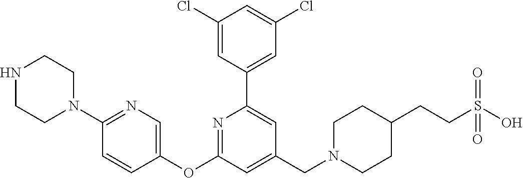 [SMILES: O=S(=O)(O)CCC1CCN(Cc2cc(Oc3ccc(N4CCNCC4)nc3)nc(-c3cc(Cl)cc(Cl)c3)c2)CC1] | 2-(1-((2-(3,5-dichlorophenyl)-6-((6- (piperazin-1-yl)pyridin-3-yl)oxy) pyridin-4-yl)methyl)piperidin-4-yl) ethanesulfonic acid |
| 8 | 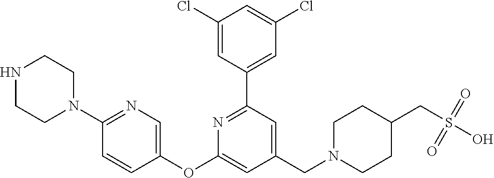 [SMILES: O=S(=O)(O)CC1CCN(Cc2cc(Oc3ccc(N4CCNCC4)nc3)nc(-c3cc(Cl)cc(Cl)c3)c2)CC1] | (1-((2-(3,5-dichlorophenyl)-6-((6- (piperazin-1-yl)pyridin-3-yl)oxy) pyridin-4-yl)methyl)piperidin-4- yl)methanesulfonic acid |
| 9 | 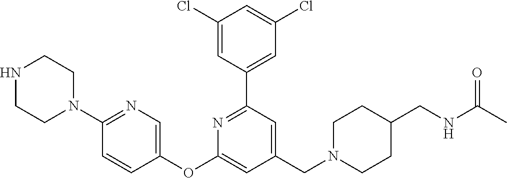 [SMILES: CC(=O)NCC1CCN(Cc2cc(Oc3ccc(N4CCNCC4)nc3)nc(-c3cc(Cl)cc(Cl)c3)c2)CC1] | N-((1-((2-(3,5-dichlorophenyl)-6-((6- (piperazin-1-yl)pyridin-3-yl)oxy) pyridin-4-yl)methyl)piperidin-4- yl)methyl)acetamide |
| 10 | 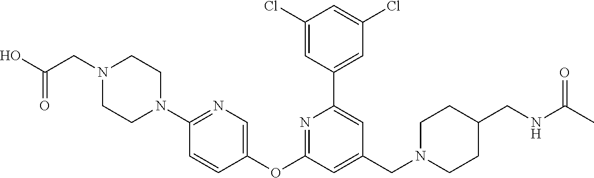 [SMILES: CC(=O)NCC1CCN(Cc2cc(Oc3ccc(N4CCN(CC(=O)O)CC4)nc3)nc(-c3cc(Cl)cc(Cl)c3)c2)CC1] | 2-(4-(5-((4-(4- (acetamidomethyl)piperidin-1-yl) methyl)-6-(3,5-dichlorophenyl) pyridin-2-yl)oxy)pyridin-2-yl) piperazin-1-yl)acetic acid |
| 11 | 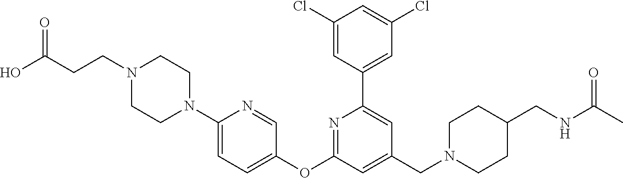 [SMILES: CC(=O)NCC1CCN(Cc2cc(Oc3ccc(N4CCN(CCC(=O)O)CC4)nc3)nc(-c3cc(Cl)cc(Cl)c3)c2)CC1] | 3-(4-(5-((4-(4- (acetamidomethyl)piperidin-1-yl) methyl)-6-(3,5-dichlorophenyl) pyridin-2-yl)oxy)pyridin-2-yl) piperazin-1-yl)propanoic acid |
| 12 | 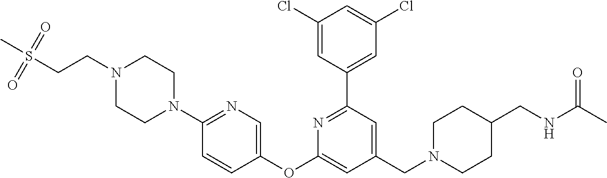 [SMILES: CC(=O)NCC1CCN(Cc2cc(Oc3ccc(N4CCN(CCS(C)(=O)=O)CC4)nc3)nc(-c3cc(Cl)cc(Cl)c3)c2)CC1] | N-((1-((2-(3,5-dischlorophenyl)-6-((6- (4-(2-(methylsulfonyl)ethyl)piperazin- 1-yl)pyridin-3-yl)oxy)pyridin-4- yl)methyl)piperidin-4- yl)methyl)acetamide |
| 13 | 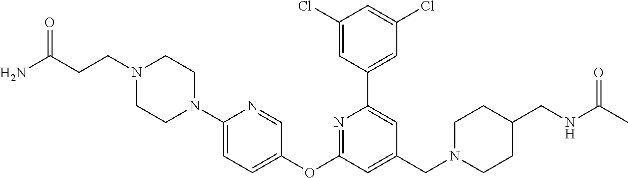 [SMILES: CC(=O)NCC1CCN(Cc2cc(Oc3ccc(N4CCN(CCC(N)=O)CC4)nc3)nc(-c3cc(Cl)cc(Cl)c3)c2)CC1] | 3-(4-(5-((4-((4- (acetamidomethyl)piperidin-1-yl) methyl)-6-(3,5-dichlorophenyl) pyridin-2-yl)oxy)pyridin-2-yl) piperazin-1-yl)propanamide |
| 14 | 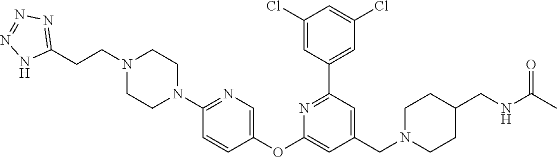 [SMILES: CC(=O)NCC1CCN(Cc2cc(Oc3ccc(N4CCN(CCc5nnn[nH]5)CC4)nc3)nc(-c3cc(Cl)cc(Cl)c3)c2)CC1] | N-((1-((2-(((6-(4-(2-(1H-tetrazol-5- yl)ethyl)piperazin-1-yl)pyridin-3-yl) oxy)-6-(3,5-dichlorophenyl)pyridin-4- yl)methyl)piperidin-4- yl)methyl)acetamide |
| 15 | 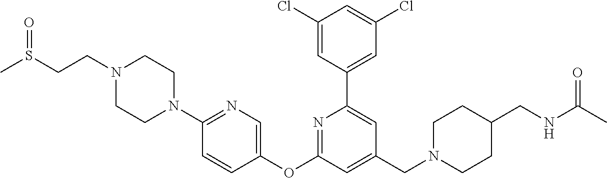 [SMILES: CC(=O)NCC1CCN(Cc2cc(Oc3ccc(N4CCN(CCS(C)=O)CC4)nc3)nc(-c3cc(Cl)cc(Cl)c3)c2)CC1] | (N-((1-((2-(3,5-dichlorophenyl)-6-((6- (4-(2-(methylsulfinyl)ethyl)piperazin- 1-yl)pyridin-3-yl)oxy)pyridin-4- yl)methyl)piperidin-4- yl)methyl)acetamide |
| 16 | 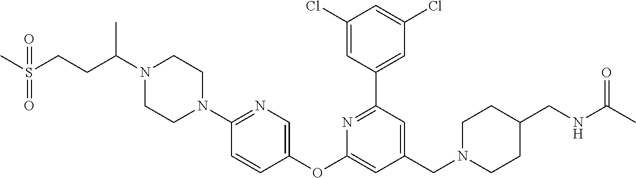 [SMILES: CC(=O)NCC1CCN(Cc2cc(Oc3ccc(N4CCN(C(C)CCS(C)(=O)=O)CC4)nc3)nc(-c3cc(Cl)cc(Cl)c3)c2)CC1] | (N-((1-((2-(3,5-dichlorophenyl)-6-((6- (4-(4-(methylsulfonyl)butan-2-yl) piperazin- 1-yl)pyridin-3-yl)oxy)pyridin-4- yl)methyl)piperidin-4- yl)methyl)acetamide |
| 17 | 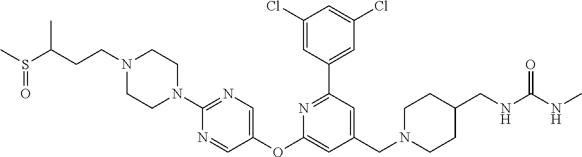 | 1-((1-((2-(3,5-chlorophenyl)-6-((2-(4- (3-(methylsulfinyl)butyl)piperazin-1- yl)pyrimidin-5-yl)oxy)pyridin-4- yl)methyl)piperidin-4-yl)methyl)-3- methylurea |
| 18 | 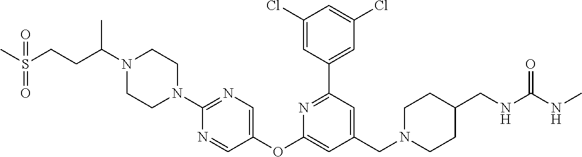 | 1-((1-((2-(3,5-dichlorophenyl)-6-((2-(4- (4-(methylsulfonyl)butan-2-yl)piperazin- 1-yl)pyrimidin-5-yl)oxy)pyridin-4- yl)methyl)piperidin-4-yl)methyl)-3- methylurea |
| 19 | 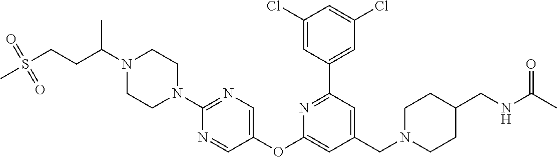 [SMILES: CC(=O)NCC1CCN(Cc2cc(Oc3cnc(N4CCN(C(C)CCS(C)(=O)=O)CC4)nc3)nc(-c3cc(Cl)cc(Cl)c3)c2)CC1] | N-((1-((2-(3,5-dichlorophenyl)-6-((2-(4- (4-(methylsulfonyl)butan-2-yl)piperazin- 1-yl)pyrimidin-5-yl)oxy)pyridin-4- yl)methyl)piperidin-4- yl)methyl)acetamide |
| 20 | 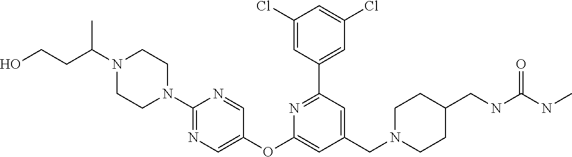 [SMILES: CNC(=O)NCC1CCN(Cc2cc(Oc3cnc(N4CCN(C(C)CCO)CC4)nc3)nc(-c3cc(Cl)cc(Cl)c3)c2)CC1] | 1-((1-((2-(3,5-dichlorophenyl)-6-((2-(4- (4-hydroxybutan-2-yl)piperazin-1- yl)pyrimidin-5-yl)oxy)pyridin-4- yl)methyl)piperidin-4-yl)methyl)-3- methylurea |
| 21 | 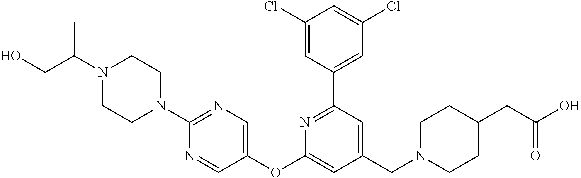 [SMILES: CC(CO)N1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2-(4- (1-hydroxypropan-2-yl)piperazin-1- yl)pyrimidin-5-yl)oxy)pyridin-4- yl)methyl)piperidin-4-yl)acetic acid |
| 22 | 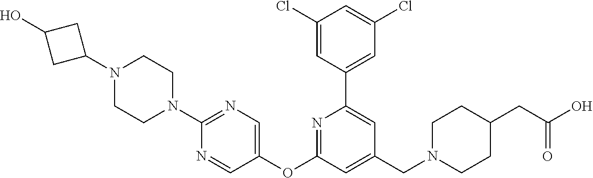 [SMILES: O=C(O)CC1CCN(Cc2cc(Oc3cnc(N4CCN(C5CC(O)C5)CC4)nc3)nc(-c3cc(Cl)cc(Cl)c3)c2)CC1] | 2-(1-((2-(3,5-dichlorophenyl)-6-((2-(4- (3-hydroxycyclobutyl)piperazin-1- yl)pyrimidin-5-yl)oxy)pyridin-4- yl)methyl)piperidin-4-yl)acetic acid |
| 23 | 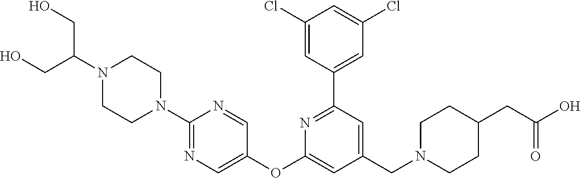 [SMILES: O=C(O)CC1CCN(Cc2cc(Oc3cnc(N4CCN(C(CO)CO)CC4)nc3)nc(-c3cc(Cl)cc(Cl)c3)c2)CC1] | 2-(1-((2-(3,5-dichlorophenyl)-6-((2-(4- (1,3-dihydroxypropan-2-yl)piperazin-1- yl)pyrimidin-5-yl)oxy)pyridin-4- yl)methyl)piperidin-4-yl)acetic acid |
| 24 | 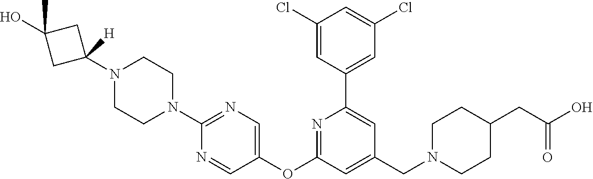 [SMILES: C[C@]1(O)C[C@@H](N2CCN(c3ncc(Oc4cc(CN5CCC(CC(=O)O)CC5)cc(-c5cc(Cl)cc(Cl)c5)n4)cn3)CC2)C1] | 2-(1-((2-(3,5-dichlorophenyl)-6-((2-(4- ((1s,3s)-3-hydroxy-3- methylcyclobutyl)piperazin-1- yl)pyrimidin-5-yl)oxy)pyridin-4- yl)methyl)piperidin-4-yl)acetic acid |
| 25 | 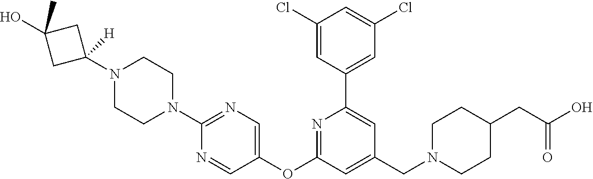 [SMILES: C[C@]1(O)C[C@H](N2CCN(c3ncc(Oc4cc(CN5CCC(CC(=O)O)CC5)cc(-c5cc(Cl)cc(Cl)c5)n4)cn3)CC2)C1] | 2-(1-((2-(3,5-dichlorophenyl)-6-((2-(4- ((1r,3r)-3-hydroxy-3- methylcyclobutyl)piperazin-1- yl)pyrimidin-5-yl)oxy)pyridin-4- yl)methyl)piperidin-4-yl)acetic acid |
| 26 | 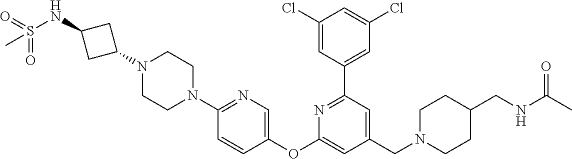 [SMILES: CC(=O)NCC1CCN(Cc2cc(Oc3ccc(N4CCN([C@H]5C[C@H](NS(C)(=O)=O)C5)CC4)nc3)nc(-c3cc(Cl)cc(Cl)c3)c2)CC1] | N-((1-((2-(3,5-dichlorophenyl)-6-((6- (4-((trans)-3- (methylsulfonamido)cyclobutyl) piperazin-1-yl)pyridin-3-yl)oxy) pyridin-4-yl)methyl)piperidin-4- yl)methyl)acetamide |
| 27 | 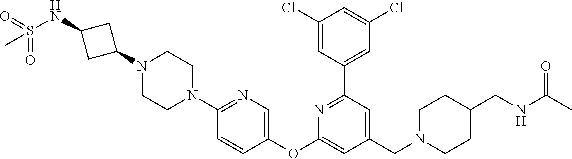 [SMILES: CC(=O)NCC1CCN(Cc2cc(Oc3ccc(N4CCN([C@H]5C[C@@H](NS(C)(=O)=O)C5)CC4)nc3)nc(-c3cc(Cl)cc(Cl)c3)c2)CC1] | N-((1-((2-(3,5-dichlorophenyl)-6-((6- (4-((cis)-3- (methylsulfonamido)cyclobutyl) piperazin-1-yl)pyridin-3-yl)oxy) pyridin-4-yl)methyl)piperidin-4- yl)methyl)acetamide |
| 28 | 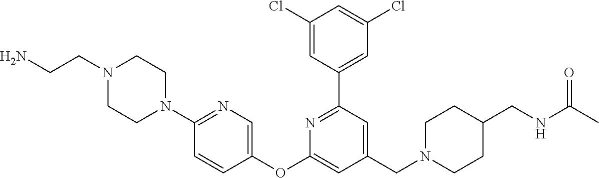 [SMILES: CC(=O)NCC1CCN(Cc2cc(Oc3ccc(N4CCN(CCN)CC4)nc3)nc(-c3cc(Cl)cc(Cl)c3)c2)CC1] | N-((1-((2-((6-(4-(2-aminoethyl) piperazin-1-yl)pyridin-3-yl)oxy)-6-(3, 5-dichlorophenyl)pyridin-4- yl)methyl)piperidin-4- yl)methyl)acetamide |
| 29 | 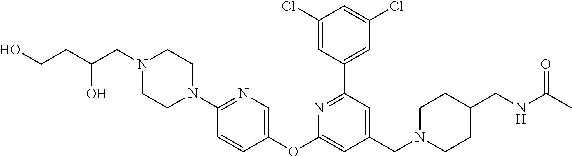 [SMILES: CC(=O)NCC1CCN(Cc2cc(Oc3ccc(N4CCN(CC(O)CCO)CC4)nc3)nc(-c3cc(Cl)cc(Cl)c3)c2)CC1] | N-((1-((2-(3,5-dichlorophenyl)-6-((6- (4-(2,4-dihydroxybutyl)piperazin-1- yl)pyridin-3-yl)oxy)pyridin-4- yl)methyl)piperidin-4- yl)methyl)acetamide |
| 30 | 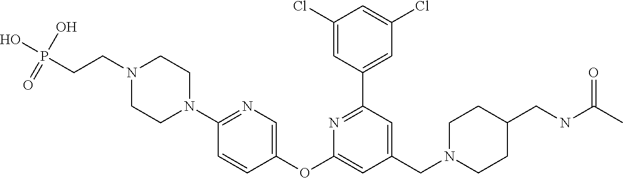 [SMILES: CC(=O)NCC1CCN(Cc2cc(Oc3ccc(N4CCN(CCP(=O)(O)O)CC4)nc3)nc(-c3cc(Cl)cc(Cl)c3)c2)CC1] | (2-(4-(5-((4-((4- (acetamidomethyl)piperidin-1-yl) methyl)-6-(3,5-dichlorophenyl) pyridin-2-yl)oxy)pyridin-2-yl) piperazin-1-yl)ethyl)phosphonic acid |
| 31 | 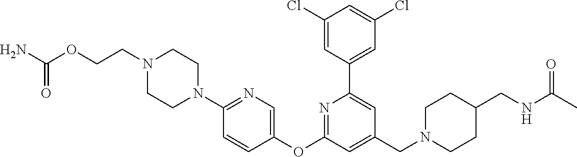 [SMILES: CC(=O)NCC1CCN(Cc2cc(Oc3ccc(N4CCN(CCOC(N)=O)CC4)nc3)nc(-c3cc(Cl)cc(Cl)c3)c2)CC1] | 2-(4-(5-((4-((4- (acetamidomethyl)piperidin-1-yl) methyl)-6-(3,5-dichlorophenyl) pyridin-2-yl)oxy)pyridin-2-yl) piperazin-1-yl)ethylcarbamate |
| 32 | 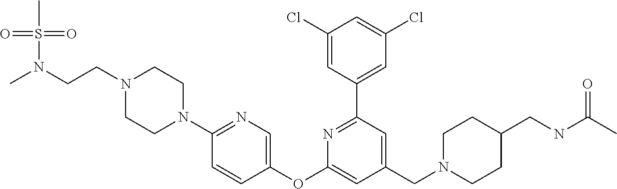 [SMILES: CC(=O)NCC1CCN(Cc2cc(Oc3ccc(N4CCN(CCN(C)S(C)(=O)=O)CC4)nc3)nc(-c3cc(Cl)cc(Cl)c3)c2)CC1] | N-((1-((2-(3,5-dichlorophenyl)-6-((6-(4- (2-(N-methylmethylsulfonamido) ethyl)piperazin-1-yl)pyridin-3-yl)oxy) pyridin-4- yl)methyl)piperidin-4- yl)methyl)acetamide |
| 33 | 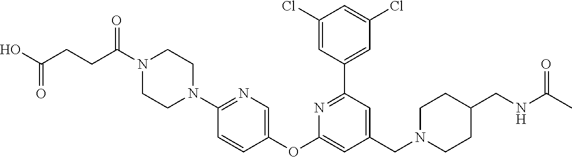 [SMILES: CC(=O)NCC1CCN(Cc2cc(Oc3ccc(N4CCN(C(=O)CCC(=O)O)CC4)nc3)nc(-c3cc(Cl)cc(Cl)c3)c2)CC1] | 4-(4-(5-((4-((4- (acetamidomethyl)piperidin-1-yl) methyl)-6-(3,5-dichlorophenyl) pyridin-2-yl)oxy)pyridin-2-yl) piperazin-1-yl)-4-oxobutanoic acid |
| 34 | 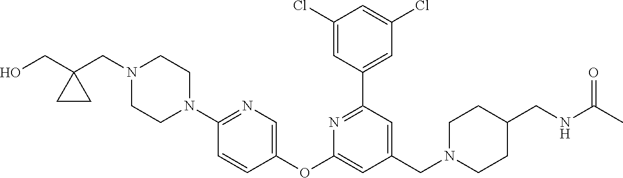 [SMILES: CC(=O)NCC1CCN(Cc2cc(Oc3ccc(N4CCN(CC5(CO)CC5)CC4)nc3)nc(-c3cc(Cl)cc(Cl)c3)c2)CC1] | N-((1-((2-(3,5-dichlorophenyl)-6-((6- (4-((1- (hydroxymethyl)cyclopropyl)methyl) piperazin-1-yl)pyridin-3-yl)oxy) pyridin-4-yl)methyl)piperidin-4- yl)methyl)acetamide |
| 35 | 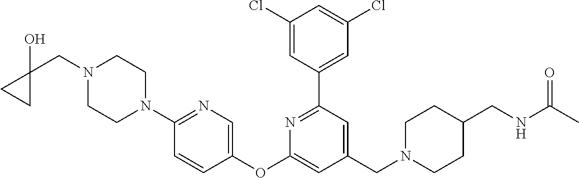 [SMILES: CC(=O)NCC1CCN(Cc2cc(Oc3ccc(N4CCN(CC5(O)CC5)CC4)nc3)nc(-c3cc(Cl)cc(Cl)c3)c2)CC1] | N-((1-((2-(3,5-dichlorophenyl)-6-((6- (4-((1-hydroxycyclopropyl)methyl) piperazin-1-yl)pyridin-3-yl)oxy) pyridin-4-yl)methyl)piperidin-4- yl)methyl)acetamide |
| 36 | 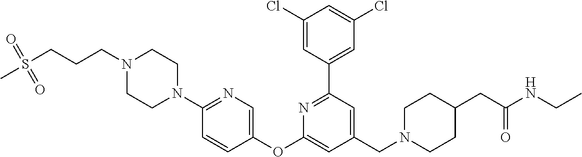 [SMILES: CCNC(=O)CC1CCN(Cc2cc(Oc3ccc(N4CCN(CCCS(C)(=O)=O)CC4)nc3)nc(-c3cc(Cl)cc(Cl)c3)c2)CC1] | 2-(1-((2-(3,5-dichlorophenyl)-6-((6-(4- (3-(methylsulfonyl)propyl)piperazin-1- yl)pyridin-3-yl)oxy)pyridin-4- yl)methyl)piperidin-4-yl)-N- ethylacetamide |
| 37 | 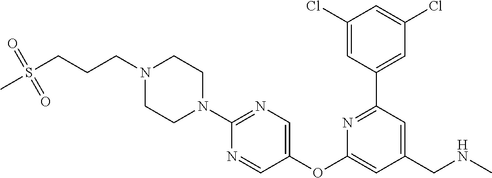 | 1-(2-(3,5-dichlorophenyl)-6-((2-(4-(3- (methylsulfonyl)propyl)piperazin-1- yl)pyrimidin-5-yl)oxy)pyridin-4-yl)-N- methylmethanamine |
| 38 | 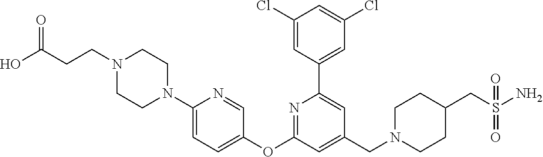 [SMILES: NS(=O)(=O)CC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)-4-((4- (sulfamoylmethyl)piperidin-1- yl)methyl)pyridin-2-yl)oxy)pyridin-2- yl)piperazin-1-yl)propanoic acid |
| 39 | 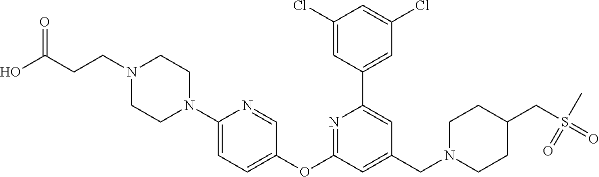 [SMILES: CS(=O)(=O)CC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)-4-((4- ((methylsulfonyl)methyl)piperidin-1- yl)methyl)pyridin-2-yl)oxy)pyridin-2- yl)piperazin-1-yl)propanoic acid |
| 40 | 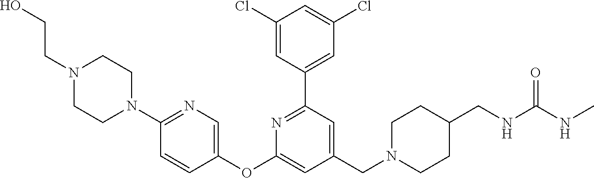 [SMILES: CNC(=O)NCC1CCN(Cc2cc(Oc3ccc(N4CCN(CCO)CC4)nc3)nc(-c3cc(Cl)cc(Cl)c3)c2)CC1] | 1-((1-((2-(3,5-dichlorophenyl)-6-((6-(4- (2-hydroxyethyl)piperazin-1-yl)pyridin- 3-yl)oxy)pyridin-4-yl)methyl)piperidin- 4-yl)methyl)-3-methylurea |
| 41 | 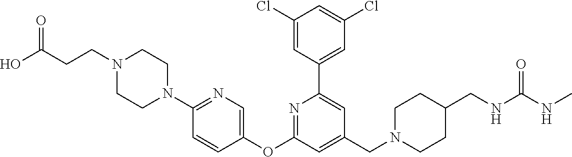 [SMILES: CNC(=O)NCC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)-4-((4- ((3-methylureido)methyl)piperidin-1- yl)methyl)pyridin-2-yl)oxy)pyridin-2- yl)piperazin-1-yl)propanoic acid |
| 42 | 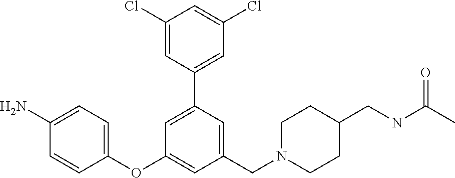 [SMILES: CC(=O)NCC1CCN(Cc2cc(Oc3ccc(N)cc3)cc(-c3cc(Cl)cc(Cl)c3)c2)CC1] | N-((1-((5-(4-aminophenoxy)-3′,5′- dichloro-[1,1′-biphenyl]-3- yl)methyl)piperidin-4- yl)methyl)acetamide |
| 43 | 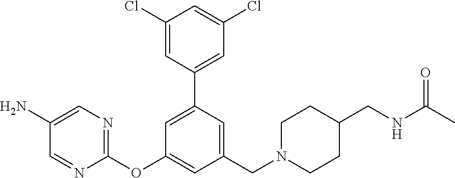 [SMILES: CC(=O)NCC1CCN(Cc2cc(Oc3ncc(N)cn3)cc(-c3cc(Cl)cc(Cl)c3)c2)CC1] | N-((1-((5-((5-aminopyrimidin-2-yl) oxy)-3′,5′-dichloro-[1,1′-biphenyl]-3- yl)methyl)piperidin-4- yl)methyl)acetamide |
| 44 | 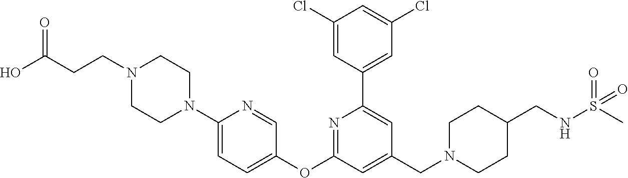 [SMILES: CS(=O)(=O)NCC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)-4-((4- (methylsulfonamidomethyl)piperidin-1- yl)methyl)pyridin-2-yl)oxy)pyridin-2- yl)piperazin-1-yl)propanoic acid |
| 45 | 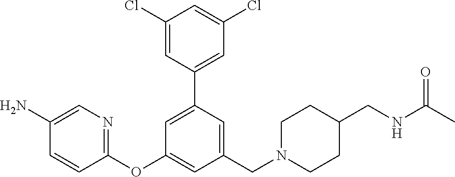 [SMILES: CC(=O)NCC1CCN(Cc2cc(Oc3ccc(N)cn3)cc(-c3cc(Cl)cc(Cl)c3)c2)CC1] | N-((1-((5-((5-aminopyridin-2-yl)oxy)- 3′,5′-dichloro-[1,1′-biphenyl]-3- yl)methyl)piperidin-4- yl)methyl)acetamide |
| 46 | 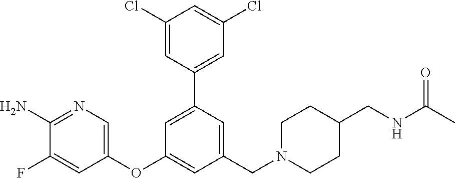 [SMILES: CC(=O)NCC1CCN(Cc2cc(Oc3cnc(N)c(F)c3)cc(-c3cc(Cl)cc(Cl)c3)c2)CC1] | N-((1-((5-((6-amino-5-fluoropyridin-3- yl)oxy)-3′,5′-dichloro-[1,1′-biphenyl]- 3-yl)methyl)piperidin-4- yl)methyl)acetamide |
| 47 | 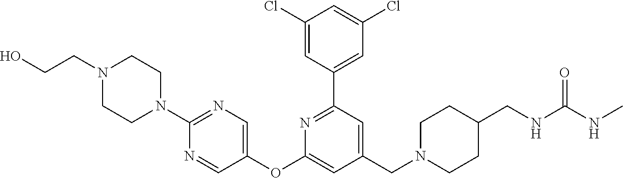 [SMILES: CNC(=O)NCC1CCN(Cc2cc(Oc3cnc(N4CCN(CCO)CC4)nc3)nc(-c3cc(Cl)cc(Cl)c3)c2)CC1] | 1-((1-((2-(3,5-dichlorophenyl)-6-((2- (4-(2-hydroxyethyl)piperazin-1-yl) pyrimidin-5-yl)oxy)pyridin-4-yl) methyl)piperidin-4- yl)methyl)-3-methylurea |
| 48 | 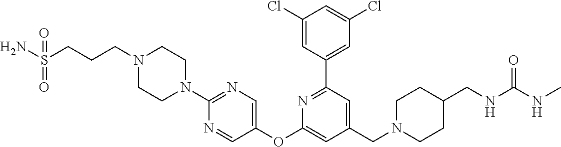 [SMILES: CNC(=O)NCC1CCN(Cc2cc(Oc3cnc(N4CCN(CCCS(N)(=O)=O)CC4)nc3)nc(-c3cc(Cl)cc(Cl)c3)c2)CC1] | 3-(4-(5-((6-(3,5-dichlorophenyl)-4-((4- ((3-methylureido)methyl)piperidin-1- yl)methyl)pyridin-2-yl)oxy)pyrimidin- 2-yl)piperazin-1-yl)propane-1- sulfonamide |
| 49 | 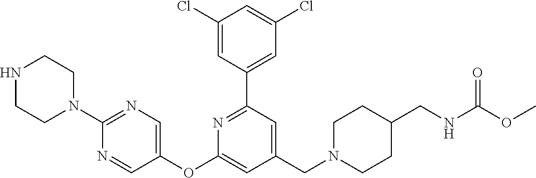 [SMILES: COC(=O)NCC1CCN(Cc2cc(Oc3cnc(N4CCNCC4)nc3)nc(-c3cc(Cl)cc(Cl)c3)c2)CC1] | methyl((1-((2-(3,5-dichlorophenyl)-6- ((2-(piperazin-1-yl)pyrimidin-5- yl)oxy)pyridin-4-yl)methyl)piperidin- 4-yl)methyl)carbamate |
| 50 | 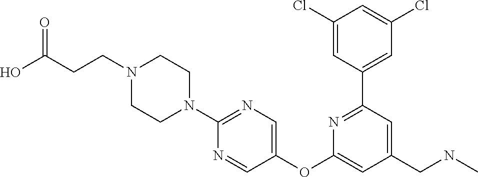 [SMILES: CNCc1cc(Oc2cnc(N3CCN(CCC(=O)O)CC3)nc2)nc(-c2cc(Cl)cc(Cl)c2)c1] | 3-(4-(5-((6-(3,5-dichlorophenyl)-4- ((methylamino)methyl)pyridin-2- yl)oxy)pyridin-2-yl)piperazin-1- yl)propanoic acid |
| 51 | 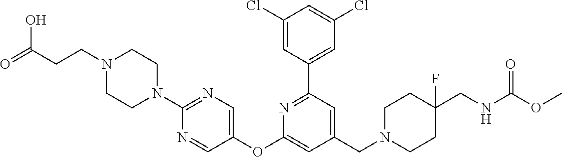 [SMILES: COC(=O)NCC1(F)CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichlorophenyl)-4-((4- fluoro-4- (((methoxycarbonyl)amino)methyl) piperidin-1-yl)methyl)pyridin-2- yl)oxy)pyrimidin-2-yl)piperazin-1- yl)propanoic acid |
| 52 | 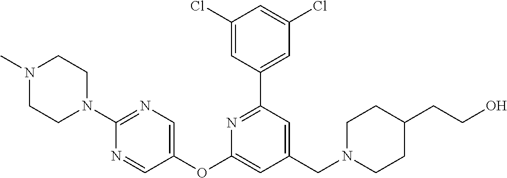 [SMILES: CN1CCN(c2ncc(Oc3cc(CN4CCC(CCO)CC4)cc(-c4cc(Cl)cc(Cl)c4)n3)cn2)CC1] | 2-(1-((2-(3,5-dichlorophenyl)-6-((2-(4- methylpiperazin-1-yl)pyrimidin-5- yl)oxy)pyridin-4-yl)methyl)piperidin-4- yl)ethanol |
| 53 | 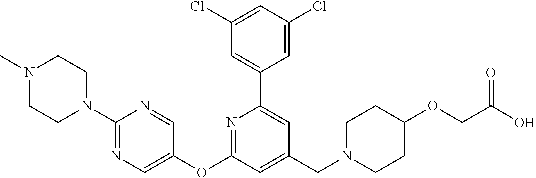 [SMILES: CN1CCN(c2ncc(Oc3cc(CN4CCC(OCC(=O)O)CC4)cc(-c4cc(Cl)cc(Cl)c4)n3)cn2)CC1] | 2-((1-((2-(3,5-dichlorophenyl)-6-((2-(4- methylpiperazin-1-yl)pyrimidin-5- yl)oxy)pyridin-4-yl)methyl)piperidin- 4-yl)oxy)acetic acid |
| 54 | 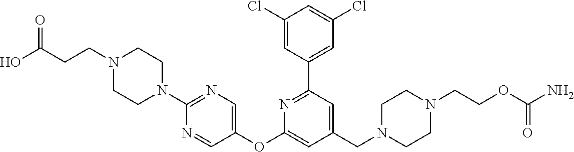 [SMILES: NC(=O)OCCN1CCN(Cc2cc(Oc3cnc(N4CCN(CCC(=O)O)CC4)nc3)nc(-c3cc(Cl)cc(Cl)c3)c2)CC1] | 3-(4-(5-((4-((4-(2- (carbamoyloxy)ethyl)piperazin-1- yl)methyl)-6-(3,5-dichlorophenyl) pyridin-2-yl)oxy)pyrimidin-2-yl) piperazin-1-yl)propanoic acid |
| 55 | 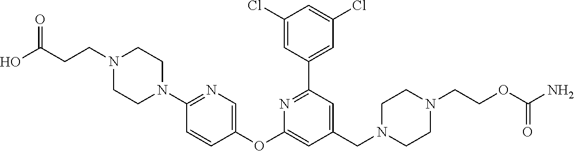 [SMILES: NC(=O)OCCN1CCN(Cc2cc(Oc3ccc(N4CCN(CCC(=O)O)CC4)nc3)nc(-c3cc(Cl)cc(Cl)c3)c2)CC1] | 3-(4-(5-((4-((4-(2- (carbamoyloxy)ethyl)piperazin-1- yl)methyl)-6-(3,5-dichlorophenyl) pyridin-2-yl)oxy)pyridin-2-yl) piperazin-1-yl)propanoic acid |
| 56 | 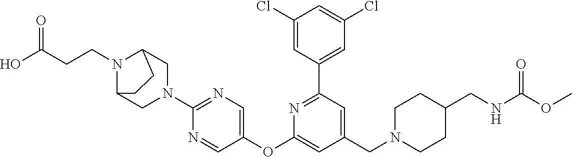 [SMILES: COC(=O)NCC1CCN(Cc2cc(Oc3cnc(N4CC5CCC(C4)N5CCC(=O)O)nc3)nc(-c3cc(Cl)cc(Cl)c3)c2)CC1] | 3-(3-(5-((6-(3,5-dichlorophenyl)-4-((4- (((methoxycarbonyl)amino)methyl) piperidin-1-yl)methyl)pyridin-2- yl)oxy)pyrimidin-2-yl)-3,8- diazabicyclo[3.2.1]octan-8-yl) propanoic acid |
| 57 | 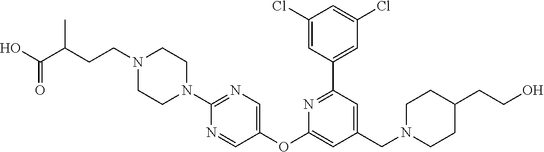 [SMILES: CC(CCN1CCN(c2ncc(Oc3cc(CN4CCC(CCO)CC4)cc(-c4cc(Cl)cc(Cl)c4)n3)cn2)CC1)C(=O)O] | 4-(4-(5-((6-(3,5-dichlorophenyl)-4-((4- (2-hydroxyethyl)piperidin-1- yl)methyl)pyridin-2-yl)oxy)pyrimidin- 2-yl)piperazin-1-yl)-2-methylbutanoic acid |
| 58 | 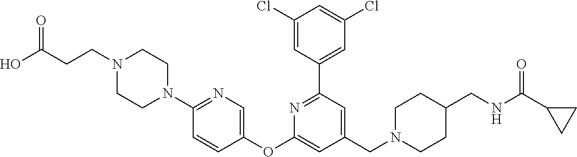 [SMILES: O=C(O)CCN1CCN(c2ccc(Oc3cc(CN4CCC(CNC(=O)C5CC5)CC4)cc(-c4cc(Cl)cc(Cl)c4)n3)cn2)CC1] | 3-(4-(5-((4-((4- (cyclopropanecarboxamidomethyl) piperidin-1-yl)methyl)-6-(3,5- dichlorophenyl)pyridin-2-yl)oxy) pyridin-2-yl)piperazin-1-yl)propanoic acid |
| 59 | 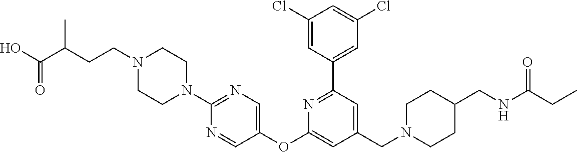 [SMILES: CCC(=O)NCC1CCN(Cc2cc(Oc3cnc(N4CCN(CCC(C)C(=O)O)CC4)nc3)nc(-c3cc(Cl)cc(Cl)c3)c2)CC1] | 4-(4-(5-((6-(3,5-dichlorophenyl)-4-((4- (propionamidomethyl)piperidin-1- yl)methyl)pyridin-2-yl)oxy)pyrimidin- 2-yl)piperazin-1-yl)-2-methylbutanoic acid |
| 60 | 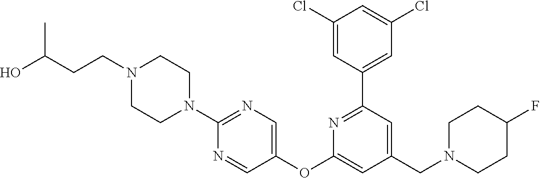 [SMILES: CC(O)CCN1CCN(c2ncc(Oc3cc(CN4CCC(F)CC4)cc(-c4cc(Cl)cc(Cl)c4)n3)cn2)CC1] | 4-(4-(5-((6-(3,5-dichlorophenyl)-4-((4- fluoropiperidin-1-yl)methyl)pyridin-2- yl)oxy)pyrimidin-2-yl)piperazin-1- yl)butan-2-ol |
| 61 | 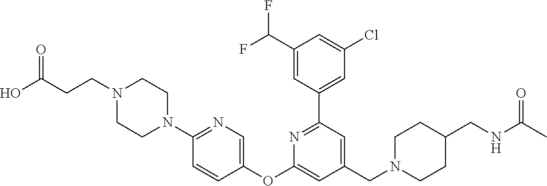 [SMILES: CC(=O)NCC1CCN(Cc2cc(Oc3ccc(N4CCN(CCC(=O)O)CC4)nc3)nc(-c3cc(Cl)cc(C(F)F)c3)c2)CC1] | 3-(4-(5-((4-((4- (acetamidomethyl)piperidin-1-yl) methyl)-6-(3-chloro-5- (difluoromethyl)phenyl)pyridin-2- yl)oxy)pyrimidin-2-yl)piperazin-1- yl)propanoic acid |
| 62 | 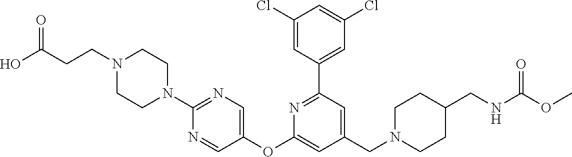 [SMILES: COC(=O)NCC1CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichlorophenyl)-4-((4- (((methoxycarbonyl)amino)methyl) piperidin-1-yl)methyl)pyridin-2- yl)oxy)pyrimidin-2-yl)piperazin-1- yl)propanoic acid |
| 63 | 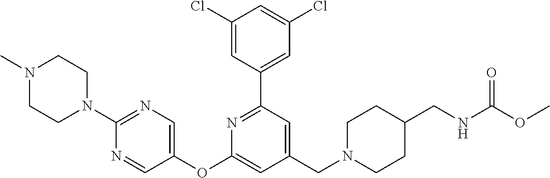 | methyl((1-((2-(3,5-dichlorophenyl)-6- ((2-(4-methylpiperazin-1-yl)pyrimidin- 5-yl)oxy)pyridin-4-yl)methyl)piperidin- 4-yl)methyl)carbamate |
| 64 | 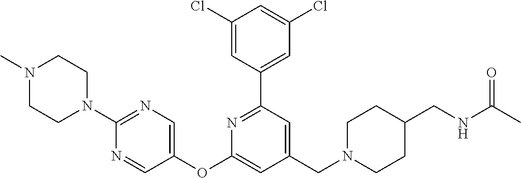 [SMILES: CC(=O)NCC1CCN(Cc2cc(Oc3cnc(N4CCN(C)CC4)nc3)nc(-c3cc(Cl)cc(Cl)c3)c2)CC1] | N-((1-((2-(3,5-dichlorophenyl)-6-((2-(4- methylpiperazin-1-yl)pyrimidin-5- yl)oxy)pyridin-4-yl)methyl)piperidin- 4-yl)methyl)acetamide |
| 65 | 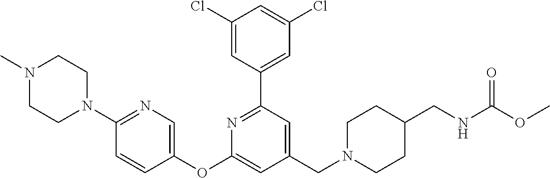 | methyl((1-((2-(3,5-dichlorophenyl)-6- ((6-(4-methylpiperazin-1-yl)pyridin-3- yl)oxy)pyridin-4-yl)methyl)piperidin- 4-yl)methyl)carbamate |
| 66 | 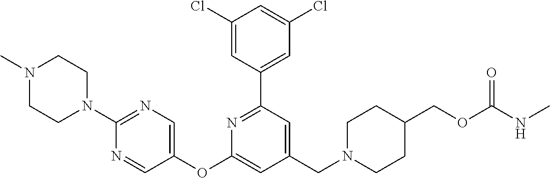 | (1-((2-(3,5-dichlorophenyl)-6-((2-(4- methylpiperazin-1-yl)pyrimidin-5- yl)oxy)pyridin-4-yl)methyl)piperidin- 4-yl)methylmethylcarbamate |
| 67 | 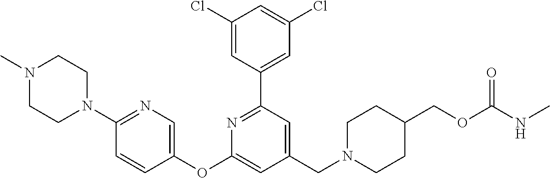 | (1-((2-(3,5-dichlorophenyl)-6-((6-(4- methylpiperazin-1-yl)pyridin-3- yl)oxy)pyridin-4-yl)methyl)piperidin-4- yl)methylmethylcarbamate |
| 68 | 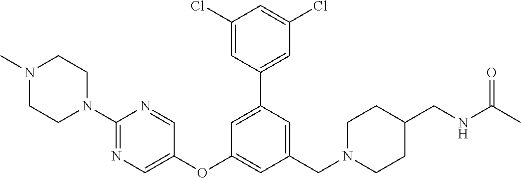 [SMILES: CC(=O)NCC1CCN(Cc2cc(Oc3cnc(N4CCN(C)CC4)nc3)cc(-c3cc(Cl)cc(Cl)c3)c2)CC1] | N-((1-((3′,5′-dichloro-5-((2-(4- methylpiperazin-1-yl)pyrimidin-5-yl) oxy)-[1,1′-biphenyl]-3-yl)methyl) piperidin-4-yl)methyl)acetamide |
| 69 | 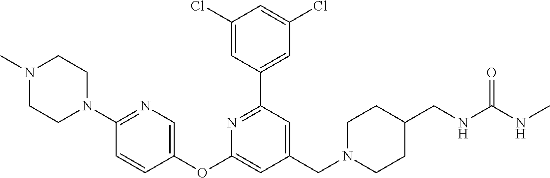 | 1-((1-((2-(3,5-dichlorophenyl)-6-((6- (4-methylpiperazin-1-yl)pyridin-3- yl)oxy)pyridin-4-yl)methyl)piperidin- 4-yl)methyl)-3-methylurea |
| 70 | 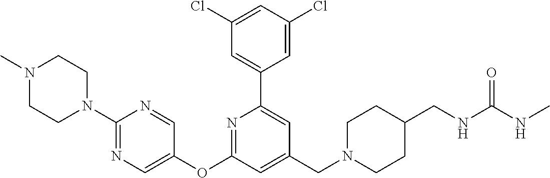 | 1-((1-((2-(3,5-dichlorophenyl)-6-((2- (4-methylpiperazin-1-yl)pyrimidin-5- yl)oxy)pyridin-4-yl)methyl)piperidin- 4-yl)methyl)-3-methylurea |
| 71 | 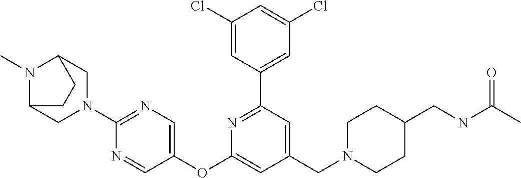 [SMILES: CC(=O)NCC1CCN(Cc2cc(Oc3cnc(N4CC5CCC(C4)N5C)nc3)nc(-c3cc(Cl)cc(Cl)c3)c2)CC1] | N-((1-((2-(3,5-dichlorophenyl)-6-((2- (8-methyl-3,8-diazabicyclo[3.2.1] octan-3- yl)pyrimidin-5-yl)oxy)pyridin-4- yl)methyl)piperidin-4- yl)methyl)acetamide |
| 72 | 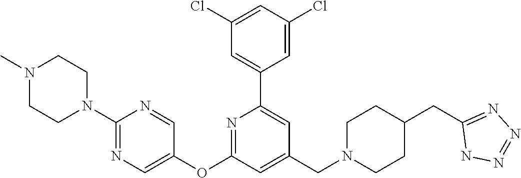 [SMILES: CN1CCN(c2ncc(Oc3cc(CN4CCC(Cc5nnn[nH]5)CC4)cc(-c4cc(Cl)cc(Cl)c4)n3)cn2)CC1] | 5-((4-((4-((1H-tetrazol-5- yl)methyl)piperidin-1-yl)methyl)-6- (3,5-dichlorophenyl)pyridin-2-yl) oxy)-2-(4- methylpiperazin-1-yl)pyrimidine |
| 73 | 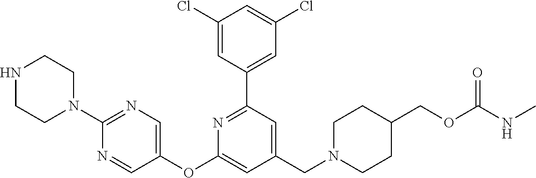 [SMILES: CNC(=O)OCC1CCN(Cc2cc(Oc3cnc(N4CCNCC4)nc3)nc(-c3cc(Cl)cc(Cl)c3)c2)CC1] | (1-((2-(3,5-dichlorophenyl)-6-((2- (piperazin-1-yl)pyrimidin-5- yl)oxy)pyridin-4-yl)methyl)piperidin-4- yl)methylmethylcarbamate |
| 74 | 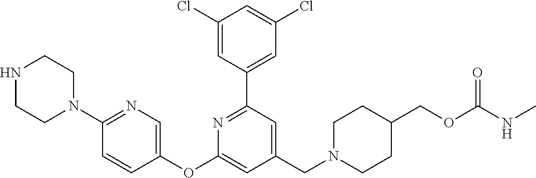 [SMILES: CNC(=O)OCC1CCN(Cc2cc(Oc3ccc(N4CCNCC4)nc3)nc(-c3cc(Cl)cc(Cl)c3)c2)CC1] | (1-((2-(3,5-dichlorophenyl)-6-((6- (piperazin-1-yl)pyridin-3-yl)oxy) pyridin-4-yl)methyl)piperidin-4- yl)methylmethylcarbamate |
| 75 | 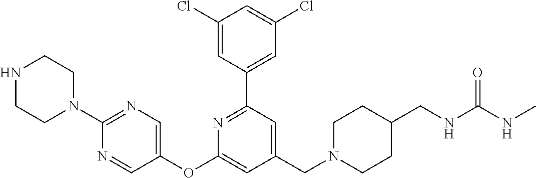 [SMILES: CNC(=O)NCC1CCN(Cc2cc(Oc3cnc(N4CCNCC4)nc3)nc(-c3cc(Cl)cc(Cl)c3)c2)CC1] | l-((1-((2-(3,5-dichlorophenyl)-6-((2- (piperazin-1-yl)pyrimidin-5- yl)oxy)pyridin-4-yl)methyl)piperidin- 4-yl)methyl)-3-methylurea |
| 76 | 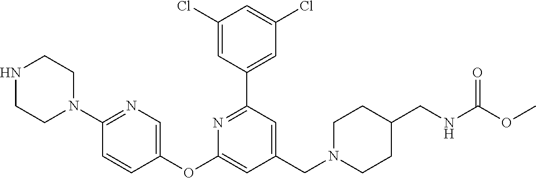 [SMILES: COC(=O)NCC1CCN(Cc2cc(Oc3ccc(N4CCNCC4)nc3)nc(-c3cc(Cl)cc(Cl)c3)c2)CC1] | methyl((1-((2-(3,5-dichlorophenyl)-6- ((6-(piperazin-1-yl)pyridin-3-yl)oxy) pyridin-4-yl)methyl)piperidin-4- yl)methyl)carbamate |
| 77 | 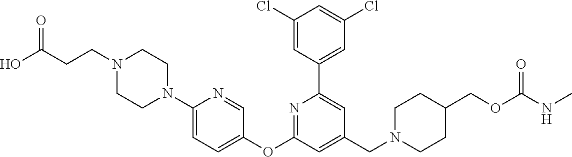 [SMILES: CNC(=O)OCC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)-4-((4- (((methylcarbamoyl)oxy)methyl) piperidin-1-yl)methyl)pyridin-2-yl) oxy)pyridin-2-yl)piperazin-1-yl) propanoic acid |
| 78 | 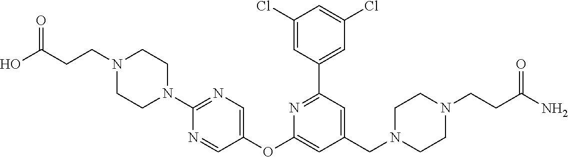 [SMILES: NC(=O)CCN1CCN(Cc2cc(Oc3cnc(N4CCN(CCC(=O)O)CC4)nc3)nc(-c3cc(Cl)cc(Cl)c3)c2)CC1] | 3-(4-(5-((4-((4-(3-amino-3- oxopropyl)piperazin-1-yl)methyl)-6-(3, 5-dichlorophenyl)pyridin-2- yl)oxy)pyrimidin-2-yl)piperazin-1- yl)propanoic acid |
| 79 | 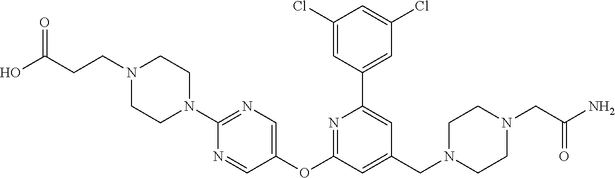 [SMILES: NC(=O)CN1CCN(Cc2cc(Oc3cnc(N4CCN(CCC(=O)O)CC4)nc3)nc(-c3cc(Cl)cc(Cl)c3)c2)CC1] | 3-(4-(5-((4-((4-(2-amino-2- oxoethyl)piperazin-1-yl)methyl)-6- (3,5-dichlorophenyl)pyridin-2- yl)oxy)pyrimidin-2-yl)piperazin-1- yl)propanoic acid |
| 80 | 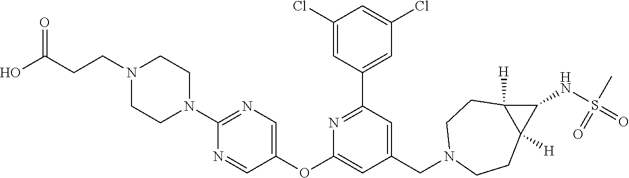 [SMILES: CS(=O)(=O)N[C@H]1[C@@H]2CCN(Cc3cc(Oc4cnc(N5CCN(CCC(=O)O)CC5)nc4)nc(-c4cc(Cl)cc(Cl)c4)c3)CC[C@@H]21] | 3-(4-(5-((6-(3,5-dichlorophenyl)-4- (((1R,7S,8r)-8-(methylsulfonamido)-4- azabicyclo[5.1.0]octan-4- yl)methyl)pyridin-2-yl)oxy)pyrimidin- 2-yl)piperazin-1-yl)propanoic acid |
| 81 | 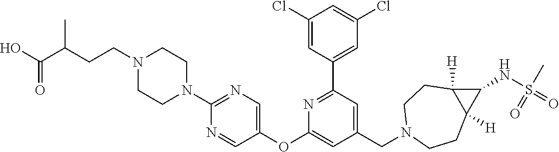 [SMILES: CC(CCN1CCN(c2ncc(Oc3cc(CN4CC[C@@H]5[C@H](CC4)[C@H]5NS(C)(=O)=O)cc(-c4cc(Cl)cc(Cl)c4)n3)cn2)CC1)C(=O)O] | 4-(4-(5-((6-(3,5-dichlorophenyl)-4- (((1R,7S,8r)-8-(methylsulfonamido)-4- azabicyclo[5.1.0]octan-4- yl)methyl)pyridin-2-yl)oxy)pyrimidin- 2-yl)piperazin-1-yl)-2-methylbutanoic acid |
| 82 | 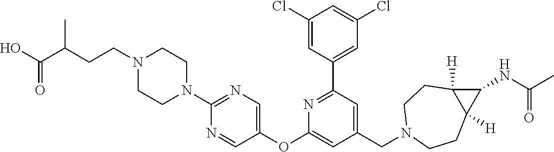 [SMILES: CC(=O)N[C@H]1[C@@H]2CCN(Cc3cc(Oc4cnc(N5CCN(CCC(C)C(=O)O)CC5)nc4)nc(-c4cc(Cl)cc(Cl)c4)c3)CC[C@@H]21] | 4-(4-(5-((4-(((1R,7S,8r)-8-acetamido-4- azabicyclo[5.1.0]octan-4-yl)methyl)-6- (3,5-dichlorophenyl)pyridin-2- yl)oxy)pyrimidin-2-yl)piperazin-1-yl)- 2-methylbutanoic acid |
| 83 | 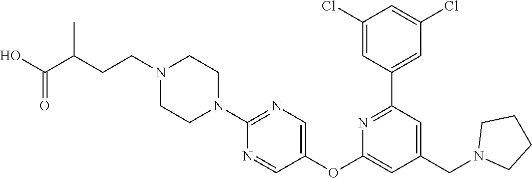 [SMILES: CC(CCN1CCN(c2ncc(Oc3cc(CN4CCCC4)cc(-c4cc(Cl)cc(Cl)c4)n3)cn2)CC1)C(=O)O] | 4-(4-(5-((6-(3,5-dichlorophenyl)-4- (pyrrolidin-1-ylmethyl)pyridin-2- yl)oxy)pyrimidin-2-yl)piperazin-1-yl)- 2-methylbutanoic acid |
| 84 | 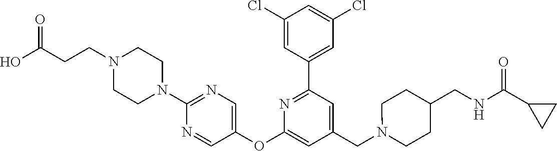 [SMILES: O=C(O)CCN1CCN(c2ncc(Oc3cc(CN4CCC(CNC(=O)C5CC5)CC4)cc(-c4cc(Cl)cc(Cl)c4)n3)cn2)CC1] | 3-(4-(5-((4-((4- (cyclopropanecarboxamidomethyl) piperidin-1-yl)methyl)-6-(3,5- dichlorophenyl)pyridin-2- yl)oxy)pyrimidin-2-yl)piperazin-1- yl)propanoic acid |
| 85 | 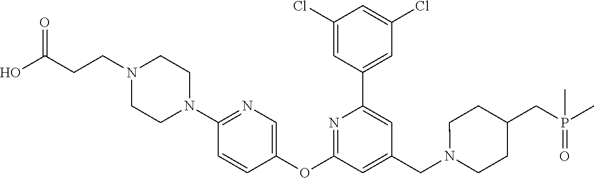 [SMILES: CP(C)(=O)CC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)-4-((4- ((dimethylphosphoryl)methyl)piperidin- 1-yl)methyl)pyridin-2-yl)oxy)pyridin- 2-yl)piperazin-1-yl)propanoic acid |
| 86 | 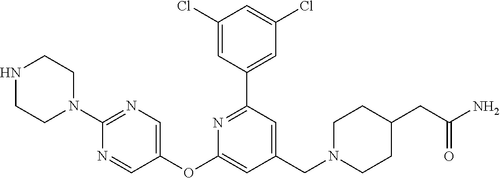 [SMILES: NC(=O)CC1CCN(Cc2cc(Oc3cnc(N4CCNCC4)nc3)nc(-c3cc(Cl)cc(Cl)c3)c2)CC1] | 2-(1-((2-(3,5-dichlorophenyl)-6-((2- (piperazin-1-yl)pyrimidin-5- yl)oxy)pyridin-4-yl)methyl)piperidin-4- yl)acetamide |
| 87 | 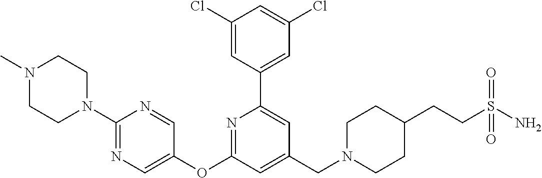 [SMILES: CN1CCN(c2ncc(Oc3cc(CN4CCC(CCS(N)(=O)=O)CC4)cc(-c4cc(Cl)cc(Cl)c4)n3)cn2)CC1] | 2-(1-((2-(3,5-dichlorophenyl)-6-((2-(4- methylpiperazin-1-yl)pyrimidin-5- yl)oxy)pyridin-4-yl)methyl)piperidin-4- yl)ethanesulfonamide |
| 88 | 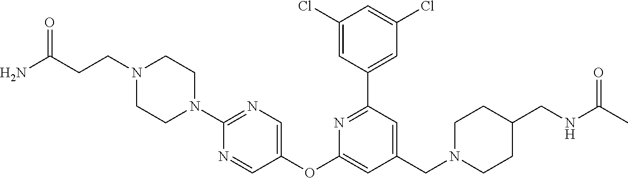 [SMILES: CC(=O)NCC1CCN(Cc2cc(Oc3cnc(N4CCN(CCC(N)=O)CC4)nc3)nc(-c3cc(Cl)cc(Cl)c3)c2)CC1] | 3-(4-(5-((4-((4- (acetamidomethyl)piperidin-1-yl) methyl)-6-(3,5-dichlorophenyl)pyridin- 2-yl)oxy)pyrimidin-2-yl)piperazin-1- yl)propanamide |
| 89 | 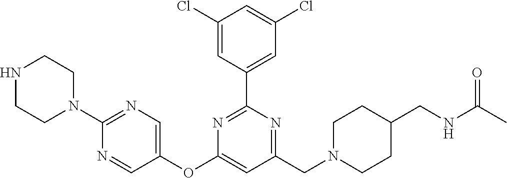 [SMILES: CC(=O)NCC1CCN(Cc2cc(Oc3cnc(N4CCNCC4)nc3)nc(-c3cc(Cl)cc(Cl)c3)n2)CC1] | N-((1-((2-(3,5-dichlorophenyl)-6-((6- (piperazin-1-yl)pyridin-3- yl)oxy)pyrimidin-4-yl)methyl) piperidin-4-yl)methyl)acetamide |
| 90 | 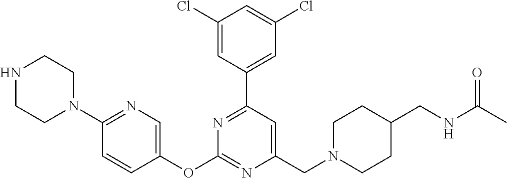 [SMILES: CC(=O)NCC1CCN(Cc2cc(-c3cc(Cl)cc(Cl)c3)nc(Oc3ccc(N4CCNCC4)nc3)n2)CC1] | N-((1-((6-(3,5-dichlorophenyl)-2-((6- (piperazin-1-yl)pyridin-3- yl)oxy)pyrimidin-4-yl)methyl) piperidin-4-yl)methyl)acetamide |
| 91 | 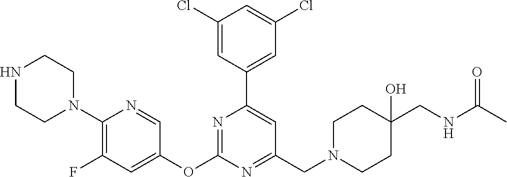 [SMILES: CC(=O)NCC1(O)CCN(Cc2cc(-c3cc(Cl)cc(Cl)c3)nc(Oc3cnc(N4CCNCC4)c(F)c3)n2)CC1] | N-((1-((2-(3,5-dichlorophenyl)-6-((5- fluoro-6-(piperazin-1-yl)pyridin-3- yl)oxy)pyridin-4-yl)methyl)-4- hydroxypiperidin-4-yl)methyl) acetamide |
| 92 | 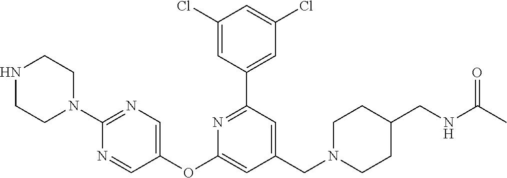 [SMILES: CC(=O)NCC1CCN(Cc2cc(Oc3cnc(N4CCNCC4)nc3)nc(-c3cc(Cl)cc(Cl)c3)c2)CC1] | N-((1-((2-(3,5-dichlorophenyl)-6-((2- (piperazin-1-yl)pyrimidin-5- yl)oxy)pyridin-4-yl)methyl)piperidin-4- yl)methyl)acetamide |
| 93 | 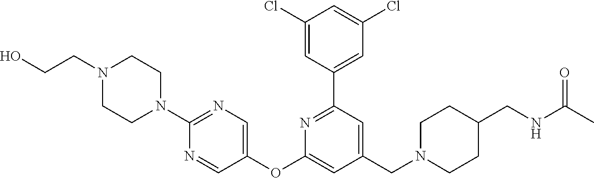 [SMILES: CC(=O)NCC1CCN(Cc2cc(Oc3cnc(N4CCN(CCO)CC4)nc3)nc(-c3cc(Cl)cc(Cl)c3)c2)CC1] | N-((1-((2-(3,5-dichlorophenyl)-6-((2- (4-(2-hydroxyethyl)piperazin-1-yl) pyrimidin-5-yl)oxy)pyridin-4-yl) methyl) piperidin-4-yl)methyl)acetamide |
| 94 | 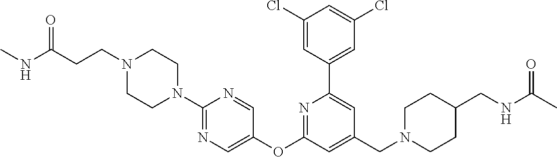 [SMILES: CNC(=O)CCN1CCN(c2ncc(Oc3cc(CN4CCC(CNC(C)=O)CC4)cc(-c4cc(Cl)cc(Cl)c4)n3)cn2)CC1] | 3-(4-(5-((4-((4- (acetamidomethyl)piperidin-1-yl) methyl)-6-(3,5-dichlorophenyl)pyridin- 2-yl)oxy)pyrimidin-2-yl)piperazin-1- yl)-N-methylpropanamide |
| 95 | 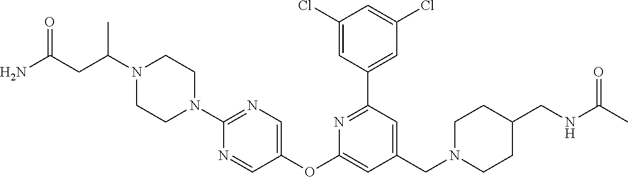 [SMILES: CC(=O)NCC1CCN(Cc2cc(Oc3cnc(N4CCN(C(C)CC(N)=O)CC4)nc3)nc(-c3cc(Cl)cc(Cl)c3)c2)CC1] | 3-(4-(5-((4-(4- (acetamidomethyl)piperidin-1-yl) methyl)-6-(3,5-dichlorophenyl) pyridin-2-yl)oxy)pyrimidin-2-yl) piperazin-1-yl)butanamide |
| 96 | 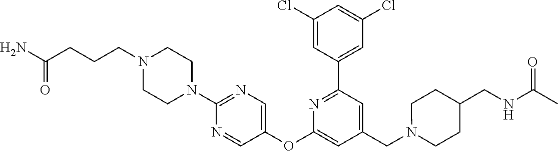 [SMILES: CC(=O)NCC1CCN(Cc2cc(Oc3cnc(N4CCN(CCCC(N)=O)CC4)nc3)nc(-c3cc(Cl)cc(Cl)c3)c2)CC1] | 4-(4-(5-((4-((4- (acetamidomethyl)piperidin-1-yl) methyl)-6-(3,5-dichlorophenyl) pyridin-2-yl)oxy)pyrimidin-2-yl) piperazin-1-yl)butanamide |
| 97 | 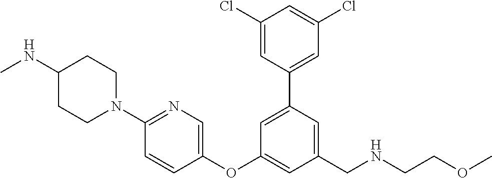 | 1-(5-((3′,5′-dichloro-5-(((2- methoxyethyl)amino)methyl)-[1,1′- biphenyl]-3-yl)oxy)pyridin-2-yl)-N- methylpiperidin-4-amine |
| 98 | 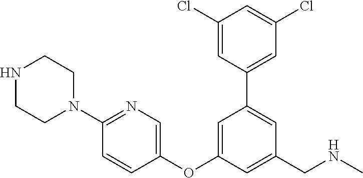 [SMILES: CNCc1cc(Oc2ccc(N3CCNCC3)nc2)cc(-c2cc(Cl)cc(Cl)c2)c1] | 1-(3′,5′-dichloro-5-((6-(piperazin-1- yl)pyridin-3-yl)oxy)-[1,1′-biphenyl]-3- yl)-N-methylmethanamine |
| 99 | 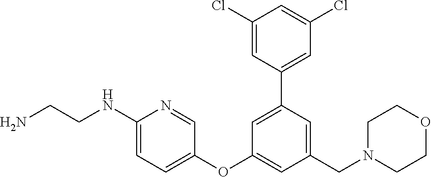 [SMILES: NCCNc1ccc(Oc2cc(CN3CCOCC3)cc(-c3cc(Cl)cc(Cl)c3)c2)cn1] | N1-(5-((3′,5′-dichloro-5- (morpholinomethyl)-[1,1′-biphenyl]-3- yl)oxy)pyridin-2-yl)ethane-1,2- diamine |
| 100 | 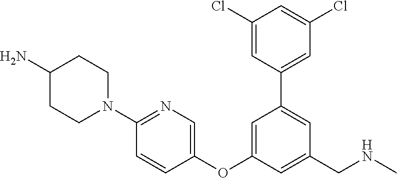 [SMILES: CNCc1cc(Oc2ccc(N3CCC(N)CC3)nc2)cc(-c2cc(Cl)cc(Cl)c2)c1] | 1-(5-((3′,5′-dichloro-5- ((methylamino)methyl)-[1,1′-biphenyl]- 3-yl)oxy)pyridin-2-yl)piperidin-4- amine |
| 101 | 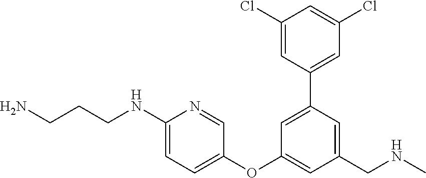 [SMILES: CNCc1cc(Oc2ccc(NCCCN)nc2)cc(-c2cc(Cl)cc(Cl)c2)c1] | N1-(5-((3′,5′-dichloro-5- ((methylamino)methyl)-[1,1′-biphenyl]- 3-yl)oxy)pyridin-2-yl)propane-1,3- diamine |
| 102 | 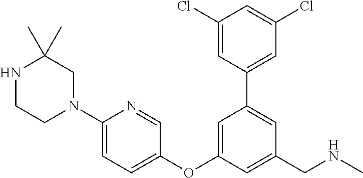 [SMILES: CNCc1cc(Oc2ccc(N3CCNC(C)(C)C3)nc2)cc(-c2cc(Cl)cc(Cl)c2)c1] | 1-(3′,5′-dichloro-5-((6-(3,3- dimethylpiperazin-1-yl)pyridin-3-yl) oxy)-[1,1′-biphenyl]-3-yl)-N- methylmethanamine |
| 103 | 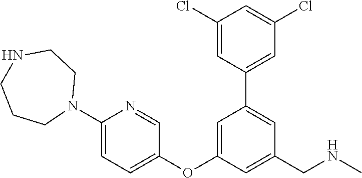 [SMILES: CNCc1cc(Oc2ccc(N3CCCNCC3)nc2)cc(-c2cc(Cl)cc(Cl)c2)c1] | 1-(5-((6-(1,4-diazepan-1-yl)pyridin-3- yl)oxy)-3′,5′-dichloro-[1,1′-biphenyl]- 3-yl)-N-methylmethanamine |
| 104 | 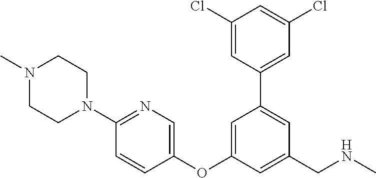 | 1-(3′,5′-dichloro-5-((6-(4- methylpiperazin-l-yl)pyridin-3-yl) oxy)-[1,1′-biphenyl]-3- yl)-N-methylmethanamine |
| 105 | 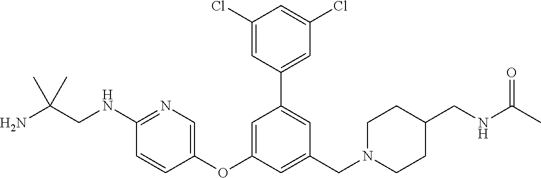 [SMILES: CC(=O)NCC1CCN(Cc2cc(Oc3ccc(NCC(C)(C)N)nc3)cc(-c3cc(Cl)cc(Cl)c3)c2)CC1] | N-((1-((5-((6-((2-amino-2- methylpropyl)amino)pyridin-3-yl)oxy)- 3′,5′-dichloro-[1,1′-biphenyl]-3- yl)methyl)piperidin-4- yl)methyl)acetamide |
| 106 | 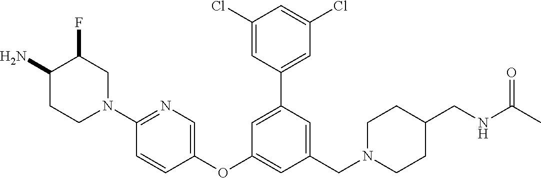 [SMILES: CC(=O)NCC1CCN(Cc2cc(Oc3ccc(N4CC[C@@H](N)[C@@H](F)C4)nc3)cc(-c3cc(Cl)cc(Cl)c3)c2)CC1] | N-((1-((5-((6-((3S,4R)-4-amino-3- fluoropiperidin-1-yl)pyridin-3-yl)oxy)- 3′,5′-dichloro-[1,1′-biphenyl]-3- yl)methyl)piperidin-4- yl)methyl)acetamide |
| 107 | 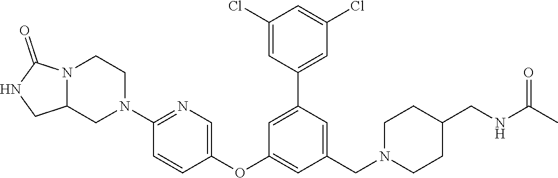 [SMILES: CC(=O)NCC1CCN(Cc2cc(Oc3ccc(N4CCN5C(=O)NCC5C4)nc3)cc(-c3cc(Cl)cc(Cl)c3)c2)CC1] | N-((1-((3′,5′-dichloro-5-((6-(3- oxohexahydroimidazo[1,5-a]pyrazin- 7(1H)-yl)pyridin-3-yl)oxy)-[1,1′- biphenyl]-3-yl)methyl)piperidin-4- yl)methyl)acetamide |
| 108 | 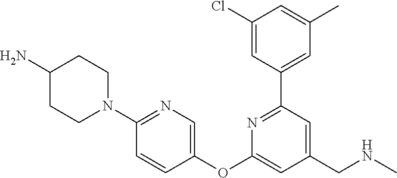 [SMILES: CNCc1cc(Oc2ccc(N3CCC(N)CC3)nc2)nc(-c2cc(C)cc(Cl)c2)c1] | 1-(5-((6-(3-chloro-5-methylphenyl)-4- ((methylamino)methyl)pyridin-2- yl)oxy)pyridin-2-yl)piperidin-4-amine |
| 109 | 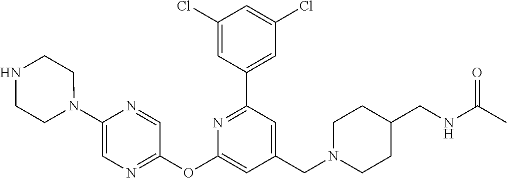 [SMILES: CC(=O)NCC1CCN(Cc2cc(Oc3cnc(N4CCNCC4)cn3)nc(-c3cc(Cl)cc(Cl)c3)c2)CC1] | N-((1-((2-(3,5-dichlorophenyl)-6-((5- (piperazin-1-yl)pyrazin-2-yl)oxy) pyridin-4-yl)methyl)piperidin-4- yl)methyl)acetamide |
| 110 | 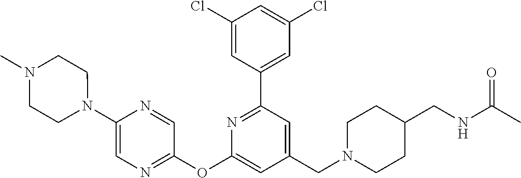 [SMILES: CC(=O)NCC1CCN(Cc2cc(Oc3cnc(N4CCN(C)CC4)cn3)nc(-c3cc(Cl)cc(Cl)c3)c2)CC1] | N-((1-((2-(3,5-dichlorophenyl)-6-((5- (4-methylpiperazin-1-yl)pyrazin-2- yl)oxy)pyridin-4-yl)methyl)piperidin- 4-yl)methyl)acetamide |
| 111 | 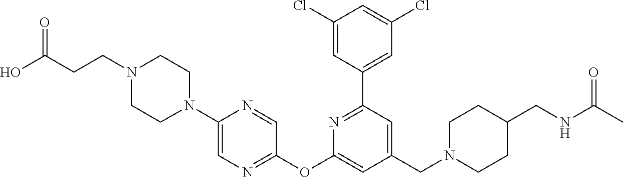 [SMILES: CC(=O)NCC1CCN(Cc2cc(Oc3cnc(N4CCN(CCC(=O)O)CC4)cn3)nc(-c3cc(Cl)cc(Cl)c3)c2)CC1] | 3-(4-(5-((4-(4- (acetamidomethyl)piperidin-1-yl) methyl)-6-(3,5-dichlorophenyl) pyridin-2-yl)oxy)pyrazin-2-yl) piperazin-1-yl)propanoic acid |
| 112 | 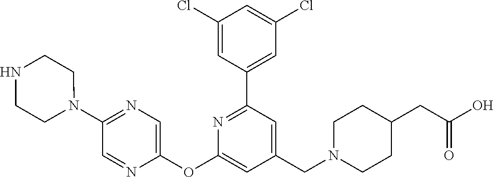 [SMILES: O=C(O)CC1CCN(Cc2cc(Oc3cnc(N4CCNCC4)cn3)nc(-c3cc(Cl)cc(Cl)c3)c2)CC1] | 2-(1-((2-(3,5-dichlorophenyl)-6-((5- (piperazin-1-yl)pyrazin-2-yl)oxy) pyridin-4-yl)methyl)piperidin-4-yl) acetic acid |
| 113 | 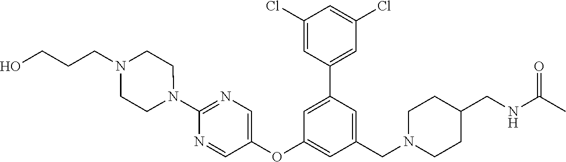 [SMILES: CC(=O)NCC1CCN(Cc2cc(Oc3cnc(N4CCN(CCCO)CC4)nc3)cc(-c3cc(Cl)cc(Cl)c3)c2)CC1] | N-((1-((3′,5′-dichloro-5-((2-(4-(3- hydroxypropyl)piperazin-1-yl) pyrimidin-5-yl)oxy)-[1,1′-biphenyl]-3- yl)methyl)piperidin-4- yl)methyl)acetamide |
| 114 | 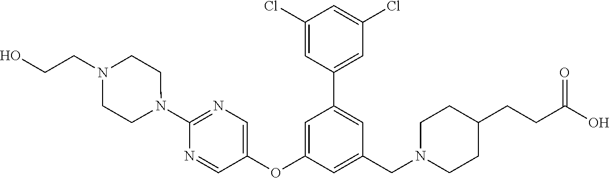 [SMILES: O=C(O)CCC1CCN(Cc2cc(Oc3cnc(N4CCN(CCO)CC4)nc3)cc(-c3cc(Cl)cc(Cl)c3)c2)CC1] | 3-(1-((3′,5′-dichloro-5-((2-(4-(2- hydroxyethyl)piperazin-1-yl)pyrimidin- 5-yl)oxy)-[1,1′-biphenyl]-3- yl)methyl)piperidin-4-yl)propanoic acid |
| 115 | 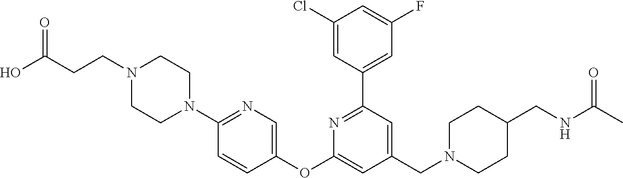 [SMILES: CC(=O)NCC1CCN(Cc2cc(Oc3ccc(N4CCN(CCC(=O)O)CC4)nc3)nc(-c3cc(F)cc(Cl)c3)c2)CC1] | 3-(4-(5-((4-((4- (acetamidomethyl)piperidin-1-yl) methyl)-6-(3-chloro-5-fluorophenyl) pyridin-2-yl)oxy)pyridin-2-yl) piperazin-1-yl)propanoic acid |
| 116 | 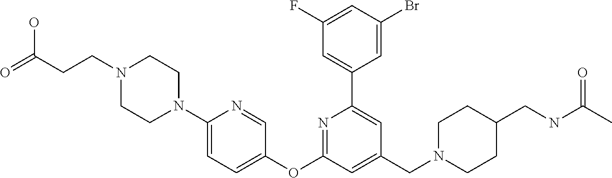 [SMILES: CC(=O)NCC1CCN(Cc2cc(Oc3ccc(N4CCN(CCC(=O)O)CC4)nc3)nc(-c3cc(F)cc(Br)c3)c2)CC1] | 3-(4-(5-((4-(4- (acetamidomethyl)piperidin-1-yl) methyl)-6-(3-bromo-5-fluorophenyl) pyridin-2-yl)oxy)pyridin-2-yl) piperazin-1-yl)propanoic acid |
| 117 | 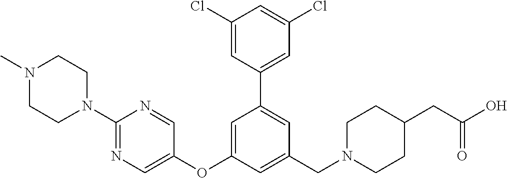 [SMILES: CN1CCN(c2ncc(Oc3cc(CN4CCC(CC(=O)O)CC4)cc(-c4cc(Cl)cc(Cl)c4)c3)cn2)CC1] | 2-(1-((3′,5′-dichloro-5-((2-(4- methylpiperazin-1-yl)pyrimidin-5-yl) oxy)-[1,1′-biphenyl]-3-yl)methyl) piperidin-4-yl)acetic acid |
| 118 | 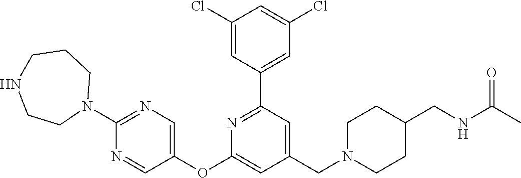 [SMILES: CC(=O)NCC1CCN(Cc2cc(Oc3cnc(N4CCCNCC4)nc3)nc(-c3cc(Cl)cc(Cl)c3)c2)CC1] | N-((1-((2-((2-(1,4-diazepan-1- yl)pyrimidin-5-yl)oxy)-6-(3,5- dichlorophenyl)pyridin-4- yl)methyl)piperidin-4- yl)methyl)acetamide |
| 119 | 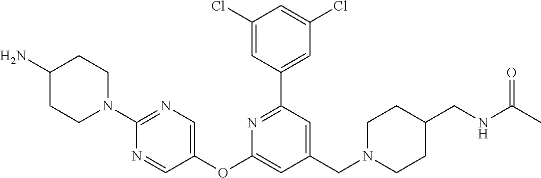 [SMILES: CC(=O)NCC1CCN(Cc2cc(Oc3cnc(N4CCC(N)CC4)nc3)nc(-c3cc(Cl)cc(Cl)c3)c2)CC1] | N-((1-((2-((2-(4-aminopiperidin-1- yl)pyrimidin-5-yl)oxy)-6-(3,5- dichlorophenyl)pyridin-4- yl)methyl)piperidin-4- yl)methyl)acetamide |
| 120 | 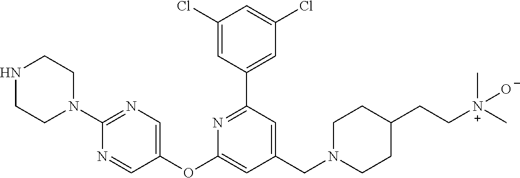 [SMILES: C[N+](C)([O-])CCC1CCN(Cc2cc(Oc3cnc(N4CCNCC4)nc3)nc(-c3cc(Cl)cc(Cl)c3)c2)CC1] | 2-(1-((2-(3,5-dichlorophenyl)-6-((6- (piperazin-1-yl)pyridin-3-yl)oxy) pyridin-4-yl)methyl)piperidin-4-yl)- N,N-dimethylethanamineoxide |
| 121 | 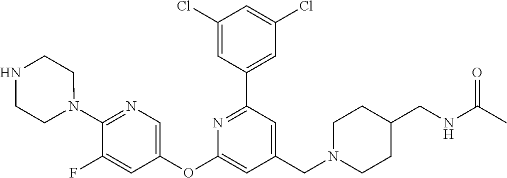 [SMILES: CC(=O)NCC1CCN(Cc2cc(Oc3cnc(N4CCNCC4)c(F)c3)nc(-c3cc(Cl)cc(Cl)c3)c2)CC1] | N-((1-((2-(3,5-dichlorophenyl)-6-((5- fluoro-6-(piperazin-1-yl)pyridin-3- yl)oxy)pyridin-4-yl)methyl)piperidin- 4-yl)methyl)acetamide |
| 122 | 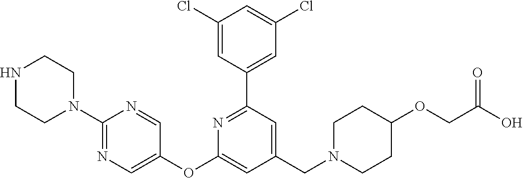 [SMILES: O=C(O)COC1CCN(Cc2cc(Oc3cnc(N4CCNCC4)nc3)nc(-c3cc(Cl)cc(Cl)c3)c2)CC1] | 2-((1-((2-(3,5-dichlorophenyl)-6-((2- (piperazin-1-yl)pyrimidin-5- yl)oxy)pyridin-4-yl)methyl)piperidin- 4-yl)oxy)acetic acid |
| 123 | 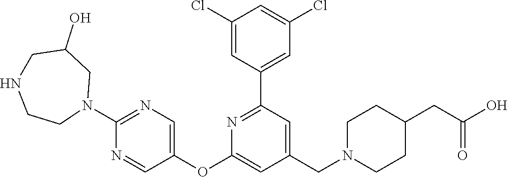 [SMILES: O=C(O)CC1CCN(Cc2cc(Oc3cnc(N4CCNCC(O)C4)nc3)nc(-c3cc(Cl)cc(Cl)c3)c2)CC1] | 2-(1-((2-(3,5-dichlorophenyl)-6-((2-(6- hydroxy-1,4-diazepan-1-yl)pyrimidin- 5-yl)oxy)pyridin-4-yl)methyl)piperidin- 4-yl)acetic acid |
| 124 | 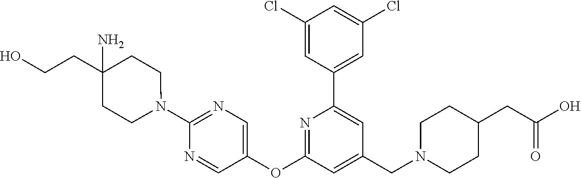 [SMILES: NC1(CCO)CCN(c2ncc(Oc3cc(CN4CCC(CC(=O)O)CC4)cc(-c4cc(Cl)cc(Cl)c4)n3)cn2)CC1] | 2-(1-((2-((2-(4-amino-4-(2- hydroxyethyl)piperidin-1-yl)pyrimidin- 5-yl)oxy)-6-(3,5-dichlorophenyl) pyridin-4-yl)methyl)piperidin-4-yl) acetic acid |
| 125 | 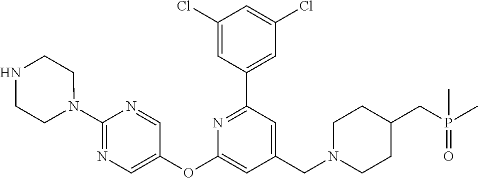 [SMILES: CP(C)(=O)CC1CCN(Cc2cc(Oc3cnc(N4CCNCC4)nc3)nc(-c3cc(Cl)cc(Cl)c3)c2)CC1] | ((1-((2-(3,5-dichlorophenyl)-6-((2- (piperazin-1-yl)pyrimidin-5- yl)oxy)pyridin-4-yl)methyl)piperidin-4- yl)methyl)dimethylphosphineoxide |
| 126 | 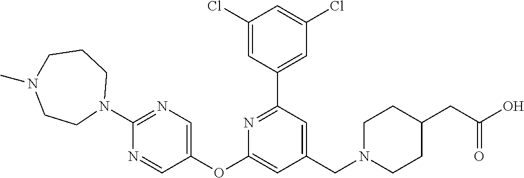 [SMILES: CN1CCCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2-(4- methyl-1,4-diazepan-1-yl)pyrimidin-5- yl)oxy)pyridin-4-yl)methyl)piperidin-4- yl)acetic acid |
| 127 | 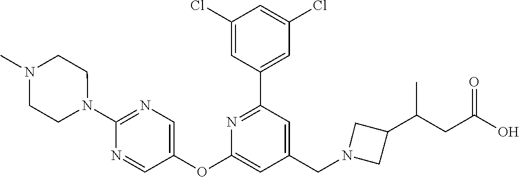 [SMILES: CC(CC(=O)O)C1CN(Cc2cc(Oc3cnc(N4CCN(C)CC4)nc3)nc(-c3cc(Cl)cc(Cl)c3)c2)C1] | 3-(1-((2-(3,5-dichlorophenyl)-6-((2-(4- methylpiperazin-1-yl)pyrimidin-5- yl)oxy)pyridin-4-yl)methyl)azetidin-3- yl)butanoic acid |
| 128 | 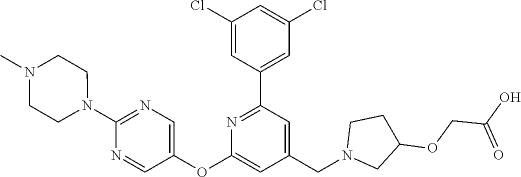 [SMILES: CN1CCN(c2ncc(Oc3cc(CN4CCC(OCC(=O)O)C4)cc(-c4cc(Cl)cc(Cl)c4)n3)cn2)CC1] | 2-((1-((2-(3,5-dichlorophenyl)-6-((2-(4- methylpiperazin-1-yl)pyrimidin-5- yl)oxy)pyridin-4-yl)methyl)pyrrolidin-3- yl)oxy)acetic acid |
| 129 | 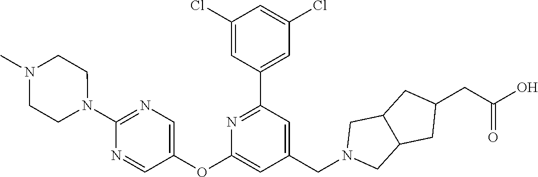 [SMILES: CN1CCN(c2ncc(Oc3cc(CN4CC5CC(CC(=O)O)CC5C4)cc(-c4cc(Cl)cc(Cl)c4)n3)cn2)CC1] | 2-(2-((2-(3,5-dichlorophenyl)-6-((2-(4- methylpiperazin-1-yl)pyrimidin-5- yl)oxy)pyridin-4- yl)methyl)octahydrocyclopenta[c] pyrrol-5-yl)acetic acid |
| 130 | 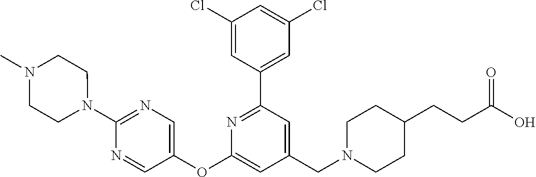 [SMILES: CN1CCN(c2ncc(Oc3cc(CN4CCC(CCC(=O)O)CC4)cc(-c4cc(Cl)cc(Cl)c4)n3)cn2)CC1] | 3-(1-((2-(3,5-dichlorophenyl)-6-((2-(4- methylpiperazin-1-yl)pyrimidin-5- yl)oxy)pyridin-4-yl)methyl)piperidin-4- yl)propanoic acid |
| 131 | 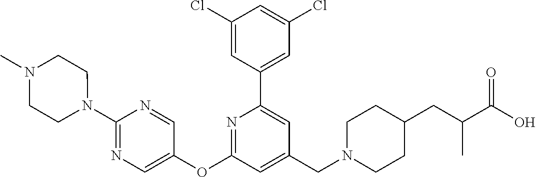 [SMILES: CC(CC1CCN(Cc2cc(Oc3cnc(N4CCN(C)CC4)nc3)nc(-c3cc(Cl)cc(Cl)c3)c2)CC1)C(=O)O] | 3-(1-((2-(3,5-dichlorophenyl)-6-((2-(4- methylpiperazin-1-yl)pyrimidin-5- yl)oxy)pyridin-4-yl)methyl)piperidin-4- yl)-2-methylpropanoic acid |
| 132 | 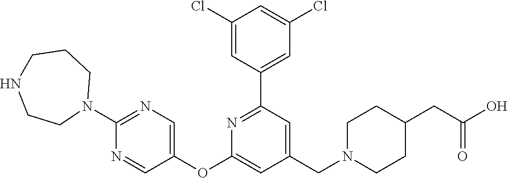 [SMILES: O=C(O)CC1CCN(Cc2cc(Oc3cnc(N4CCCNCC4)nc3)nc(-c3cc(Cl)cc(Cl)c3)c2)CC1] | 2-(1-((2-((2-(1,4-diazepan-1-yl) pyrimidin-5-yl)oxy)-6-(3,5- dichlorophenyl)pyridin- 4-yl)methyl)piperidin-4-yl) acetic acid |
| 133 | 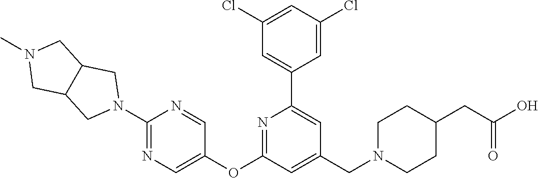 [SMILES: CN1CC2CN(c3ncc(Oc4cc(CN5CCC(CC(=O)O)CC5)cc(-c5cc(Cl)cc(Cl)c5)n4)cn3)CC2C1] | 2-(1-((2-(3,5-dichlorophenyl)-6-((2-(5- methylhexahydropyrrolo[3,4-c]pyrrol- 2(1H)-yl)pyrimidin-5-yl)oxy)pyridin-4- yl)methyl)piperidin-4-yl)acetic acid |
| 134 | 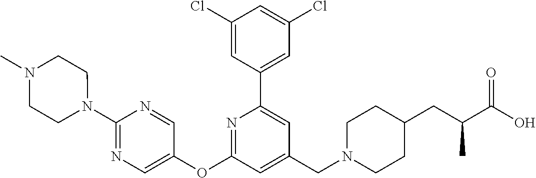 [SMILES: C[C@@H](CC1CCN(Cc2cc(Oc3cnc(N4CCN(C)CC4)nc3)nc(-c3cc(Cl)cc(Cl)c3)c2)CC1)C(=O)O] | (S)-3-(1-((2-(3,5-dichlorophenyl)-6-((2- (4-methylpiperazin-1-yl)pyrimidin-5- yl)oxy)pyridin-4-yl)methyl)piperidin-4- yl)-2-methylpropanoic acid |
| 135 | 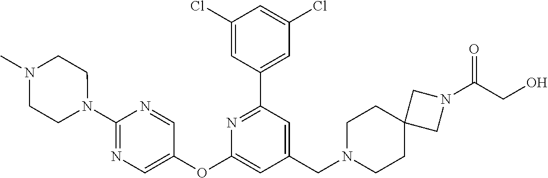 [SMILES: CN1CCN(c2ncc(Oc3cc(CN4CCC5(CC4)CN(C(=O)CO)C5)cc(-c4cc(Cl)cc(Cl)c4)n3)cn2)CC1] | 1-(7-((2-(3,5-dichlorophenyl)-6-((2-(4- methylpiperazin-1-yl)pyrimidin-5- yl)oxy)pyridin-4-yl)methyl)-2,7- diazaspiro[3.5]nonan-2-yl)-2- hydroxyethanone |
| 136 | 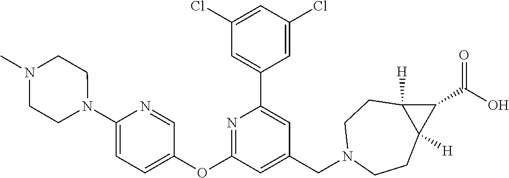 [SMILES: CN1CCN(c2ccc(Oc3cc(CN4CC[C@@H]5[C@H](CC4)[C@H]5C(=O)O)cc(-c4cc(Cl)cc(Cl)c4)n3)cn2)CC1] | (1R,7S,8r)-4-((2-(3,5-dichlorophenyl)- 6-((6-(4-methylpiperazin-1-yl)pyridin- 3-yl)oxy)pyridin-4-yl)methyl)-4- azabicyclo[5.1.0]octane-8-carboxylic acid |
| 137 | 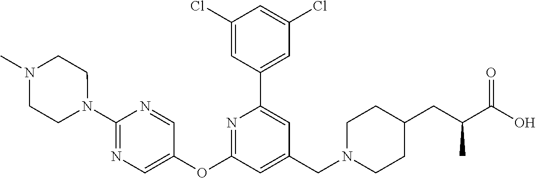 [SMILES: C[C@@H](CC1CCN(Cc2cc(Oc3cnc(N4CCN(C)CC4)nc3)nc(-c3cc(Cl)cc(Cl)c3)c2)CC1)C(=O)O] | (R)-3-(1-((2-(3,5-dichlorophenyl)-6-((2- (4-methylpiperazin-1-yl)pyrimidin-5- yl)oxy)pyridin-4-yl)methyl)piperidin-4- yl)-2-methylpropanoic acid |
| 138 | 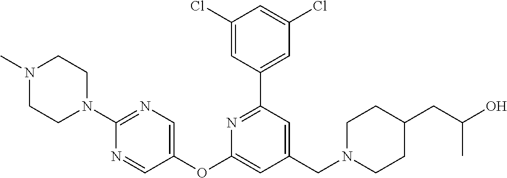 [SMILES: CC(O)CC1CCN(Cc2cc(Oc3cnc(N4CCN(C)CC4)nc3)nc(-c3cc(Cl)cc(Cl)c3)c2)CC1] | 1-(1-((2-(3,5-dichlorophenyl)-6-((2-(4- methylpiperazin-1-yl)pyrimidin-5- yl)oxy)pyridin-4-yl)methyl)piperidin-4- yl)propan-2-ol |
| 139 | 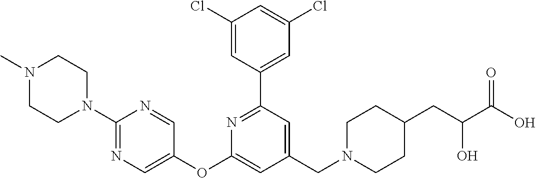 [SMILES: CN1CCN(c2ncc(Oc3cc(CN4CCC(CC(O)C(=O)O)CC4)cc(-c4cc(Cl)cc(Cl)c4)n3)cn2)CC1] | 3-(1-((2-(3,5-dichlorophenyl)-6-((2-(4- methylpiperazin-1-yl)pyrimidin-5- yl)oxy)pyridin-4-yl)methyl)piperidin-4- yl)-2-hydroxypropanoic acid |
| 140 | 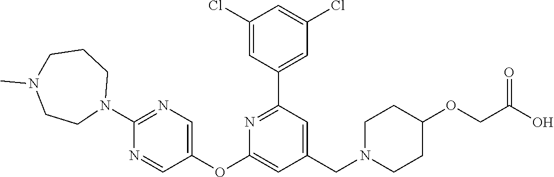 [SMILES: CN1CCCN(c2ncc(Oc3cc(CN4CCC(OCC(=O)O)CC4)cc(-c4cc(Cl)cc(Cl)c4)n3)cn2)CC1] | 2-((1-((2-(3,5-dichlorophenyl)-6-((2-(4- methyl-1,4-diazepan-1-yl)pyrimidin-5- yl)oxy)pyridin-4-yl)methyl)piperidin-4- yl)oxy)acetic acid |
| 141 | 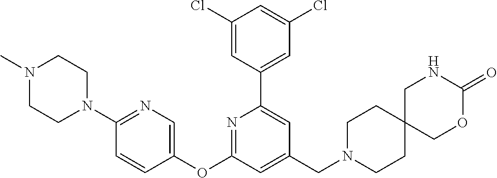 [SMILES: CN1CCN(c2ccc(Oc3cc(CN4CCC5(CC4)CNC(=O)OC5)cc(-c4cc(Cl)cc(Cl)c4)n3)cn2)CC1] | 9-((2-(3,5-dichlorophenyl)-6-((6-(4- methylpiperazin-1-yl)pyridin-3- yl)oxy)pyridin-4-yl)methyl)-2-oxa-4,9- diazaspiro[5.5]undecan-3-one |
| 142 | 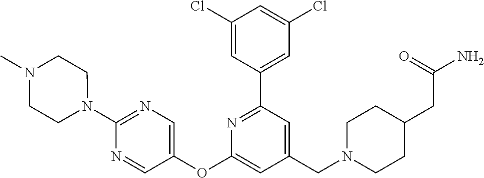 [SMILES: CN1CCN(c2ncc(Oc3cc(CN4CCC(CC(N)=O)CC4)cc(-c4cc(Cl)cc(Cl)c4)n3)cn2)CC1] | 2-(1-((2-(3,5-dichlorophenyl)-6-((2-(4- methylpiperazin-1-yl)pyrimidin-5- yl)oxy)pyridin-4-yl)methyl)piperidin-4- yl)acetamide |
| 143 | 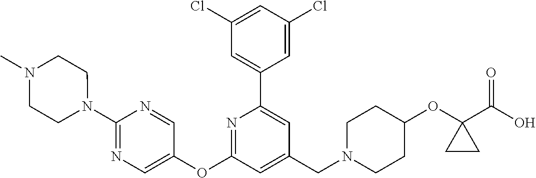 [SMILES: CN1CCN(c2ncc(Oc3cc(CN4CCC(OC5(C(=O)O)CC5)CC4)cc(-c4cc(Cl)cc(Cl)c4)n3)cn2)CC1] | 1-((1-((2-(3,5-dichlorophenyl)-6-((2-(4- methylpiperazin-1-yl)pyrimidin-5- yl)oxy)pyridin-4-yl)methyl)piperidin-4- yl)oxy)cyclopropanecarboxylic acid |
| 144 | 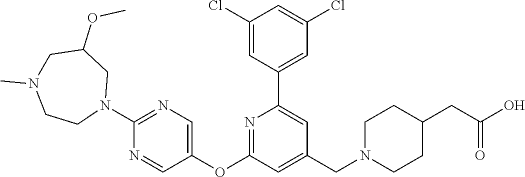 [SMILES: COC1CN(C)CCN(c2ncc(Oc3cc(CN4CCC(CC(=O)O)CC4)cc(-c4cc(Cl)cc(Cl)c4)n3)cn2)C1] | 2-(1-((2-(3,5-dichlorophenyl)-6-((2-(6- methoxy-4-methyl-1,4-diazepan-1- yl)pyrimidin-5-yl)oxy)pyridin-4- yl)methyl)piperidin-4-yl)acetic acid |
| 145 | 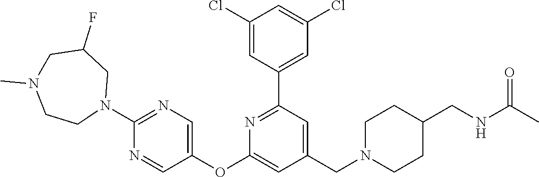 [SMILES: CC(=O)NCC1CCN(Cc2cc(Oc3cnc(N4CCN(C)CC(F)C4)nc3)nc(-c3cc(Cl)cc(Cl)c3)c2)CC1] | N-((1-((2-(3,5-dichlorophenyl)-6-((6- (6-fluoro-4-methyl-1,4-diazepan-1- yl)pyridin-3-yl)oxy)pyridin-4- yl)methyl)piperidin-4- yl)methyl)acetamide |
| 146 | 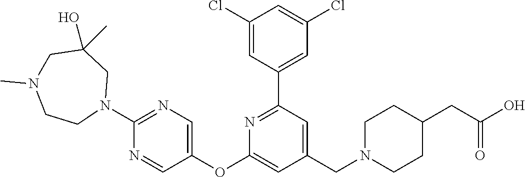 [SMILES: CN1CCN(c2ncc(Oc3cc(CN4CCC(CC(=O)O)CC4)cc(-c4cc(Cl)cc(Cl)c4)n3)cn2)CC(C)(O)C1] | 2-(1-((2-(3,5-dichlorophenyl)-6-((2-(6- hydroxy-4,6-dimethyl-1,4-diazepan-1- yl)pyrimidin-5-yl)oxy)pyridin-4- yl)methyl)piperidin-4-yl)acetic acid |
| 147 | 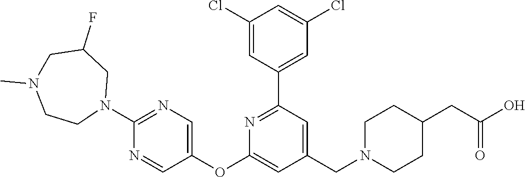 [SMILES: CN1CCN(c2ncc(Oc3cc(CN4CCC(CC(=O)O)CC4)cc(-c4cc(Cl)cc(Cl)c4)n3)cn2)CC(F)C1] | 2-(1-((2-(3,5-dichlorophenyl)-6-((2-(6- fluoro-4-methyl-1,4-diazepan-1- yl)pyrimidin-5-yl)oxy)pyridin-4- yl)methyl)piperidin-4-yl)acetic acid |
| 148 | 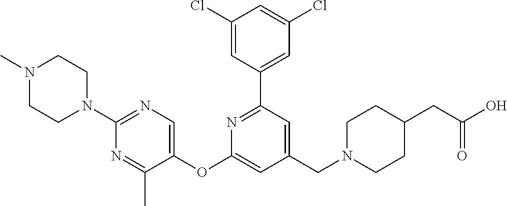 [SMILES: Cc1nc(N2CCN(C)CC2)ncc1Oc1cc(CN2CCC(CC(=O)O)CC2)cc(-c2cc(Cl)cc(Cl)c2)n1] | 2-(1-((2-(3,5-dichlorophenyl)-6-((4- methyl-2-(4-methylpiperazin-1- yl)pyrimidin-5-yl)oxy)pyridin-4- yl)methyl)piperidin-4-yl)acetic acid |
| 149 | 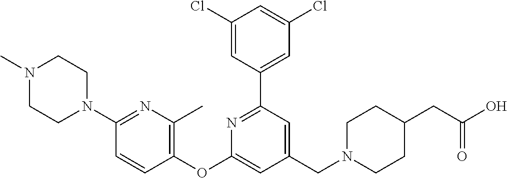 [SMILES: Cc1nc(N2CCN(C)CC2)ccc1Oc1cc(CN2CCC(CC(=O)O)CC2)cc(-c2cc(Cl)cc(Cl)c2)n1] | 2-(1-((2-(3,5-dichlorophenyl)-6-((2- methyl-6-(4-methylpiperazin-1-yl) pyridin-3-yl)oxy)pyridin-4-yl)methyl) piperidin-4-yl)acetic acid |
| 150 | 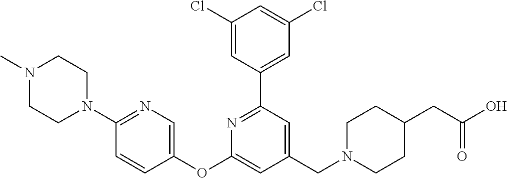 [SMILES: CN1CCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)-6-(4-(4- methylpiperazin-1-yl)phenoxy)pyridin- 4-yl)methyl)piperidin-4-yl)acetic acid |
| 151 | 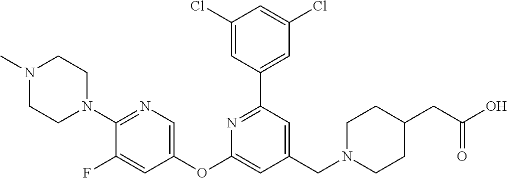 [SMILES: CN1CCN(c2ncc(Oc3cc(CN4CCC(CC(=O)O)CC4)cc(-c4cc(Cl)cc(Cl)c4)n3)cc2F)CC1] | 2-(1-((2-(3,5-dichlorophenyl)-6-((5- fluoro-6-(4-methylpiperazin-1-yl) pyridin-3-yl)oxy)pyridin-4-yl)methyl) piperidin-4-yl)acetic acid |
| 152 | 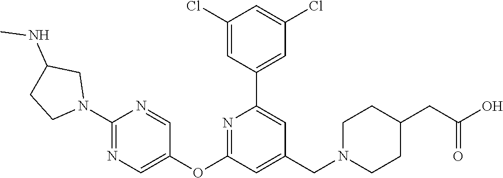 [SMILES: CNC1CCN(c2ncc(Oc3cc(CN4CCC(CC(=O)O)CC4)cc(-c4cc(Cl)cc(Cl)c4)n3)cn2)C1] | 2-(1-((2-(3,5-dichlorophenyl)-6-((2-(3- (methylamino)pyrrolidin-1-yl) pyrimidin-5-yl)oxy)pyridin-4-yl) methyl)piperidin-4-yl)acetic acid |
| 153 | 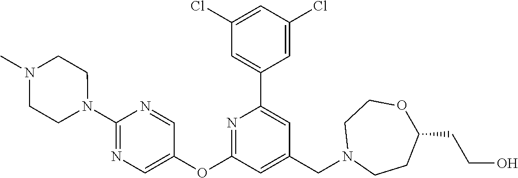 [SMILES: CN1CCN(c2ncc(Oc3cc(CN4CCO[C@H](CCO)CC4)cc(-c4cc(Cl)cc(Cl)c4)n3)cn2)CC1] | (S)-2-(4-((2-(3,5-dichlorophenyl)-6-((2- (4-methylpiperazin-1-yl)pyrimidin-5- yl)oxy)pyridin-4-yl)methyl)-1,4- oxazepan-7-yl)ethanol |
| 154 | 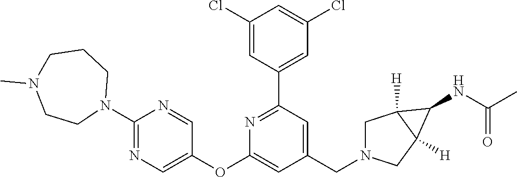 [SMILES: CC(=O)N[C@@H]1[C@@H]2CN(Cc3cc(Oc4cnc(N5CCCN(C)CC5)nc4)nc(-c4cc(Cl)cc(Cl)c4)c3)C[C@@H]21] | N-((1R,5S,6r)-3-((2-(3,5-dichloro- phenyl)-6-((2-(4-methyl-1,4- diazepan-1-yl)pyrimidin-5-yl)oxy) pyridin-4-yl)methyl)-3-azabicyclo [3.1.0]hexan-6-yl)acetamide |
| 155 | 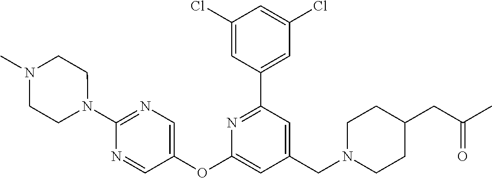 [SMILES: CC(=O)CC1CCN(Cc2cc(Oc3cnc(N4CCN(C)CC4)nc3)nc(-c3cc(Cl)cc(Cl)c3)c2)CC1] | 1-(1-((2-(3,5-dichlorophenyl)-6-((2-(4- methylpiperazin-1-yl)pyrimidin-5- yl)oxy)pyridin-4-yl)methyl)piperidin- 4-yl)propan-2-one |
| 156 | 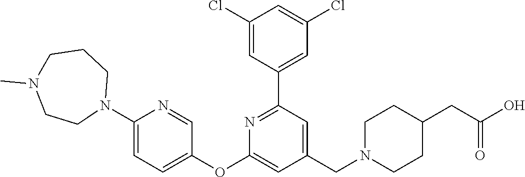 [SMILES: CN1CCCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((6-(4- methyl-1,4-diazepan-1-yl)pyridin-3- yl)oxy)pyridin-4-yl)methyl)piperidin- 4-yl)acetic acid |
| 157 | 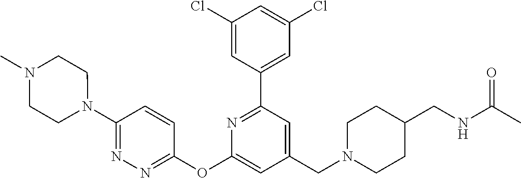 [SMILES: CC(=O)NCC1CCN(Cc2cc(Oc3ccc(N4CCN(C)CC4)nn3)nc(-c3cc(Cl)cc(Cl)c3)c2)CC1] | N-((1-((2-(3,5-dichlorophenyl)-6-((6- (4-methylpiperazin-1-yl)pyridazin-3- yl)oxy)pyridin-4-yl)methyl)piperidin- 4-yl)methyl)acetamide |
| 158 | 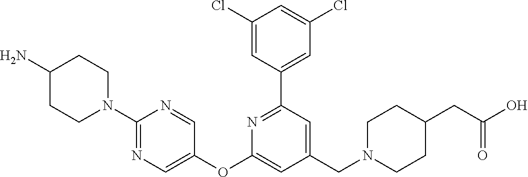 [SMILES: NC1CCN(c2ncc(Oc3cc(CN4CCC(CC(=O)O)CC4)cc(-c4cc(Cl)cc(Cl)c4)n3)cn2)CC1] | 2-(1-((2-((2-(4-aminopiperidin-1- yl)pyrimidin-5-yl)oxy)-6-(3,5- dichlorophenyl)pyridin-4- yl)methyl)piperidin-4-yl)acetic acid |
| 159 | 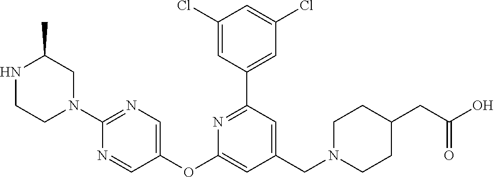 [SMILES: C[C@H]1CN(c2ncc(Oc3cc(CN4CCC(CC(=O)O)CC4)cc(-c4cc(Cl)cc(Cl)c4)n3)cn2)CCN1] | (S)-2-(1-((2-(3,5-dichlorophenyl)-6-((2- (3-methylpiperazin-1-yl)pyrimidin-5- yl)oxy)pyridin-4-yl)methyl)piperidin-4- yl)acetic acid |
| 160 | 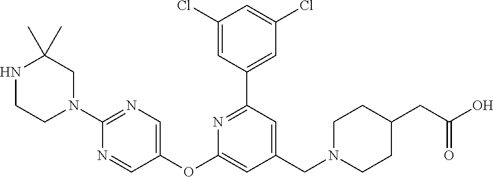 [SMILES: CC1(C)CN(c2ncc(Oc3cc(CN4CCC(CC(=O)O)CC4)cc(-c4cc(Cl)cc(Cl)c4)n3)cn2)CCN1] | 2-(1-((2-(3,5-dichlorophenyl)-6-((2-(3, 3-dimethylpiperazin-1-yl)pyrimidin-5- yl)oxy)pyridin-4-yl)methyl)piperidin-4- yl)acetic acid |
| 161 | 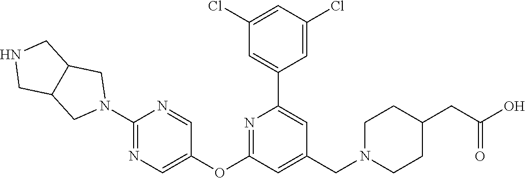 [SMILES: O=C(O)CC1CCN(Cc2cc(Oc3cnc(N4CC5CNCC5C4)nc3)nc(-c3cc(Cl)cc(Cl)c3)c2)CC1] | 2-(1-((2-(3,5-dichlorophenyl)-6-((2- (hexahydropyrrolo[3,4-c]pyrrol-2(1H)- yl)pyrimidin-5-yl)oxy)pyridin-4- yl)methyl)piperidin-4-yl)acetic acid |
| 162 | 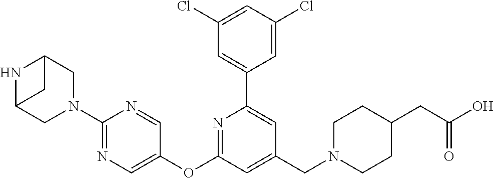 [SMILES: O=C(O)CC1CCN(Cc2cc(Oc3cnc(N4CC5CC(C4)N5)nc3)nc(-c3cc(Cl)cc(Cl)c3)c2)CC1] | 2-(1-((2-((2-(3,6- diazabicyclo[3.1.1]heptan-3-yl) pyrimidin-5-yl)oxy)-6-(3,5- dichlorophenyl)pyridin- 4-yl)methyl)piperidin-4-yl) acetic acid |
| 163 | 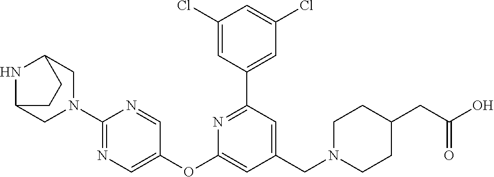 [SMILES: O=C(O)CC1CCN(Cc2cc(Oc3cnc(N4CC5CCC(C4)N5)nc3)nc(-c3cc(Cl)cc(Cl)c3)c2)CC1] | 2-(1-((2-((2-(3,8-diazabicyclo[3.2.1] octan-3-yl)pyrimidin-5-yl)oxy)-6-(3,5- dichlorophenyl)pyridin-4- yl)methyl)piperidin-4-yl)acetic acid |
| 164 | 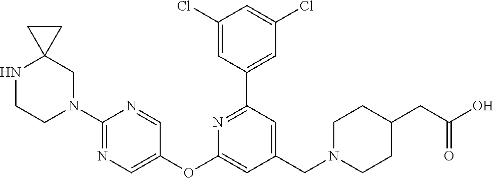 [SMILES: O=C(O)CC1CCN(Cc2cc(Oc3cnc(N4CCNC5(CC5)C4)nc3)nc(-c3cc(Cl)cc(Cl)c3)c2)CC1] | 2-(1-((2-((2-(4,7-diazaspiro[2.5]octan- 7-yl)pyrimidin-5-yl)oxy)-6-(3,5- dichlorophenyl)pyridin-4- yl)methyl)piperidin-4-yl)acetic acid |
| 165 | 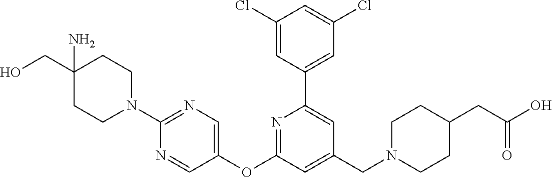 [SMILES: NC1(CO)CCN(c2ncc(Oc3cc(CN4CCC(CC(=O)O)CC4)cc(-c4cc(Cl)cc(Cl)c4)n3)cn2)CC1] | 2-(1-((2-((2-(4-amino-4- (hydroxymethyl)piperidin-1-yl) pyrimidin-5-yl)oxy)-6-(3,5- dichlorophenyl)pyridin- 4-yl)methyl)piperidin-4-yl) acetic acid |
| 166 | 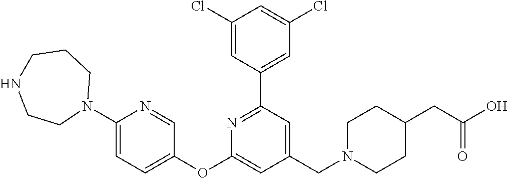 [SMILES: O=C(O)CC1CCN(Cc2cc(Oc3ccc(N4CCCNCC4)nc3)nc(-c3cc(Cl)cc(Cl)c3)c2)CC1] | 2-(1-((2-((6-(1,4-diazepan-1-yl) pyridin-3-yl)oxy)-6-(3,5- dichlorophenyl)pyridin-4- yl)methyl)piperidin-4-yl) acetic acid |
| 167 | 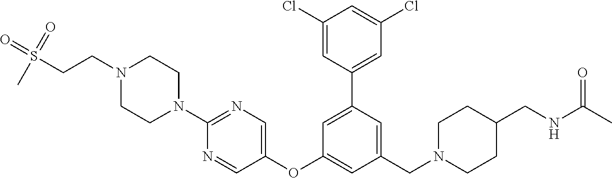 [SMILES: CC(=O)NCC1CCN(Cc2cc(Oc3cnc(N4CCN(CCS(C)(=O)=O)CC4)nc3)cc(-c3cc(Cl)cc(Cl)c3)c2)CC1] | N-((1-((3′,5′-dichloro-5-((2-(4-(2- (methylsulfonyl)ethyl)piperazin-1- yl)pyrimidin-5-yl)oxy)-[1,1′- biphenyl]-3-yl)methyl)piperidin-4- yl)methyl)acetamide |
| 168 | 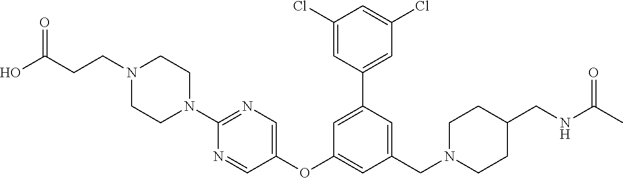 [SMILES: CC(=O)NCC1CCN(Cc2cc(Oc3cnc(N4CCN(CCC(=O)O)CC4)nc3)cc(-c3cc(Cl)cc(Cl)c3)c2)CC1] | 3-(4-(5-((5-(4- (acetamidomethyl)piperidin-1-yl) methyl)-3′,5′-dichloro-[1,1′- biphenyl]-3-yl)oxy)pyrimidin-2- yl)piperazin-1- yl)propanoic acid |
| 169 | 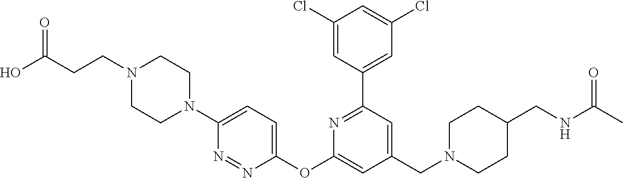 [SMILES: CC(=O)NCC1CCN(Cc2cc(Oc3ccc(N4CCN(CCC(=O)O)CC4)nn3)nc(-c3cc(Cl)cc(Cl)c3)c2)CC1] | 3-(4-(6-((4-((4- (acetamidomethyl)piperidin-1-yl) methyl)-6-(3,5-dichlorophenyl) pyridin-2-yl)oxy)pyridazin-3-yl) piperazin-1-yl)propanoic acid |
| 170 | 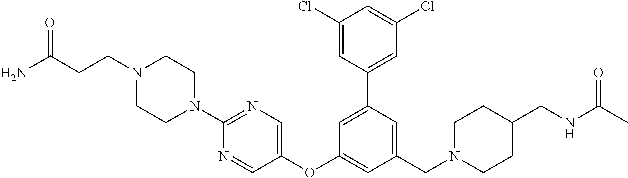 [SMILES: CC(=O)NCC1CCN(Cc2cc(Oc3cnc(N4CCN(CCC(N)=O)CC4)nc3)cc(-c3cc(Cl)cc(Cl)c3)c2)CC1] | 3-(4-(5-((5-((4- (acetamidomethyl)piperidin-1-yl) methyl)-3′,5′-dichloro-[1,1′- biphenyl]-3-yl)oxy)pyrimidin-2- yl)piperazin-1-yl)propanamide |
| 171 | 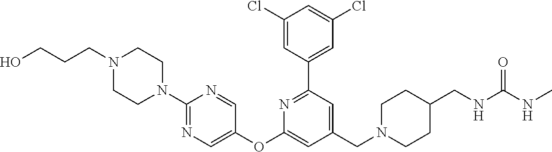 [SMILES: CNC(=O)NCC1CCN(Cc2cc(Oc3cnc(N4CCN(CCCO)CC4)nc3)nc(-c3cc(Cl)cc(Cl)c3)c2)CC1] | 1-((1-((2-(3,5-dichlorophenyl)-6-((2-(4- (3-hydroxypropyl)piperazin-1-yl) pyrimidin-5-yl)oxy)pyridin-4-yl) methyl)piperidin-4- yl)methyl)-3-methylurea |
| 172 | 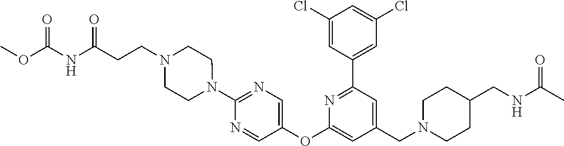 [SMILES: COC(=O)NC(=O)CCN1CCN(c2ncc(Oc3cc(CN4CCC(CNC(C)=O)CC4)cc(-c4cc(Cl)cc(Cl)c4)n3)cn2)CC1] | methyl(3-(4-(5-((4-((4- (acetamidomethyl)piperidin-1-yl) methyl)- 6-(3,5-dichlorophenyl)pyridin-2- yl)oxy)pyrimidin-2-yl)piperazin-1- yl)propanoyl)carbamate |
| 173 | 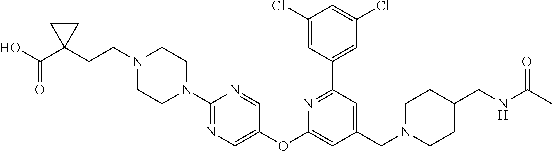 [SMILES: CC(=O)NCC1CCN(Cc2cc(Oc3cnc(N4CCN(CCC5(C(=O)O)CC5)CC4)nc3)nc(-c3cc(Cl)cc(Cl)c3)c2)CC1] | 1-(2-(4-(5-((4-((4- (acetamidomethyl)piperidin-1-yl) methyl)- 6-(3,5-dichlorophenyl)pyridin-2- yl)oxy)pyrimidin-2-yl)piperazin-1- yl)ethyl)cyclopropanecarboxylic acid |
| 174 | 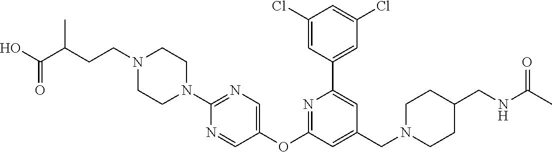 [SMILES: CC(=O)NCC1CCN(Cc2cc(Oc3cnc(N4CCN(CCC(C)C(=O)O)CC4)nc3)nc(-c3cc(Cl)cc(Cl)c3)c2)CC1] | 4-(4-(5-((4-((4- (acetamidomethyl)piperidin-1-yl) methyl)- 6-(3,5-dichlorophenyl)pyridin-2- yl)oxy)pyridin-2-yl)piperazin-1-yl)-2- methylbutanoic acid |
| 175 | 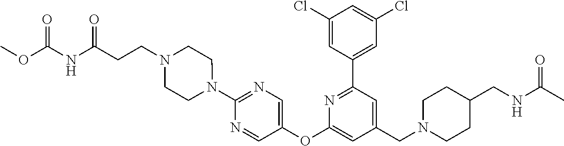 [SMILES: COC(=O)NC(=O)CCN1CCN(c2ncc(Oc3cc(CN4CCC(CNC(C)=O)CC4)cc(-c4cc(Cl)cc(Cl)c4)n3)cn2)CC1] | methyl(3-(4-(5-((4-((4- (acetamidomethyl)piperidin-1-yl) methyl)-6-(3,5-dichlorophenyl) pyridin-2-yl)oxy)pyrimidin-2-yl) piperazin-1-yl)propanoyl)carbamate |
| 176 | 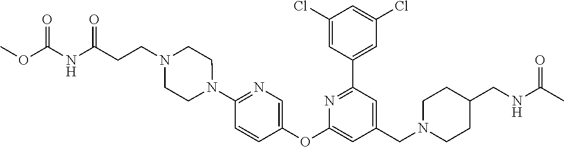 [SMILES: COC(=O)NC(=O)CCN1CCN(c2ccc(Oc3cc(CN4CCC(CNC(C)=O)CC4)cc(-c4cc(Cl)cc(Cl)c4)n3)cn2)CC1] | methyl(3-(4-(5-((4-((4- (acetamidomethyl)piperidin-1-yl) methyl)- 6-(3,5-dichlorophenyl)pyridin-2- yl)oxy)pyridin-2-yl)piperazin-1- yl)propanoyl)carbamate |
| 177 | 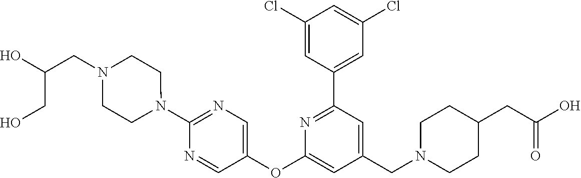 [SMILES: O=C(O)CC1CCN(Cc2cc(Oc3cnc(N4CCN(CC(O)CO)CC4)nc3)nc(-c3cc(Cl)cc(Cl)c3)c2)CC1] | 2-(1-((2-(3,5-dichlorophenyl)-6-((2-(4- (2,3-dihydroxypropyl)piperazin-1- yl)pyrimidin-5-yl)oxy)pyridin-4- yl)methyl)piperidin-4-yl)acetic acid |
| 178 | 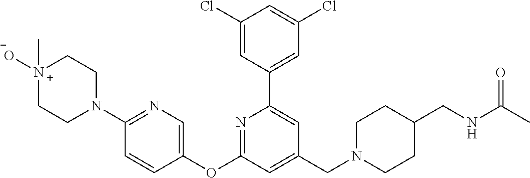 [SMILES: CC(=O)NCC1CCN(Cc2cc(Oc3ccc(N4CC[N+](C)([O-])CC4)nc3)nc(-c3cc(Cl)cc(Cl)c3)c2)CC1] | 4-(5-((4-((4-(acetamidomethyl) piperidin-1-yl)methyl)-6-(3,5- dichlorophenyl)pyridin-2-yl)oxy) pyridin-2-yl)-1- methylpiperazinel-oxide |
| 179 | 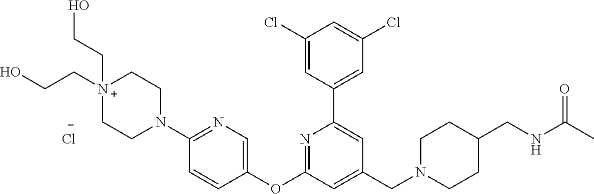 [SMILES: CC(=O)NCC1CCN(Cc2cc(Oc3ccc(N4CC[N+](CCO)(CCO)CC4)nc3)nc(-c3cc(Cl)cc(Cl)c3)c2)CC1 ; [Cl-]] | 4-(5-((4-((4-(acetamidomethyl) piperidin-1-yl)methyl)-6-(3,5- dichlorophenyl)pyridin-2-yl)oxy) pyridin-2-yl)-1,1-bis(2-hydroxyethyl) piperazin-1-ium |
| 180 | 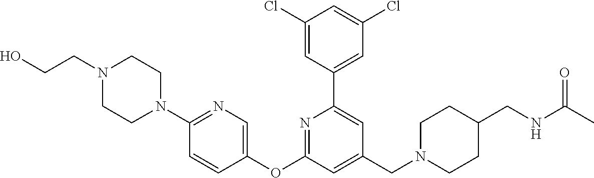 [SMILES: CC(=O)NCC1CCN(Cc2cc(Oc3ccc(N4CCN(CCO)CC4)nc3)nc(-c3cc(Cl)cc(Cl)c3)c2)CC1] | N-((1-((2-(3,5-dichlorophenyl)-6-((6- (4-(2-hydroxyethyl)piperazin-1-yl) pyridin-3-yl)oxy)pyridin-4-yl)methyl) piperidin-4-yl)methyl)acetamide |
| 181 | 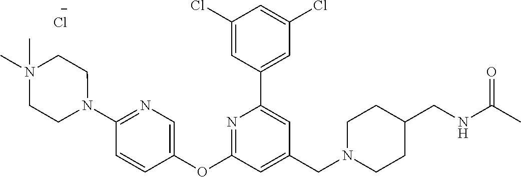 | 4-(5-((4-((4-(acetamidomethyl) piperidin- 1-yl)methyl)-6-(3,5- dichlorophenyl)pyridin-2-yl)oxy) pyridin-2-yl)-1,1-dimethylpiperazin- 1-ium |
| 182 | 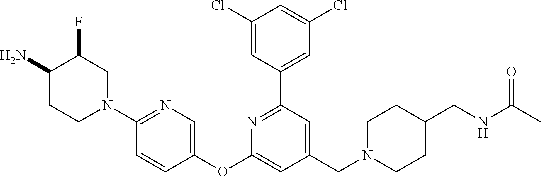 [SMILES: CC(=O)NCC1CCN(Cc2cc(Oc3ccc(N4CC[C@@H](N)[C@@H](F)C4)nc3)nc(-c3cc(Cl)cc(Cl)c3)c2)CC1] | N-((1-((2-((6-(4-amino-3- fluoropiperidin- 1-yl)pyridin-3-yl)oxy)-6-(3,5- dichlorophenyl)pyridin-4- yl)methyl)piperidin-4- yl)methyl)acetamide |
| 183 | 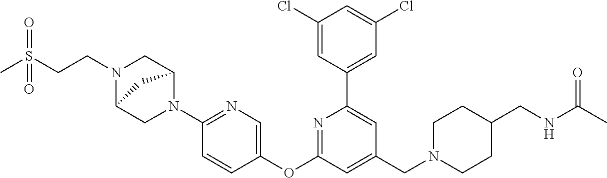 [SMILES: CC(=O)NCC1CCN(Cc2cc(Oc3ccc(N4C[C@@H]5C[C@H]4CN5CCS(C)(=O)=O)nc3)nc(-c3cc(Cl)cc(Cl)c3)c2)CC1] | N-((1-((2-(3,5-dichlorophenyl)-6-((6- ((1S,4S)-5-(2-(methylsulfonyl)ethyl)- 2,5-diazabicyclo[2.2.1]heptan-2-yl) pyridin-3-yl)oxy)pyridin-4-yl)methyl) piperidin-4-yl)methyl)acetamide |
| 184 | 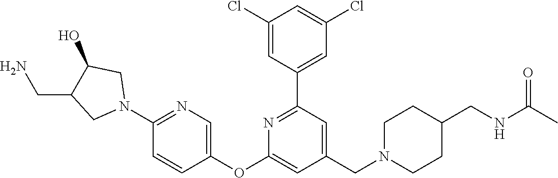 [SMILES: CC(=O)NCC1CCN(Cc2cc(Oc3ccc(N4CC(CN)[C@@H](O)C4)nc3)nc(-c3cc(Cl)cc(Cl)c3)c2)CC1] | N-((1-((2-((6-((3S,4R)-3- (aminomethyl)- 4-hydroxypyrrolidin-1-yl)pyridin-3- yl)oxy)-6-(3,5-dichlorophenyl)pyridin- 4-yl)methyl)piperidin-4- yl)methyl)acetamide |
| 185 | 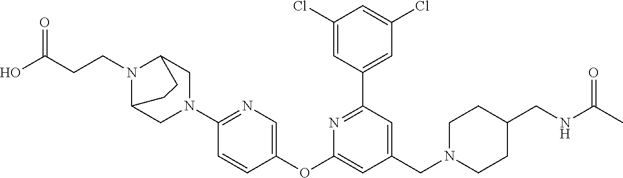 [SMILES: CC(=O)NCC1CCN(Cc2cc(Oc3ccc(N4CC5CCC(C4)N5CCC(=O)O)nc3)nc(-c3cc(Cl)cc(Cl)c3)c2)CC1] | 3-((1R,5S)-3-(5-((4-((4- (acetamidomethyl)piperidin-1-yl) methyl)- 6-(3,5-dichlorophenyl)pyridin-2- yl)oxy)pyridin-2-yl)-3,8- diazabicyclo[3.2.1]octan-8-yl) propanoic acid |
| 186 | 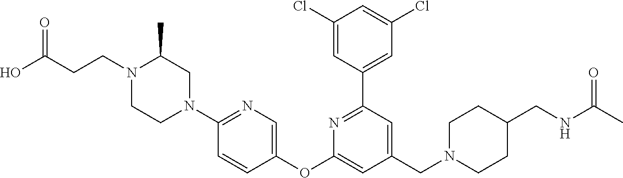 [SMILES: CC(=O)NCC1CCN(Cc2cc(Oc3ccc(N4CCN(CCC(=O)O)[C@@H](C)C4)nc3)nc(-c3cc(Cl)cc(Cl)c3)c2)CC1] | (S)-3-(4-(5-((4-(4- (acetamidomethyl)piperidin-1-yl) methyl)- 6-(3,5-dichlorophenyl)pyridin-2- yl)oxy)pyridin-2-yl)-2- methylpiperazin-1-yl)propanoic acid |
| 187 | 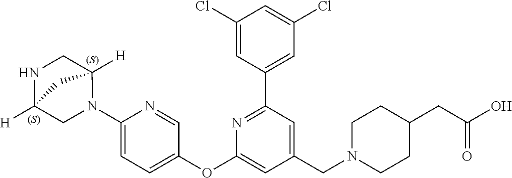 | 2-(1-((2-((6-((1S,4S)-2,5- diazabicyclo[2.2.1]heptan-2-yl)pyridin- 3-yl)oxy)-6-(3,5-dichlorophenyl) pyridin-4-yl)methyl)piperidin-4-yl) acetic acid |
| 188 | 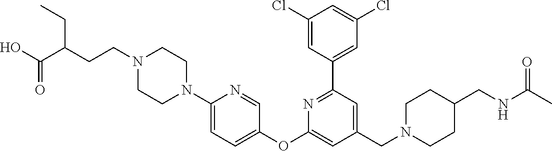 [SMILES: CCC(CCN1CCN(c2ccc(Oc3cc(CN4CCC(CNC(C)=O)CC4)cc(-c4cc(Cl)cc(Cl)c4)n3)cn2)CC1)C(=O)O] | 4-(4-(5-((4-((4- (acetamidomethyl)piperidin-1-yl) methyl)- 6-(3,5-dichlorophenyl)pyridin-2- yl)oxy)pyridin-2-yl)piperazin-1-yl)-2- ethylbutanoic acid |
| 189 | 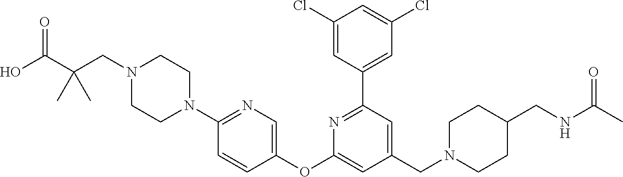 [SMILES: CC(=O)NCC1CCN(Cc2cc(Oc3ccc(N4CCN(CC(C)(C)C(=O)O)CC4)nc3)nc(-c3cc(Cl)cc(Cl)c3)c2)CC1] | 3-(4-(5-((4-(4- (acetamidomethyl)piperidin-1-yl) methyl)- 6-(3,5-dichlorophenyl)pyridin-2- yl)oxy)pyridin-2-yl)piperazin-1-yl)- 2,2-dimethylpropanoic acid |
| 190 | 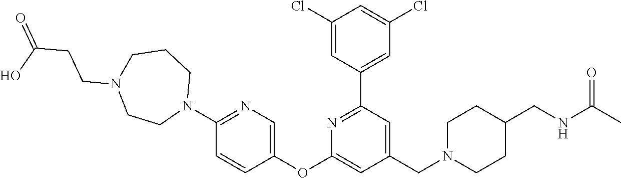 [SMILES: CC(=O)NCC1CCN(Cc2cc(Oc3ccc(N4CCCN(CCC(=O)O)CC4)nc3)nc(-c3cc(Cl)cc(Cl)c3)c2)CC1] | 3-(4-(5-((4-((4- (acetamidomethyl)piperidin-1-yl) methyl)- 6-(3,5-dichlorophenyl)pyridin-2- yl)oxy)pyridin-2-yl)-1,4-diazepan-1- yl)propanoic acid |
| 191 | 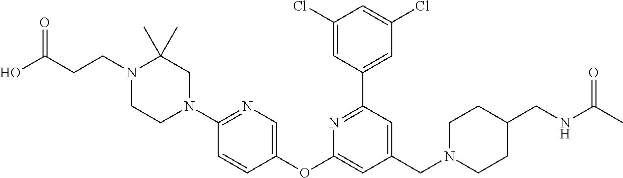 [SMILES: CC(=O)NCC1CCN(Cc2cc(Oc3ccc(N4CCN(CCC(=O)O)C(C)(C)C4)nc3)nc(-c3cc(Cl)cc(Cl)c3)c2)CC1] | 3-(4-(5-((4-(4- (acetamidomethyl)piperidin-1-yl) methyl)- 6-(3,5-dichlorophenyl)pyridin-2- yl)oxy)pyridin-2-yl)-2,2- dimethylpiperazin-1-yl)propanoic acid |
| 192 | 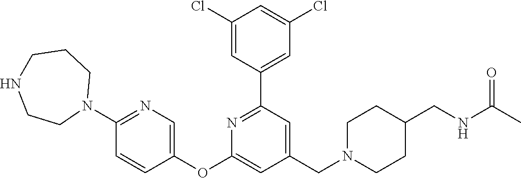 [SMILES: CC(=O)NCC1CCN(Cc2cc(Oc3ccc(N4CCCNCC4)nc3)nc(-c3cc(Cl)cc(Cl)c3)c2)CC1] | N-((1-((2-((6-(1,4-diazepan-1-yl) pyridin-3-yl)oxy)-6-(3,5- dichlorophenyl)pyridin- 4-yl)methyl)piperidin-4- yl)methyl)acetamide |
| 193 | 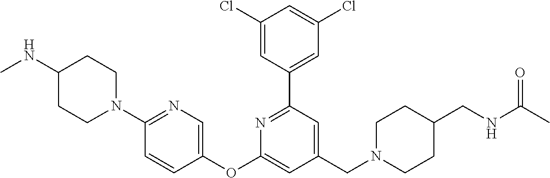 [SMILES: CNC1CCN(c2ccc(Oc3cc(CN4CCC(CNC(C)=O)CC4)cc(-c4cc(Cl)cc(Cl)c4)n3)cn2)CC1] | N-((1-((2-(3,5-dichlorophenyl)-6-((6-(4- (methylamino)piperidin-1-yl)pyridin-3- yl)oxy)pyridin-4-yl)methyl)piperidin-4- yl)methyl)acetamide |
| 194 | 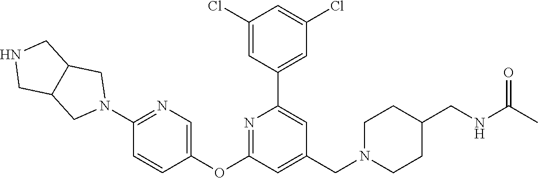 [SMILES: CC(=O)NCC1CCN(Cc2cc(Oc3ccc(N4CC5CNCC5C4)nc3)nc(-c3cc(Cl)cc(Cl)c3)c2)CC1] | N-((1-((2-(3,5-dichlorophenyl)-6-((6- (hexahydropyrrolo[3,4-c]pyrrol-2(1H)- yl)pyridin-3-yl)oxy)pyridin-4- yl)methyl)piperidin-4- yl)methyl)acetamide |
| 195 | 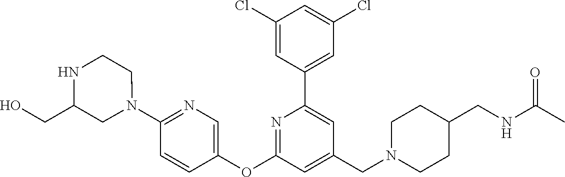 [SMILES: CC(=O)NCC1CCN(Cc2cc(Oc3ccc(N4CCNC(CO)C4)nc3)nc(-c3cc(Cl)cc(Cl)c3)c2)CC1] | N-((1-((2-(3,5-dichlorophenyl)-6-((6-(3- (hydroxymethyl)piperazin-1-yl)pyridin- 3-yl)oxy)pyridin-4-yl)methyl)piperidin- 4-yl)methyl)acetamide |
| 196 | 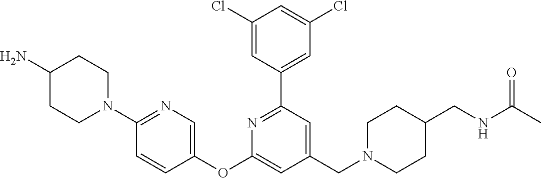 [SMILES: CC(=O)NCC1CCN(Cc2cc(Oc3ccc(N4CCC(N)CC4)nc3)nc(-c3cc(Cl)cc(Cl)c3)c2)CC1] | N-((1-((2-((6-(4-aminopiperidin-1- yl)pyridin-3-yl)oxy)-6-(3,5- dichlorophenyl)pyridin-4- yl)methyl)piperidin-4- yl)methyl)acetamide |
| 197 | 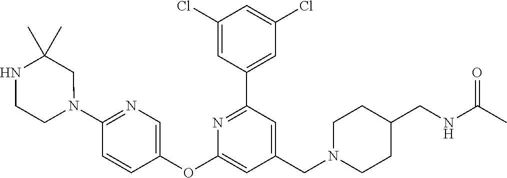 [SMILES: CC(=O)NCC1CCN(Cc2cc(Oc3ccc(N4CCNC(C)(C)C4)nc3)nc(-c3cc(Cl)cc(Cl)c3)c2)CC1] | N-((1-((2-(3,5-dichlorophenyl)-6-((6- (3,3-dimethylpiperidin-1-yl)pyridin-3- yl)oxy)pyridin-4-yl)methyl)piperidin-4- yl)methyl)acetamide |
| 198 | 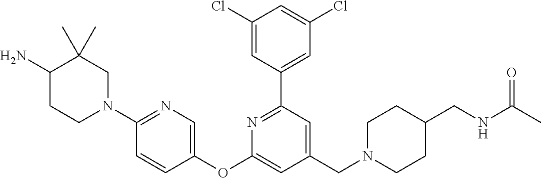 [SMILES: CC(=O)NCC1CCN(Cc2cc(Oc3ccc(N4CCC(N)C(C)(C)C4)nc3)nc(-c3cc(Cl)cc(Cl)c3)c2)CC1] | N-((1-((2-((6-(4-amino-3,3- dimethylpiperidin-1-yl)pyridin-3-yl) oxy)-6-(3,5-dichlorophenyl)pyridin-4- yl)methyl)piperidin-4- yl)methyl)acetamide |
| 199 | 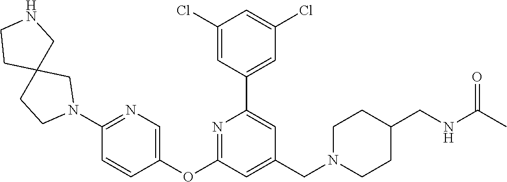 [SMILES: CC(=O)NCC1CCN(Cc2cc(Oc3ccc(N4CCC5(CCNC5)C4)nc3)nc(-c3cc(Cl)cc(Cl)c3)c2)CC1] | N-((1-((2-((6-(2,7-diazaspiro[4.4]- nonan2-yl)pyridin-3-yl)oxy)-6-(3,5- dichlorophenyl)pyridin-4- yl)methyl)piperidin-4- yl)methyl)acetamide |
| 200 | 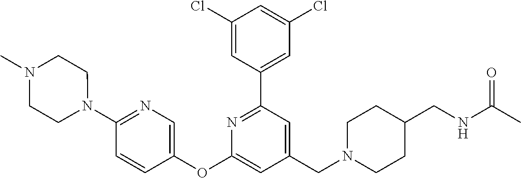 [SMILES: CC(=O)NCC1CCN(Cc2cc(Oc3ccc(N4CCN(C)CC4)nc3)nc(-c3cc(Cl)cc(Cl)c3)c2)CC1] | N-((1-((2-(3,5-dichlorophenyl)-6-((6-(4- methylpiperidin-1-yl)pyridin-3-yl)oxy) pyridin-4-yl)methyl)piperidin-4- yl)methyl)acetamide |
| 201 | 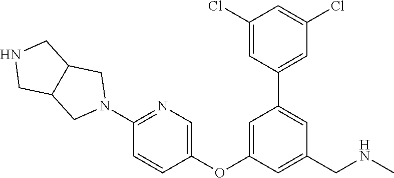 [SMILES: CNCc1cc(Oc2ccc(N3CC4CNCC4C3)nc2)cc(-c2cc(Cl)cc(Cl)c2)c1] | 1-(3′,5′-dichloro-5-((6- (hexahydropyrrolo[3,4-c]pyrrol-2(1H)- yl)pyridin-3-yl)oxy)-[1,1′-biphenyl]-3- yl)-N-methylmethanamine |
| 202 | 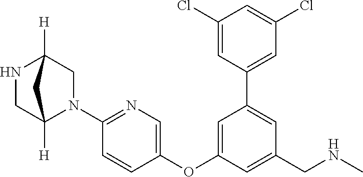 [SMILES: CNCc1cc(Oc2ccc(N3C[C@@H]4C[C@H]3CN4)nc2)cc(-c2cc(Cl)cc(Cl)c2)c1] | 1-(5-((6-((1S,4S)-2,5- diazabicyclo[2.2.1]heptan-2-yl)pyridin- 3-yl)oxy)-3′,5′-dichloro-[1,1′-biphenyl]- 3-yl)-N-methylmethanamine |
| 203 | 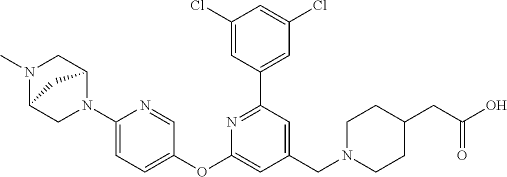 [SMILES: CN1C[C@@H]2C[C@H]1CN2c1ccc(Oc2cc(CN3CCC(CC(=O)O)CC3)cc(-c3cc(Cl)cc(Cl)c3)n2)cn1] | 2-(1-((2-(3,5-dichlorophenyl)-6-((6- ((1S,4S)-5-methyl-2,5- diazabicyclo[2.2.1]heptan-2-yl)pyridin- 3-yl)oxy)pyridin-4-yl)methyl)piperidin- 4-yl)acetic acid |
| 204 | 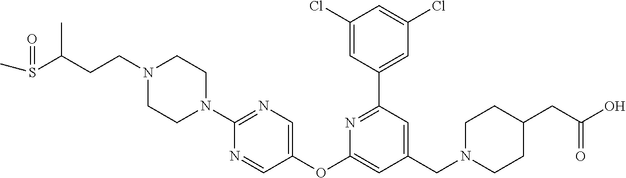 [SMILES: CC(CCN1CCN(c2ncc(Oc3cc(CN4CCC(CC(=O)O)CC4)cc(-c4cc(Cl)cc(Cl)c4)n3)cn2)CC1)S(C)=O] | 2-(1-((2-(3,5-dichlorophenyl)-6-((2-(4- (3-(methylsulfonyl)butyl)piperazin-1- yl)pyrimidin-5-yl)oxy)pyridin-4- yl)methyl)piperidin-4-yl)acetic acid |
| 205 | 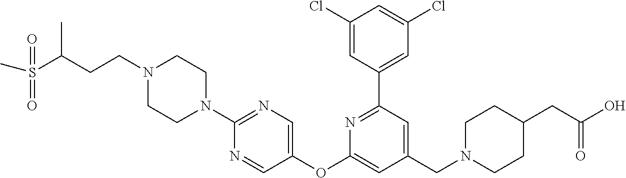 [SMILES: CC(CCN1CCN(c2ncc(Oc3cc(CN4CCC(CC(=O)O)CC4)cc(-c4cc(Cl)cc(Cl)c4)n3)cn2)CC1)S(C)(=O)=O] | 2-(1-((2-(3,5-dichlorophenyl)-6-((2-(4- (3-(methylsulfonyl)butyl)piperazin-1- yl)pyrimidin-5-yl)oxy)pyridin-4- yl)methyl)piperidin-4-yl)acetic acid |
| 206 | 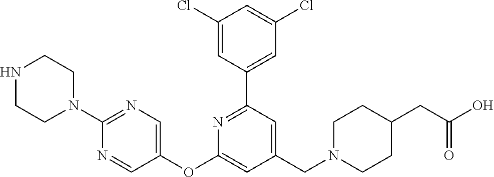 [SMILES: O=C(O)CC1CCN(Cc2cc(Oc3cnc(N4CCNCC4)nc3)nc(-c3cc(Cl)cc(Cl)c3)c2)CC1] | 2-(1-((2-(3,5-dichlorophenyl)-6-((2- (piperazin-1-yl)pyrimidin-5- yl)oxy)pyridin-4-yl)methyl)piperidin- 4-yl)acetic acid |
| 207 | 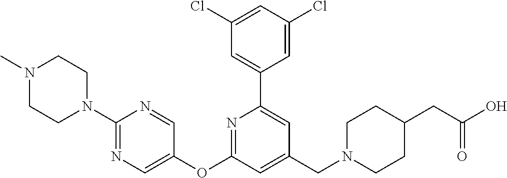 [SMILES: CN1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2-(4- methylpiperazin-1-yl)pyrimidin-5- yl)oxy)pyridin-4-yl)methyl)piperidin- 4-yl)acetic acid |
| 208 | 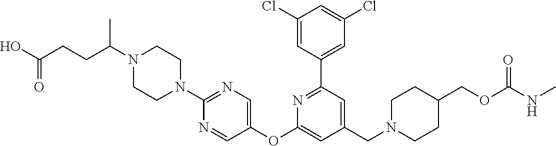 [SMILES: CNC(=O)OCC1CCN(Cc2cc(Oc3cnc(N4CCN(C(C)CCC(=O)O)CC4)nc3)nc(-c3cc(Cl)cc(Cl)c3)c2)CC1] | 4-(4-(5-((6-(3,5-dichlorophenyl)-4-((4- (((methylcarbamoyl)oxy)methyl) piperidin-1-yl)methyl)pyridin-2-yl) oxy)pyrimidin- 2-yl)piperazin-1-yl)pentanoic acid |
| 209 | 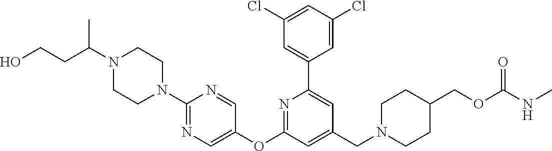 [SMILES: CNC(=O)OCC1CCN(Cc2cc(Oc3cnc(N4CCN(C(C)CCO)CC4)nc3)nc(-c3cc(Cl)cc(Cl)c3)c2)CC1] | (1-((2-(3,5-dichlorophenyl)-6-((2-(4-(4- hydroxybutan-2-yl)piperazin-1- yl)pyrimidin-5-yl)oxy)pyridin-4- yl)methyl)piperidin-4- yl)methylmethylcarbamate |
| 210 | 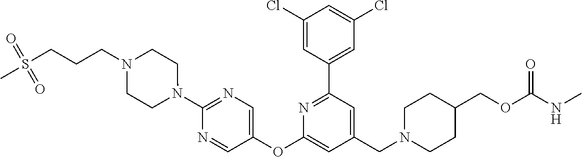 | (1-((2-(3,5-dichlorophenyl)-6-((2-(4-(3- (methylsulfonyl)propyl)piperazin-1- yl)pyrimidin-5-yl)oxy)pyridin-4- yl)methyl)piperidin-4- yl)methylmethylcarbamate |
| 211 | 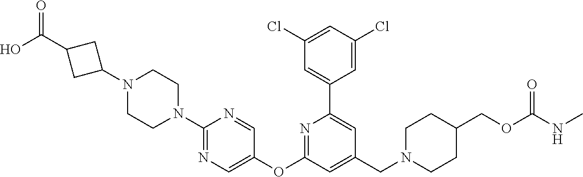 [SMILES: CNC(=O)OCC1CCN(Cc2cc(Oc3cnc(N4CCN(C5CC(C(=O)O)C5)CC4)nc3)nc(-c3cc(Cl)cc(Cl)c3)c2)CC1] | 3-(4-(5-((6-(3,5-dichlorophenyl)-4-((4- (((methylcarbamoyl)oxy)methyl) piperidin-1-yl)methyl)pyridin-2-yl) oxy)pyrimidin-2-yl)piperazin- 1-yl)cyclobutanecarboxylic acid |
| 212 | 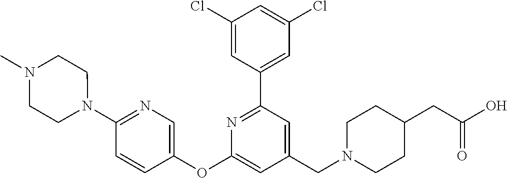 [SMILES: CN1CCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((6-(4- methylpiperazin-1-yl)pyridin-3- yl)oxy)pyridin-4-yl)methyl)piperidin- 4-yl)acetic acid |
| 213 | 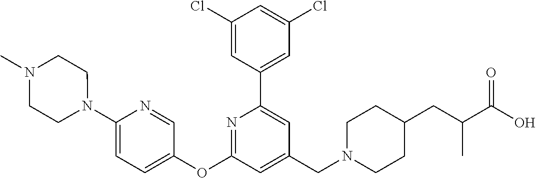 [SMILES: CC(CC1CCN(Cc2cc(Oc3ccc(N4CCN(C)CC4)nc3)nc(-c3cc(Cl)cc(Cl)c3)c2)CC1)C(=O)O] | 3-(1-((2-(3,5-dichlorophenyl)-6-((6-(4- methylpiperazin-1-yl)pyridin-3- yl)oxy)pyridin-4-yl)methyl)piperidin- 4-yl)-2-methylpropanoic acid |
| 214 | 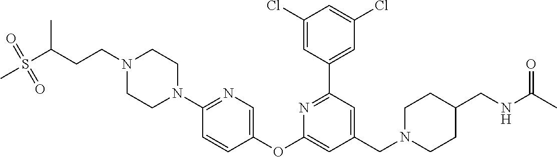 [SMILES: CC(=O)NCC1CCN(Cc2cc(Oc3ccc(N4CCN(CCC(C)S(C)(=O)=O)CC4)nc3)nc(-c3cc(Cl)cc(Cl)c3)c2)CC1] | N-((1-((2-(3,5-dichlorophenyl)-6-((2- (4-(3-(methylsulfonyl)butyl)piperazin- 1-yl)pyrimidin-5-yl)oxy)pyridin-4- yl)methyl)piperidin-4- yl)methyl)acetamide |
| 215 | 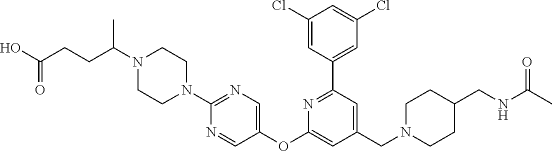 [SMILES: CC(=O)NCC1CCN(Cc2cc(Oc3cnc(N4CCN(C(C)CCC(=O)O)CC4)nc3)nc(-c3cc(Cl)cc(Cl)c3)c2)CC1] | 4-(4-(5-((4-((4- (acetamidomethyl)piperidin-1-yl) methyl)- 6-(3,5-dichlorophenyl)pyridin-2- yl)oxy)pyrimidin-2-yl)piperazin-1- yl)pentanoic acid |
| 216 | 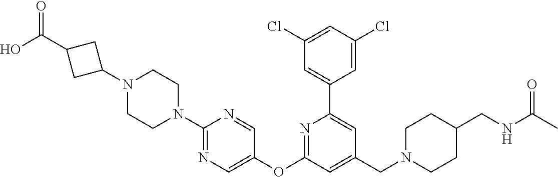 [SMILES: CC(=O)NCC1CCN(Cc2cc(Oc3cnc(N4CCN(C5CC(C(=O)O)C5)CC4)nc3)nc(-c3cc(Cl)cc(Cl)c3)c2)CC1] | 3-(4-(5-((4-(4- (acetamidomethyl)piperidin-1-yl) methyl)- 6-(3,5-dichlorophenyl)pyridin-2- yl)oxy)pyrimidin-2-yl)piperazin-1- yl)cyclobutanecarboxylic acid |
| 217 | 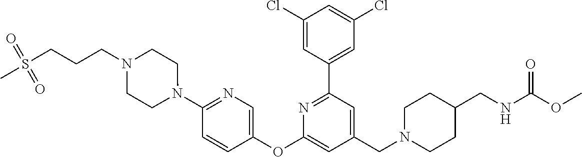 | methyl((1-((2-(3,5-dichlorophenyl)-6- ((6-(4-(3-(methylsulfonyl)butyl) piperazin-1-yl)pyrimidin-5-yl)oxy) pyridin-4-yl)methyl)piperidin-4- yl)methyl)carbamate |
| 218 | 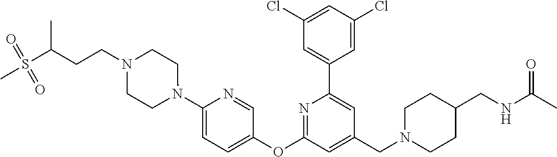 [SMILES: CC(=O)NCC1CCN(Cc2cc(Oc3ccc(N4CCN(CCC(C)S(C)(=O)=O)CC4)nc3)nc(-c3cc(Cl)cc(Cl)c3)c2)CC1] | N-((1-((2-(3,5-dichlorophenyl)-6-((6-(4- (3-(methylsulfonyl)butyl)piperazin-1- yl)pyrimidin-5-yl)oxy)pyridin-4- yl)methyl)piperidin-4- yl)methyl)acetamide |
| 219 | 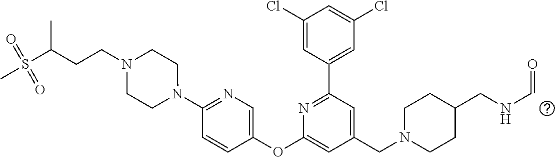 [SMILES: CC(CCN1CCN(c2ccc(Oc3cc(CN4CCC(CNC=O)CC4)cc(-c4cc(Cl)cc(Cl)c4)n3)cn2)CC1)S(C)(=O)=O] | 1-((1-((2-(3,5-dichlorophenyl)-6-((6-(4- (3-(methylsulfonyl)butyl)piperazin-1- yl)pyridin-3-yl)oxy)pyridin-4- yl)methyl)piperidin-4-yl)methyl)-3- methylurea |
| 220 | 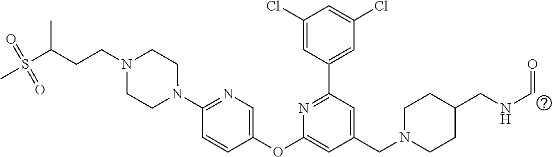 [SMILES: CC(CCN1CCN(c2ccc(Oc3cc(CN4CCC(CNC=O)CC4)cc(-c4cc(Cl)cc(Cl)c4)n3)cn2)CC1)S(C)(=O)=O] | 1-((1-((2-(3,5-dichlorophenyl)-6-((2-(4- (3-(methylsulfonyl)butyl)piperazin-1- yl)pyrimidin-5-yl)oxy)pyridin-4- yl)methyl)piperidin-4-yl)methyl)-3- methylurea |
| 221 | 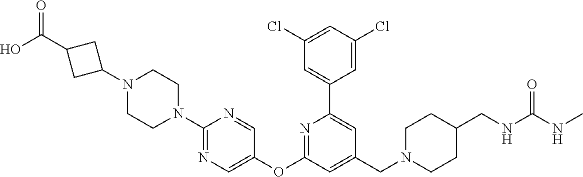 [SMILES: CNC(=O)NCC1CCN(Cc2cc(Oc3cnc(N4CCN(C5CC(C(=O)O)C5)CC4)nc3)nc(-c3cc(Cl)cc(Cl)c3)c2)CC1] | 3-(4-(5-((6-(3,5-dichlorophenyl)-4-((4- ((3-methylureido)methyl)piperidin-1- yl)methyl)pyridin-2-yl)oxy)pyrimidin- 2-yl)piperazin-1-yl) cyclobutanecarboxylic acid |
| 222 | 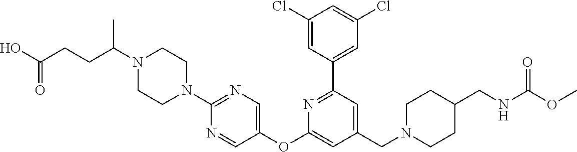 [SMILES: COC(=O)NCC1CCN(Cc2cc(Oc3cnc(N4CCN(C(C)CCC(=O)O)CC4)nc3)nc(-c3cc(Cl)cc(Cl)c3)c2)CC1] | 4-(4-(5-((6-(3,5-dichlorophenyl)-4-((4- (((methoxycarbonyl)amino)methyl) piperidin-1-yl)methyl)pyridin-2- yl)oxy)pyrimidin-2-yl)piperazin-1- yl)pentanoic acid |
| 223 | 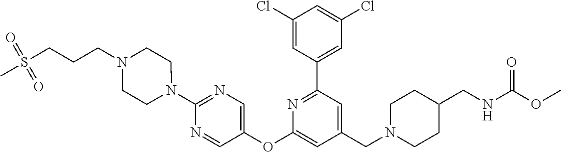 | methyl((1-((2-(3,5-dichlorophenyl)-6- ((2-(4-(3-(methylsulfonyl)propyl) piperazin-1-yl)pyrimidin-5-yl)oxy) pyridin-4- yl)methyl)piperidin-4- yl)methyl)carbamate |
| 224 | 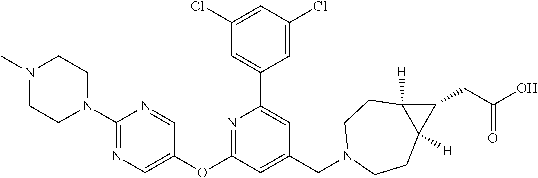 [SMILES: CN1CCN(c2ncc(Oc3cc(CN4CC[C@@H]5[C@H](CC4)[C@H]5CC(=O)O)cc(-c4cc(Cl)cc(Cl)c4)n3)cn2)CC1] | 2-((1R,7S,8r)-4-((2-(3,5-dichloro- phenyl)-6-((2-(4-methylpiperazin-1-yl) pyrimidin-5-yl)oxy)pyridin-4-yl) methyl)-4- azabicyclo[5.1.0]octan-8-yl)acetic acid |
| 225 | 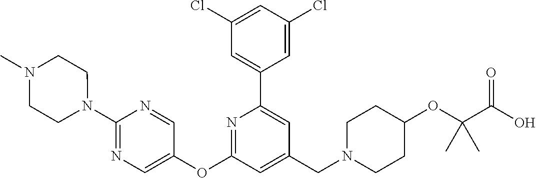 [SMILES: CN1CCN(c2ncc(Oc3cc(CN4CCC(OC(C)(C)C(=O)O)CC4)cc(-c4cc(Cl)cc(Cl)c4)n3)cn2)CC1] | 2-((1-((2-(3,5-dichlorophenyl)-6-((2-(4- methylpiperazin-1-yl)pyrimidin-5- yl)oxy)pyridin-4-yl)methyl)piperidin- 4-yl)oxy)-2-methylpropanoic acid |
| 226 | 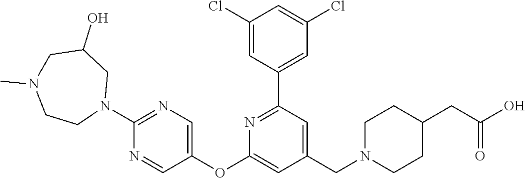 [SMILES: CN1CCN(c2ncc(Oc3cc(CN4CCC(CC(=O)O)CC4)cc(-c4cc(Cl)cc(Cl)c4)n3)cn2)CC(O)C1] | 2-(1-((2-(3,5-dichlorophenyl)-6-((2-(6- hydroxy-4-methyl-1,4-diazepan-1- yl)pyrimidin-5-yl)oxy)pyridin-4- yl)methyl)piperidin-4-yl)acetic acid |
| 227 | 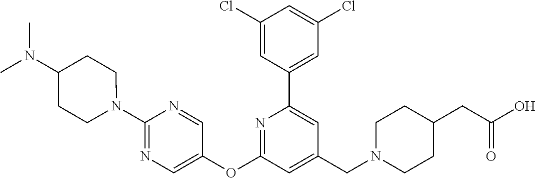 [SMILES: CN(C)C1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2-(4- (dimethylamino)piperidin-1-yl) pyrimidin-5-yl)oxy)pyridin-4-yl) methyl)piperidin-4-yl)acetic acid |
| 228 | 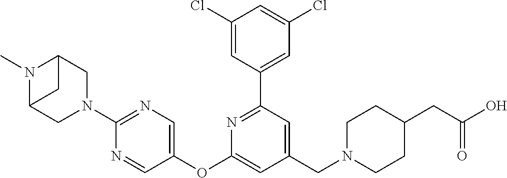 [SMILES: CN1C2CC1CN(c1ncc(Oc3cc(CN4CCC(CC(=O)O)CC4)cc(-c4cc(Cl)cc(Cl)c4)n3)cn1)C2] | 2-(1-((2-(3,5-dichlorophenyl)-6-((2-(6- methyl-3,6-diazabicyclo[3.1.l]heptan-3- yl)pyrimidin-5-yl)oxy)pyridin-4- yl)methyl)piperidin-4-yl)acetic acid |
| 229 | 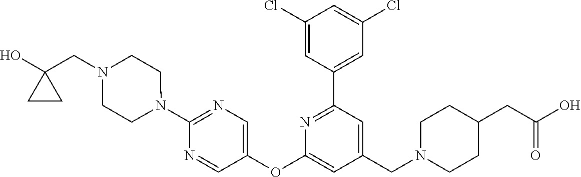 [SMILES: O=C(O)CC1CCN(Cc2cc(Oc3cnc(N4CCN(CC5(O)CC5)CC4)nc3)nc(-c3cc(Cl)cc(Cl)c3)c2)CC1] | 2-(1-((2-(3,5-dichlorophenyl)-6-((2-(4- ((1-hydroxycyclopropyl)methyl) piperazin-1-yl)pyrimidin-5-yl)oxy) pyridin-4-yl)methyl)piperidin-4- yl)acetic acid |
| 230 | 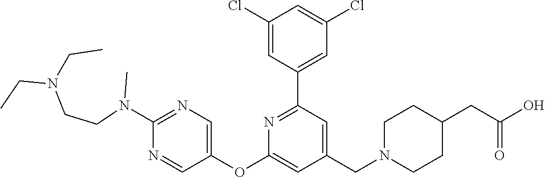 [SMILES: CCN(CC)CCN(C)c1ncc(Oc2cc(CN3CCC(CC(=O)O)CC3)cc(-c3cc(Cl)cc(Cl)c3)n2)cn1] | 2-(1-((2-(3,5-dichlorophenyl)-6-((2-(4- ethyl-1,4-diazepan-1-yl)pyrimidin-5- yl)oxy)pyridin-4-yl)methyl)piperidin- 4-yl)acetic acid |
| 231 | 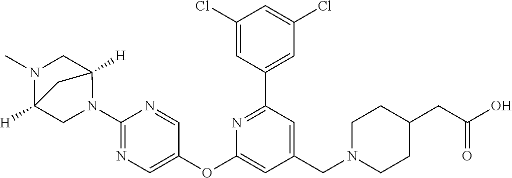 [SMILES: CN1C[C@@H]2C[C@H]1CN2c1ncc(Oc2cc(CN3CCC(CC(=O)O)CC3)cc(-c3cc(Cl)cc(Cl)c3)n2)cn1] | 2-(1-((2-(3,5-dichlorophenyl)-6-((2- ((1S,4S)-5-methyl-2,5- diazabicyclo[2.2.1]heptan-2-yl) pyrimidin-5-yl)oxy)pyridin-4-yl) methyl)piperidin-4-yl)acetic acid |
| 232 | 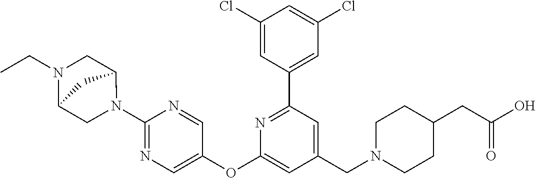 [SMILES: CCN1C[C@@H]2C[C@H]1CN2c1ncc(Oc2cc(CN3CCC(CC(=O)O)CC3)cc(-c3cc(Cl)cc(Cl)c3)n2)cn1] | 2-(1-((2-(3,5-dichlorophenyl)-6-((2- ((1S,4S)-5-ethyl-2,5- diazabicyclo[2.2.1]heptan-2-yl) pyrimidin-5-yl)oxy)pyridin-4-yl) methyl)piperidin-4-yl)acetic acid |
| 233 | 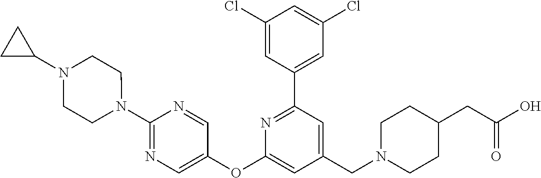 [SMILES: O=C(O)CC1CCN(Cc2cc(Oc3cnc(N4CCN(C5CC5)CC4)nc3)nc(-c3cc(Cl)cc(Cl)c3)c2)CC1] | 2-(1-((2-((2-(4-cyclopropylpiperazin-1- yl)pyrimidin-5-yl)oxy)-6-(3,5- dichlorophenyl)pyridin-4- yl)methyl)piperidin-4-yl)acetic acid |
| 234 | 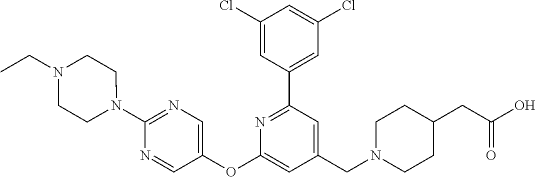 [SMILES: CCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2-(4- ethylpiperazin-1-yl)pyrimidin-5- yl)oxy)pyridin-4-yl)methyl)piperidin- 4-yl)acetic acid |
| 235 | 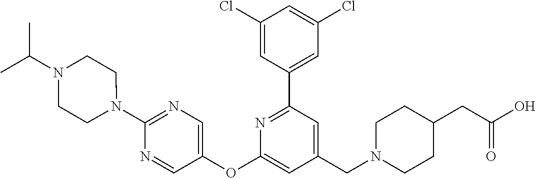 [SMILES: CC(C)N1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2-(4- isopropylpiperazin-1-yl)pyrimidin-5- yl)oxy)pyridin-4-yl)methyl)piperidin-4- yl)acetic acid |
| 236 | 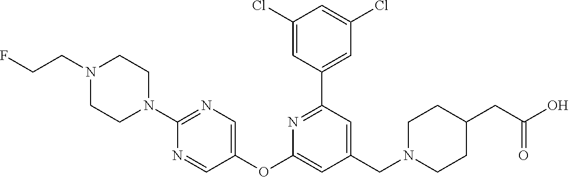 [SMILES: O=C(O)CC1CCN(Cc2cc(Oc3cnc(N4CCN(CCF)CC4)nc3)nc(-c3cc(Cl)cc(Cl)c3)c2)CC1] | 2-(1-((2-(3,5-dichlorophenyl)-6-((2- (4-(2-fluoroethyl)piperazin-1-yl) pyrimidin-5-yl)oxy)pyridin-4-yl) methyl)piperidin-4-yl)acetic acid |
| 237 | 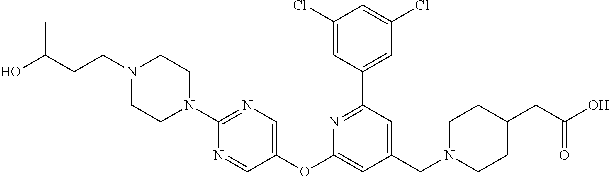 [SMILES: CC(O)CCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2- (4-(3-hydroxybutyl)piperazin-1-yl) pyrimidin-5-yl)oxy)pyridin-4-yl) methyl)piperidin-4-yl)acetic acid |
| 238 | 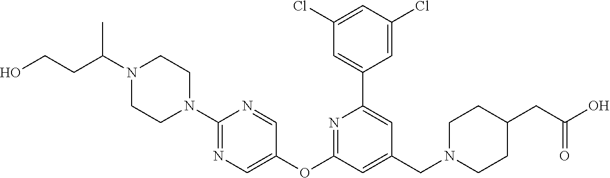 [SMILES: CC(CCO)N1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2- (4-(4-hydroxybutan-2-yl)piperazin-1- yl)pyrimidin-5-yl)oxy)pyridin-4- yl)methyl)piperidin-4-yl)acetic acid |
| 239 | 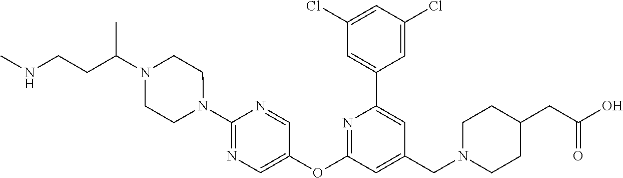 [SMILES: CNCCC(C)N1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2- (4-(4-(methylamino)butan-2-yl) piperazin-1-yl)pyrimidin-5-yl)oxy) pyridin-4-yl)methyl)piperidin-4- yl)acetic acid |
| 240 | 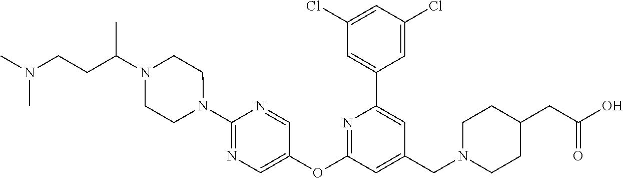 [SMILES: CC(CCN(C)C)N1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2- (4-(4-(dimethylamino)butan-2-yl) piperazin-1-yl)pyrimidin-5-yl)oxy) pyridin-4- yl)methyl)piperidin-4-yl)acetic acid |
| 241 | 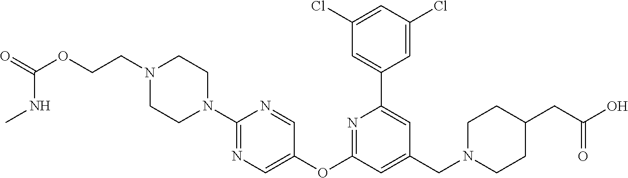 [SMILES: CNC(=O)OCCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2- (4-(2-((methylcarbamoyl)oxy)ethyl) piperazin-1-yl)pyrimidin-5-yl)oxy) pyridin-4-yl)methyl)piperidin-4- yl)acetic acid |
| 242 | 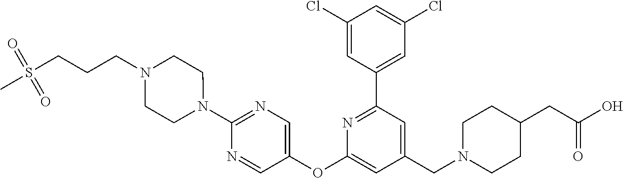 [SMILES: CS(=O)(=O)CCCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2- (4-(3-(methylsulfonyl)propyl) piperazin-1-yl)pyrimidin-5-yl)oxy) pyridin-4- yl)methyl)piperidin-4-yl)acetic acid |
| 243 | 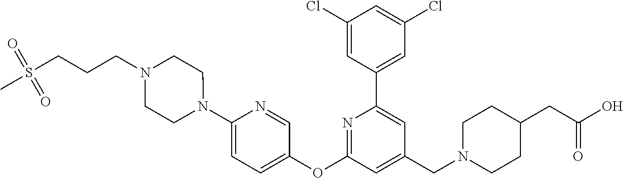 [SMILES: CS(=O)(=O)CCCN1CCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((6- (4-(3-(methylsulfonyl)propyl) piperazin-1- yl)pyridin-3-yl)oxy)pyridin-4- yl)methyl)piperidin-4-yl)acetic acid |
| 244 | 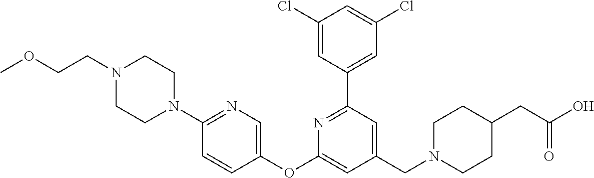 [SMILES: COCCN1CCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((6- (4-(2-methoxyethyl)piperazin-1-yl) pyridin-3-yl)oxy)pyridin-4-yl)methyl) piperidin-4- yl)acetic acid |
| 245 | 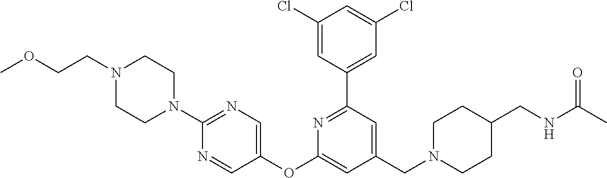 [SMILES: COCCN1CCN(c2ncc(Oc3cc(CN4CCC(CNC(C)=O)CC4)cc(-c4cc(Cl)cc(Cl)c4)n3)cn2)CC1] | N-((1-((2-(3,5-dichlorophenyl)-6-((2- (4-(2-methoxyethyl)piperazin-1- yl)pyrimidin-5-yl)oxy)pyridin-4- yl)methyl)piperidin-4- yl)methyl)acetamide |
| 246 | 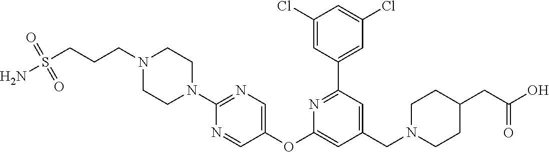 [SMILES: NS(=O)(=O)CCCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2-(4- (3-sulfamoylpropyl)piperazin-1- yl)pyrimidin-5-yl)oxy)pyridin-4- yl)methyl)piperidin-4-yl)acetic acid |
| 247 | 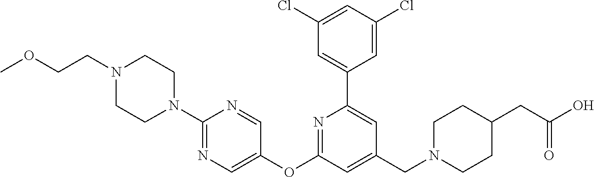 [SMILES: COCCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2- (4-(2-methoxyethyl)piperazin-1-yl) pyrimidin-5-yl)oxy)pyridin-4-yl) methyl)piperidin-4-yl)acetic acid |
| 248 | 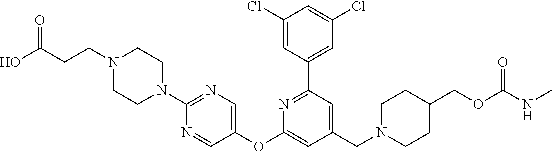 [SMILES: CNC(=O)OCC1CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichlorophenyl)-4-((4- (((methylcarbamoyl)oxy)methyl) piperidin-1-yl)methyl)pyridin-2-yl)oxy) pyrimidin-2-yl)piperazin-1-yl) propanoic acid |
| 249 | 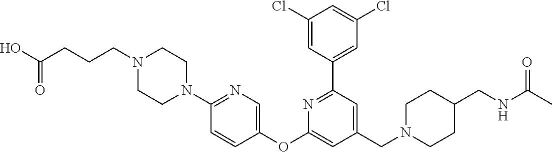 [SMILES: CC(=O)NCC1CCN(Cc2cc(Oc3ccc(N4CCN(CCCC(=O)O)CC4)nc3)nc(-c3cc(Cl)cc(Cl)c3)c2)CC1] | 4-(4-(5-((4-((4- (acetamidomethyl)piperidin-1-yl) methyl)- 6-(3,5-dichlorophenyl)pyridin-2- yl)oxy)pyridin-2-yl)piperazin-1- yl)butanoic acid |
| 250 | 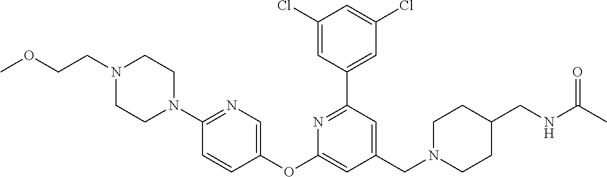 [SMILES: COCCN1CCN(c2ccc(Oc3cc(CN4CCC(CNC(C)=O)CC4)cc(-c4cc(Cl)cc(Cl)c4)n3)cn2)CC1] | N-((1-((2-(3,5-dichlorophenyl)-6-((6-(4- (2-methoxyethyl)piperazin-1-yl)pyridin- 3-yl)oxy)pyridin-4-yl)methyl)piperidin- 4-yl)methyl)acetamide |
| 251 | 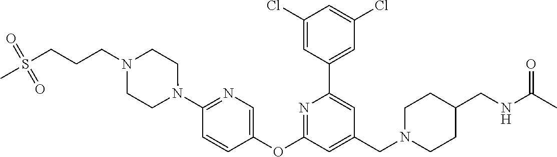 [SMILES: CC(=O)NCC1CCN(Cc2cc(Oc3ccc(N4CCN(CCCS(C)(=O)=O)CC4)nc3)nc(-c3cc(Cl)cc(Cl)c3)c2)CC1] | N-((1-((2-(3,5-dichlorophenyl)-6-((6-(4- (3-(methylsulfonyl)propyl)piperazin-1- yl)pyridin-3-yl)oxy)pyridin-4- yl)methyl)piperidin-4- yl)methyl)acetamide |
| 252 | 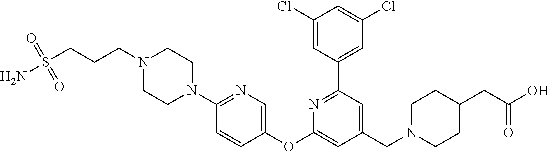 [SMILES: NS(=O)(=O)CCCN1CCN(c2ccc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((6- (4-(3-sulfamoylpropyl)piperazin-1-yl) pyridin-3-yl)oxy)pyridin-4-yl)methyl) piperidin-4-yl)acetic acid |
| 253 | 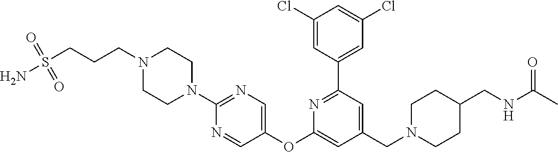 [SMILES: CC(=O)NCC1CCN(Cc2cc(Oc3cnc(N4CCN(CCCS(N)(=O)=O)CC4)nc3)nc(-c3cc(Cl)cc(Cl)c3)c2)CC1] | N-((1-((2-(3,5-dichlorophenyl)-6-((2- (4-(3-sulfamoylpropyl)piperazin-1- yl)pyrimidin-5-yl)oxy)pyridin-4- yl)methyl)piperidin-4- yl)methyl)acetamide |
| 254 | 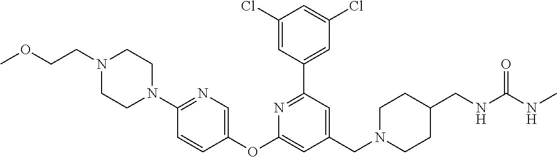 | 1-((1-((2-(3,5-dichlorophenyl)-6-((6-(4- (2-methoxyethyl)piperazin-1-yl)pyridin- 3-yl)oxy)pyridin-4-yl)methyl)piperidin- 4-yl)methyl)-3-methylurea |
| 255 | 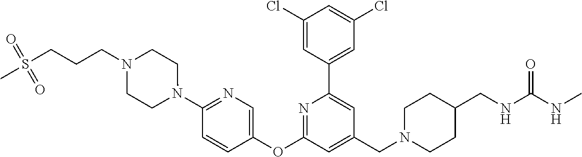 | 1-((1-((2-(3,5-dichlorophenyl)-6-((6-(4- (3-(methylsulfonyl)propyl)piperazin-1- yl)pyridin-3-yl)oxy)pyridin-4- yl)methyl)piperidin-4-yl)methyl)-3- methylurea |
| 256 | 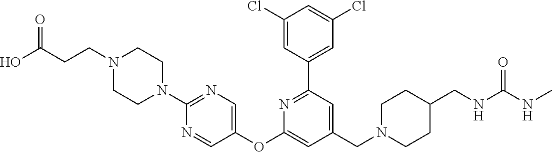 [SMILES: CNC(=O)NCC1CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichlorophenyl)-4-((4- ((3-methylureido)methyl)piperidin-1- yl)methyl)pyridin-2-yl)oxy)pyrimidin- 2-yl)piperazin-1-yl)propanoic acid |
| 257 | 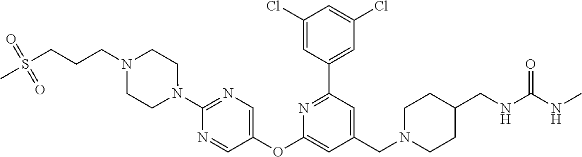 | 1-((1-((2-(3,5-dichlorophenyl)-6-((2-(4- (3-(methylsulfonyl)propyl)piperazin-1- yl)pyrimidin-5-yl)oxy)pyridin-4- yl)methyl)piperidin-4-yl)methyl)-3- methylurea |
| 258 | 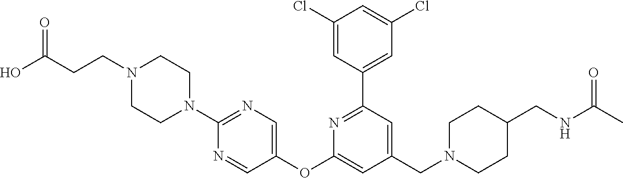 [SMILES: CC(=O)NCC1CCN(Cc2cc(Oc3cnc(N4CCN(CCC(=O)O)CC4)nc3)nc(-c3cc(Cl)cc(Cl)c3)c2)CC1] | 3-(4-(5-((5-((4- (acetamidomethyl)piperidin-1-yl) methyl)-3′,5′-dichloro-[1,1′-biphenyl]- 3-yl)oxy)pyridin-2-yl)piperazin-1- yl)propanoic acid |
| 259 | 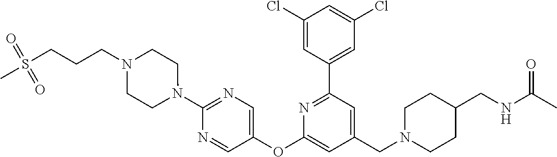 [SMILES: CC(=O)NCC1CCN(Cc2cc(Oc3cnc(N4CCN(CCCS(C)(=O)=O)CC4)nc3)nc(-c3cc(Cl)cc(Cl)c3)c2)CC1] | N-((1-((2-(3,5-dichlorophenyl)-6-((2- (4-(3-(methylsulfonyl)propyl) piperazin-1-yl)pyrimidin-5-yl)oxy) pyridin-4-yl)methyl)piperidin-4- yl)methyl)acetamide |
| 260 | 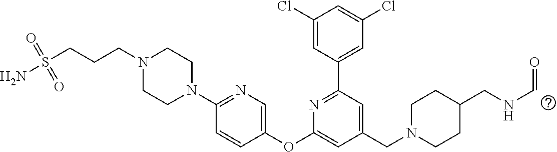 [SMILES: NS(=O)(=O)CCCN1CCN(c2ccc(Oc3cc(CN4CCC(CNC=O)CC4)cc(-c4cc(Cl)cc(Cl)c4)n3)cn2)CC1] | 3-(4-(5-((6-(3,5-dichlorophenyl)-4-((4- ((3-methylureido)methyl)piperidin-1- yl)methyl)pyridin-2-yl)oxy)pyridin-2- yl)piperazin-1-yl)propane-1- sulfonamide |
| 261 | 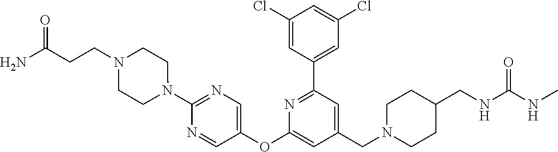 [SMILES: CNC(=O)NCC1CCN(Cc2cc(Oc3cnc(N4CCN(CCC(N)=O)CC4)nc3)nc(-c3cc(Cl)cc(Cl)c3)c2)CC1] | 3-(4-(5-((6-(3,5-dichlorophenyl)-4-((4- ((3-methylureido)methyl)piperidin-1- yl)methyl)pyridin-2-yl)oxy) pyrimidin-2-yl)piperazin-1-yl) propanamide |
| 262 | 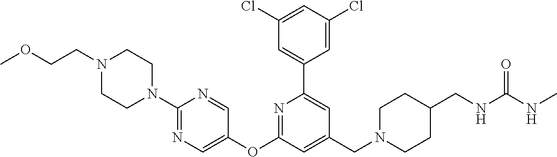 | 1-((1-((2-(3,5-dichlorophenyl)-6-((2- (4-(2-methoxyethyl)piperazin-1-yl) pyrimidin-5-yl)oxy)pyridin-4-yl) methyl)piperidin-4- yl)methyl)-3-methylurea |
| 263 | 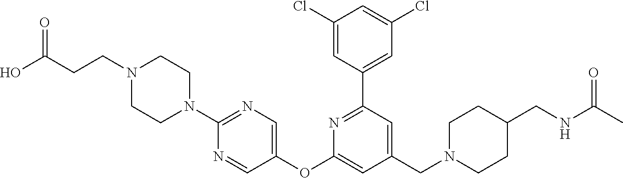 [SMILES: CC(=O)NCC1CCN(Cc2cc(Oc3cnc(N4CCN(CCC(=O)O)CC4)nc3)nc(-c3cc(Cl)cc(Cl)c3)c2)CC1] | 3-(4-(5-((4-((4- (acetamidomethyl)piperidin-1-yl) methyl)-6-(3,5-dichlorophenyl) pyridin-2-yl)oxy)pyrimidin-2-yl) piperazin-1-yl)propanoic acid |
| 264 | 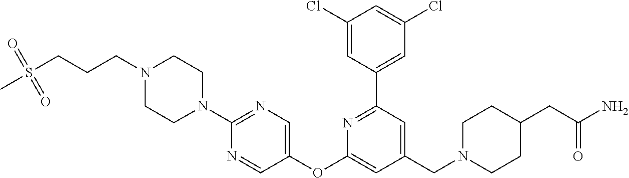 [SMILES: CS(=O)(=O)CCCN1CCN(c2ncc(Oc3cc(CN4CCC(CC(N)=O)CC4)cc(-c4cc(Cl)cc(Cl)c4)n3)cn2)CC1] | 2-(1-((2-(3,5-dichlorophenyl)-6-((6- (4-(3-(methylsulfonyl)propyl) piperazin-1-yl)pyridin-3-yl)oxy) pyridin-4-yl)methyl)piperidin-4- yl)acetamide |
| 265 | 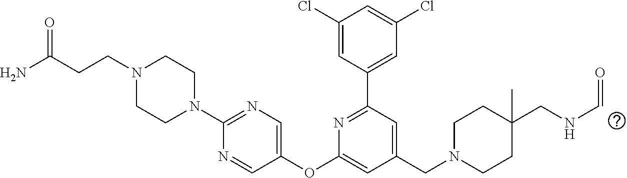 [SMILES: CC1(CNC=O)CCN(Cc2cc(Oc3cnc(N4CCN(CCC(N)=O)CC4)nc3)nc(-c3cc(Cl)cc(Cl)c3)c2)CC1] | 3-(4-(5-((6-(3,5-dichlorophenyl)-4-((4- hydroxy-4-((3- methylureido)methyl)piperidin-1- yl)methyl)pyridin-2-yl)oxy) pyrimidin-2-yl)piperazin-1-yl) propanamide |
| 266 | 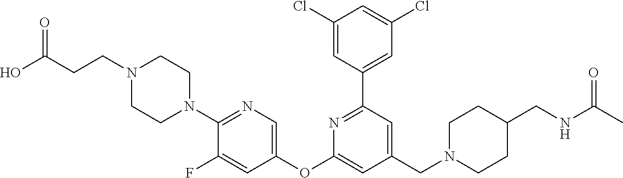 [SMILES: CC(=O)NCC1CCN(Cc2cc(Oc3cnc(N4CCN(CCC(=O)O)CC4)c(F)c3)nc(-c3cc(Cl)cc(Cl)c3)c2)CC1] | 3-(4-(5-((4-(4- (acetamidomethyl)piperidin-1-yl) methyl)-6-(3,5-dichlorophenyl) pyridin-2-yl)oxy)- 3-fluoropyridin-2-yl)piperazin-1- yl)propanoic acid |
| 267 | 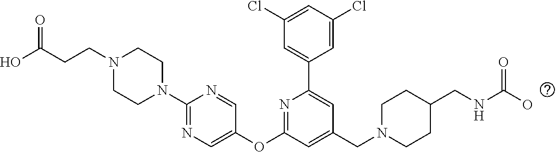 [SMILES: O=C(O)CCN1CCN(c2ncc(Oc3cc(CN4CCC(CNC(=O)O)CC4)cc(-c4cc(Cl)cc(Cl)c4)n3)cn2)CC1] | 3-(4-(5-((6-(3,5-dichlorophenyl)-4-((4- (((ethoxycarbonyl)amino)methyl) piperidin-1-yl)methyl)pyridin-2-yl) oxy)pyrimidin-2-yl)piperazin-1-yl) propanoic acid |
| 268 | 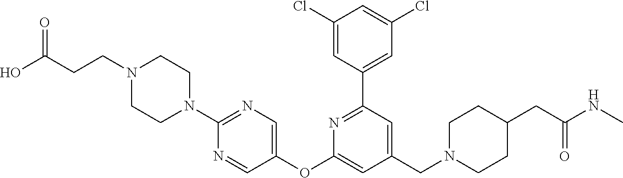 [SMILES: CNC(=O)CC1CCN(Cc2cc(Oc3cnc(N4CCN(CCC(=O)O)CC4)nc3)nc(-c3cc(Cl)cc(Cl)c3)c2)CC1] | 3-(4-(5-((6-(3,5-dichlorophenyl)-4-((4- (2-(ethylamino)-2-oxoethyl)piperidin- 1-yl)methyl)pyridin-2-yl)oxy) pyrimidin-2-yl)piperazin-1-yl) propanoic acid |
| 269 | 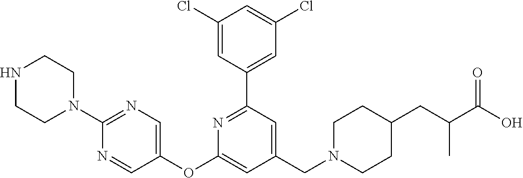 [SMILES: CC(CC1CCN(Cc2cc(Oc3cnc(N4CCNCC4)nc3)nc(-c3cc(Cl)cc(Cl)c3)c2)CC1)C(=O)O] | 3-(1-((2-(3,5-dichlorophenyl)-6-((2- (piperazin-1-yl)pyrimidin-5- yl)oxy)pyridin-4-yl)methyl)piperidin- 4-yl)-2-methylpropanoic acid |
| 270 | 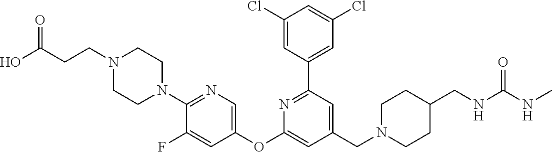 [SMILES: CNC(=O)NCC1CCN(Cc2cc(Oc3cnc(N4CCN(CCC(=O)O)CC4)c(F)c3)nc(-c3cc(Cl)cc(Cl)c3)c2)CC1] | 3-(4-(5-((6-(3,5-dichlorophenyl)-4-((4- ((3-methylureido)methyl)piperidin-1- yl)methyl)pyridin-2-yl)oxy)-3- fluoropyridin-2-yl)piperazin-1- yl)propanoic acid |
| 271 | 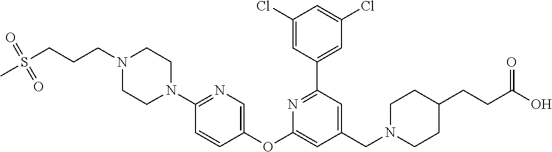 [SMILES: CS(=O)(=O)CCCN1CCN(c2ccc(Oc3cc(CN4CCC(CCC(=O)O)CC4)cc(-c4cc(Cl)cc(Cl)c4)n3)cn2)CC1] | 3-(1-((2-(3,5-dichlorophenyl)-6-((6-(4- (3-(methylsulfonyl)propyl)piperazin-1- yl)pyridin-3-yl)oxy)pyridin-4- yl)methyl)piperidin-4-yl)propanoic acid |
| 272 | 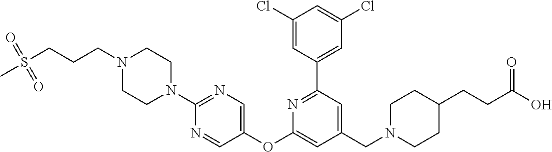 [SMILES: CS(=O)(=O)CCCN1CCN(c2ncc(Oc3cc(CN4CCC(CCC(=O)O)CC4)cc(-c4cc(Cl)cc(Cl)c4)n3)cn2)CC1] | 3-(1-((2-(3,5-dichlorophenyl)-6-((2-(4- (3-(methylsulfonyl)propyl)piperazin-1- yl)pyrimidin-5-yl)oxy)pyridin-4- yl)methyl)piperidin-4-yl)propanoic acid |
| 273 | 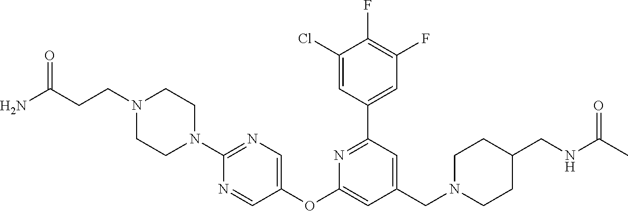 [SMILES: CC(=O)NCC1CCN(Cc2cc(Oc3cnc(N4CCN(CCC(N)=O)CC4)nc3)nc(-c3cc(F)c(F)c(Cl)c3)c2)CC1] | 3-(4-(5-((4-(4- (acetamidomethyl)piperidin-1-yl) methyl)-6-(3-chloro-4,5- difluorophenyl)pyridin-2- yl)oxy)pyrimidin-2-yl)piperazin-1- yl)propanamide |
| 274 | 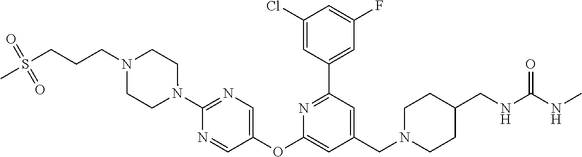 | 1-((1-((2-(3-chloro-5-fluorophenyl)-6- ((2-(4-(3-(methylsulfonyl)propyl) piperazin-1-yl)pyrimidin-5-yl)oxy) pyridin-4-yl)methyl)piperidin-4-yl) methyl)-3-methylurea |
| 275 | 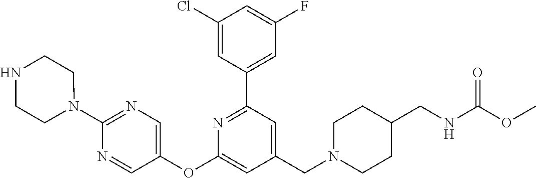 [SMILES: COC(=O)NCC1CCN(Cc2cc(Oc3cnc(N4CCNCC4)nc3)nc(-c3cc(F)cc(Cl)c3)c2)CC1] | methyl((1-((2-(3-chloro-5- fluorophenyl)-6-((2-(piperazin-1-yl) pyrimidin-5-yl)oxy)pyridin-4-yl) methyl)piperidin-4- yl)methyl)carbamate |
| 276 | 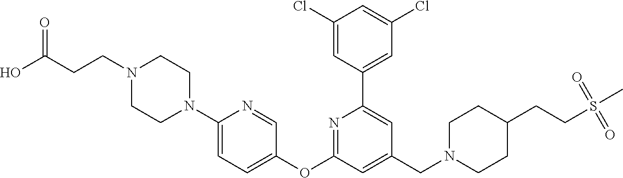 [SMILES: CS(=O)(=O)CCC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)-4- ((4-(2-(methylsulfonyl)ethyl) piperidin-1-yl)methyl)pyridin-2-yl) oxy)pyridin-2- yl)piperazin-1-yl)propanoic acid |
| 277 | 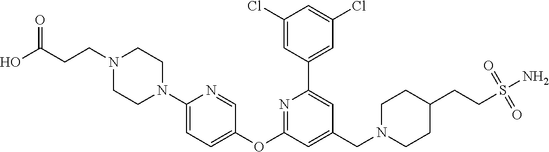 [SMILES: NS(=O)(=O)CCC1CCN(Cc2cc(Oc3ccc(N4CCN(CCC(=O)O)CC4)nc3)nc(-c3cc(Cl)cc(Cl)c3)c2)CC1] | 3-(4-(5-((6-(3,5-dichlorophenyl)-4- ((4-(2-sulfamoylethyl)piperidin-1- yl)methyl)pyridin-2-yl)oxy)pyridin-2- yl)piperazin-1-yl)propanoic acid |
| 278 | 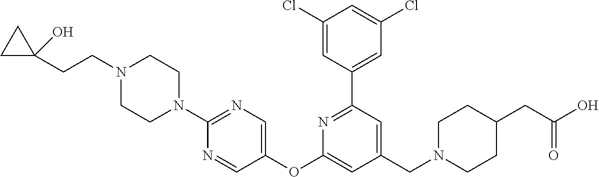 [SMILES: O=C(O)CC1CCN(Cc2cc(Oc3cnc(N4CCN(CCC5(O)CC5)CC4)nc3)nc(-c3cc(Cl)cc(Cl)c3)c2)CC1] | 2-(1-((2-(3,5-dichlorophenyl)-6-((2- (4-(2-(1-hydroxycyclopropyl)ethyl) piperazin-1-yl)pyrimidin-5-yl)oxy) pyridin-4- yl)methyl)piperidin-4-yl)acetic acid |
| 279 | 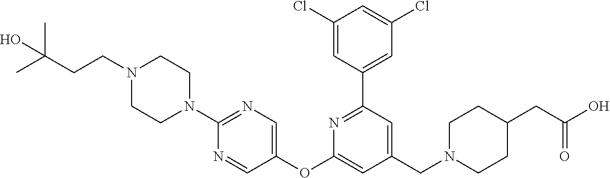 [SMILES: CC(C)(O)CCN1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2- (4-(3-hydroxy-3-methylbutyl) piperazin-1- yl)pyrimidin-5-yl)oxy)pyridin-4- yl)methyl)piperidin-4-yl)acetic acid |
| 280 | 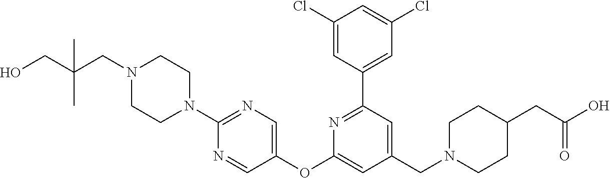 [SMILES: CC(C)(CO)CN1CCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((2- (4-(3-hydroxy-2,2-dimethylpropyl) piperazin-1- yl)pyrimidin-5-yl)oxy)pyridin-4- yl)methyl)piperidin-4-yl)acetic acid |
| 281 | 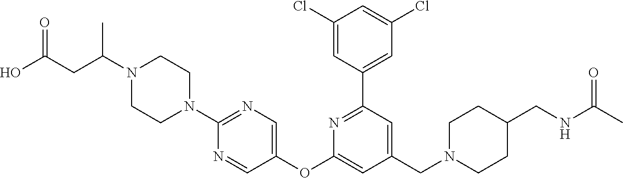 [SMILES: CC(=O)NCC1CCN(Cc2cc(Oc3cnc(N4CCN(C(C)CC(=O)O)CC4)nc3)nc(-c3cc(Cl)cc(Cl)c3)c2)CC1] | 3-(4-(5-((4-((4- (acetamidomethyl)piperidin-1-yl) methyl)-6-(3,5-dichlorophenyl) pyridin-2-yl)oxy)pyridin-2-yl) piperazin-1-yl)butanoic acid |
| 282 | 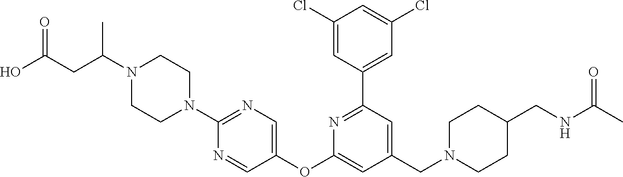 [SMILES: CC(=O)NCC1CCN(Cc2cc(Oc3cnc(N4CCN(C(C)CC(=O)O)CC4)nc3)nc(-c3cc(Cl)cc(Cl)c3)c2)CC1] | 3-(4-(5-((4-((4- (acetamidomethyl)piperidin-1-yl) methyl)- 6-(3,5-dichlorophenyl)pyridin-2- yl)oxy)pyrimidin-2-yl)piperazin-1- yl)butanoic acid |
| 283 | 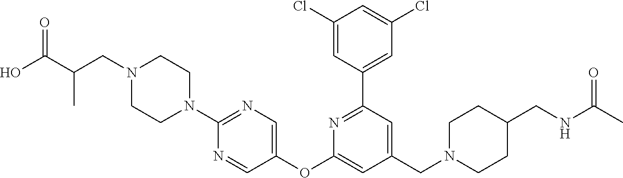 [SMILES: CC(=O)NCC1CCN(Cc2cc(Oc3cnc(N4CCN(CC(C)C(=O)O)CC4)nc3)nc(-c3cc(Cl)cc(Cl)c3)c2)CC1] | 3-(4-(5-((4-((4- (acetamidomethyl)piperidin-1-yl) methyl)- 6-(3,5-dichlorophenyl)pyridin-2- yl)oxy)pyrimidin-2-yl)piperazin-1- yl)-2-methylpropanoic acid |
| 284 | 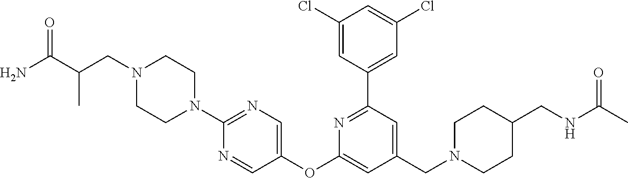 [SMILES: CC(=O)NCC1CCN(Cc2cc(Oc3cnc(N4CCN(CC(C)C(N)=O)CC4)nc3)nc(-c3cc(Cl)cc(Cl)c3)c2)CC1] | 3-(4-(5-((4-(4- (acetamidomethyl)piperidin-1-yl) methyl)- 6-(3,5-dichlorophenyl)pyridin-2- yl)oxy)pyrimidin-2-yl)piperazin- 1-yl)-2-methylpropan amide |
| 285 | 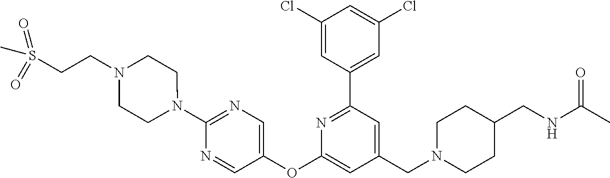 [SMILES: CC(=O)NCC1CCN(Cc2cc(Oc3cnc(N4CCN(CCS(C)(=O)=O)CC4)nc3)nc(-c3cc(Cl)cc(Cl)c3)c2)CC1] | N-((1-((2-(3,5-dichlorophenyl)-6-((2- (4-(2-(methylsulfonyl)ethyl)piperazin- 1-yl)pyrimidin-5-yl)oxy)pyridin-4- yl)methyl)piperidin-4- yl)methyl)acetamide |
| 286 | 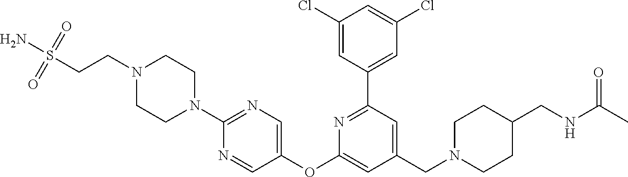 [SMILES: CC(=O)NCC1CCN(Cc2cc(Oc3cnc(N4CCN(CCS(N)(=O)=O)CC4)nc3)nc(-c3cc(Cl)cc(Cl)c3)c2)CC1] | N-((1-((2-(3,5-dichlorophenyl)-6-((2- (4-(2-sulfamoylethyl)piperazin-1- yl)pyrimidin-5-yl)oxy)pyridin-4- yl)methyl)piperidin-4- yl)methyl)acetamide |
| 287 | 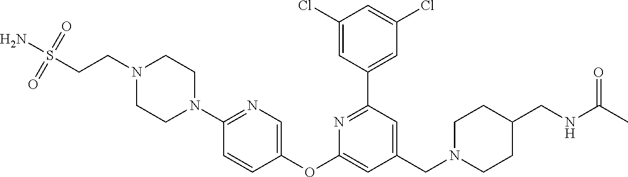 [SMILES: CC(=O)NCC1CCN(Cc2cc(Oc3ccc(N4CCN(CCS(N)(=O)=O)CC4)nc3)nc(-c3cc(Cl)cc(Cl)c3)c2)CC1] | 2-(4-(5-((4-(4- (acetamidomethyl)piperidin-1-yl) methyl)- 6-(3,5-dichlorophenyl)pyridin-2- yl)oxy)pyridin-2-yl)piperazin-1- yl)ethanesulfonic acid |
| 288 | 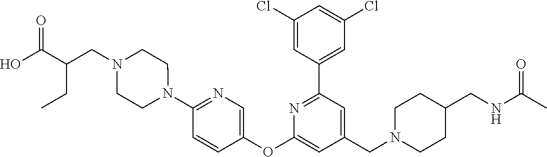 [SMILES: CCC(CN1CCN(c2ccc(Oc3cc(CN4CCC(CNC(C)=O)CC4)cc(-c4cc(Cl)cc(Cl)c4)n3)cn2)CC1)C(=O)O] | 2-((4-(5-((4-((4- (acetamidomethyl)piperidin-1-yl) methyl)- 6-(3,5-dichlorophenyl)pyridin-2- yl)oxy)pyridin-2-yl)piperazin-1- yl)methyl)butanoic acid |
| 289 | 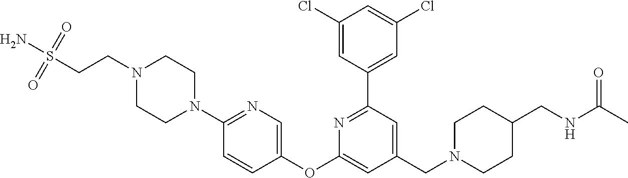 [SMILES: CC(=O)NCC1CCN(Cc2cc(Oc3ccc(N4CCN(CCS(N)(=O)=O)CC4)nc3)nc(-c3cc(Cl)cc(Cl)c3)c2)CC1] | N-((1-((2-(3,5-dichlorophenyl)-6-((6- (4-(2-sulfamoylethyl)piperazin-1-yl) pyridin-3-yl)oxy)pyridin-4-yl)methyl) piperidin-4-yl)methyl)acetamide |
| 290 | 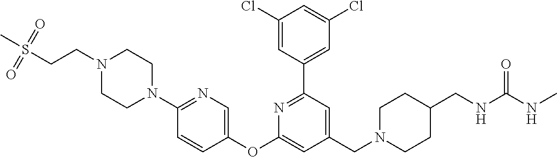 | 1-((1-((2-(3,5-dichlorophenyl)-6-((6- (4-(2-(methylsulfonyl)ethyl)piperazin- 1-yl)pyridin-3-yl)oxy)pyridin-4- yl)methyl)piperidin-4-yl)methyl)-3- methylurea |
| 291 | 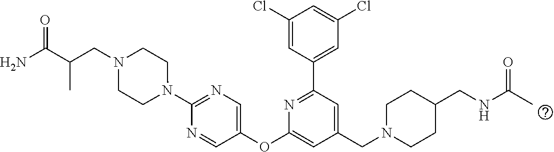 [SMILES: CC(=O)NCC1CCN(Cc2cc(Oc3cnc(N4CCN(CC(C)C(N)=O)CC4)nc3)nc(-c3cc(Cl)cc(Cl)c3)c2)CC1] | 3-(4-(5-((6-(3,5-dichlorophenyl)-4-((4- ((3-methylureido)methyl)piperidin-1- yl)methyl)pyridin-2-yl)oxy)pyrimidin- 2-yl)piperazin-1-yl)-2-methyl- propanamide |
| 292 | 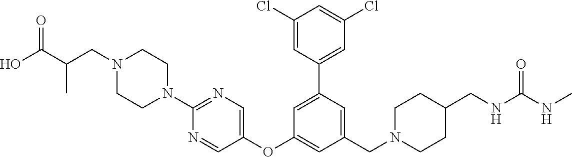 [SMILES: CNC(=O)NCC1CCN(Cc2cc(Oc3cnc(N4CCN(CC(C)C(=O)O)CC4)nc3)cc(-c3cc(Cl)cc(Cl)c3)c2)CC1] | 3-(4-(5-((3′,5′-dichloro-5-((4-((3- methylureido)methyl)piperidin-1- yl)methyl)-[1,1′-biphenyl]-3- yl)oxy)pyrimidin-2-yl)piperazin-1-yl)- 2-methylpropanoic acid |
| 293 | 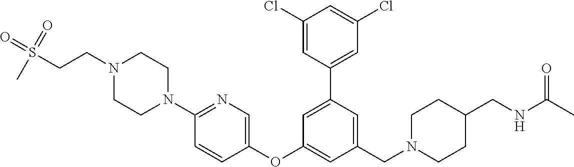 [SMILES: CC(=O)NCC1CCN(Cc2cc(Oc3ccc(N4CCN(CCS(C)(=O)=O)CC4)nc3)cc(-c3cc(Cl)cc(Cl)c3)c2)CC1] | N-((1-((3′,5′-dichloro-5-((6-(4-(2- (methylsulfonyl)ethyl)piperazin-1- yl)pyridin-3-yl)oxy)-[1,1′-biphenyl]-3- yl)methyl)piperidin-4- yl)methyl)acetamide |
| 294 | 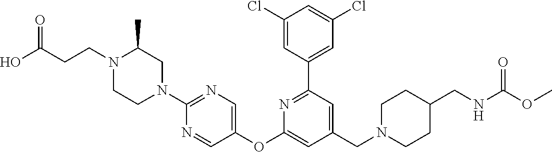 [SMILES: COC(=O)NCC1CCN(Cc2cc(Oc3cnc(N4CCN(CCC(=O)O)[C@@H](C)C4)nc3)nc(-c3cc(Cl)cc(Cl)c3)c2)CC1] | (S)-3-(4-(5-((6-(3,5-dichlorophenyl)- 4-((4- (((methoxycarbonyl)amino)methyl) piperidin-1-yl)methyl)pyridin-2- yl)oxy)pyrimidin-2-yl)-2-methyl- piperazin-1-yl)propanoic acid |
| 295 | 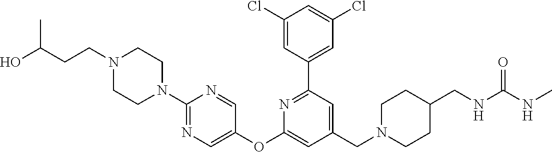 [SMILES: CNC(=O)NCC1CCN(Cc2cc(Oc3cnc(N4CCN(CCC(C)O)CC4)nc3)nc(-c3cc(Cl)cc(Cl)c3)c2)CC1] | 1-((1-((2-(3,5-dichlorophenyl)-6-((2- (4-(3-hydroxybutyl)piperazin-1-yl) pyrimidin-5-yl)oxy)pyridin-4-yl) methyl) piperidin-4-yl)methyl)-3-methylurea |
| 296 | 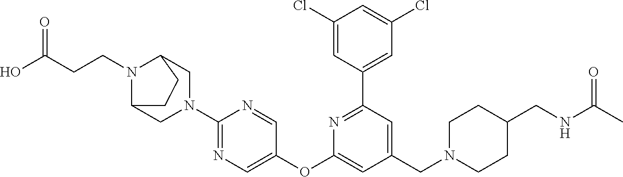 [SMILES: CC(=O)NCC1CCN(Cc2cc(Oc3cnc(N4CC5CCC(C4)N5CCC(=O)O)nc3)nc(-c3cc(Cl)cc(Cl)c3)c2)CC1] | 3-(3-(5-((4-(4- (acetamidomethyl)piperidin-1-yl) methyl)- 6-(3,5-dichlorophenyl)pyridin-2- yl)oxy)pyrimidin-2-yl)-3,8- diazabicyclo[3.2.1]octan-8-yl) propanoic acid |
| 297 | 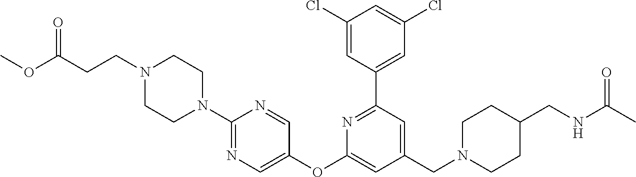 [SMILES: COC(=O)CCN1CCN(c2ncc(Oc3cc(CN4CCC(CNC(C)=O)CC4)cc(-c4cc(Cl)cc(Cl)c4)n3)cn2)CC1] | methyl3-(4-(5-((4-((4- (acetamidomethyl)piperidin-1-yl) methyl)- 6-(3,5-dichlorophenyl)pyridin-2- yl)oxy)pyrimidin-2-yl)piperazin-1- yl)propanoate |
| 298 | 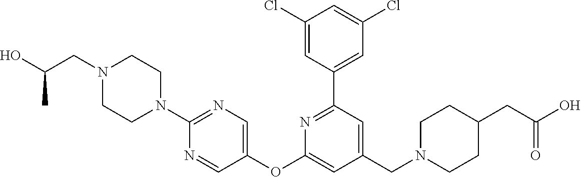 [SMILES: C[C@@H](O)CN1CCN(c2ncc(Oc3cc(CN4CCC(CC(=O)O)CC4)cc(-c4cc(Cl)cc(Cl)c4)n3)cn2)CC1] | (R)-2-(1-((2-(3,5-dichlorophenyl)-6- ((2-(4-(2-hydroxypropyl)piperazin-1- yl)pyrimidin-5-yl)oxy)pyridin-4- yl)methyl)piperidin-4-yl)acetic acid |
| 299 | 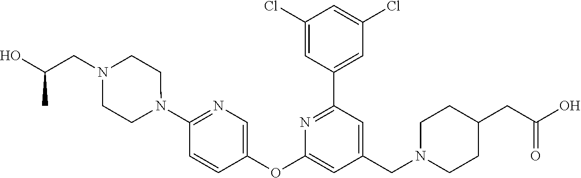 [SMILES: C[C@@H](O)CN1CCN(c2ccc(Oc3cc(CN4CCC(CC(=O)O)CC4)cc(-c4cc(Cl)cc(Cl)c4)n3)cn2)CC1] | (R)-2-(1-((2-(3,5-dichlorophenyl)-6- ((6-(4-(2-hydroxypropyl)piperazin-1- yl)pyridin-3-yl)oxy)pyridin-4- yl)methyl)piperidin-4-yl)acetic acid |
| 300 | 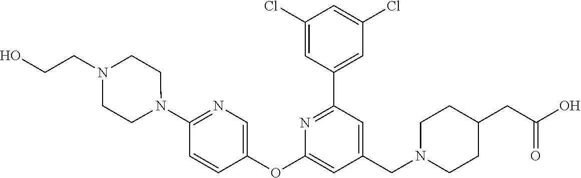 [SMILES: O=C(O)CC1CCN(Cc2cc(Oc3ccc(N4CCN(CCO)CC4)nc3)nc(-c3cc(Cl)cc(Cl)c3)c2)CC1] | 2-(1-((2-(3,5-dichlorophenyl)-6-((6-(4- (2-hydroxyethyl)piperazin-1-yl) pyridin-3-yl)oxy)pyridin-4-yl)methyl) piperidin-4-yl)acetic acid |
| 301 | 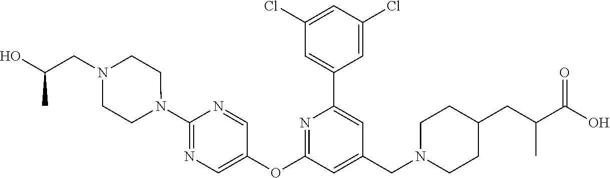 [SMILES: CC(CC1CCN(Cc2cc(Oc3cnc(N4CCN(C[C@@H](C)O)CC4)nc3)nc(-c3cc(Cl)cc(Cl)c3)c2)CC1)C(=O)O] | 3-(1-((2-(3,5-dichlorophenyl)-6-((2-(4- ((R)-2-hydroxypropyl)piperazin-1- yl)pyrimidin-5-yl)oxy)pyridin-4- yl)methyl)piperidin-4-yl)-2- methylpropanoic acid |
| 302 | 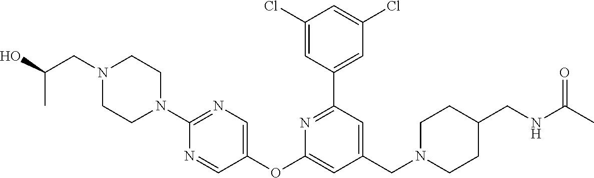 [SMILES: CC(=O)NCC1CCN(Cc2cc(Oc3cnc(N4CCN(C[C@@H](C)O)CC4)nc3)nc(-c3cc(Cl)cc(Cl)c3)c2)CC1] | (R)-N-((1-((2-(3,5-dichlorophenyl)-6- ((2-(4-(2-hydroxypropyl)piperazin-1- yl)pyrimidin-5-yl)oxy)pyridin-4- yl)methyl)piperidin-4- yl)methyl)acetamide |
| 303 | 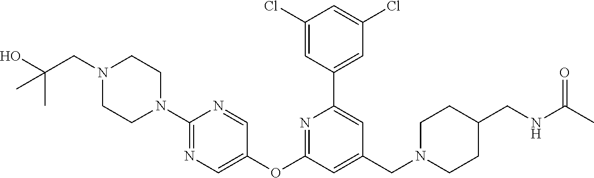 [SMILES: CC(=O)NCC1CCN(Cc2cc(Oc3cnc(N4CCN(CC(C)(C)O)CC4)nc3)nc(-c3cc(Cl)cc(Cl)c3)c2)CC1] | N-((1-((2-(3,5-dichlorophenyl)-6-((2- (4-(2-hydroxy-2-methylpropyl) piperazin-1- yl)pyrimidin-5-yl)oxy)pyridin-4- yl)methyl)piperidin-4- yl)methyl)acetamide |
| 304 | 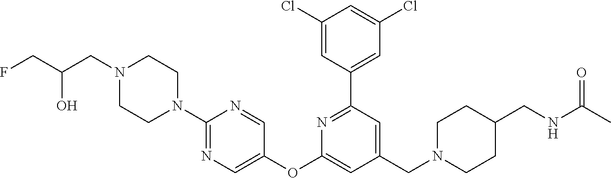 [SMILES: CC(=O)NCC1CCN(Cc2cc(Oc3cnc(N4CCN(CC(O)CF)CC4)nc3)nc(-c3cc(Cl)cc(Cl)c3)c2)CC1] | N-((1-((2-(3,5-dichlorophenyl)-6-((2- (4-(3-fluoro-2-hydroxypropyl) piperazin-1- yl)pyrimidin-5-yl)oxy)pyridin-4- yl)methyl)piperidin-4- yl)methyl)acetamide |
| 305 | 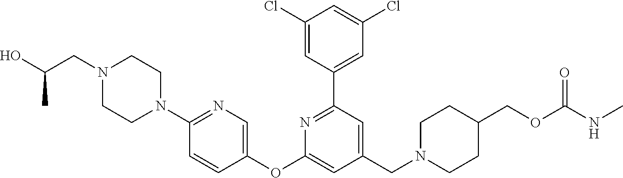 [SMILES: CNC(=O)OCC1CCN(Cc2cc(Oc3ccc(N4CCN(C[C@@H](C)O)CC4)nc3)nc(-c3cc(Cl)cc(Cl)c3)c2)CC1] | (R)-(1-((2-(3,5-dichlorophenyl)-6-((6- (4-(2-hydroxypropyl)piperazin-1-yl) pyridin-3-yl)oxy)pyridin-4-yl)methyl) piperidin-4-yl)methylmethylcarbamate |
| 306 | 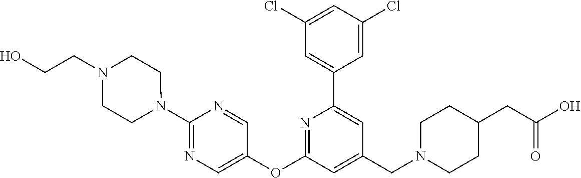 [SMILES: O=C(O)CC1CCN(Cc2cc(Oc3cnc(N4CCN(CCO)CC4)nc3)nc(-c3cc(Cl)cc(Cl)c3)c2)CC1] | 2-(1-((2-(3,5-dichlorophenyl)-6-((2-(4- (2-hydroxyethyl)piperazin-1-yl) pyrimidin-5-yl)oxy)pyridin-4-yl) methyl)piperidin-4- yl)acetic acid |
| 307 | 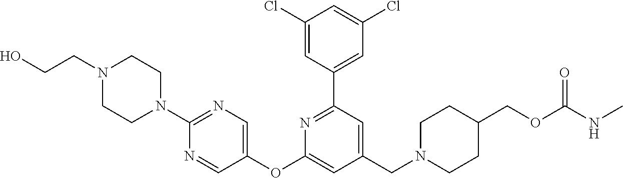 [SMILES: CNC(=O)OCC1CCN(Cc2cc(Oc3cnc(N4CCN(CCO)CC4)nc3)nc(-c3cc(Cl)cc(Cl)c3)c2)CC1] | (1-((2-(3,5-chlorophenyl)-6-((2-(4-(2- hydroxyethyl)piperazin-1-yl)pyrimidin- 5-yl)oxy)pyridin-4-yl)methyl)piperidin- 4-yl)methylmethylcarbamate |
| 308 | 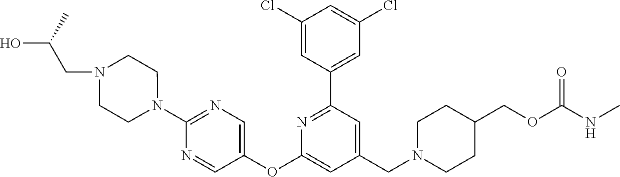 [SMILES: CNC(=O)OCC1CCN(Cc2cc(Oc3cnc(N4CCN(C[C@@H](C)O)CC4)nc3)nc(-c3cc(Cl)cc(Cl)c3)c2)CC1] | (R)-(1-((2-(3,5-dichlorophenyl)-6-((2- (4-(2-hydroxypropyl)piperazin-1- yl)pyrimidin-5-yl)oxy)pyridin-4- yl)methyl)piperidin-4- yl)methylmethylcarbamate |
| 309 | 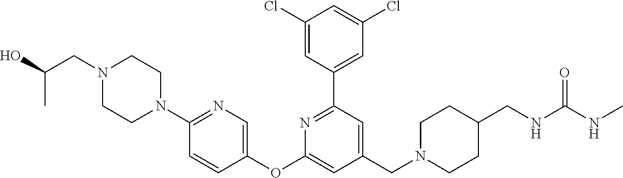 [SMILES: CNC(=O)NCC1CCN(Cc2cc(Oc3ccc(N4CCN(C[C@@H](C)O)CC4)nc3)nc(-c3cc(Cl)cc(Cl)c3)c2)CC1] | (R)-1-((1-((2-(3,5-dichlorophenyl)-6- ((6-(4-(2-hydroxypropyl)piperazin-1- yl)pyridin-3-yl)oxy)pyridin-4- yl)methyl)piperidin-4-yl)methyl)-3- methylurea |
| 310 | 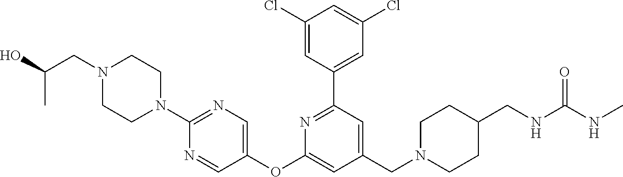 [SMILES: CNC(=O)NCC1CCN(Cc2cc(Oc3cnc(N4CCN(C[C@@H](C)O)CC4)nc3)nc(-c3cc(Cl)cc(Cl)c3)c2)CC1] | (R)-1-((1-((2-(3,5-dichlorophenyl)-6- ((2-(4-(2-hydroxypropyl)piperazin-1- yl)pyrimidin-5-yl)oxy)pyridin-4- yl)methyl)piperidin-4-yl)methyl)-3- methylurea |
| 311 | 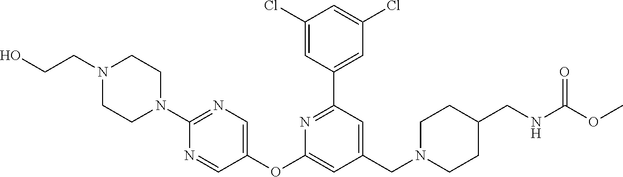 [SMILES: COC(=O)NCC1CCN(Cc2cc(Oc3cnc(N4CCN(CCO)CC4)nc3)nc(-c3cc(Cl)cc(Cl)c3)c2)CC1] | methyl((1-((2-(3,5-dichlorophenyl)-6- ((2-(4-(2-hydroxyethyl)piperazin-1- yl)pyrimidin-5-yl)oxy)pyridin-4- yl)methyl)piperidin-4- yl)methyl)carbamate |
| 312 | 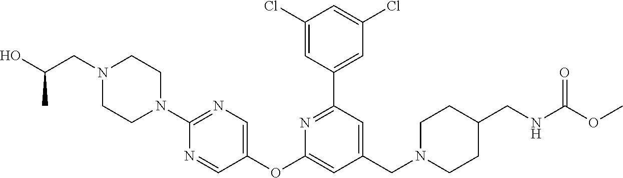 [SMILES: COC(=O)NCC1CCN(Cc2cc(Oc3cnc(N4CCN(C[C@@H](C)O)CC4)nc3)nc(-c3cc(Cl)cc(Cl)c3)c2)CC1] | (R)-methyl((1-((2-(3,5-dichlorophenyl)- 6-((2-(4-(2-hydroxypropyl)piperazin-1- yl)pyrimidin-5-yl)oxy)pyridin-4- yl)methyl)piperidin-4- yl)methyl)carbamate |
| 313 | 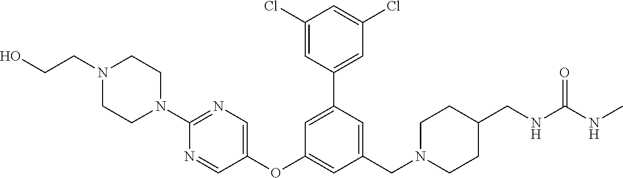 [SMILES: CNC(=O)NCC1CCN(Cc2cc(Oc3cnc(N4CCN(CCO)CC4)nc3)cc(-c3cc(Cl)cc(Cl)c3)c2)CC1] | 1-((1-((3′,5′-dichloro-5-((2-(4-(2- hydroxyethyl)piperazin-1-yl)pyrimidin- 5-yl)oxy)-[1,1′-biphenyl]-3- yl)methyl)piperidin-4-yl)methyl)-3- methylurea |
| 314 | 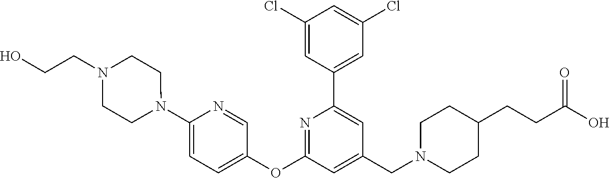 [SMILES: O=C(O)CCC1CCN(Cc2cc(Oc3ccc(N4CCN(CCO)CC4)nc3)nc(-c3cc(Cl)cc(Cl)c3)c2)CC1] | 3-(1-((2-(3,5-dichlorophenyl)-6-((6-(4- (2-hydroxyethyl)piperazin-1-yl) pyridin-3-yl)oxy)pyridin-4-yl)methyl) piperidin-4-yl)propanoic acid |
| 315 | 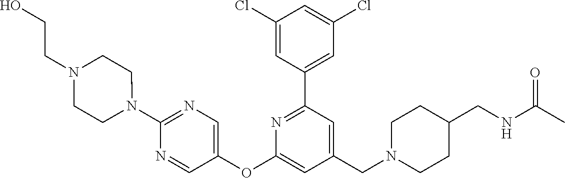 [SMILES: CC(=O)NCC1CCN(Cc2cc(Oc3cnc(N4CCN(CCO)CC4)nc3)nc(-c3cc(Cl)cc(Cl)c3)c2)CC1] | N-((1-((2-(3-chloro-5-fluorophenyl)-6- ((2-(4-(2-hydroxyethyl)piperazin-1- yl)pyrimidin-5-yl)oxy)pyridin-4- yl)methyl)piperidin-4- yl)methyl)acetamide |
| 316 | 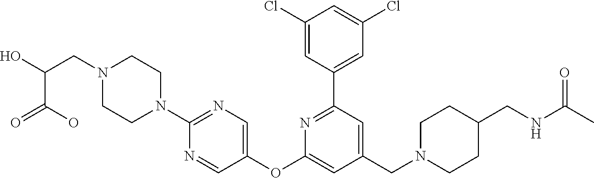 [SMILES: CC(=O)NCC1CCN(Cc2cc(Oc3cnc(N4CCN(CC(O)C(=O)O)CC4)nc3)nc(-c3cc(Cl)cc(Cl)c3)c2)CC1] | 3-(4-(5-((4-((4- (acetamidomethyl)piperidin-1-yl) methyl)- 6-(3,5-dichlorophenyl)pyridin-2- yl)oxy)pyrimidin-2-yl)piperazin- 1-yl)-2-hydroxypropanoic acid |
| 317 | 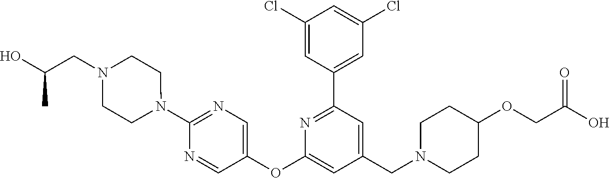 [SMILES: C[C@@H](O)CN1CCN(c2ncc(Oc3cc(CN4CCC(OCC(=O)O)CC4)cc(-c4cc(Cl)cc(Cl)c4)n3)cn2)CC1] | (R)-2-((1-((2-(3,5-dichlorophenyl)-6- ((2-(4-(2-hydroxypropyl)piperazin-1- yl)pyrimidin-5-yl)oxy)pyridin-4- yl)methyl)piperidin-4-yl)oxy)acetic acid |
| 318 | 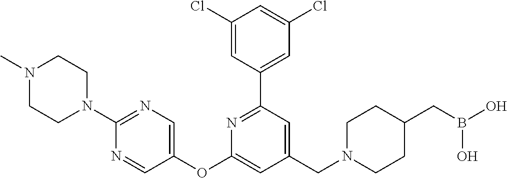 [SMILES: CN1CCN(c2ncc(Oc3cc(CN4CCC(CB(O)O)CC4)cc(-c4cc(Cl)cc(Cl)c4)n3)cn2)CC1] | ((1-((2-(3,5-dichlorophenyl)-6-((2-(4- methylpiperazin-1-yl)pyrimidin-5- yl)oxy)pyridin-4-yl)methyl)piperidin-4- yl)methyl)boronic acid |
| 319 | 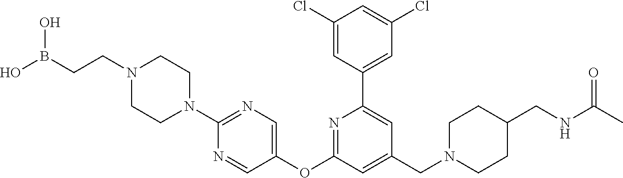 [SMILES: CC(=O)NCC1CCN(Cc2cc(Oc3cnc(N4CCN(CCB(O)O)CC4)nc3)nc(-c3cc(Cl)cc(Cl)c3)c2)CC1] | (2-(4-(5-((4-((4- (acetamidomethyl)piperidin-1-yl) methyl)-6-(3,5-dichlorophenyl)pyridin- 2-yl)oxy)pyrimidin-2-yl)piperazin-1- yl)ethyl)boronic acid |
| 320 | 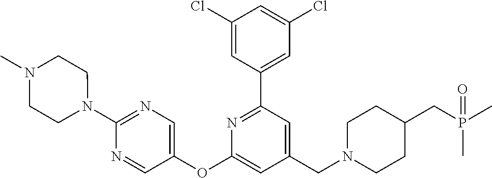 | ((1-((2-(3,5-dichlorophenyl)-6-((2-(4- methylpiperazin-1-yl)pyrimidin-5- yl)oxy)pyridin-4-yl)methyl)piperidin- 4-yl)methyl)dimethylphosphineoxide |
| 321 | 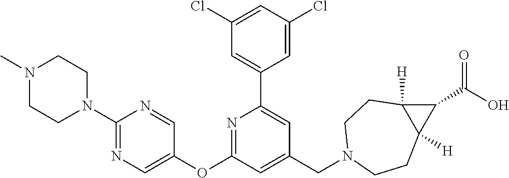 [SMILES: CN1CCN(c2ncc(Oc3cc(CN4CC[C@@H]5[C@H](CC4)[C@H]5C(=O)O)cc(-c4cc(Cl)cc(Cl)c4)n3)cn2)CC1] | (1R,7S,8r)-4-((2-(3,5-dichlorophenyl)- 6-((2-(4-methylpiperazin-1-yl) pyrimidin-5-yl)oxy)pyridin-4-yl) methyl)-4-azabicyclo[5.1.0]octane- 8-carboxylic acid |
| 322 | 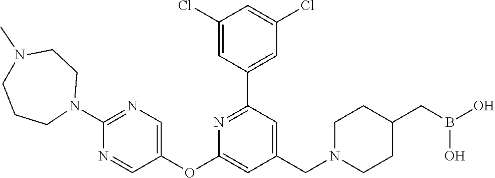 [SMILES: CN1CCCN(c2ncc(Oc3cc(CN4CCC(CB(O)O)CC4)cc(-c4cc(Cl)cc(Cl)c4)n3)cn2)CC1] | ((1-((2-(3,5-dichlorophenyl)-6-((2-(4- methyl-1,4-diazepan-1-yl)pyrimidin-5- yl)oxy)pyridin-4-yl)methyl)piperidin-4- yl)methyl)boronic acid |
| 323 | 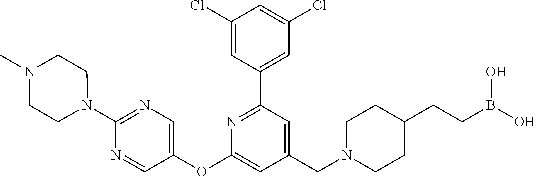 [SMILES: CN1CCN(c2ncc(Oc3cc(CN4CCC(CCB(O)O)CC4)cc(-c4cc(Cl)cc(Cl)c4)n3)cn2)CC1] | (2-(1-((2-(3,5-dichlorophenyl)-6-((2-(4- methylpiperazin-1-yl)pyrimidin-5- yl)oxy)pyridin-4-yl)methyl)piperidin-4- yl)ethyl)boronic acid |
| 324 | 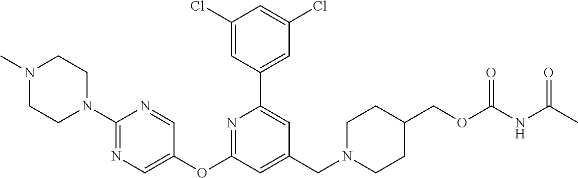 [SMILES: CC(=O)NC(=O)OCC1CCN(Cc2cc(Oc3cnc(N4CCN(C)CC4)nc3)nc(-c3cc(Cl)cc(Cl)c3)c2)CC1] | (1-((2-(3,5-dichlorophenyl)-6-((6-(4- methylpiperazin-1-yl)pyridin-3- yl)oxy)pyridin-4-yl)methyl)piperidin- 4-yl)methylacetylcarbamate |
| 325 | 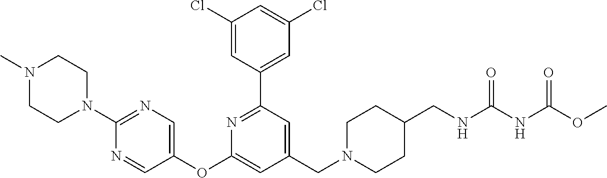 | N-1-((1-((2-(3,5-dichlorophenyl)-6-((2- (4-methylpiperazin-1-yl)pyrimidin-5- yl)oxy)pyridin-4-yl)methyl)piperidin- 4-yl)methyl)-N′-methoxyl- carbonylurea |
| 326 | 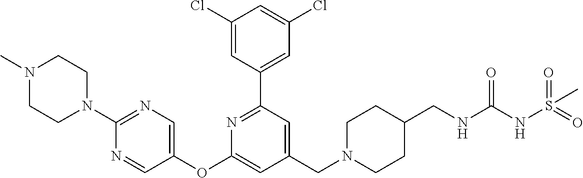 | N-(((1-((2-(3,5-dichlorophenyl)-6-((2- (4-methylpiperazin-1-yl)pyrimidin-5- yl)oxy)pyridin-4-yl)methyl)piperidin- 4-yl)methyl)carbamoyl)methane- sulfonamide |
| 327 | 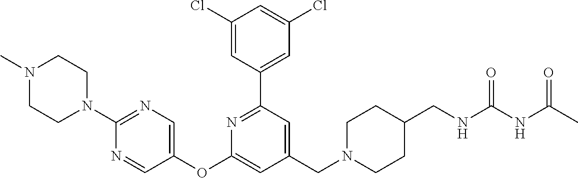 [SMILES: CC(=O)NC(=O)NCC1CCN(Cc2cc(Oc3cnc(N4CCN(C)CC4)nc3)nc(-c3cc(Cl)cc(Cl)c3)c2)CC1] | N-(((1-((2-(3,5-dichlorophenyl)-6-((2- (4-methylpiperazin-1-yl)pyrimidin-5- yl)oxy)pyridin-4-yl)methyl)piperidin- 4-yl)methyl)carbamoyl)acetamide |
| 328 | 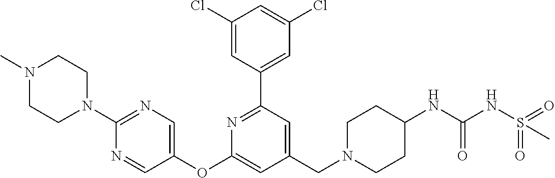 | N-((1-((2-(3,5-dichlorophenyl)-6-((2-(4- methylpiperazin-1-yl)pyrimidin-5- yl)oxy)pyridin-4-yl)methyl)piperidin-4- yl)carbamoyl)methanesulfonamide |
| 329 | 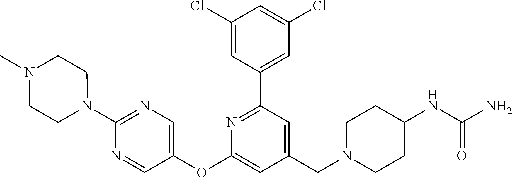 [SMILES: CN1CCN(c2ncc(Oc3cc(CN4CCC(NC(N)=O)CC4)cc(-c4cc(Cl)cc(Cl)c4)n3)cn2)CC1] | 1-(1-((2-(3,5-dichlorophenyl)-6-((2-(4- methylpiperazin-1-yl)pyrimidin-5- yl)oxy)pyridin-4-yl)methyl)piperidin-4- yl)urea |
| 330 | 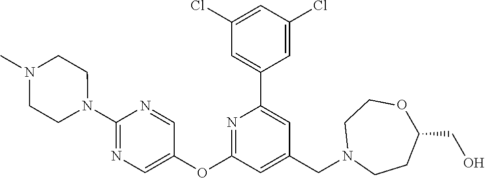 [SMILES: CN1CCN(c2ncc(Oc3cc(CN4CCO[C@H](CO)CC4)cc(-c4cc(Cl)cc(Cl)c4)n3)cn2)CC1] | (S)-(4-((2-(3,5-dichlorophenyl)-6-((2- (4-methylpiperazin-1-yl)pyrimidin-5- yl)oxy)pyridin-4-yl)methyl)-1,4- oxazepan-7-yl)methanol |
| 331 | 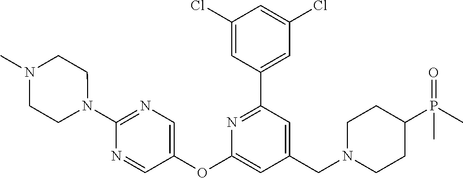 | (1-((2-(3,5-dichlorophenyl)-6-((2-(4- methylpiperazin-1-yl)pyrimidin-5- yl)oxy)pyridin-4-yl)methyl)piperidin-4- yl)dimethylphosphineoxide |
| 332 | 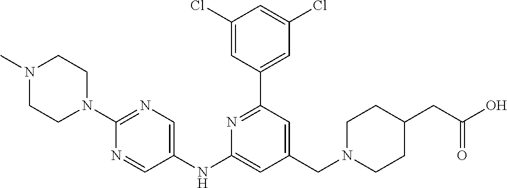 [SMILES: CN1CCN(c2ncc(Nc3cc(CN4CCC(CC(=O)O)CC4)cc(-c4cc(Cl)cc(Cl)c4)n3)cn2)CC1] | 2-(1-((2-(3,5-dichlorophenyl)-6-((2-(4- methylpiperazin-1-yl)pyrimidin-5- yl)amino)pyridin-4-yl)methyl) piperidin-4-yl)acetic acid |
| 333 | 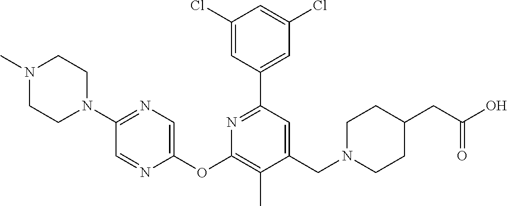 [SMILES: Cc1c(CN2CCC(CC(=O)O)CC2)cc(-c2cc(Cl)cc(Cl)c2)nc1Oc1cnc(N2CCN(C)CC2)cn1] | 2-(1-((6-(3,5-dichlorophenyl)-3-methyl- 2-((5-(4-methylpiperazin-1-yl)pyrazin- 2-yl)oxy)pyridin-4-yl)methyl)piperidin- 4-yl)acetic acid |
| 334 | 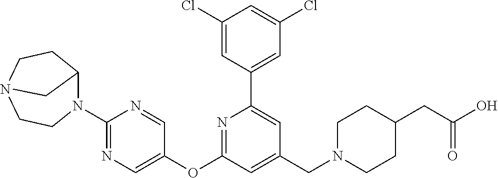 [SMILES: O=C(O)CC1CCN(Cc2cc(Oc3cnc(N4CCN5CCC4C5)nc3)nc(-c3cc(Cl)cc(Cl)c3)c2)CC1] | 2-(1-((2-((2-(1,4-diazabicyclo[3.2.1] octan-4-yl)pyrimidin-5-yl)oxy)-6-(3,5- dichlorophenyl)pyridin-4- yl)methyl)piperidin-4-yl)acetic acid |
| 335 | 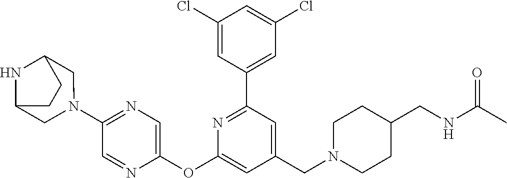 [SMILES: CC(=O)NCC1CCN(Cc2cc(Oc3cnc(N4CC5CCC(C4)N5)cn3)nc(-c3cc(Cl)cc(Cl)c3)c2)CC1] | N-((1-((2-((2-(3,8- diazabicyclo[3.2.1]octan-3-yl) pyrimidin-5-yl)oxy)-6-(3,5- dichlorophenyl)pyridin- 4-yl)methyl)piperidin-4- yl)methyl)acetamide |
| 336 | 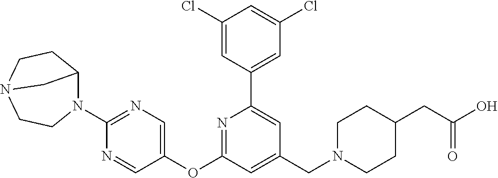 [SMILES: O=C(O)CC1CCN(Cc2cc(Oc3cnc(N4CCN5CCC4C5)nc3)nc(-c3cc(Cl)cc(Cl)c3)c2)CC1] | methyl((1-((2-((2-(1,4- diazabicyclo[3.2.1]octan-4-yl) pyrimidin-5-yl)oxy)-6-(3,5- dichlorophenyl)pyridin- 4-yl)methyl)piperidin-4- yl)methyl)carbamate |
| 337 | 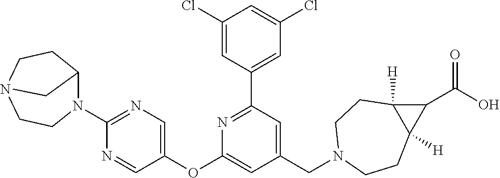 [SMILES: O=C(O)C1[C@H]2CCN(Cc3cc(Oc4cnc(N5CCN6CCC5C6)nc4)nc(-c4cc(Cl)cc(Cl)c4)c3)CC[C@@H]12] | (1R,7S,8r)-4-((2-((2-(1,4- diazabicyclo[3.2.1]octan-4-yl) pyrimidin-5-yl)oxy)-6-(3,5- dichlorophenyl)pyridin- 4-yl)methyl)-4-azabicyclo[5.1.0] octane-8-carboxylic acid |
| 338 | 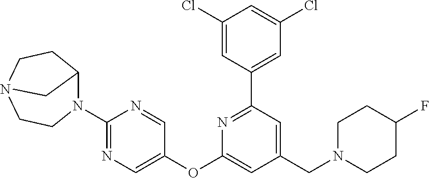 [SMILES: FC1CCN(Cc2cc(Oc3cnc(N4CCN5CCC4C5)nc3)nc(-c3cc(Cl)cc(Cl)c3)c2)CC1] | 4-(5-((6-(3,5-dichlorophenyl)-4-((4- fluoropiperidin-1-yl)methyl)pyridin-2- yl)oxy)pyrimidin-2-yl)-1,4- diazabicyclo[3.2.1]octane |
| 339 | 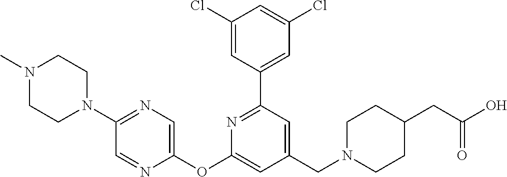 [SMILES: CN1CCN(c2cnc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((5-(4- methylpiperazin-1-yl)pyrazin-2- yl)oxy)pyridin-4-yl)methyl)piperidin-4- yl)acetic acid |
| 340 | 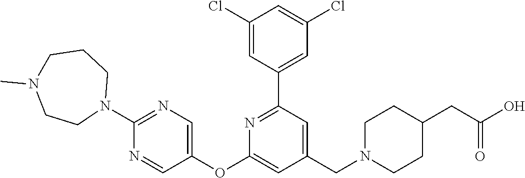 [SMILES: CN1CCCN(c2ncc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((5-(4- methyl-1,4-diazepan-1-yl)pyrazin-2- yl)oxy)pyridin-4-yl)methyl)piperidin-4- yl)acetic acid |
| 341 | 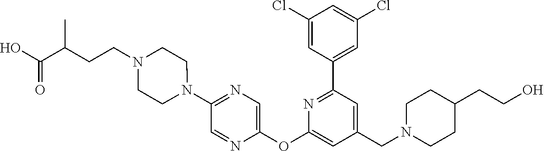 [SMILES: CC(CCN1CCN(c2cnc(Oc3cc(CN4CCC(CCO)CC4)cc(-c4cc(Cl)cc(Cl)c4)n3)cn2)CC1)C(=O)O] | 4-(4-(5-((6-(3,5-dichlorophenyl)-4-((4- (2-hydroxyethyl)piperidin-1- yl)methyl)pyridin-2-yl)oxy)pyrazin- 2-yl)piperazin-1-yl)-2-methylbutanoic acid |
| 342 | 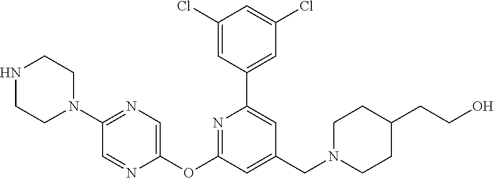 [SMILES: OCCC1CCN(Cc2cc(Oc3cnc(N4CCNCC4)cn3)nc(-c3cc(Cl)cc(Cl)c3)c2)CC1] | 2-(1-((2-(3,5-dichlorophenyl)-6-((5- (piperazin-1-yl)pyrazin-2-yl)oxy) pyridin-4-yl)methyl)piperidin-4-yl) ethanol |
| 343 | 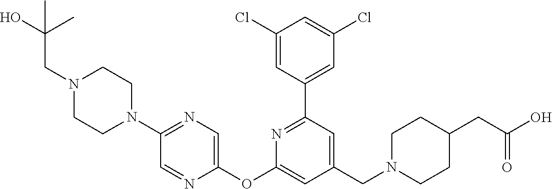 [SMILES: CC(C)(O)CN1CCN(c2cnc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((5-(4- (2-hydroxy-2-methylpropyl)piperazin- 1-yl)pyrazin-2-yl)oxy)pyridin-4- yl)methyl)piperidin-4-yl)acetic acid |
| 344 | 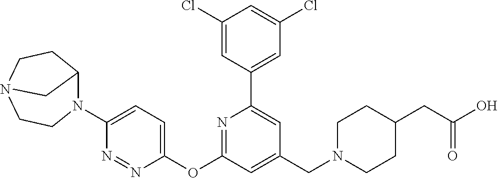 [SMILES: O=C(O)CC1CCN(Cc2cc(Oc3ccc(N4CCN5CCC4C5)nn3)nc(-c3cc(Cl)cc(Cl)c3)c2)CC1] | 2-(1-((2-((6-(1,4-diazabicyclo[3.2.1] octan-4-yl)pyridazin-3-yl)oxy)-6-(3,5- dichlorophenyl)pyridin-4- yl)methyl)piperidin-4-yl)acetic acid |
| 345 | 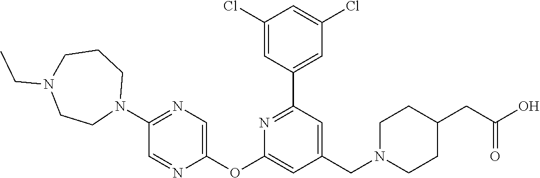 [SMILES: CCN1CCCN(c2cnc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((5-(4- ethyl-1,4-diazepan-1-yl)pyrazin-2- yl)oxy)pyridin-4-yl)methyl)piperidin- 4-yl)acetic acid |
| 346 | 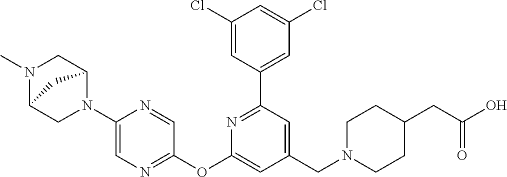 [SMILES: CN1C[C@@H]2C[C@H]1CN2c1cnc(Oc2cc(CN3CCC(CC(=O)O)CC3)cc(-c3cc(Cl)cc(Cl)c3)n2)cn1] | 2-(1-((2-(3,5-dichlorophenyl)-6-((5- ((1S,4S)-5-methyl-2,5- diazabicyclo[2.2.1]heptan-2-yl) pyrazin-2-yl)oxy)pyridin-4-yl)methyl) piperidin-4-yl)acetic acid |
| 347 | 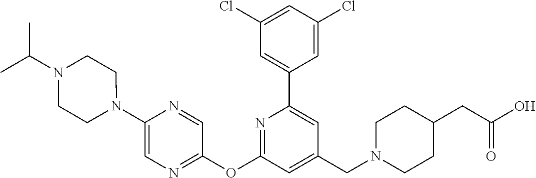 [SMILES: CC(C)N1CCN(c2cnc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((5- (4-isopropylpiperazin-1-yl)pyrazin-2- yl)oxy)pyridin-4-yl)methyl)piperidin- 4-yl)acetic acid |
| 348 | 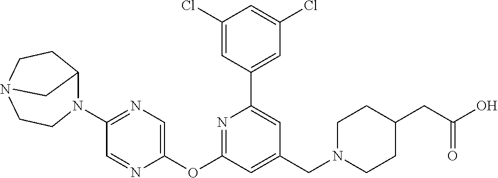 [SMILES: O=C(O)CC1CCN(Cc2cc(Oc3cnc(N4CCN5CCC4C5)cn3)nc(-c3cc(Cl)cc(Cl)c3)c2)CC1] | 2-(1-((2-((5-(1,4-diazabicyclo[3.2.1] octan-4-yl)pyrazin-2-yl)oxy)-6-(3,5- dichlorophenyl)pyridin-4- yl)methyl)piperidin-4-yl)acetic acid |
| 349 | 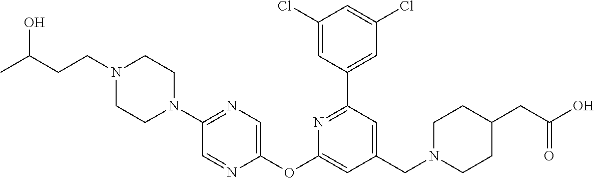 [SMILES: CC(O)CCN1CCN(c2cnc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((5-(4- (3-hydroxybutyl)piperazin-1-yl) pyrazin-2-yl)oxy)pyridin-4-yl)methyl) piperidin-4-yl)acetic acid |
| 350 | 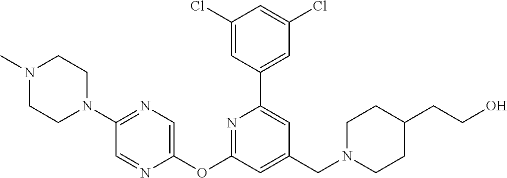 [SMILES: CN1CCN(c2cnc(Oc3cc(CN4CCC(CCO)CC4)cc(-c4cc(Cl)cc(Cl)c4)n3)cn2)CC1] | 2-(1-((2-(3,5-dichlorophenyl)-6-((5-(4- methylpiperazin-1-yl)pyrazin-2- yl)oxy)pyridin-4-yl)methyl)piperidin-4- yl)ethanol |
| 351 | 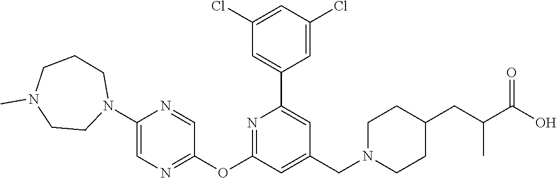 [SMILES: CC(CC1CCN(Cc2cc(Oc3cnc(N4CCCN(C)CC4)cn3)nc(-c3cc(Cl)cc(Cl)c3)c2)CC1)C(=O)O] | 3-(1-((2-(3,5-dichlorophenyl)-6-((5-(4- methyl-1,4-diazepan-1-yl)pyrazin-2- yl)oxy)pyridin-4-yl)methyl)piperidin-4- yl)-2-methylpropanoic acid |
| 352 | 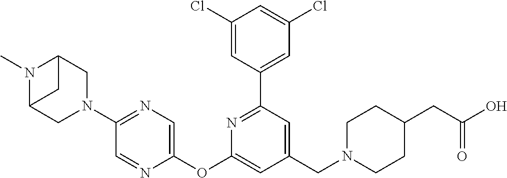 [SMILES: CN1C2CC1CN(c1cnc(Oc3cc(CN4CCC(CC(=O)O)CC4)cc(-c4cc(Cl)cc(Cl)c4)n3)cn1)C2] | 2-(1-((2-(3,5-dichlorophenyl)-6-((5-(6- methyl-3,6-diazabicyclo[3.1.l]heptan-3- yl)pyrazin-2-yl)oxy)pyridin-4- yl)methyl)piperidin-4-yl)acetic acid |
| 353 | 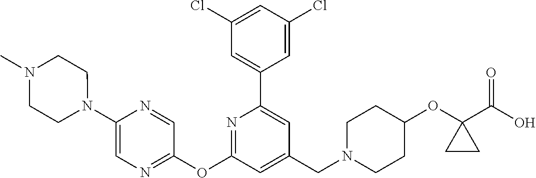 [SMILES: CN1CCN(c2cnc(Oc3cc(CN4CCC(OC5(C(=O)O)CC5)CC4)cc(-c4cc(Cl)cc(Cl)c4)n3)cn2)CC1] | 1-((1-((2-(3,5-dichlorophenyl)-6-((5-(4- methylpiperazin-1-yl)pyrazin-2- yl)oxy)pyridin-4-yl)methyl)piperidin-4- yl)oxy)cyclopropanecarboxylic acid |
| 354 | 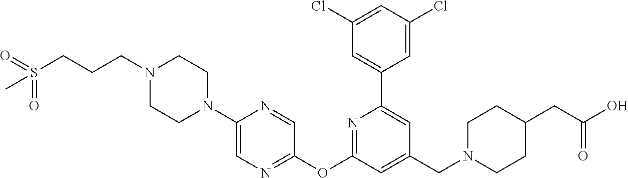 [SMILES: CS(=O)(=O)CCCN1CCN(c2cnc(Oc3cc(CN4CCC(CC(=O)O)CC4)cc(-c4cc(Cl)cc(Cl)c4)n3)cn2)CC1] | 2-(1-((2-(3,5-dichlorophenyl)-6-((5-(4- (3-(methylsulfonyl)propyl)piperazin-1- yl)pyrazin-2-yl)oxy)pyridin-4- yl)methyl)piperidin-4-yl)acetic acid |
| 355 | 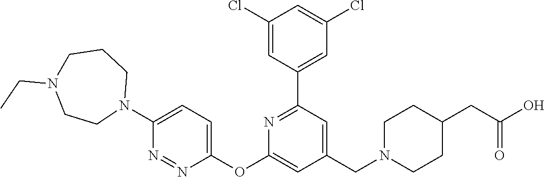 [SMILES: CCN1CCCN(c2ccc(Oc3cc(CN4CCC(CC(=O)O)CC4)cc(-c4cc(Cl)cc(Cl)c4)n3)nn2)CC1] | 2-(1-((2-(3,5-dichlorophenyl)-6-((6-(4- ethyl-1,4-diazepan-1-yl)pyridazin-3- yl)oxy)pyridin-4-yl)methyl)piperidin-4- yl)acetic acid |
| 356 | 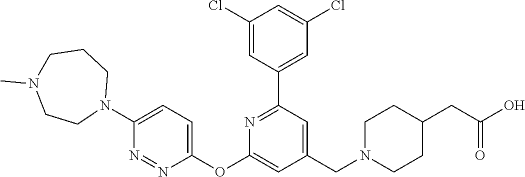 [SMILES: CN1CCCN(c2ccc(Oc3cc(CN4CCC(CC(=O)O)CC4)cc(-c4cc(Cl)cc(Cl)c4)n3)nn2)CC1] | 2-(1-((2-(3,5-dichlorophenyl)-6-((6-(4- methyl-1,4-diazepan-1-yl)pyridazin-3- yl)oxy)pyridin-4-yl)methyl)piperidin-4- yl)acetic acid |
| 357 | 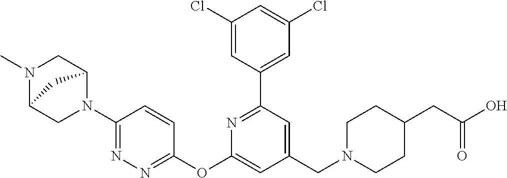 [SMILES: CN1C[C@@H]2C[C@H]1CN2c1ccc(Oc2cc(CN3CCC(CC(=O)O)CC3)cc(-c3cc(Cl)cc(Cl)c3)n2)nn1] | 2-(1-((2-(3,5-dichlorophenyl)-6-((6- ((1S,4S)-5-methyl-2,5- diazabicyclo[2.2.1]heptan-2-yl) pyridazin-3-yl)oxy)pyridin-4-yl) methyl)piperidin-4-yl)acetic acid |
| 358 | 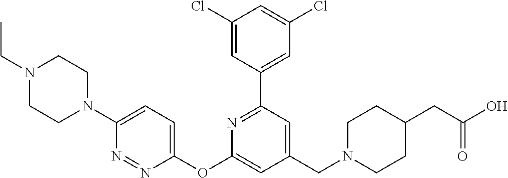 [SMILES: CCN1CCN(c2ccc(Oc3cc(CN4CCC(CC(=O)O)CC4)cc(-c4cc(Cl)cc(Cl)c4)n3)nn2)CC1] | 2-(1-((2-(3,5-dichlorophenyl)-6-((6-(4- ethylpiperazin-1-yl)pyridazin-3- yl)oxy)pyridin-4-yl)methyl)piperidin- 4-yl)acetic acid |
| 359 | 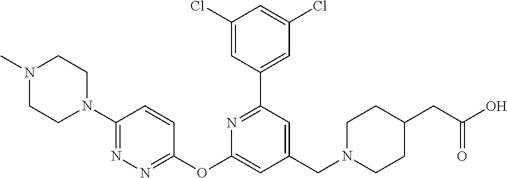 [SMILES: CN1CCN(c2ccc(Oc3cc(CN4CCC(CC(=O)O)CC4)cc(-c4cc(Cl)cc(Cl)c4)n3)nn2)CC1] | 2-(1-((2-(3,5-dichlorophenyl)-6-((6-(4- methylpiperazin-1-yl)pyridazin-3- yl)oxy)pyridin-4-yl)methyl)piperidin- 4-yl)acetic acid |
| 360 | 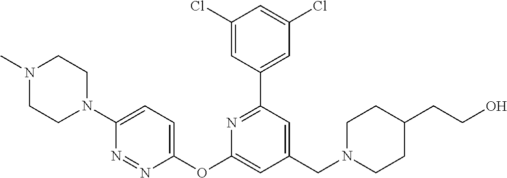 [SMILES: CN1CCN(c2ccc(Oc3cc(CN4CCC(CCO)CC4)cc(-c4cc(Cl)cc(Cl)c4)n3)nn2)CC1] | 2-(1-((2-(3,5-dichlorophenyl)-6-((6-(4- methylpiperazin-1-yl)pyridazin-3- yl)oxy)pyridin-4-yl)methyl)piperidin- 4-yl)ethanol |
| 361 | 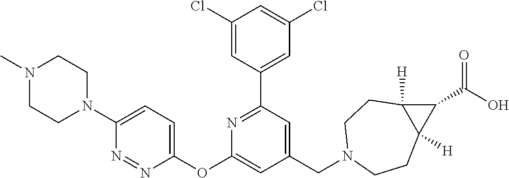 [SMILES: CN1CCN(c2ccc(Oc3cc(CN4CC[C@@H]5[C@H](CC4)[C@H]5C(=O)O)cc(-c4cc(Cl)cc(Cl)c4)n3)nn2)CC1] | (1R,7S,8r)-4-((2-(3,5-dichlorophenyl)- 6-((6-(4-methylpiperazin-1-yl) pyridazin-3- yl)oxy)pyridin-4-yl)methyl)-4- azabicyclo[5.1.0]octane-8- carboxylic acid |
| 362 | 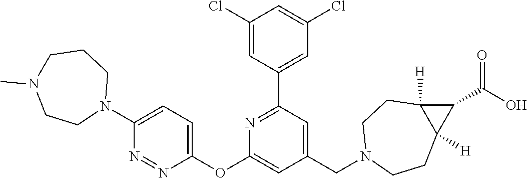 [SMILES: CN1CCCN(c2ccc(Oc3cc(CN4CC[C@@H]5[C@H](CC4)[C@H]5C(=O)O)cc(-c4cc(Cl)cc(Cl)c4)n3)nn2)CC1] | (1R,7S,8r)-4-((2-(3,5-dichlorophenyl)- 6-((6-(4-methyl-1,4-diazepan-1- yl)pyridazin-3-yl)oxy)pyridin-4- yl)methyl)-4-azabicyclo[5.1.0]octane- 8-carboxylic acid |
| 363 | 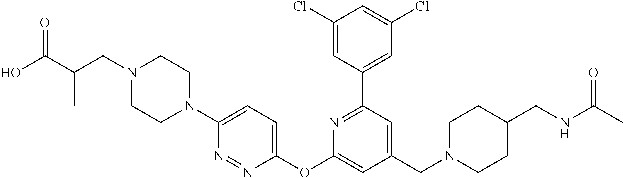 [SMILES: CC(=O)NCC1CCN(Cc2cc(Oc3ccc(N4CCN(CC(C)C(=O)O)CC4)nn3)nc(-c3cc(Cl)cc(Cl)c3)c2)CC1] | 3-(4-(6-((4-((4- (acetamidomethyl)piperidin-1-yl) methyl)-6-(3,5-dichlorophenyl) pyridin-2-yl)oxy)pyridazin-3-yl) piperazin-1-yl)-2- methylpropanoic acid |
| 364 | 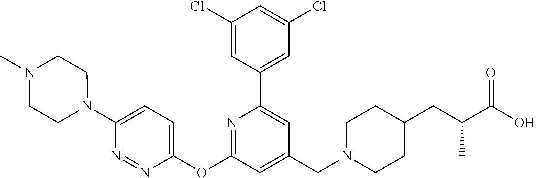 [SMILES: C[C@H](CC1CCN(Cc2cc(Oc3ccc(N4CCN(C)CC4)nn3)nc(-c3cc(Cl)cc(Cl)c3)c2)CC1)C(=O)O] | (R)-3-(1-((2-(3,5-dichlorophenyl)-6- ((6-(4-methylpiperazin-1-yl)pyridazin- 3-yl)oxy)pyridin-4-yl)methyl) piperidin-4-yl)-2-methylpropanoic acid |
| 365 | 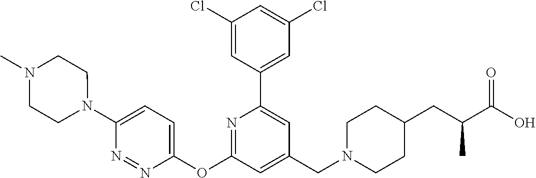 [SMILES: C[C@@H](CC1CCN(Cc2cc(Oc3ccc(N4CCN(C)CC4)nn3)nc(-c3cc(Cl)cc(Cl)c3)c2)CC1)C(=O)O] | (S)-3-(1-((2-(3,5-dichlorophenyl)-6- ((6-(4-methylpiperazin-1-yl) pyridazin-3-yl)oxy)pyridin-4-yl) methyl)piperidin-4- yl)-2-methylpropanoic acid |
| 366 | 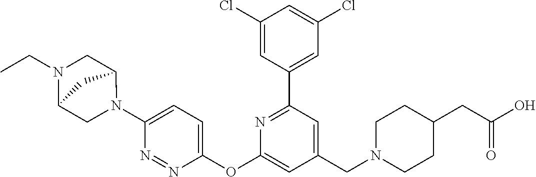 [SMILES: CCN1C[C@@H]2C[C@H]1CN2c1ccc(Oc2cc(CN3CCC(CC(=O)O)CC3)cc(-c3cc(Cl)cc(Cl)c3)n2)nn1] | 2-(1-((2-(3,5-dichlorophenyl)-6-((6- ((1S,4S)-5-ethyl-2,5- diazabicyclo[2.2.1]heptan-2-yl) pyridazin-3-yl)oxy)pyridin-4-yl) methyl)piperidin-4- yl)acetic acid |
| 367 | 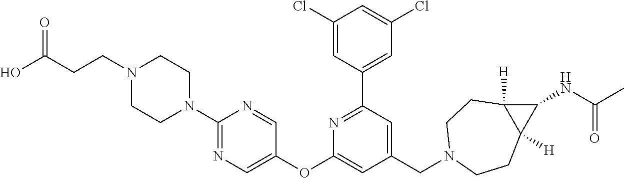 [SMILES: CC(=O)N[C@H]1[C@@H]2CCN(Cc3cc(Oc4cnc(N5CCN(CCC(=O)O)CC5)nc4)nc(-c4cc(Cl)cc(Cl)c4)c3)CC[C@@H]21] | 3-(4-(5-((4-(((1R,7S,8r)-8-acetamido- 4-azabicyclo[5.1.0]octan-4-yl)methyl)- 6-(3,5-dichlorophenyl)pyridin-2- yl)oxy)pyrimidin-2-yl)piperazin-1- yl)propanoic acid |
| 368 | 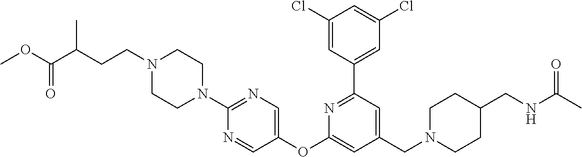 [SMILES: COC(=O)C(C)CCN1CCN(c2ncc(Oc3cc(CN4CCC(CNC(C)=O)CC4)cc(-c4cc(Cl)cc(Cl)c4)n3)cn2)CC1] | methyl4-(4-(5-((4-((4- (acetamidomethyl)piperidin-1-yl) methyl)- 6-(3,5-dichlorophenyl)pyridin-2- yl)oxy)pyrimidin-2-yl)piperazin- 1-yl)-2-methylbutanoate |
| 369 | 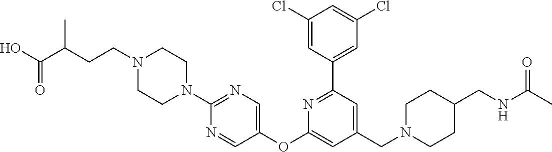 [SMILES: CC(=O)NCC1CCN(Cc2cc(Oc3cnc(N4CCN(CCC(C)C(=O)O)CC4)nc3)nc(-c3cc(Cl)cc(Cl)c3)c2)CC1] | 4-(4-(5-((4-((4- (acetamidomethyl)piperidin-1-yl) methyl)- 6-(3,5-dichlorophenyl)pyridin-2- yl)oxy)pyrimidin-2-yl)piperazin- 1-yl)-2-methylbutanoic acid |
| 370 | 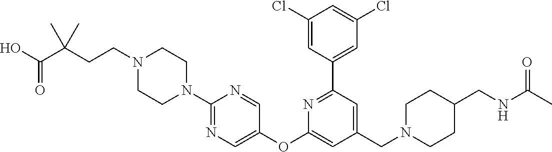 [SMILES: CC(=O)NCC1CCN(Cc2cc(Oc3cnc(N4CCN(CCC(C)(C)C(=O)O)CC4)nc3)nc(-c3cc(Cl)cc(Cl)c3)c2)CC1] | 4-(4-(5-((4-((4- (acetamidomethyl)piperidin-1-yl) methyl)- 6-(3,5-dichlorophenyl)pyridin-2- yl)oxy)pyrimidin-2-yl)piperazin- 1-yl)-2,2-dimethylbutanoic acid |
| 371 | 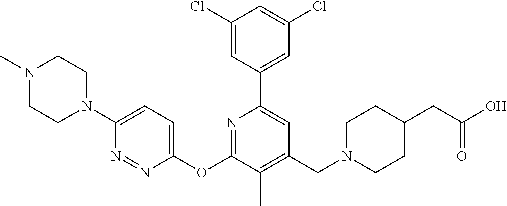 [SMILES: Cc1c(CN2CCC(CC(=O)O)CC2)cc(-c2cc(Cl)cc(Cl)c2)nc1Oc1ccc(N2CCN(C)CC2)nn1] | 2-(1-((6-(3,5-dichlorophenyl)-3- methyl-2-((6-(4-methylpiperazin- 1-yl)pyridazin-3-yl)oxy)pyridin- 4-yl)methyl)piperidin- 4-yl)acetic acid |
| 372 | 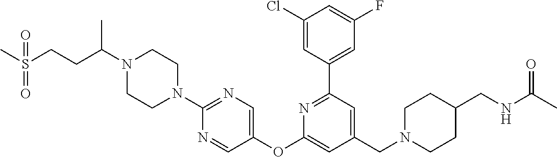 [SMILES: CC(=O)NCC1CCN(Cc2cc(Oc3cnc(N4CCN(C(C)CCS(C)(=O)=O)CC4)nc3)nc(-c3cc(F)cc(Cl)c3)c2)CC1] | N-((1-((2-(3-chloro-5-fluorophenyl)-6- ((2-(4-(4-(methylsulfonyl)butan-2- yl)piperazin-1-yl)pyrimidin-5- yl)oxy)pyridin-4-yl)methyl)piperidin- 4-yl)methyl)acetamide |
| 373 | 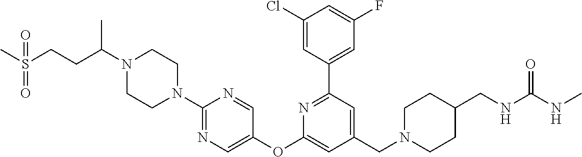 | 1-((1-((2-(3-chloro-5-fluorophenyl)-6- ((2-(4-(4-(methylsulfonyl)butan-2- yl)piperazin-1-yl)pyrimidin-5- yl)oxy)pyridin-4-yl)methyl)piperidin- 4-yl)methyl)-3-methylurea |
| 374 | 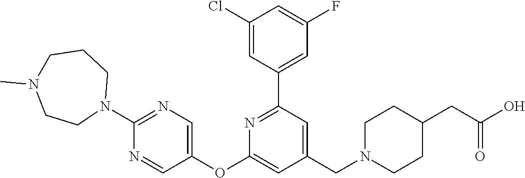 [SMILES: CN1CCCN(c2ncc(Oc3cc(CN4CCC(CC(=O)O)CC4)cc(-c4cc(F)cc(Cl)c4)n3)cn2)CC1] | 2-(1-((2-(3-chloro-5-fluorophenyl)-6- ((2-(4-methyl-1,4-diazepan-1-yl)p yrimidin-5-yl)oxy)pyridin-4-yl) methyl)piperidin-4- yl)acetic acid |
| 375 | 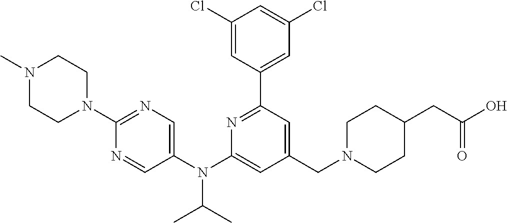 [SMILES: CC(C)N(c1cnc(N2CCN(C)CC2)nc1)c1cc(CN2CCC(CC(=O)O)CC2)cc(-c2cc(Cl)cc(Cl)c2)n1] | 2-(1-((2-(3,5-dichlorophenyl)-6- (isopropyl(2-(4-methylpiperazin-1- yl)pyrimidin-5-yl)amino)pyridin-4- yl)methyl)piperidin-4-yl)acetic acid |
| 376 | 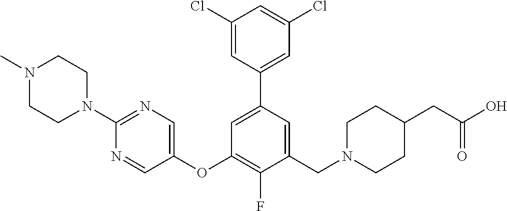 [SMILES: CN1CCN(c2ncc(Oc3cc(-c4cc(Cl)cc(Cl)c4)cc(CN4CCC(CC(=O)O)CC4)c3F)cn2)CC1] | 2-(1-((3′,5′-dichloro-4-fluoro-5-((2-(4- methylpiperazin-1-yl)pyrimidin-5-yl) oxy)-[1,1′-biphenyl]-3-yl)methyl) piperidin-4-yl)acetic acid |
| 377 | 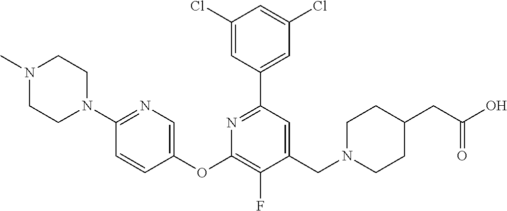 [SMILES: CN1CCN(c2ccc(Oc3nc(-c4cc(Cl)cc(Cl)c4)cc(CN4CCC(CC(=O)O)CC4)c3F)cn2)CC1] | 2-(1-((6-(3,5-dichlorophenyl)-3- fluoro-2-((6-(4-methylpiperazin-1-yl) pyridin-3-yl)oxy)pyridin-4-yl)methyl) piperidin-4-yl)acetic acid |
| 378 | 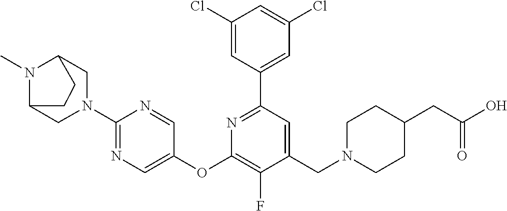 [SMILES: CN1C2CCC1CN(c1ncc(Oc3nc(-c4cc(Cl)cc(Cl)c4)cc(CN4CCC(CC(=O)O)CC4)c3F)cn1)C2] | 2-(1-((6-(3,5-dichlorophenyl)-3- fluoro-2-((2-(8-methyl-3,8- diazabicyclo[3.2.1]octan-3-yl) pyrimidin-5-yl)oxy)pyridin-4-yl) methyl)piperidin- 4-yl)acetic acid |
| 379 | 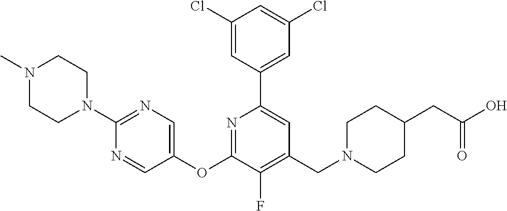 [SMILES: CN1CCN(c2ncc(Oc3nc(-c4cc(Cl)cc(Cl)c4)cc(CN4CCC(CC(=O)O)CC4)c3F)cn2)CC1] | 2-(1-((6-(3,5-dichlorophenyl)-3- fluoro-2-((2-(4-methylpiperazin-1-yl) pyrimidin-5-yl)oxy)pyridin-4-yl) methyl)piperidin- 4-yl)acetic acid |
| 380 | 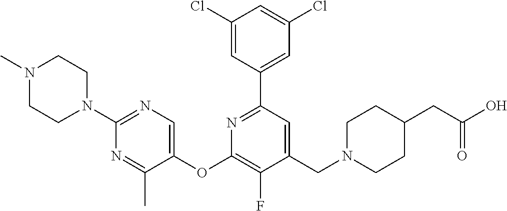 [SMILES: Cc1nc(N2CCN(C)CC2)ncc1Oc1nc(-c2cc(Cl)cc(Cl)c2)cc(CN2CCC(CC(=O)O)CC2)c1F] | 2-(1-((6-(3,5-dichlorophenyl)-3- fluoro-2-((4-methyl-2-(4-methyl- piperazin-1- yl)pyrimidin-5-yl)oxy)pyridin-4- yl)methyl)piperidin-4-yl)acetic acid |
| 381 | 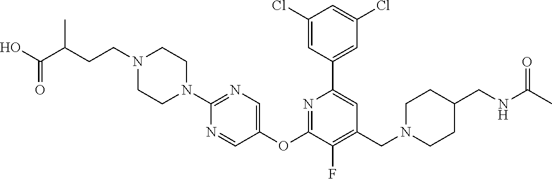 [SMILES: CC(=O)NCC1CCN(Cc2cc(-c3cc(Cl)cc(Cl)c3)nc(Oc3cnc(N4CCN(CCC(C)C(=O)O)CC4)nc3)c2F)CC1] | 4-(4-(5-((4-((4- (acetamidomethyl)piperidin-1-yl) methyl)-6-(3,5-dichlorophenyl)-3- fluoropyridin-2-yl)oxy)pyrimidin- 2-yl)piperazin-1-yl)-2- methylbutanoic acid |
| 382 | 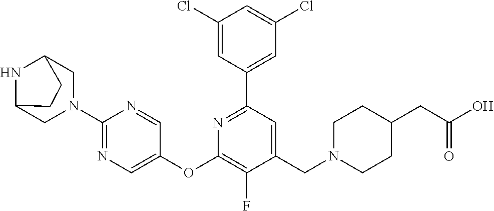 [SMILES: O=C(O)CC1CCN(Cc2cc(-c3cc(Cl)cc(Cl)c3)nc(Oc3cnc(N4CC5CCC(C4)N5)nc3)c2F)CC1] | 2-(1-((2-((2-(3,8-diazabicyclo[3.2.1] octan-3-yl)pyrimidin-5-yl)oxy)-6-(3,5- dichlorophenyl)-3-fluoropyridin-4- yl)methyl)piperidin-4-yl)acetic acid |
| 383 | 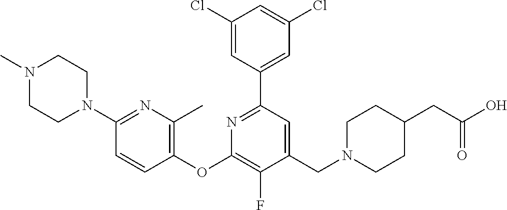 [SMILES: Cc1nc(N2CCN(C)CC2)ccc1Oc1nc(-c2cc(Cl)cc(Cl)c2)cc(CN2CCC(CC(=O)O)CC2)c1F] | 2-(1-((6-(3,5-dichlorophenyl)-3- fluoro-2- ((2-methyl-6-(4-methylpiperazin-1- yl)pyridin-3-yl)oxy)pyridin-4- yl)methyl)piperidin-4-yl)acetic acid |
| 384 | 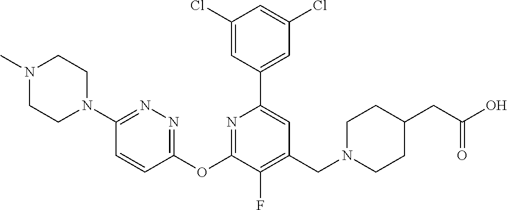 [SMILES: CN1CCN(c2ccc(Oc3nc(-c4cc(Cl)cc(Cl)c4)cc(CN4CCC(CC(=O)O)CC4)c3F)nn2)CC1] | 2-(1-((6-(3,5-dichlorophenyl)-3- fluoro-2-((6-(4-methylpiperazin-1- yl)pyridazin-3- yl)oxy)pyridin-4-yl)methyl) piperidin-4- yl)acetic acid |
| 385 | 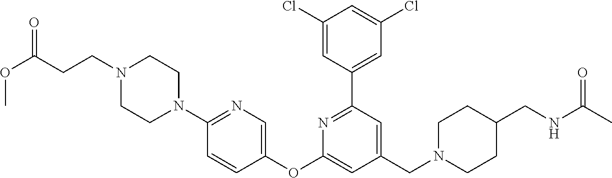 [SMILES: COC(=O)CCN1CCN(c2ccc(Oc3cc(CN4CCC(CNC(C)=O)CC4)cc(-c4cc(Cl)cc(Cl)c4)n3)cn2)CC1] | methyl3-(4-(5-((4-((4- (acetamidomethyl)piperidin-1-yl) methyl)-6-(3,5-dichlorophenyl) pyridin-2-yl)oxy)pyridin-2-yl) piperazin-1-yl)propanoate |
| 386 | 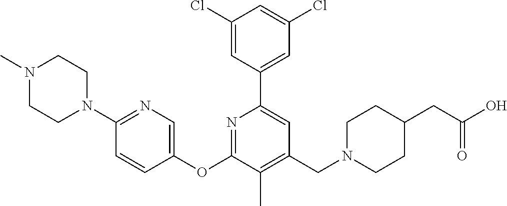 [SMILES: Cc1c(CN2CCC(CC(=O)O)CC2)cc(-c2cc(Cl)cc(Cl)c2)nc1Oc1ccc(N2CCN(C)CC2)nc1] | 2-(1-((6-(3,5-dichlorophenyl)-3- methyl-2-((6-(4-methylpiperazin- 1-yl)pyridin-3- yl)oxy)pyridin-4-yl)methyl) piperidin-4-yl)acetic acid |
| 387 | 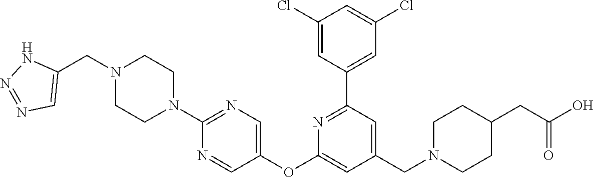 [SMILES: O=C(O)CC1CCN(Cc2cc(Oc3cnc(N4CCN(Cc5cnn[nH]5)CC4)nc3)nc(-c3cc(Cl)cc(Cl)c3)c2)CC1] | 2-(1-((2-((2-(4-((1H-l,2,3-triazol-5- yl)methyl)piperazin-1-yl)pyrimidin-5- yl)oxy)-6-(3,5-dichlorophenyl)pyridin- 4-yl)methyl)piperidin-4-yl)acetic acid |
| 388 | 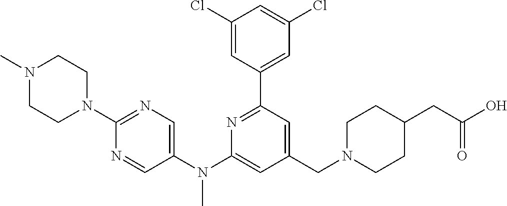 [SMILES: CN1CCN(c2ncc(N(C)c3cc(CN4CCC(CC(=O)O)CC4)cc(-c4cc(Cl)cc(Cl)c4)n3)cn2)CC1] | 2-(1-((2-(3,5-dichlorophenyl)-6- (methyl(2-(4-methylpiperazin-1- yl)pyrimidin-5-yl)amino)pyridin-4- yl)methyl)piperidin-4-yl)acetic acid |
| 389 | 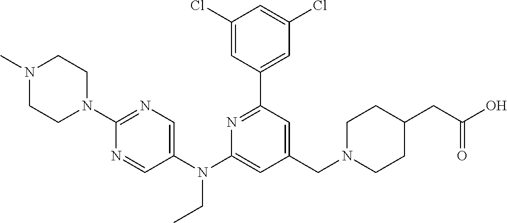 [SMILES: CCN(c1cnc(N2CCN(C)CC2)nc1)c1cc(CN2CCC(CC(=O)O)CC2)cc(-c2cc(Cl)cc(Cl)c2)n1] | 2-(1-((2-(3,5-dichlorophenyl)-6-(ethyl (2-(4-methylpiperazin-1-yl)pyrimidin- y5-l)amino)pyridin-4-yl)methyl) piperidin-4-yl)acetic acid |
| 390 | 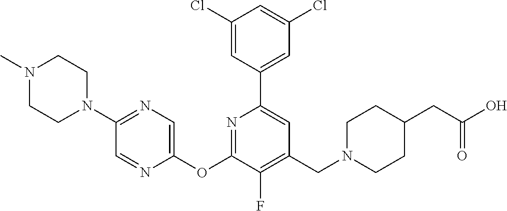 [SMILES: CN1CCN(c2cnc(Oc3nc(-c4cc(Cl)cc(Cl)c4)cc(CN4CCC(CC(=O)O)CC4)c3F)cn2)CC1] | 2-(1-((6-(3,5-dichlorophenyl)-3-fluoro- 2-((5-(4-methylpiperazin-1-yl)pyrazin- 2-yl)oxy)pyridin-4-yl)methyl)piperidin- 4-yl)acetic acid |
| 391 | 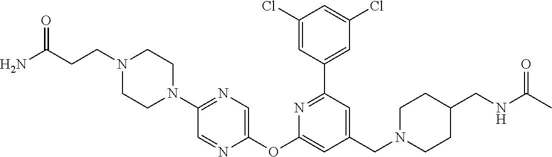 [SMILES: CC(=O)NCC1CCN(Cc2cc(Oc3cnc(N4CCN(CCC(N)=O)CC4)cn3)nc(-c3cc(Cl)cc(Cl)c3)c2)CC1] | 3-(4-(5-((4-(4- (acetamidomethyl)piperidin-1-yl) methyl)-6-(3,5-dichlorophenyl) pyridin-2-yl)oxy)pyrazin-2-yl) piperazin-1-yl)propanamide |
| 392 | 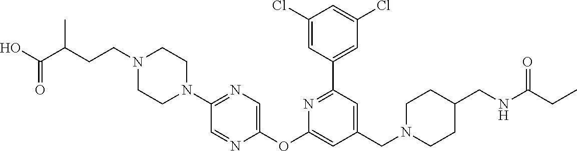 [SMILES: CCC(=O)NCC1CCN(Cc2cc(Oc3cnc(N4CCN(CCC(C)C(=O)O)CC4)cn3)nc(-c3cc(Cl)cc(Cl)c3)c2)CC1] | 4-(4-(5-((6-(3,5-dichlorophenyl)-4-((4- (propionamidomethyl)piperidin-1- yl)methyl)pyridin-2-yl)oxy)pyrazin-2- yl)piperazin-1-yl)-2-methylbutanoic acid |
| 393 | 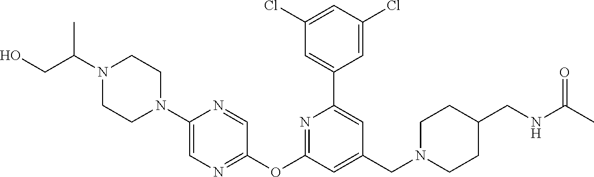 [SMILES: CC(=O)NCC1CCN(Cc2cc(Oc3cnc(N4CCN(C(C)CO)CC4)cn3)nc(-c3cc(Cl)cc(Cl)c3)c2)CC1] | N-((1-((2-(3,5-dichlorophenyl)-6-((5-(4- (1-hydroxypropan-2-yl)piperazin-1- yl)pyrazin-2-yl)oxy)pyridin-4- yl)methyl)piperidin-4- yl)methyl)acetamide |
| 394 | 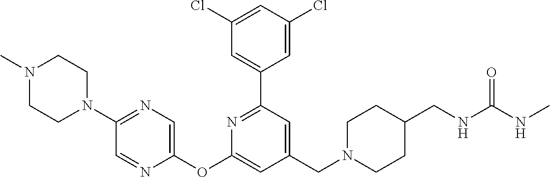 | 1-((1-((2-(3,5-dichlorophenyl)-6-((5-(4- methylpiperazin-1-yl)pyrazin-2- yl)oxy)pyridin-4-yl)methyl)piperidin-4- yl)methyl)-3-methylurea |
| 395 | 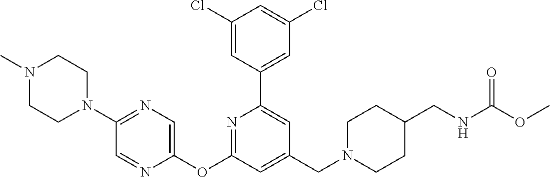 | methyl((1-((2-(3,5-dichlorophenyl)-6- ((5-(4-methylpiperazin-1-yl)pyrazin-2- yl)oxy)pyridin-4-yl)methyl)piperidin- 4-yl)methyl)carbamate |
| 396 | 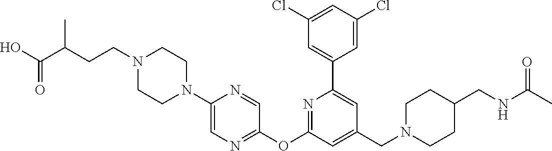 [SMILES: CC(=O)NCC1CCN(Cc2cc(Oc3cnc(N4CCN(CCC(C)C(=O)O)CC4)cn3)nc(-c3cc(Cl)cc(Cl)c3)c2)CC1] | 4-(4-(5-((4-((4- (acetamidomethyl)piperidin-1-yl) methyl)-6-(3,5-dichlorophenyl) pyridin-2- yl)oxy)pyrazin-2-yl)piperazin-1-yl)- 2-methylbutanoic acid |
| 397 | 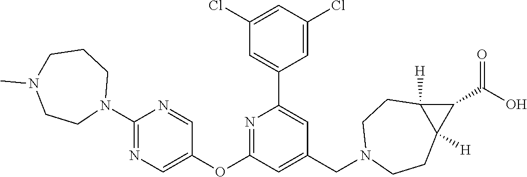 [SMILES: CN1CCCN(c2ncc(Oc3cc(CN4CC[C@@H]5[C@H](CC4)[C@H]5C(=O)O)cc(-c4cc(Cl)cc(Cl)c4)n3)cn2)CC1] | (1R,7S,8r)-4-((2-(3,5-dichlorophenyl)- 6-((2-(4-methyl-1,4-diazepan-1- yl)pyrimidin-5-yl)oxy)pyridin-4- yl)methyl)-4-azabicyclo[5.1.0]octane- 8-carboxylic acid |
| 398 | 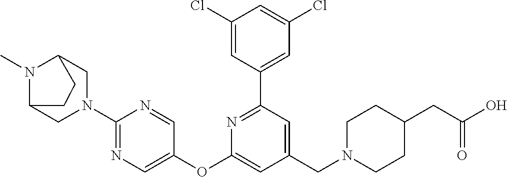 [SMILES: CN1C2CCC1CN(c1ncc(Oc3cc(CN4CCC(CC(=O)O)CC4)cc(-c4cc(Cl)cc(Cl)c4)n3)cn1)C2] | 2-(1-((2-(3,5-dichlorophenyl)-6-((2-(8- methyl-3,8-diazabicyclo[3.2.1]octan-3- yl)pyrimidin-5-yl)oxy)pyridin-4- yl)methyl)piperidin-4-yl)acetic acid |
| 399 | 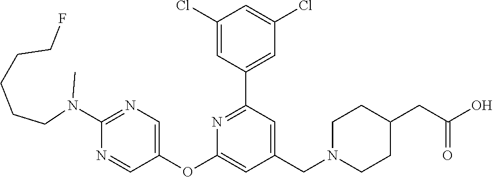 [SMILES: CN(CCCCCF)c1ncc(Oc2cc(CN3CCC(CC(=O)O)CC3)cc(-c3cc(Cl)cc(Cl)c3)n2)cn1] | 2-(1-((2-(3,5-dichlorophenyl)-6-((2-(6- fluoro-1,4-diazepan-1-yl)pyrimidin-5- yl)oxy)pyridin-4-yl)methyl)piperidin-4- yl)acetic acid |

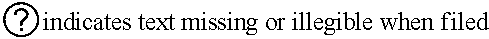

Typically, but not absolutely, the salts of the present disclosure are pharmaceutically acceptable salts. Salts of the disclosed compounds containing a basic amine or other basic functional group may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycolates, tartrates mandelates, and sulfonates, such as xylenesulfonates, methanesulfonates, propanesulfonates, naphthalene-1-sulfonates, and naphthalene-2-sulfonates.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases, such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

Other salts, which are not pharmaceutically acceptable, may be useful in the preparation of compounds of this disclosure and these should be considered to form a further aspect of this disclosure. These salts, such as oxalic or trifluoroacetate, while not in themselves pharmaceutically acceptable, may be useful in the preparation of salts useful as intermediates in obtaining the compounds of this disclosure and their pharmaceutically acceptable salts.

This disclosure further provides a pharmaceutical composition useful in the present disclosure (also referred to as pharmaceutical formulation) comprising a compound of Formula (I) or pharmaceutically acceptable salt thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of this disclosure once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s). The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, the duration between the first dose and last dose of the multiple doses is three months, six months, or one year. In certain embodiments, the duration between the first dose and last dose of the multiple doses is the lifetime of the subject. In certain embodiments, a dose (e.g., a single dose, or any dose of multiple doses) described herein includes independently between 0.1 pg and 1 pg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A therapeutically effective amount of a compound of the present disclosure will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of Formula (I) for the treatment of a viral infection (e.g., resulting from a virus (e.g., dengue, Marburg, Chikungunya viruses) will generally be in the range of 0.001 to 100 mg/kg body weight of recipient per day, suitably in the range of 0.01 to 10 mg/kg body weight per day. For a 70 kg adult mammal, the actual amount per day would suitably be from 7 to 700 mg and this amount may be given in a single dose per day or in a number (such as two, three, four, five or six) of sub-doses per day such that the total daily dose is the same. Inhaled daily dosages range from 10 μg-10 mg/day, with preferred 10 μg-2 mg/day, and more preferred 50 μg-500 μg/day. An effective amount of a salt or solvate, etc., may be determined as a proportion of the effective amount of the compound of Formula (I) per se. It is envisaged that similar dosages would be appropriate for treatment of the other conditions referred to above.

Also encompassed by the present disclosure are kits (e.g., pharmaceutical packs). In certain embodiments, the kit comprises a compound or pharmaceutical composition described herein, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, each of the first or second containers are independently a vial, ampule, bottle, syringe, dispenser package, tube, or inhaler.

In certain embodiments, a kit described herein includes a first container comprising a compound of Formula (I) or a pharmaceutical composition as described herein. In certain embodiments, a kit described herein is useful in treating and/or preventing a viral infection, such as a viral infection resulting from Marburg virus. In certain embodiments, a kit described herein is useful in treating and/or preventing a viral infection, such as a viral infection resulting from dengue virus. In certain embodiments, a kit described herein is useful in treating and/or preventing a viral infection, such as a viral infection resulting from Chikungunya virus.

In certain embodiments, the kit comprises a compound of Formula (I), or a pharmaceutical composition thereof; and instructions for using the compound or pharmaceutical composition.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a viral infection (e.g., an infection resulting from Marburg virus, dengue virus, Chikungunya virus).

In certain embodiments, the instructions are for administering the compound or pharmaceutical composition to a subject (e.g., a subject in need of treatment or prevention of a disease described herein). In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

### EXAMPLES

### Example 1. Percent Inhibition of Different Viruses Using the Compounds of Formula (I)

### Cell Preparation

Each compound was tested at 1 μM dilution. One day before cell infection, Vero cells were seeded in a 96-well plate at 1.00E+04 μM per well. One day later, the cell medium was aspirated and 99 μL of medium was added to wells testing 1 μM compound. Next, 1 μL of compound was added to bring the total volume to 200 μM, and the medium was incubated for 1 hour.

### Virus Dilutions and Incubation

Virus dilutions were prepared with infection medium in a 15 or 50 mL conical tube. The virus dilution was poured into a sterile reservoir. 100 μL of the virus dilutions was added to the wells in the 96-well plate and the plates incubated at 37° C. for 2-5 days: 2 days for Chikungunya virus (CHIKV), 3 days for Marburg virus (MARV), and 5 days for dengue virus (DENV).

50 μL crystal violet solution was added to each well, and fixed/stained for 30-60 minutes at room temperature. The crystal violet stain was removed over the sink and the wells rinsed with tap water. The plates were tapped on absorbent paper and read on a plate reader at 570 nM.

The data in Table 2 represents the percentage of inhibition detected for each tested compound (from 2 technical replicates) at the dilution 1 μM with CHIKV, DENV, and MARV.

TABLE 2
Percent viral inhibition of selected
compounds of Formula (1)

| Example | CHKV | DENY | MARV |
|---|---|---|---|
| 1 | 6.99 | 0.00 | 0.00 |
| 2 | 33.78 | 34.28 | 60.40 |
| 4 | 18.38 | 16.63 | 70.75 |
| 6 | 0.00 | 0.00 | 0.00 |
| 7 | 9.36 | 12.89 | 51.94 |
| 8 | 1.77 | 14.65 | 10.94 |
| 10 | 13.11 | 8.50 | 34.76 |
| 11 | 17.90 | 30.16 | 41.64 |
| 12 | 24.68 | 58.29 | 44.53 |
| 13 | 15.92 | 26.52 | 25.45 |
| 14 | 3.02 | 0.00 | 17.31 |
| 16 | 4.14 | 41.66 | 28.57 |
| 17 | 7.16 | 57.38 | 52.85 |
| 19 | 21.61 | 35.51 | 79.75 |
| 21 | 26.19 | 8.18 | 46.43 |
| 22 | 26.75 | 64.39 | 52.49 |
| 23 | 21.57 | 10.96 | 52.53 |
| 24 | 34.56 | 25.78 | 63.07 |
| 25 | 26.32 | 13.69 | 65.55 |
| 27 | 26.83 | 51.12 | 50.27 |
| 30 | 10.09 | 44.33 | 15.46 |
| 31 | 18.68 | 38.93 | 61.39 |
| 32 | 49.44 | 34.22 | 63.47 |
| 33 | 36.54 | 49.41 | 67.27 |
| 34 | 1.42 | 46.79 | 30.29 |
| 36 | 5.78 | 38.02 | 52.49 |
| 37 | 32.92 | 100.00 | 56.56 |
| 38 | 39.09 | 14.17 | 50.05 |
| 39 | 23.73 | 54.71 | 60.13 |
| 40 | 3.24 | 0.00 | 0.00 |
| 41 | 30.07 | 68.72 | 42.99 |
| 42 | 7.42 | 23.96 | 0.00 |
| 44 | 35.50 | 69.04 | 49.05 |
| 45 | 0.00 | 0.00 | 18.40 |
| 48 | 15.23 | 26.04 | 15.73 |
| 50 | 4.75 | 32.73 | 35.62 |
| 51 | 21.14 | 56.68 | 43.58 |
| 52 | 26.27 | 45.56 | 29.88 |
| 53 | 29.98 | 42.51 | 53.53 |
| 54 | 36.80 | 70.53 | 62.70 |
| 55 | 16.82 | 3.58 | 40.19 |
| 56 | 31.28 | 57.11 | 60.85 |
| 57 | 21.14 | 37.65 | 54.43 |
| 59 | 35.12 | 53.74 | 57.50 |
| 60 | 38.78 | 78.98 | 53.57 |
| 61 | 22.04 | 65.40 | 29.20 |
| 62 | 35.25 | 19.09 | 19.85 |
| 63 | 0.00 | 2.35 | 14.74 |
| 64 | 6.90 | 36.95 | 37.79 |
| 66 | 7.64 | 57.86 | 60.67 |
| 70 | 0.00 | 0.00 | 5.52 |
| 72 | 33.69 | 11.18 | 55.79 |
| 77 | 42.02 | 20.59 | 57.19 |
| 80 | 25.02 | 0.00 | 64.60 |
| 81 | 26.14 | 50.53 | 73.73 |
| 82 | 18.42 | 19.79 | 53.21 |
| 83 | 33.39 | 28.88 | 53.80 |
| 87 | 16.91 | 30.16 | 30.29 |
| 88 | 10.87 | 54.65 | 58.63 |
| 89 | 0.00 | 35.51 | 0.00 |
| 90 | 44.18 | 54.92 | 75.45 |
| 91 | 41.54 | 48.29 | 55.11 |
| 93 | 0.00 | 56.68 | 10.44 |
| 94 | 31.28 | 41.50 | 66.00 |
| 95 | 46.64 | 58.07 | 59.04 |
| 98 | 0.00 | 0.00 | 0.00 |
| 99 | 0.00 | 15.4 | 0.00 |
| 105 | 0.00 | 19.95 | 0.00 |
| 108 | 16.39 | 0.00 | 65.69 |
| 109 | 7.33 | 0.00 | 32.87 |
| 110 | 9.88 | 13.58 | 59.45 |
| 111 | 22.30 | 48.82 | 29.79 |
| 114 | 26.62 | 38.07 | 74.19 |
| 115 | 4.53 | 2.30 | 59.18 |
| 116 | 10.4 | 6.7 | 4.3 |
| 117 | 17.99 | 28.56 | 43.58 |
| 118 | 0.00 | 15.61 | 0.00 |
| 120 | 0.00 | 47.01 | 22.33 |
| 122 | 35.46 | 33.69 | 23.55 |
| 123 | 19.24 | 39.89 | 50.59 |
| 124 | 21.18 | 1.28 | 63.83 |
| 126 | 23.64 | 8.34 | 38.52 |
| 127 | 25.97 | 0.00 | 52.12 |
| 128 | 3.67 | 7.97 | 51.40 |
| 129 | 23.64 | 38.50 | 29.75 |
| 130 | 28.43 | 32.99 | 33.27 |
| 131 | 3.11 | 20.70 | 52.44 |
| 132 | 35.16 | 32.83 | 46.75 |
| 133 | 43.36 | 47.81 | 54.02 |
| 134 | 0.00 | 43.16 | 45.12 |
| 135 | 12.81 | 56.20 | 51.27 |
| 136 | 39.82 | 17.97 | 57.78 |
| 138 | 21.70 | 54.92 | 32.37 |
| 140 | 32.73 | 14.06 | 51.54 |
| 141 | 20.92 | 27.71 | 66.64 |
| 142 | 15.36 | 51.55 | 49.10 |
| 143 | 34.43 | 34.49 | 40.46 |
| 144 | 32.61 | 35.03 | 56.65 |
| 145 | 23.25 | 32.83 | 30.83 |
| 146 | 23.12 | 41.82 | 66.18 |
| 147 | 31.71 | 42.83 | 44.21 |
| 150 | 17.00 | 59.95 | 56.92 |
| 152 | 16.31 | 18.98 | 44.12 |
| 153 | 14.06 | 41.55 | 56.78 |
| 155 | 14.54 | 8.98 | 42.04 |
| 156 | 38.35 | 43.90 | 13.16 |
| 157 | 25.28 | 53.42 | 77.53 |
| 158 | 2.85 | 19.30 | 17.99 |
| 159 | 22.91 | 17.49 | 49.77 |
| 160 | 21.74 | 49.47 | 35.13 |
| 161 | 28.52 | 32.99 | 44.94 |
| 162 | 30.80 | 41.44 | 72.78 |
| 163 | 22.04 | 32.46 | 34.40 |
| 164 | 34.60 | 45.51 | 36.12 |
| 165 | 18.64 | 36.90 | 50.86 |
| 166 | 35.33 | 21.28 | 45.03 |
| 167 | 0.00 | 41.28 | 0.00 |
| 168 | 29.64 | 38.88 | 60.26 |
| 169 | 0.00 | 0.00 | 16.37 |
| 170 | 7.29 | 57.59 | 10.31 |
| 171 | 0.00 | 41.71 | 0.00 |
| 174 | 19.76 | 28.56 | 46.88 |
| 177 | 14.28 | 24.01 | 37.03 |
| 178 | 30.41 | 71.07 | 57.69 |
| 179 | 5.87 | 42.09 | 27.40 |
| 180 | 0.00 | 10.37 | 25.41 |
| 181 | 11.73 | 49.09 | 12.52 |
| 183 | 16.39 | 21.02 | 74.77 |
| 185 | 22.86 | 0.00 | 64.15 |
| 186 | 19.93 | 53.90 | 30.65 |
| 187 | 22.43 | 83.96 | 24.10 |
| 190 | 2.11 | 41.82 | 44.80 |
| 191 | 14.15 | 71.18 | 50.63 |
| 195 | 18.12 | 39.36 | 38.20 |
| 199 | 21.57 | 49.04 | 18.54 |
| 200 | 31.67 | 37.11 | 41.59 |
| 203 | 23.64 | 7.17 | 55.20 |
| 204 | 9.15 | 37.33 | 50.81 |
| 205 | 23.68 | 57.59 | 77.62 |
| 206 | 34.43 | 29.30 | 50.77 |
| 207 | 26.27 | 47.86 | 42.22 |
| 208 | 46.29 | 68.61 | 55.24 |
| 210 | 3.75 | 21.02 | 32.64 |
| 211 | 44.69 | 13.26 | 62.52 |
| 213 | 26.53 | 27.97 | 49.28 |
| 214 | 3.02 | 18.13 | 18.13 |
| 215 | 32.53 | 24.44 | 56.69 |
| 216 | 21.40 | 8.34 | 38.52 |
| 217 | 3.19 | 34.87 | 8.18 |
| 218 | 0.00 | 5.51 | 0.00 |
| 220 | 27.91 | 62.46 | 46.88 |
| 221 | 44.39 | 25.94 | 63.25 |
| 222 | 25.02 | 17.65 | 60.04 |
| 224 | 33.09 | 71.98 | 52.26 |
| 225 | 24.25 | 33.26 | 52.31 |
| 226 | 26.66 | 58.93 | 41.37 |
| 227 | 40.55 | 53.96 | 54.48 |
| 228 | 17.26 | 31.87 | 36.17 |
| 229 | 43.18 | 54.81 | 23.73 |
| 230 | 35.16 | 4.22 | 49.41 |
| 231 | 25.84 | 44.17 | 66.64 |
| 232 | 37.06 | 19.52 | 43.40 |
| 233 | 16.26 | 55.72 | 57.14 |
| 234 | 26.66 | 36.90 | 49.10 |
| 235 | 27.44 | 0.00 | 69.76 |
| 236 | 39.82 | 64.49 | 68.49 |
| 237 | 36.97 | 52.14 | 37.70 |
| 238 | 35.76 | 66.10 | 56.83 |
| 240 | 28.52 | 36.79 | 67.41 |
| 241 | 32.44 | 43.37 | 73.10 |
| 242 | 31.92 | 16.42 | 55.47 |
| 243 | 14.62 | 37.91 | 46.79 |
| 244 | 6.34 | 14.01 | 61.44 |
| 245 | 24.07 | 53.32 | 53.89 |
| 246 | 24.59 | 50.80 | 39.87 |
| 247 | 31.10 | 50.05 | 49.68 |
| 248 | 34.81 | 81.23 | 49.64 |
| 249 | 32.48 | 48.56 | 51.40 |
| 250 | 25.93 | 55.03 | 0.00 |
| 251 | 0.00 | 45.45 | 38.88 |
| 252 | 33.18 | 23.26 | 53.53 |
| 253 | 36.15 | 27.33 | 52.08 |
| 254 | 5.74 | 56.95 | 40.37 |
| 255 | 16.13 | 10.32 | 40.14 |
| 256 | 30.41 | 47.17 | 49.32 |
| 257 | 10.27 | 46.52 | 26.54 |
| 258 | 24.50 | 48.18 | 59.63 |
| 259 | 14.97 | 46.95 | 67.18 |
| 261 | 0.69 | 53.58 | 56.78 |
| 262 | 18.98 | 10.27 | 39.29 |
| 263 | 28.86 | 39.73 | 48.46 |
| 264 | 19.20 | 46.84 | 32.46 |
| 266 | 22.61 | 48.72 | 56.28 |
| 267 | 34.56 | 37.01 | 65.01 |
| 268 | 18.38 | 57.70 | 68.08 |
| 269 | 24.46 | 39.47 | 35.04 |
| 270 | 30.72 | 61.60 | 53.62 |
| 271 | 22.52 | 0.00 | 59.67 |
| 272 | 30.28 | 42.41 | 45.48 |
| 273 | 23.43 | 25.35 | 58.54 |
| 274 | 44.43 | 56.15 | 49.01 |
| 275 | 0.00 | 0.00 | 0.00 |
| 276 | 31.84 | 27.27 | 51.81 |
| 277 | 11.73 | 21.71 | 61.80 |
| 278 | 45.60 | 45.51 | 81.74 |
| 279 | 37.83 | 27.49 | 18.76 |
| 281 | 31.62 | 55.99 | 38.79 |
| 282 | 8.20 | 21.82 | 63.74 |
| 283 | 29.77 | 43.80 | 49.59 |
| 285 | 35.29 | 22.89 | 80.38 |
| 286 | 0.00 | 0.00 | 19.17 |
| 287 | 7.89 | 29.41 | 56.69 |
| 288 | 28.17 | 52.19 | 49.14 |
| 289 | 28.95 | 43.48 | 40.64 |
| 290 | 0.00 | 18.88 | 50.59 |
| 292 | 15.92 | 25.99 | 54.25 |
| 293 | 0.00 | 0.00 | 0.00 |
| 294 | 28.73 | 57.70 | 54.25 |
| 298 | 20.66 | 57.01 | 55.11 |
| 300 | 18.25 | 0.00 | 45.03 |
| 301 | 28.82 | 56.15 | 68.40 |
| 302 | 39.04 | 26.58 | 56.87 |
| 303 | 33.39 | 34.44 | 49.64 |
| 304 | 0.00 | 41.76 | 12.43 |
| 306 | 13.59 | 7.27 | 51.08 |
| 308 | 0.00 | 0.00 | 23.73 |
| 310 | 2.85 | 19.30 | 17.99 |
| 311 | 8.41 | 0.00 | 17.54 |
| 314 | 32.05 | 7.86 | 52.12 |
| 315 | 26.19 | 41.28 | 89.15 |
| 316 | 30.59 | 55.94 | 54.43 |
| 317 | 14.75 | 20.21 | 53.62 |
| 318 | 15.31 | 44.60 | 31.65 |
| 320 | 17.47 | 15.29 | 52.49 |
| 321 | 23.81 | 27.27 | 43.44 |
| 323 | 5.87 | 0.00 | 30.74 |
| 325 | 0.00 | 5.08 | 0.00 |
| 326 | 22.74 | 45.45 | 63.92 |
| 327 | 0.00 | 0.00 | 63.79 |
| 328 | 37.53 | 55.83 | 49.50 |
| 329 | 22.17 | 19.25 | 42.31 |
| 330 | 24.59 | 26.42 | 58.68 |
| 331 | 22.61 | 56.47 | 69.30 |
| 334 | 29.55 | 20.75 | 41.41 |
| 337 | 15.92 | 0.00 | 37.57 |
| 338 | 19.33 | 55.35 | 12.21 |
| 339 | 23.60 | 0.00 | 52.08 |
| 340 | 21.66 | 40.32 | 41.73 |
| 341 | 29.25 | 31.18 | 50.50 |
| 343 | 35.72 | 28.61 | 50.95 |
| 344 | 26.62 | 77.91 | 43.49 |
| 345 | 22.13 | 44.01 | 56.96 |
| 346 | 9.36 | 54.60 | 56.83 |
| 347 | 24.85 | 56.84 | 57.73 |
| 348 | 35.81 | 55.78 | 46.65 |
| 349 | 26.19 | 57.59 | 64.51 |
| 350 | 0.00 | 22.83 | 29.93 |
| 351 | 22.74 | 42.78 | 60.44 |
| 352 | 31.02 | 13.10 | 47.92 |
| 353 | 16.61 | 51.34 | 35.44 |
| 354 | 17.17 | 40.80 | 45.16 |
| 355 | 22.04 | 30.16 | 52.35 |
| 356 | 30.80 | 49.30 | 54.29 |
| 357 | 38.61 | 61.23 | 100.00 |
| 358 | 20.36 | 67.11 | 48.96 |
| 359 | 28.90 | 45.29 | 62.03 |
| 360 | 0.00 | 2.83 | 3.44 |
| 361 | 26.57 | 12.09 | 42.00 |
| 362 | 14.93 | 50.16 | 50.90 |
| 363 | 0.00 | 0.00 | 2.80 |
| 364 | 31.36 | 40.05 | 47.60 |
| 365 | 0.00 | 32.41 | 17.45 |
| 366 | 6.17 | 48.45 | 23.06 |
| 367 | 27.39 | 14.97 | 54.70 |
| 368 | 32.48 | 65.83 | 65.37 |
| 369 | 32.92 | 33.26 | 47.15 |
| 370 | 24.63 | 41.66 | 56.96 |
| 372 | 18.25 | 81.82 | 45.34 |
| 373 | 28.82 | 53.42 | 67.77 |
| 374 | 17.64 | 38.50 | 48.60 |
| 376 | 13.46 | 0.00 | 12.70 |
| 379 | 41.50 | 35.24 | 51.18 |
| 385 | 14.88 | 48.50 | 56.74 |
| 387 | 36.32 | 39.89 | 41.41 |
| 391 | 14.02 | 51.44 | 35.44 |
| 392 | 15.75 | 34.87 | 60.58 |
| 395 | 0.00 | 88.24 | 37.25 |
| 396 | 21.48 | 46.26 | 26.67 |
| 397 | 25.06 | 38.72 | 46.16 |
| 399 | 21.83 | 76.15 | 61.39 |

### Example 2. In-Vitro Antiviral Activity of Example 369

In vitro studies of the antiviral activity were also carried out for several viruses using Example 369. The results are summarized in Table 3.

TABLE 3
In vitro viral inhibition using Example 369

| | | EC₅₀ (ug/mL) |
|---|---|---|
| | EC₅₀ (ug/mL) | (Neutral Red (NR) |
| Virus (strain; cell) | (VIS Drug Assay) | Assay) |
| Chikungunya (S27; Vero 76) | 0.12 | 0.18 |
| Dengue Virus 2 (New Guinea | <0.1 | <0.1 |
| C; Huh7) | | |
| Eastern equine encephalitis | <0.1 | <0.1 |
| (FL39-939; Vero 76) | | |
| Japanese encephalitis virus | 0.32 | 3.0 |
| (SA-14; Vero 76) | | |
| Mayaro virus (BE AR 505411; | 0.32 | 0.28 |
| Vero 76) | | |
| Measles (CC; Vero 76) | <0.1 | <0.1 |
| Powassan virus (LB; BHK-21) | <0.1 | <0.1 |
| Respiratory syncytial virus (A₂; | 028 | 0.3 |
| RD) | | |
| Usutu virus (TC-508; Vero 76) | 2.7 | 5.8 |
| Venezuelan equine encephalitis | 0.55 | 0.77 |
| (TC-83; Vero 76) | | |
| Western equine encephalitis | <0.1 | <0.1 |
| (California; Vero 76) | | |
| Yellow Fever (YFV 17D; | 0.18 | 0.18 |
| Huh7) | | |

### Example 3. Compound Preparation

The compounds of this disclosure may be made by a variety of methods, including well-known standard synthetic methods. Illustrative general synthetic methods are set out below (e.g., see Schemes 1, 2, and 3). The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts, (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention. Starting materials are commercially available or are made from commercially available starting materials using methods known to those skilled in the art.

Abbreviations

| | |
|---|---|
| Ac₂O | acetic anhydride |
| AcOH | acetic acid |
| AIBN | azobisisobutyronitrile |
| aq. | aqueous |
| BBr₃ | boron tribromide |
| BF₃•OEt₂ | boron trifluoride diethyl etherate |
| BH₃•DMS | borane dimethyl sulfide complex |
| (±)-BINAP | racemic 2,2'-bis(diphenylphosphino)-1,1'- |
| | binaphthalene |
| Bn | benzyl |
| BnOH | benzyl alcohol |
| Boc₂O | di-tert-butyl decarbonate |
| BPin | 4,4,5,5-tetramethyl-1,3,2-dioxaborolane |
| Br₂ | bromine |
| CaCl₂ | calcium chloride |
| CBr₄ | carbon tetrabromide |
| CbzCl | benzyl chloroformate |
| CCl₄ | carbon tetrachloride |
| CDI | 1,1'-carbonyldiimidazole |
| Cl₂ | chlorine gas |
| Cs₂CO₃ | cesium carbonate |
| CuI | copper(I) iodide |
| CuSO₄ | copper(II) sulfate |
| DAST | diethylaminosulfur trifluoride |
| DCE | dichloroethane |
| DCM or CH₂Cl₂ | dichloromethane |
| DEAD | diethyl azodicarboxylate |
| Dess-Martin | 1,1,1-tris(acetyloxy)-1,1-dihydro-1,2-benziodoxol- |
| | 3-(1H)-one |
| DIAD | diisopropyl azodicarboxylate |
| DIEA | diisopropylethylamine |
| DMF | N, N-dimethylformamide |
| DMSO | dimethylsulfoxide |
| DPPA | diphenylphosphoryl azide |
| EA or EtOAc | ethyl acetate |
| EDC | 1-ethyl-3-(3-dimethylaminopropyl)carbodiimide |
| ES-LCMS | electrospray liquid chromatography-mass |
| | spectrometry |
| EtI | ethyl iodide |
| EtMgBr | ethylmagnesium bromide |
| Et₃N | triethylamine |
| EtOH | ethanol |
| g | gram(s) |
| Grubbs I | benzylidene-bis(tricyclohexylphosphine) |
| | dichlororuthenium |
| h | hour(s) |
| H2 | hydrogen gas |
| HATU | O-(7-azabenzotriazol-1-yl)-N, N, N’, N”- |
| | tetramethyluronium hexafluorophosphate |
| HCl | hydrochloric acid |
| H₂O | water |
| HOBt | hydroxybenzotriazole |
| HPLC | high performance liquid chromatography |
| in vacuo | under vacuum |
| i-PrOH | isopropyl alcohol |
| [Ir(COD)OMe]₂ | di-μ-methoxobis(1,5-cyclooctadiene)diiridium(I) |
| KCN | potassium cyanide |
| K₂CO₃ | potassium carbonate |
| KI | potassium iodide |
| KOAc | potassium acetate |
| K₃PO₄ | potassium phosphate tribasic |
| L | liter(s) |
| LAH or LiAlH₄ | lithium aluminium hydride |
| LCMS | liquid chromatography-mass spectrometry |
| LiHMDS | lithium bis(trimethylsilyl)amide |
| LiOH | lithium hydroxide |
| LiOH•H₂O | lithium hydroxide monohydrate |
| M | molar |
| m-CPBA | meta-chloroperoxybenzoic acid |
| MeCN | acetonitrile |
| MeI | methyl iodide |
| MeMgBr | methylmagnesium bromide |
| MeNH₂ | methylamine |
| MeOH | methanol |
| MgSO₄ | magnesium sulfate |
| min | minute(s) |
| mL | milliliter(s) |
| mmol | millimole(s) |
| mol | mole(s) |
| MsCl | methanesulfonyl chloride |
| MTBE | methyl tert-butyl ether |
| N | normal |
| N₂ | nitrogen gas |
| NaBH₄ | sodium borohydride |
| NaBH₃CN | sodium cyanoborohydride |
| NaBH(OAc)₃ | sodium triacetoxyborohydride |
| NaCN | sodium cyanide |
| NaH | sodium hydride |
| NaHCO₃ | sodium bicarbonate |
| NaOH | sodium hydroxide |
| Na₂SO₄ | sodium sulfate |
| NBS | N-bromosuccinimide |
| n-BuLi | n-butyllithium |
| n-BuMgCl | n-butylmagnesium chloride |
| NH₃ | ammonia |
| NH₄Cl | ammonium chloride |
| NH₄OAc | ammonium acetate |
| NH₄OH | ammonium hydroxide |
| NMP | N-methyl-2-pyrrolidone |
| NMR | nuclear magnetic resonance |
| OTf | trifluoromethanesulfonate |
| Oxone ® | potassium peroxymonosulfate |
| Pd(OAc)₂ | palladium(II) acetate |
| Pd/C | palladium on carbon |
| PdCl₂(dppf) | [1,1′-bis(diphenylphosphino)ferrocene] |
| | dichloropalladium(II) |
| Pd₂(dba)₃ | tris(dibenzylideneacetone)dipalladium(0) |
| Pd(OH)₂ | palladium(II) hydroxide |
| Pd(PPh₃)₂Cl₂ | bis(triphenylphosphine)palladium(II) dichloride |
| PE | petroleum ether |
| POCl₃ | phosphoryl chloride |
| PPh₃ | triphenylphosphine |
| p-TsCl | para-toluenesulfonyl chloride |
| p-TsOH | para-toluenesulfonic acid |
| SFC | supercritical fluid chromatography |
| SOCl₂ | thionyl chloride |
| TBAF | tetra-n-butylammonium fluoride |
| TBS | tert-butyldimethylsilyl |
| TBSCl | tert-butyldimethylsilyl chloride |
| t-BuOH | tert-butyl alcohol |
| t-BuOK | potassium tert-butoxide |
| t-BuONa | sodium tert-butoxide |
| TFA | trifluoroacetic acid |
| Tf₂O | trifluoromethanesulfonic anhydride |
| THF | tetrahydrofuran |
| TLC | thin layer chromotrography |
| TMS-N₃ | trimethylsilyl azide |
| TosMIC | para-toluenesulfonylmethyl isocyanide |
| Xantphos | 4,5-bis(diphenylphosphino)-9,9-dimethylxanthene |
| Zn | zinc metal |

Certain compounds of Formula (I) can be prepared according to Schemes 1, 2, or 3, or analogous or other methods known in the art.

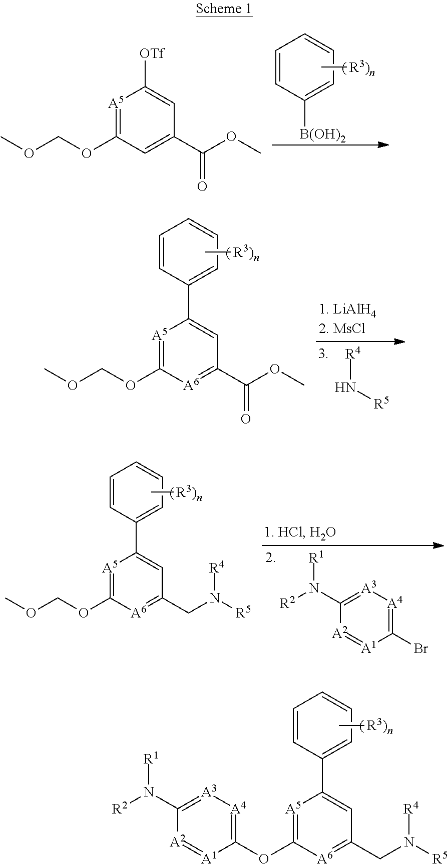

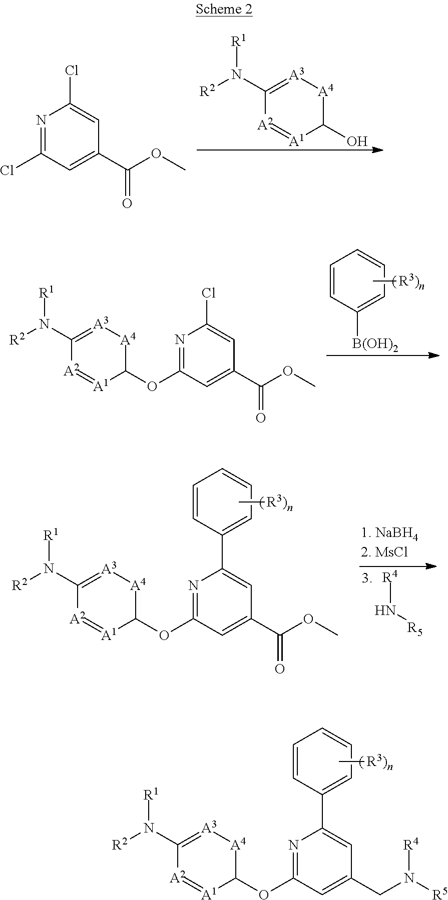

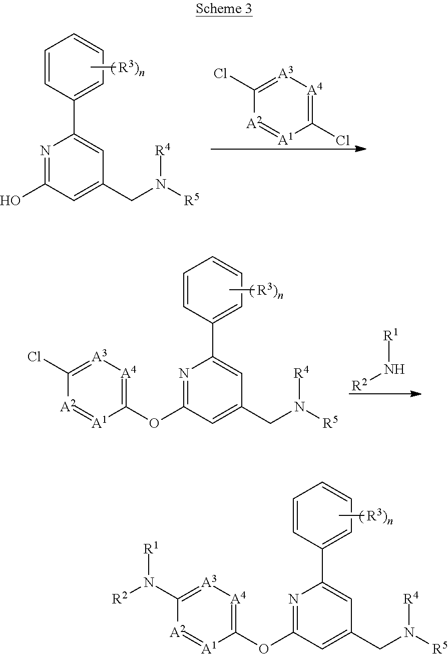

### Experimentals

These examples are not intended to limit the scope of the present disclosure, but rather to provide guidance to the skilled artisan to prepare and use the compounds in the methods and uses of the disclosure. While particular embodiments of the present disclosure are described, the skilled artisan will appreciate that various changes and modifications can be made without departing from the spirit and scope of the disclosure. Unless otherwise noted, reagents are commercially available or are prepared according to procedures in the literature. The symbols and conventions used in the descriptions of processes, schemes, and examples are consistent with those used in the contemporary scientific literature, for example, the Journal of the American Chemical Society or the Journal of Biological Chemistry.

Preparative HPLC was performed on a Gilson UV/VIS-156 with UV detection at 220/254 nm Gilson 281 automatic collection. HPLC column commonly used ASB-C18 21.2×150 mm or Phenomenex 21.2×150 mm. HPLC Gradient (acidic condition, 0.01% HCl or 0.1% formic acid) used 0-100% acetonitrile with water and corresponding acid, the gradient shape was optimized for individual separations. Unless specially mentioned, compounds are isolated in HCl system and thus obtained as HCl salts. However, the compounds can also be isolated and used as the free base. HPLC Gradient (basic condition, 0.05% NH₃—H₂O or neutral condition, 0.01% NH₄HCO₃) was optimized for individual separation.

Chemical shifts are expressed in parts per million (ppm) units. Coupling constants (J) are in units of hertz (Hz). Splitting patterns describe apparent multiplicities and are designated as s (single), d (double), t (triplet), dd (double doublet), dt (double triplet), dq (double quartet), m (multiplet), and br (broad).

Flash column chromatography was performed using silica gel.

The naming programs used are ACDLABs 11.0 Namebatch, ACD IUPAC, or ChemDraw.

### Intermediate 1: tert-Butyl 4-(5-((6-(3,5-dichlorophenyl)-4-(((methylsulfonyl)oxy)methyl)pyridin-2-yl)oxy)pyrimidin-2-yl)piperazine-1-carboxylate

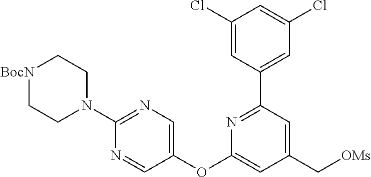 [SMILES: CC(C)(C)OC(=O)N1CCN(c2ncc(Oc3cc(COS(C)(=O)=O)cc(-c4cc(Cl)cc(Cl)c4)n3)cn2)CC1]

Intermediate 1 was prepared according to the following steps.

### Step 1: 5-(Benzyloxy)-2-chloropyrimidine

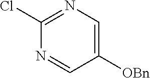 [SMILES: Clc1ncc(OCc2ccccc2)cn1]

To a mixture of 2-chloropyrimidin-5-ol (45 g, 345 mmol) in DMF (1 L) was added Cs₂CO₃ (337 g, 1034 mmol) and (bromomethyl)benzene (49.1 mL, 414 mmol). The mixture was stirred at 15° C. for 8 h under N₂ atmosphere. Then the mixture was concentrated and saturated aqueous NaHCO₃ solution (150 mL) was added. The aqueous layer was extracted with EtOAc (500 mL×2), and the combined extracts were washed with brine (150 mL×2), dried over Na₂SO₄, filtered and concentrated to yield a yellow oil of 5-(benzyloxy)-2-chloropyrimidine (78 g, 318 mmol, 92.0% yield): ¹H NMR (400 MHz, CD₃OD) δ ppm 8.45 (s, 2H), 7.52-7.29 (m, 5H), 5.23 (s, 2H); ES-LCMS m/z 221.2, 223.1 [M+H]⁺.

### Step 2: tert-Butyl 4-(5-(benzyloxy)pyrimidin-2-yl)piperazine-1-carboxylate

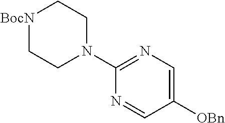 [SMILES: CC(C)(C)OC(=O)N1CCN(c2ncc(OCc3ccccc3)cn2)CC1]

To a mixture of 5-(benzyloxy)-2-chloropyrimidine (15 g, 61.2 mmol) and tert-butyl piperazine-1-carboxylate (17.09 g, 92 mmol) in DMF (200 mL) was added Cs₂CO₃ (59.8 g, 184 mmol). The mixture was stirred at 120° C. for 10 h. The reaction mixture was concentrated and purified by silica gel column chromatography (PE/EtOAc=5/1). All fractions found to contain product by TLC (PE/EA=3/1, R_{f}=0.6) were combined and concentrated to yield a white solid of tert-butyl 4-(5-(benzyloxy)pyrimidin-2-yl)piperazine-1-carboxylate (10 g, 25.6 mmol, 41.9% yield): ¹H NMR (400 MHz, CDCl₃) δ ppm 8.13 (s, 2H), 7.43-7.31 (m, 5H), 5.03 (s, 2H), 3.75-3.65 (m, 4H), 3.56-3.43 (m, 4H), 1.49 (s, 9H); ES-LCMS m/z 371.3 [M+H]⁺.

### Step 3: tert-Butyl 4-(5-hydroxypyrimidin-2-yl)piperazine-1-carboxylate

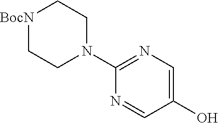 [SMILES: CC(C)(C)OC(=O)N1CCN(c2ncc(O)cn2)CC1]

To a solution of tert-butyl 4-(5-(benzyloxy)pyrimidin-2-yl)piperazine-1-carboxylate (10 g, 25.6 mmol) in MeOH (30 mL) was added Pd/C (10 wt %, 2.73 g, 2.56 mmol). The mixture was stirred under 1 atm H₂ atmosphere at 15° C. for 0.5 h. The mixture was filtered and concentrated to yield a light yellow solid of tert-butyl 4-(5-hydroxypyrimidin-2-yl)piperazine-1-carboxylate (7.5 g, 22.74 mmol, 89.0% yield): ¹H NMR (400 MHz, CD₃OD) δ ppm 8.03 (s, 2H), 3.70-3.59 (m, 4H), 3.50 (d, J=4.5 Hz, 4H), 1.50 (s, 9H); ES-LCMS m/z 225.2 [M-t-Bu+H]⁺.

### Step 4: tert-Butyl 4-(5-((6-chloro-4-(methoxycarbonyl)pyridin-2-yl)oxy)pyrimidin-2-yl)piperazine-1-carboxylate

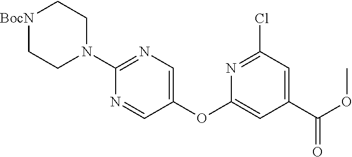 [SMILES: COC(=O)c1cc(Cl)nc(Oc2cnc(N3CCN(C(=O)OC(C)(C)C)CC3)nc2)c1]

To a mixture of methyl 2,6-dichloroisonicotinate, hydrochloride (6.7 g, 27.4 mmol) and tert-butyl 4-(5-hydroxypyrimidin-2-yl)piperazine-1-carboxylate (7.4 g, 22.44 mmol) in DMF (80 mL) was added K₂CO₃ (9.30 g, 67.3 mmol). The mixture was stirred at 50° C. for 10 h. Then the solution was concentrated and saturated aqueous NaHCO₃ solution (150 mL) was added. The aqueous layer was extracted with DCM (500 mL×2), and the combined extracts were washed with brine (150 mL×2), dried over Na₂SO₄, filtered and concentrated. The crude material was purified by silica gel column chromatography (PE/EtOAc=5/1). All fractions found to contain product by TLC (PE/EA=3/1, R_{f}=0.5) were combined and concentrated to yield a yellow oil of tert-butyl 4-(5-((6-chloro-4-(methoxycarbonyl)pyridin-2-yl)oxy)pyrimidin-2-yl)piperazine-1-carboxylate (8.4 g, 16.80 mmol, 74.9% yield): ¹H NMR (400 MHz, CD₃OD) δ ppm 8.31 (s, 2H), 7.62 (s, 1H), 7.51 (s, 1H), 3.99 (s, 3H), 3.90-3.80 (m, 4H), 3.55 (m, 4H), 1.51 (s, 9H); ES-LCMS m/z 394.2, 396.1 [M-t-Bu+H]⁺.

### Step 5: tert-Butyl 4-(5-((6-(3,5-dichlorophenyl)-4-(methoxycarbonyl)pyridin-2-yl)oxy)pyrimidin-2-yl)piperazine-1-carboxylate

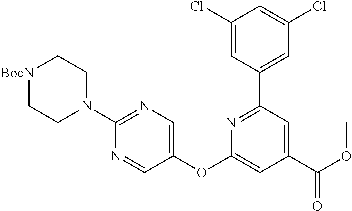 [SMILES: COC(=O)c1cc(Oc2cnc(N3CCN(C(=O)OC(C)(C)C)CC3)nc2)nc(-c2cc(Cl)cc(Cl)c2)c1]

To a mixture of tert-butyl 4-(5-((6-chloro-4-(methoxycarbonyl)pyridin-2-yl)oxy)pyrimidin-2-yl)piperazine-1-carboxylate (8 g, 16.00 mmol) and (3,5-dichlorophenyl)boronic acid (7.63 g, 40.0 mmol) in DMF (100 mL) was added K₂CO₃ (6.64 g, 48.0 mmol) and PdCl₂(dppf) (0.586 g, 0.800 mmol). The mixture was stirred at 80° C. for 2 h under N₂ atmosphere. The mixture was concentrated and purified by silica gel column chromatography (PE/EtOAc=5/1). All fractions found to contain product by TLC (PE/EA=3/1, R_{f}=0.4) were combined and concentrated to yield a colorless oil of tert-butyl 4-(5-((6-(3,5-dichlorophenyl)-4-(methoxycarbonyl)pyridine-2-yl)oxy)pyrimidin-2-yl)piperazine-1-carboxylate (7.4 g, 11.22 mmol, 70.1% yield): ¹H NMR (400 MHz, CDCl₃) δ ppm 8.30 (s, 2H), 7.95 (s, 1H), 7.75 (d, J=1.3 Hz, 2H), 7.48 (s, 1H), 7.37 (s, 1H), 4.00 (s, 3H), 3.89-3.75 (m, 4H), 3.60-3.45 (m, 4H), 1.49 (s, 9H); ES-LCMS m/z 504.1, 506.1 [M-t-Bu+H]⁺.

### Step 6: tert-Butyl 4-(5-((6-(3,5-dichlorophenyl)-4-(hydroxymethyl)pyridin-2-yl)oxy)pyrimidin-2-yl)piperazine-1-carboxylate

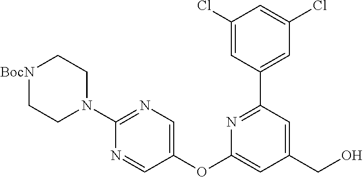 [SMILES: CC(C)(C)OC(=O)N1CCN(c2ncc(Oc3cc(CO)cc(-c4cc(Cl)cc(Cl)c4)n3)cn2)CC1]

To a mixture of tert-butyl 4-(5-((6-(3,5-dichlorophenyl)-4-(methoxycarbonyl)pyridin-2-yl)oxy)pyrimidin-2-yl)piperazine-1-carboxylate (7.4 g, 11.22 mmol) in MeOH (150 mL) was added NaBH₄ (2.123 g, 56.1 mmol). The mixture was stirred at 15° C. for 10 min under N₂ atmosphere then concentrated and saturated aqueous NaHCO₃ solution (150 mL) was added. The aqueous layer was extracted with DCM (500 mL×2) and the combined extracts were washed with brine (150 mL×2), dried over Na₂SO₄, filtered and concentrated to yield a yellow oil of tert-butyl 4-(5-((6-(3,5-dichlorophenyl)-4-(hydroxymethyl)pyridine-2-yl)oxy)pyrimidin-2-yl)piperazine-1-carboxylate (7.2 g, 10.82 mmol, 96.0% yield): ¹H NMR (400 MHz, CD₃OD) δ ppm 8.35 (s, 2H), 7.81 (s, 2H), 7.60 (s, 1H), 7.44 (s, 1H), 7.06 (s, 1H), 4.75 (s, 2H), 3.89-3.82 (m, 4H), 3.54 (m, 4H), 1.51 (s, 9H); ES-LCMS m/z 532.2, 534.2 [M+H]⁺.

### Step 7: tert-Butyl 4-(5-((6-(3,5-dichlorophenyl)-4-(((methylsulfonyl)oxy)methyl)pyridin-2-yl)oxy)pyrimidin-2-yl)piperazine-1-carboxylate

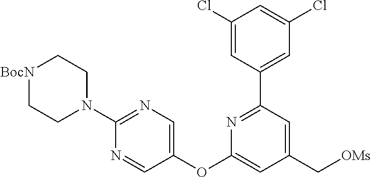 [SMILES: CC(C)(C)OC(=O)N1CCN(c2ncc(Oc3cc(COS(C)(=O)=O)cc(-c4cc(Cl)cc(Cl)c4)n3)cn2)CC1]

To a mixture of tert-butyl 4-(5-((6-(3,5-dichlorophenyl)-4-(hydroxymethyl)pyridin-2-yl)oxy)pyrimidin-2-yl)piperazine-1-carboxylate (4.5 g, 6.76 mmol) in DCM (150 mL) was added MsCl (0.79 mL, 10.14 mmol) and DIEA (3.54 mL, 20.28 mmol). The mixture was stirred at 15° C. for 10 min under N₂ atmosphere. Then the solution was concentrated and saturated aqueous NaHCO₃ solution (150 mL) was added. The aqueous layer was extracted with DCM (500 mL×2), and the combined extracts were washed with brine (150 mL×2), dried over Na₂SO₄, filtered and concentrated to yield a yellow oil of tert-butyl 4-(5-((6-(3,5-dichlorophenyl)-4-(((methylsulfonyl)oxy)methyl)pyridin-2-yl)oxy)pyrimidin-2-yl)piperazine-1-carboxylate (5 g, 6.55 mmol, 97.0% yield): ¹H NMR (400 MHz, CD₃OD) δ ppm 8.37 (s, 2H), 7.85 (s, 2H), 7.48 (s, 1H), 7.41 (s, 1H), 7.16 (s, 1H), 3.86 (d, J=5.5 Hz, 6H), 3.55 (s, 4H), 3.25-3.23 (m, 3H), 1.51 (s, 9H); ES-LCMS m/z 554.2, 556.2 [M-t-Bu+H]⁺.

### Example 92: N-((1-((2-(3,5-Dichlorophenyl)-6-((2-(piperazin-1-yl)pyrimidin-5-yl)oxy)pyridin-4-yl)methyl)piperidin-4-yl)methyl)acetamide

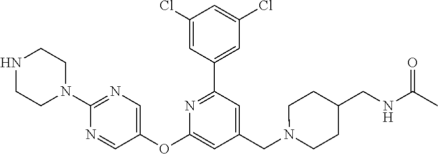 [SMILES: CC(=O)NCC1CCN(Cc2cc(Oc3cnc(N4CCNCC4)nc3)nc(-c3cc(Cl)cc(Cl)c3)c2)CC1]

Example 92 was prepared according to the following steps.

### Step 1: tert-Butyl 4-(5-((4-((4-(acetamidomethyl)piperidin-1-yl)methyl)-6-(3,5-dichlorophenyl)pyridin-2-yl)oxy)pyrimidin-2-yl)piperazine-1-carboxylate

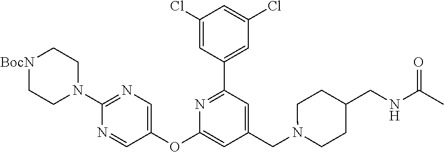 [SMILES: CC(=O)NCC1CCN(Cc2cc(Oc3cnc(N4CCN(C(=O)OC(C)(C)C)CC4)nc3)nc(-c3cc(Cl)cc(Cl)c3)c2)CC1]

To a mixture of tert-butyl 4-(5-((6-(3,5-dichlorophenyl)-4-(((methylsulfonyl)oxy)methyl)pyridin-2-yl)oxy)pyrimidin-2-yl)piperazine-1-carboxylate (200 mg, 0.328 mmol), N-(piperidin-4-ylmethyl)acetamide hydrochloride (127 mg, 0.655 mmol) in DMF (5 mL) was added K₂CO₃ (181 mg, 1.310 mmol). The reaction was stirred at 50° C. for 8 h under N₂ atmosphere then concentrated. Saturated aqueous NaHCO₃ solution (15 mL) was added and the aqueous layer was extracted with DCM (150 mL×2). The combined extracts were washed with brine (15 mL×2), dried over Na₂SO₄, filtered and concentrated to yield a yellow oil of tert-butyl 4-(5-((4-((4-(acetamidomethyl)piperidin-1-yl)methyl)-6-(3,5-dichlorophenyl)pyridin-2-yl)oxy)pyrimidin-2-yl)piperazine-1-carboxylate (200 mg, 0.18 mmol, 54.6% yield): ¹H NMR (400 MHz, CDCl₃) δ ppm 8.33-8.21 (m, 3H), 7.78-7.64 (m, 2H), 7.48-7.28 (m, 2H), 3.82 (br. s, 4H), 3.52 (br. s, 4H), 3.25-3.07 (m, 4H), 2.85-2.90 (m, 4H), 1.99 (d, J=2.5 Hz, 3H), 1.90-1.80 (m, 5H), 1.49-1.40 (m, 9H); ES-LCMS m/z 670.3, 672.3 [M+H]⁺.

### Step 2: N-((1-((2-(3,5-Dichlorophenyl)-6-((2-(piperazin-1-yl)pyrimidin-5-yl)oxy)pyridin-4-yl)methyl)piperidin-4-yl)methyl)acetamide, 4 hydrochloride

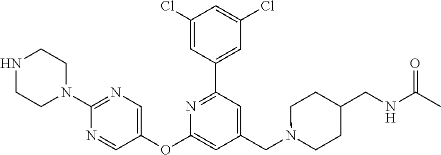 [SMILES: CC(=O)NCC1CCN(Cc2cc(Oc3cnc(N4CCNCC4)nc3)nc(-c3cc(Cl)cc(Cl)c3)c2)CC1]

To a mixture of tert-butyl 4-(5-((4-((4-(acetamidomethyl)piperidin-1-yl)methyl)-6-(3,5-dichlorophenyl)pyridin-2-yl)oxy)pyrimidin-2-yl)piperazine-1-carboxylate (100 mg, 0.149 mmol) in DCM (10 mL) was added TFA (0.287 mL, 3.73 mmol). The reaction was stirred at 15° C. for 10 min under N₂ atmosphere then concentrated and purified by preparative HPLC (MeCN/H₂O as eluents, acidic condition) to yield a yellow solid of N-((1-((2-(3,5-dichlorophenyl)-6-((2-(piperazin-1-yl)pyrimidin-5-yl)oxy)pyridine-4-yl)methyl)piperidin-4-yl)methyl)acetamide, 4 hydrochloride (5.39 mg, 7.45 μmol, 5.0% yield): ¹H NMR (400 MHz, CD₃OD) δ ppm 8.45 (s, 2H), 7.91 (s, 1H), 7.87 (d, J=1.8 Hz, 2H), 7.51 (t, J=1.8 Hz, 1H), 7.34-7.27 (m, 1H), 4.43 (s, 2H), 4.15-4.10 (m, 4H), 3.60 (d, J=12.3 Hz, 2H), 3.34 (d, J=5.3 Hz, 4H), 3.14-3.05 (m, 4H), 2.02-1.94 (m, 5H), 1.85 (br. s, 1H), 1.62-1.51 (m, 2H); ES-LCMS m/z 570.3, 572.3 [M+H]⁺.

### Example 368: methyl4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylbutanoate

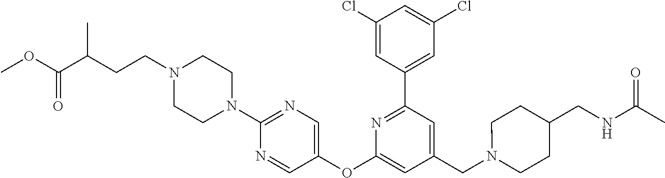 [SMILES: COC(=O)C(C)CCN1CCN(c2ncc(Oc3cc(CN4CCC(CNC(C)=O)CC4)cc(-c4cc(Cl)cc(Cl)c4)n3)cn2)CC1]

To a solution of N-((1-((2-(3,5-dichlorophenyl)-6-((2-(piperazin-1-yl)pyrimidin-5-yl)oxy)pyridin-4-yl)methyl)piperidin-4-yl)methyl)acetamide, 4 hydrochloride (3.3 g, 4.38 mmol) and methyl 4-bromo-2-methylbutanoate (2.85 g, 13.13 mmol) in DMF (50 mL) was added K₂CO₃ (4.84 g, 35.0 mmol). Then the reaction mixture was stirred at 80° C. for 12 h. The solid was filtered off and solvent was removed in vacuo to give the crude product which was purified by chromatography (from pure DCM to DCM/MeOH=10/1, TLC: DCM/MeOH=10/1, R_{f}=0.45) to yield a yellow solid of methyl 4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylbutanoate (2.3 g, 2.88 mmol, 65.9% yield): ¹H NMR (400 MHz, CDCl₃) δ ppm 8.31-8.22 (m, 2H), 7.73-7.64 (m, 2H), 7.43-7.36 (m, 1H), 7.34-7.27 (m, 1H), 6.85 (s, 1H), 5.62 (t, J=5.4 Hz, 1H), 3.80 (t, J=4.7 Hz, 4H), 3.66 (s, 3H), 3.48 (s, 2H), 3.13 (t, J=6.3 Hz, 2H), 2.85 (d, J=11.2 Hz, 2H), 2.60-2.43 (m, 5H), 2.37 (t, J=7.2 Hz, 2H), 2.09-1.89 (m, 7H), 1.66 (d, J=12.1 Hz, 2H), 1.54-1.46 (m, 1H), 1.36-1.25 (m, 2H), 1.19-1.13 (m, 3H); ES-LCMS m/z 684.4, 686.4 [M+H]⁺.

### Example 369: 4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylbutanoic acid

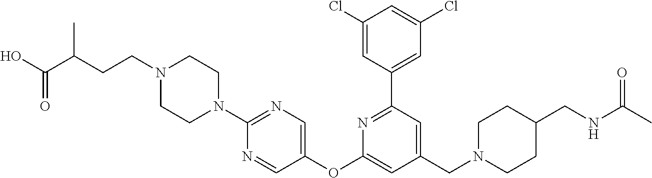 [SMILES: CC(=O)NCC1CCN(Cc2cc(Oc3cnc(N4CCN(CCC(C)C(=O)O)CC4)nc3)nc(-c3cc(Cl)cc(Cl)c3)c2)CC1]

To a solution of methyl4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylbutanoate (1.7 g, 2.130 mmol) in MeOH (20 mL) and H₂O (3 mL) was added LiOH (0.153 g, 6.39 mmol). Then the reaction mixture was stirred at 25° C. for 0.5 h. The solvent was removed in vacuo to give the crude product which was dissolved with MeCN (10 mL) and H₂O (10 mL). 1 N HCl was added to adjust pH=6-7. The mixture was purified by preparative HPLC (MeCN/H₂O as eluents, acidic condition) and lyophilized to yield 4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylbutanoic acid, 4 hydrochloride (1186.17 mg, 1.447 mmol, 67.9% yield) as a pale yellow solid: ¹H NMR (400 MHz, CD₃OD) δ ppm 8.63 (s, 2H), 8.05 (s, 1H), 7.88 (d, J=1.8 Hz, 2H), 7.47 (s, 1H), 7.44-7.38 (m, 1H), 4.88 (d, J=14.6 Hz, 2H), 4.47 (s, 2H), 3.75 (d, J=12.1 Hz, 2H), 3.64-3.51 (m, 4H), 3.43-3.29 (m, 2H), 3.27-3.07 (m, 6H), 2.66-2.51 (m, 1H), 2.22-2.08 (m, 4H), 2.01-1.88 (m, 4H), 1.77-1.61 (m, 2H), 1.29-1.20 (m, 3H); ES-LCMS m/z 670.4, 672.4 [M+H]⁺.

### Intermediate 2. N-((1-((2-((6-Bromopyridin-3-yl)oxy)-6-(3,5-dichlorophenyl)pyridin-4-yl)methyl)piperidin-4-yl)methyl)acetamide

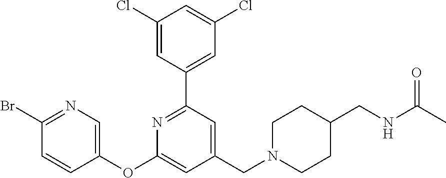 [SMILES: CC(=O)NCC1CCN(Cc2cc(Oc3ccc(Br)nc3)nc(-c3cc(Cl)cc(Cl)c3)c2)CC1]

Intermediate 2 was synthesized according to the following the steps.

### Step 1: Methyl 2-(benzyloxy)-6-chloroisonicotinate

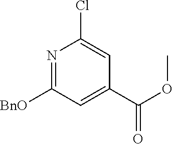 [SMILES: COC(=O)c1cc(Cl)nc(OCc2ccccc2)c1]

To a solution of phenylmethanol (15.75 g, 146 mmol) in DMF (500 mL) was added NaH (7.57 g, 189 mmol) at 25° C. After the mixture was stirred at 25° C. for 0.5 h, methyl 2,6-dichloroisonicotinate (30 g, 146 mmol) in DMF (100 mL) was added and the mixture was stirred at 25° C. for 12 h. The mixture was filtered and the filtrate was concentrated to yield a residue which was purified by column chromatography to yield a colorless oil of methyl 2-(benzyloxy)-6-chloroisonicotinate (16 g, 57.6 mmol, 39.6% yield): ¹H NMR (400 MHz, CDCl₃) δ ppm 7.43-7.36 (m, 3H), 7.36-7.26 (m, 4H), 5.32 (s, 2H), 3.87 (s, 3H); ES-LCMS m/z 278.1 [M+H]⁺.

### Step 2: Methyl 2-(benzyloxy)-6-(3, 5-dichlorophenyl)isonicotinate

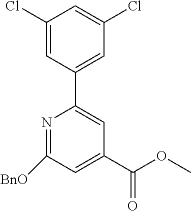 [SMILES: COC(=O)c1cc(OCc2ccccc2)nc(-c2cc(Cl)cc(Cl)c2)c1]

A mixture of methyl 2-(benzyloxy)-6-chloroisonicotinate (12 g, 43.2 mmol), (3,5-dichlorophenyl)boronic acid (12.37 g, 64.8 mmol), PdCl₂(dppf) (6.32 g, 8.64 mmol) and K₂CO₃ (11.94 g, 86 mmol) in 1,4-dioxane (200 mL) was stirred at 80° C. for 12 h under N₂ atmosphere. The mixture was filtered and the filtrate was concentrated. The residue was purified by column chromatography to yield a colorless oil of methyl 2-(benzyloxy)-6-(3,5-dichlorophenyl)isonicotinate (13 g, 33.5 mmol, 77.0% yield): ¹H NMR (400 MHz, CDCl₃) δ ppm 7.96-7.92 (m, 1H), 7.91-7.87 (m, 2H), 7.85-7.80 (m, 2H), 7.42-7.38 (m, 5H), 5.50 (s, 2H), 3.92 (d, J=2.0 Hz, 3H); ES-LCMS m/z 388.0, 389.9 [M+H]⁺.

### Step 3: (2-(Benzyloxy)-6-(3, 5-dichlorophenyl)pyridin-4-yl)methanol

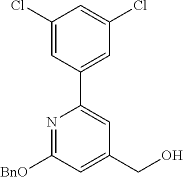 [SMILES: OCc1cc(OCc2ccccc2)nc(-c2cc(Cl)cc(Cl)c2)c1]

To a solution of methyl 2-(benzyloxy)-6-(3,5-dichlorophenyl)isonicotinate (12 g, 30.9 mmol) in THF (300 mL) was added LiAlH₄ (2.35 g, 61.8 mmol) at −78° C. The mixture was allowed to warm up to 25° C. for 12 h. The reaction was quenched by addition of aqueous NaOH (20%, 10 mL) at 0° C. then was filtered and concentrated. The residue was purified by column chromatography to yield a yellow oil of (2-(benzyloxy)-6-(3,5-dichlorophenyl)pyridin-4-yl)methanol (10.7 g, 29.7 mmol, 96.0% yield): ¹H NMR (400 MHz, CDCl₃) δ ppm 7.83-7.78 (m, 1H), 7.46-7.41 (m, 1H), 7.40-7.36 (m, 1H), 7.30 (d, J=4.0 Hz, 4H), 7.23 (s, 3H), 5.29 (s, 2H), 4.68 (d, J=4.9 Hz, 2H); ES-LCMS m/z 359.9, 362.0 [M+H]⁺.

### Step 4: (2-(Benzyloxy)-6-(3, 5-dichlorophenyl)pyridin-4-yl)methyl methanesulfonate

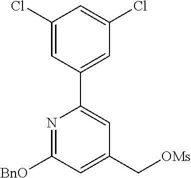 [SMILES: CS(=O)(=O)OCc1cc(OCc2ccccc2)nc(-c2cc(Cl)cc(Cl)c2)c1]

To a solution of (2-(benzyloxy)-6-(3,5-dichlorophenyl)pyridin-4-yl)methanol (3.0 g, 8.33 mmol) and DIEA (2.91 mL, 16.66 mmol) in DCM (40 mL) was added MsCl (0.779 mL, 9.99 mmol) at 0° C. The mixture was stirred at 25° C. for 3 h. DCM (100 mL) was added, washed with water (30 mL×3) and dried over Na₂SO₄. The organic phase was concentrated to yield a yellow oil of (2-(benzyloxy)-6-(3,5-dichlorophenyl)pyridin-4-yl)methyl methanesulfonate (3.0 g, 6.84 mmol, 82.0% yield): ¹H NMR (400 MHz, CDCl₃) δ ppm 7.80-7.78 (m, 2H), 7.43-7.41 (m, 2H), 7.40-7.36 (m, 1H), 7.36-7.31 (m, 5H), 5.42 (s, 2H), 5.16 (s, 2H), 3.01 (s, 3H); ES-LCMS m/z 437.9, 439.9 [M+H]⁺.

### Step 5: N-((1-((2-(benzyloxy)-6-(3,5-dichlorophenyl)pyridin-4-yl)methyl)piperidin-4-yl)methyl)acetamide

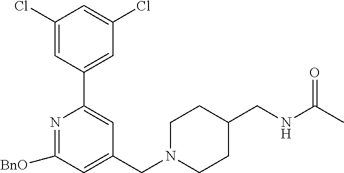 [SMILES: CC(=O)NCC1CCN(Cc2cc(OCc3ccccc3)nc(-c3cc(Cl)cc(Cl)c3)c2)CC1]

To a solution of (2-(benzyloxy)-6-(3,5-dichlorophenyl)pyridin-4-yl)methyl methanesulfonate (3.0 g, 6.84 mmol) and K₂CO₃ (1.892 g, 13.69 mmol) in DMF (30 mL) was added N-(piperidin-4-ylmethyl)acetamide (1.069 g, 6.84 mmol). The mixture was stirred at 80° C. for 12 h. After cooling to room temperature, the mixture was filtered and the filtrate was concentrated. The residue was purified by column chromatography to yield a yellow oil of N-((1-((2-(benzyloxy)-6-(3,5-dichlorophenyl)pyridin-4-yl)methyl)piperidin-4-yl)methyl)acetamide (3.0 g, 6.02 mmol, 88.0% yield): ¹H NMR (400 MHz, CDCl₃) δ ppm 7.93-7.84 (m, 2H), 7.53-7.47 (m, 2H), 7.46-7.42 (m, 1H), 7.41-7.34 (m, 4H), 7.34-7.30 (m, 1H), 5.47-5.44 (m, 2H), 3.54-3.50 (m, 2H), 3.18-3.11 (m, 2H), 2.91-2.77 (m, 4H), 2.11-2.07 (m, 3H), 1.71-1.67 (m, 2H), 1.55-1.49 (m, 1H), 1.32-1.23 (m, 2H); ES-LCMS m/z 498.1, 500.1 [M+H]⁺.

### Step 6: N-((1-((2-(3,5-dichlorophenyl)-6-hydroxypyridin-4-yl)methyl)piperidin-4-yl)methyl)acetamide

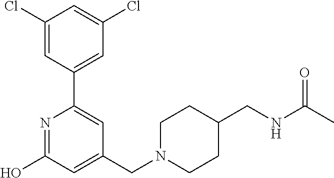 [SMILES: CC(=O)NCC1CCN(Cc2cc(O)nc(-c3cc(Cl)cc(Cl)c3)c2)CC1]

To a solution of N-((1-((2-(benzyloxy)-6-(3,5-dichlorophenyl)pyridin-4-yl)methyl)piperidin-4-yl)methyl)acetamide (2.0 g, 4.01 mmol) in THF (20 mL) was added concentrated HCl (15 mL, 180 mmol). The mixture was stirred at 80° C. for 4 h then concentrated. The residue was purified by column chromatography to yield a brown solid of N-((1-((2-(3,5-dichlorophenyl)-6-hydroxypyridin-4-yl)methyl)piperidin-4-yl)methyl)acetamide (1.0 g, 2.449 mmol, 61.0% yield): ¹H NMR (400 MHz, CD₃OD) δ ppm 7.74-7.66 (m, 2H), 7.61-7.54 (m, 1H), 6.81-6.72 (m, 1H), 6.57-6.48 (m, 1H), 3.44 (s, 2H), 3.09-3.02 (m, 2H), 2.95-2.86 (m, 2H), 2.12-2.00 (m, 2H), 1.92 (s, 3H), 1.76-1.65 (m, 2H), 1.60-1.45 (m, 1H), 1.38-1.21 (m, 2H); ES-LCMS m/z 408.2, 410.1 [M+H]⁺.

### Step 7: N-((1-((2-((6-Bromopyridin-3-yl)oxy)-6-(3,5-dichlorophenyl)pyridin-4-yl)methyl)piperidin-4-yl)methyl)acetamide

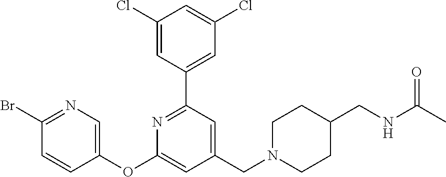 [SMILES: CC(=O)NCC1CCN(Cc2cc(Oc3ccc(Br)nc3)nc(-c3cc(Cl)cc(Cl)c3)c2)CC1]

A mixture of N-((1-((2-(3,5-dichlorophenyl)-6-hydroxypyridin-4-yl)methyl)piperidin-4-yl)methyl)acetamide (1 g, 2.449 mmol), 2-bromo-5-fluoropyridine (0.646 g, 3.67 mmol) and Cs₂CO₃ (3.99 g, 12.25 mmol) in NMP (15 mL) was stirred at 130° C. for 16 h. The mixture was cooled to room temperature and filtered. The filtrate was concentrated and the residue was purified twice by silica gel column chromatography (MeOH/DCM=1/10). All fractions found to contain product by TLC (MeOH/DCM=1/10) were combined and concentrated to yield a brown solid of N-((1-((2-((6-bromopyridin-3-yl)oxy)-6-(3,5-dichlorophenyl)pyridin-4-yl)methyl)piperidin-4yl)methyl)acetamide (580 mg, 0.504 mmol, 20.6% yield): ¹H NMR (400 MHz, CDCl₃) δ ppm 8.36-8.30 (m, 1H), 7.73 (d, J=1.7 Hz, 1H), 7.68 (d, J=1.7 Hz, 1H), 7.54-7.51 (m, 1H), 7.50-7.47 (m, 1H), 7.45 (s, 1H), 7.35-7.31 (m, 1H), 7.01-6.93 (m, 1H), 3.53 (s, 2H), 3.18-3.14 (m, 2H), 2.88 (d, J=10.8 Hz, 2H), 2.00-1.96 (m, 5H), 1.73-1.60 (m, 2H), 1.53 (d, J=4.2 Hz, 1H), 1.32 (d, J=7.6 Hz, 2H); ES-LCMS m/z 563.0, 564.9 [M+H]⁺.

### Example 9: N-((1-((2-(3,5-Dichlorophenyl)-6-((6-(piperazin-1-yl)pyridin-3-yl)oxy)pyridin-4-yl)methyl)piperidin-4-yl)methyl)acetamide, 4 hydrochloride

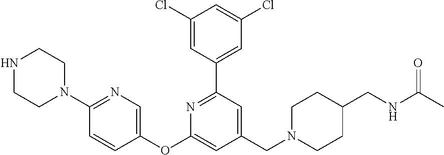 [SMILES: CC(=O)NCC1CCN(Cc2cc(Oc3ccc(N4CCNCC4)nc3)nc(-c3cc(Cl)cc(Cl)c3)c2)CC1]

Example 9 was synthesized according to the following steps.

### Step 1: tert-Butyl 4-(5-((4-((4-(acetamidomethyl)piperidin-1-yl)methyl)-6-(3,5-dichlorophenyl)pyridin-2-yl)oxy)pyridin-2-yl)piperazine-1-carboxylate

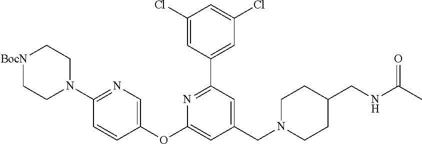 [SMILES: CC(=O)NCC1CCN(Cc2cc(Oc3ccc(N4CCN(C(=O)OC(C)(C)C)CC4)nc3)nc(-c3cc(Cl)cc(Cl)c3)c2)CC1]

A mixture of N-((1-((2-((6-bromopyridin-3-yl)oxy)-6-(3,5-dichlorophenyl)pyridin-4-yl)methyl)piperidin-4-yl)methyl)acetamide (580 mg, 1.028 mmol), tert-butyl piperazine-1-carboxylate (574 mg, 3.08 mmol), (±)-BINAP (12.80 mg, 0.021 mmol), 18-crown-6 (815 mg, 3.08 mmol), Pd₂(dba)₃ (47.1 mg, 0.051 mmol) and sodium tert-butoxide (296 mg, 3.08 mmol) in THF (15 mL) was stirred at 65° C. under N₂ atmosphere for 2 h. The mixture was filtered and the filtrate was concentrated. The residue was dissolved in DCM (50 mL) and washed with brine (50 mL), dried over MgSO₄, filtered and concentrated. The crude material was purified by silica gel column chromatography (MeOH/DCM=1/10) then further purified by preparative HPLC(MeCN/H₂O as eluents, acidic condition) to yield a pale yellow solid of tert-butyl 4-(5-((4-((4-(acetamidomethyl)piperidin-1-yl)methyl)-6-(3,5-dichlorophenyl)pyridin-2-yl)oxy)pyridin-2-yl)piperazine-1-carboxylate (81 mg, 0.094 mmol, 9.1% yield): ¹H NMR (400 MHz, CD₃OD) δ ppm 8.24-8.17 (m, 1H), 8.00 (d, J=6.8 Hz, 1H), 7.94 (s, 1H), 7.91-7.86 (m, 2H), 7.54 (t, J=1.9 Hz, 1H), 7.44-7.39 (m, 1H), 7.36 (s, 1H), 4.49-4.43 (m, 2H), 4.01-3.94 (m, 4H), 3.62 (d, J=12.3 Hz, 2H), 3.50-3.44 (m, 4H), 3.18-3.07 (m, 4H), 2.06-1.96 (m, 6H), 1.87 (m, 1H), 1.67-1.50 (m, 10H); ES-LCMS m/z 669.3, 671.3 [M+H]⁺.

### Step 2: N-((1-((2-(3,5-Dichlorophenyl)-6-((6-(piperazin-1-yl)pyridin-3-yl)oxy)pyridin-4-yl)methyl)piperidin-4-yl)methyl)acetamide, 4 hydrochloride

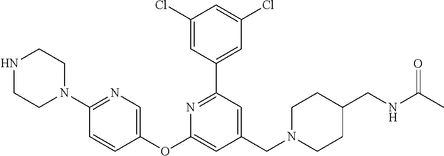 [SMILES: CC(=O)NCC1CCN(Cc2cc(Oc3ccc(N4CCNCC4)nc3)nc(-c3cc(Cl)cc(Cl)c3)c2)CC1]

A mixture of tert-butyl 4-(5-((4-((4-(acetamidomethyl)piperidin-1-yl)methyl)-6-(3,5-dichlorophenyl)pyridin-2-yl)oxy)pyridin-2-yl)piperazine-1-carboxylate (81 mg, 0.121 mmol) and TFA (2 mL, 26.0 mmol) in DCM (8 mL) was stirred at 25° C. for 0.5 h. Then the mixture was concentrated and purified by preparative HPLC (MeCN/H₂O as eluents, acidic condition) and lyophilized to yield a white solid N-((1-((2-(3,5-dichlorophenyl)-6-((6-(piperazin-1-yl)pyridin-3-yl)oxy)pyridin-4-yl)methyl)piperidin-4-yl)methyl)acetamide, 4 hydrochloride (37.43 mg, 0.052 mmol, 43.1% yield): ¹H NMR (400 MHz, CD₃OD) δ ppm 8.19 (d, J=2.4 Hz, 1H), 8.06 (d, J=9.5 Hz, 1H), 7.92 (s, 1H), 7.86 (d, J=1.7 Hz, 2H), 7.51 (s, 1H), 7.45 (d, J=9.8 Hz, 1H), 7.35 (s, 1H), 4.43 (s, 2H), 4.01-3.93 (m, 4H), 3.59 (d, J=12.7 Hz, 2H), 3.49-3.41 (m, 4H), 3.15-3.03 (m, 4H), 2.03-1.91 (m, 5H), 1.83 (br. s, 1H), 1.63-1.50 (m, 2H); ES-LCMS m/z 569.0, 571.0 [M+H]⁺.

### Example 11: 3-(4-(5-((4-((4-(Acetamidomethyl)piperidin-1-yl)methyl)-6-(3,5-dichlorophenyl)pyridin-2-yl)oxy)pyridin-2-yl)piperazin-1-yl)propanoic acid, 4 hydrochloride

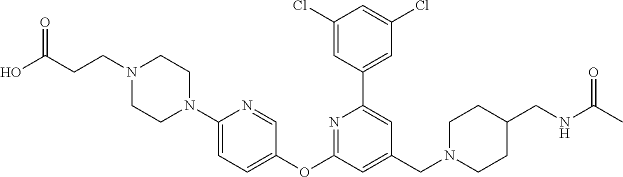 [SMILES: CC(=O)NCC1CCN(Cc2cc(Oc3ccc(N4CCN(CCC(=O)O)CC4)nc3)nc(-c3cc(Cl)cc(Cl)c3)c2)CC1]

Example 11 was synthesized according to the following steps.

### Step 1: Ethyl 3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanoate

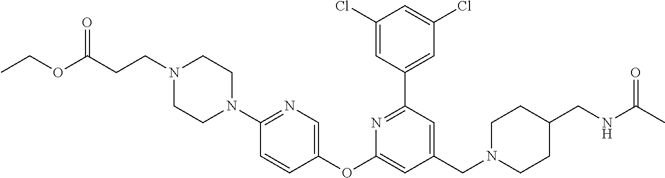 [SMILES: CCOC(=O)CCN1CCN(c2ccc(Oc3cc(CN4CCC(CNC(C)=O)CC4)cc(-c4cc(Cl)cc(Cl)c4)n3)cn2)CC1]

To a solution of N-((1-((2-(3,5-dichlorophenyl)-6-((6-(piperazin-1-yl)pyridin-3-yl)oxy)pyridin-4-yl)methyl)piperidin-4-yl)methyl)acetamide (20 g, 33.4 mmol) and ethyl 3-bromopropanoate (18.12 g, 100 mmol) in DMF (350 mL) was added K₂CO₃ (13.83 g, 100 mmol). Then the reaction mixture was stirred at 80° C. for 12 h. The solid was filtered off and solution was concentrated to yield a pale yellow solid of ethyl 3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanoate (17.6 g, 24.97 mmol, 74.8% yield): ¹H NMR (400 MHz, CDCl₃) δ ppm 8.10 (d, J=3.1 Hz, 1H), 7.74 (d, J=1.8 Hz, 2H), 7.43-7.35 (m, 2H), 7.31 (s, 1H), 6.78 (s, 1H), 6.70 (d, J=9.3 Hz, 1H), 5.55 (br s, 1H), 4.14 (q, J=7.1 Hz, 2H), 3.58-3.50 (m, 4H), 3.47 (s, 2H), 3.14 (t, J=6.4 Hz, 2H), 2.88-2.82 (m, 2H), 2.77-2.70 (m, 2H), 2.64-2.56 (m, 4H), 2.55-2.49 (m, 2H), 2.04-1.94 (m, 5H), 1.66 (d, J=12.8 Hz, 2H), 1.54-1.46 (m, 1H), 1.35-1.21 (m, 5H); ES-LCMS m/z 669.3, 671.3 [M+H]⁺.

### Step 2: 3-(4-(5-((4-((4-(Acetamidomethyl)piperidin-1-yl)methyl)-6-(3,5-dichlorophenyl)pyridin-2-yl)oxy)pyridin-2-yl)piperazin-1-yl)propanoic acid, 4 hydrochloride

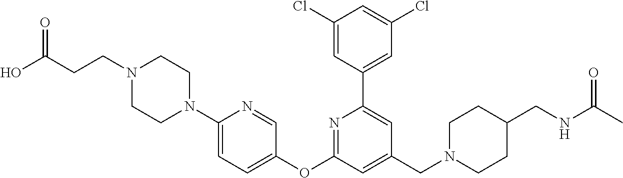 [SMILES: CC(=O)NCC1CCN(Cc2cc(Oc3ccc(N4CCN(CCC(=O)O)CC4)nc3)nc(-c3cc(Cl)cc(Cl)c3)c2)CC1]

To a solution of ethyl 3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanoate (17.6 g, 24.97 mmol) in THF (200 mL) was added LiOH.H₂O (2.096 g, 49.9 mmol) and water (2 mL). Then the reaction mixture was stirred at 25° C. for 12 h. 1 N HCl was added to adjust pH to 6 then concentrated to yield the crude product, which was washed with EA/MeOH=10/1 (500 mL) and THF (500 mL). The solid was collected to yield the crude product which was purified by preparative HPLC (MeCN/H₂O as eluents, acidic condition). Concentrated HCl was added to the combined purified fractions to adjust to pH to 2, and lyophilized to yield a pale yellow solid of 3-(4-(5-((4-((4-(acetamidomethyl)piperidin-1-yl)methyl)-6-(3,5-dichlorophenyl)pyridin-2-yl)oxy)pyridine-2-yl)piperazin-1-yl)propanoic acid, 4 hydrochloride (15 g, 23.09 mmol, 92.0% yield): ¹H NMR (400 MHz, CD₃OD) δ ppm 8.23 (s, 1H), 8.20-8.16 (m, 1H), 7.99 (s, 1H), 7.89 (s, 2H), 7.55 (d, J=12 Hz, 1H), 7.51 (s, 1H), 7.40 (s, 1H), 4.46 (s, 2H), 3.80-3.40 (m, 10H), 3.30-3.20 (m, 2H), 3.15-3.07 (m, 4H), 2.96-2.94 (m, 2H), 2.00-1.92 (m, 5H), 1.90-1.79 (m, 1H), 1.62-1.53 (m, 2H); ES-LCMS m/z 641.3, 643.2 [M+H]⁺.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of treating a viral infection in a subject in need thereof comprising administering to the subject a compound of Formula (I), or a pharmaceutically acceptable salt thereof, wherein the compound of Formula (I) is of the formula: 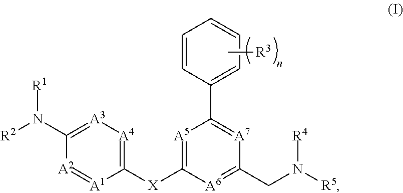
A¹, A², A³, A⁴, A⁵, A⁶, and A⁷ are each independently —N═ or —C(R⁶)═;
X is —O— or —N(R⁸)—;
R¹ and R² are each independently H or optionally substituted (C₁-C₄)alkyl;
optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl;
optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

2. A method of inhibiting the replication of a virus in a subject in a subject in need thereof, comprising administering to the subject an effective amount of a compound of Formula (I), wherein the compound of Formula (I) is of the formula: 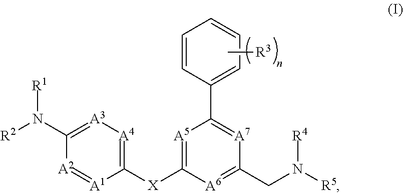
A¹, A², A³, A⁴, A⁵, A⁶, and A⁷ are each independently —N═ or —C(R⁶)═;
X is —O— or —N(R⁸)—;
R¹ and R² are each independently H or optionally substituted (C₁-C₄)alkyl;
optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl;
optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

3. A compound of Formula (I), or a pharmaceutically acceptable salt thereof, for use in treating and/or preventing a viral infection in a subject in need thereof, wherein the compound of Formula (I) is of the formula: 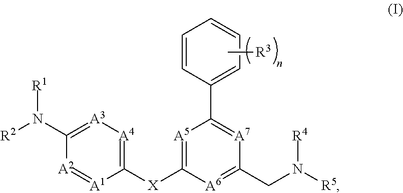
A¹, A², A³, A⁴, A⁵, A⁶, and A⁷ are each independently —N═ or —C(R⁶)═;
X is —O— or —N(R⁸)—;
R¹ and R² are each independently H or optionally substituted (C₁-C₄)alkyl;
optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl;
optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

4. Use of a compound of Formula (I), or a pharmaceutically acceptable salt thereof, in the manufacture of a medicament for use in the treatment and/or prevention of a viral infection in a subject in need thereof, wherein the compound of Formula (I) is of the formula: 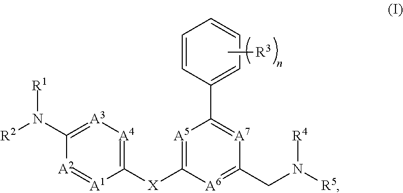
A¹, A², A³, A⁴, A⁵, A⁶, and A⁷ are each independently —N═ or —C(R⁶)═;
X is —O— or —N(R⁸)—;
R¹ and R² are each independently H or optionally substituted (C₁-C₄)alkyl;
optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl;
optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

5. A pharmaceutical composition comprising a compound of Formula (I), for use in the methods or uses of any one of the preceding claims, wherein the compound of Formula (I) is of the formula: 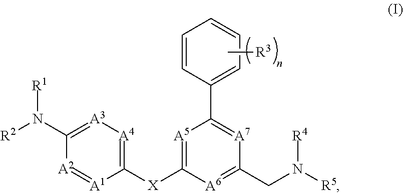
A¹, A², A³, A⁴, A⁵, A⁶, and A⁷ are each independently —N═ or —C(R⁶)═;
X is —O— or —N(R⁸)—;
R¹ and R² are each independently H or optionally substituted (C₁-C₄)alkyl;
optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl;
optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

6. The method or use of any one of claims 1 to 5, wherein the virus is a togaviridae family virus.

7. The method or use of claim 6, wherein the togaviridae family virus is an alphavirus.

8. The method or use of claim 7, wherein the alphavirus is Chikungunya virus, Eastern equine encephalitis virus, Mayaro virus, Onyong-nyong virus, Ross River virus, Semliki Forest virus, Sindbis virus, Venezuelan equine encephalitis virus, or Western equine encephalitis virus.

9. The method or use of any one of claims 6 to 8, wherein the virus is Chikungunya virus.

10. The method or use of any one of claims 1 to 5, wherein the virus is a flavivirus.

11. The method or use of any one of claim 10, wherein the virus is dengue virus.

12. The method or use of any one of claim 10, wherein the virus is Usutu virus.

13. The method or use of any one of claims 1 to 5, wherein the virus is a filoviradae family virus.

14. The method or use of claim 13, wherein the filoviradae family virus is a Marburgvirus.

15. The method or use of claim 14, wherein the virus is Marburg virus.

16. The method or use of any one of claims 1 to 5, wherein the virus is human respiratory syncytial virus.

17. The method or use of any one of claims 1 to 5, wherein the virus is an orthoparamyxovirinae virus.

18. The method or use of any one of claims 1 to 5, or 17 wherein the virus is Measles morbillivirus.

19. The method or use of any one of claims 1 to 5, or 17 wherein the virus is Nipah virus.

20. A method of treating and/or preventing a disorder due to a microbial toxin in a subject in need thereof comprising administering to the subject a therapeutically effective amount of a compound of Formula (I), wherein the compound of Formula (I) is of the formula: 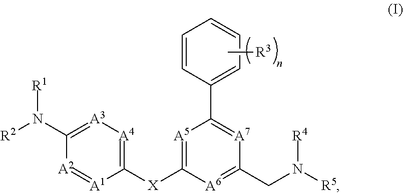
A¹, A², A³, A⁴, A⁵, A⁶, and A⁷ are each independently —N═ or —C(R⁶)═;
X is —O— or —N(R⁸)—;
R¹ and R² are each independently H or optionally substituted (C₁-C₄)alkyl;
optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl;
optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

21. A method of preventing the activation of a toxin in a subject in need thereof comprising administering to the subject a therapeutically effective amount of a compound of Formula (I), wherein the compound of Formula (I) is of the formula: 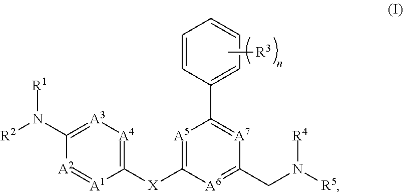
A¹, A², A³, A⁴, A⁵, A⁶, and A⁷ are each independently —N═ or —C(R⁶)═;
X is —O— or —N(R⁸)—;
R¹ and R² are each independently H or optionally substituted (C₁-C₄)alkyl;
optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl;
optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

22. A compound of Formula (I) for use in treating and/or preventing a disorder due to a microbial toxin in a subject in need thereof, wherein the compound of Formula (I) is of the formula: 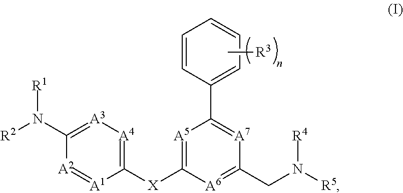
A¹, A², A³, A⁴, A⁵, A⁶, and A⁷ are each independently —N═ or —C(R⁶)═;
X is —O— or —N(R⁸)—;
R¹ and R² are each independently H or optionally substituted (C₁-C₄)alkyl;
optionally, R¹ and R² taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷;
each R³ is independently halogen, —CN, —O(C₁-C₄)alkyl, or optionally substituted (C₁-C₄)alkyl;
R⁴ and R⁵ are each independently H or optionally substituted (C₁-C₄)alkyl;
optionally, R⁴ and R⁵ taken together with the nitrogen atom to which they are attached form a 4-11 membered monocyclic, fused bicyclic, bridged, or spiro-bicyclic saturated ring, optionally containing one or two additional heteroatoms independently selected from oxygen, nitrogen, and sulfur, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹;
each R⁶ is independently H, halogen, optionally substituted (C₁-C₄)alkyl, —OH, or optionally substituted (C₁-C₄)alkoxy;
each R⁷ is independently (C₁-C₆)alkyl, (C₂-C₆)alkenyl, halo(C₁-C₆)alkyl, (C₃-C₆)cycloalkyl, or (C₁-C₄)alkyl(C₃-C₆)cycloalkyl, each of which is optionally substituted with one or two of triazolyl, tetrazolyl, —CO₂R⁸, —CONR⁸R⁹, —CON(R⁸)CO₂(C₁-C₄)alkyl, hydroxyl, oxo, —(C₁-C₄)alkoxy, —OCONR⁸R⁹, —OCON(R⁸)C(O)R⁹, (C₁-C₄)alkyl, (C₁-C₄)alkylOH, —NR⁸R⁹, —N(O)R⁸R⁹, —N(R⁸)C(O)R⁹, —N(R⁸)CO₂(C₁-C₄)alkyl, —N(R⁸)CH₂CO₂R⁹, —N(R⁸)CONR⁸R⁹, —N(R⁸)CON(R⁸)C(O)R⁹, —N(R⁸)CON(R⁸)CO₂(C₁-C₄)alkyl, —N(R⁸)SO₂R⁹, —N(R⁸)CON(R⁸)SO₂R⁹, —SO(C₁-C₄)alkyl, —SO₂(C₁-C₄)alkyl, —SO₃R⁸, —SO₂NR⁸R⁹, —B(OH)₂, —P(O)R⁸R⁹, or —P(O)(OR⁸)(OR⁹);
each of R⁸ and R⁹ is independently H, optionally substituted (C₁-C₄)alkyl, or optionally substituted (C₃-C₆)cycloalkyl; and
n is 1, 2, 3, or 4.

23. The method or use of any one of claims 20 to 22, wherein the toxin is P. aeruginosa toxin A.

24. The method or use of any one of claims 20 to 22 wherein the toxin is Clostridium septicum alpha-toxin.

25. The method or use of any one of claims 20 to 22 wherein the toxin is diphtheria toxin.

26. The method or use of any one of claims 20 to 22 wherein the toxin is a shiga toxin.

27. The method or use of any one of claims 1 to 26, wherein the compound of Formula (I), or pharmaceutically acceptable salt thereof, is of the formula:

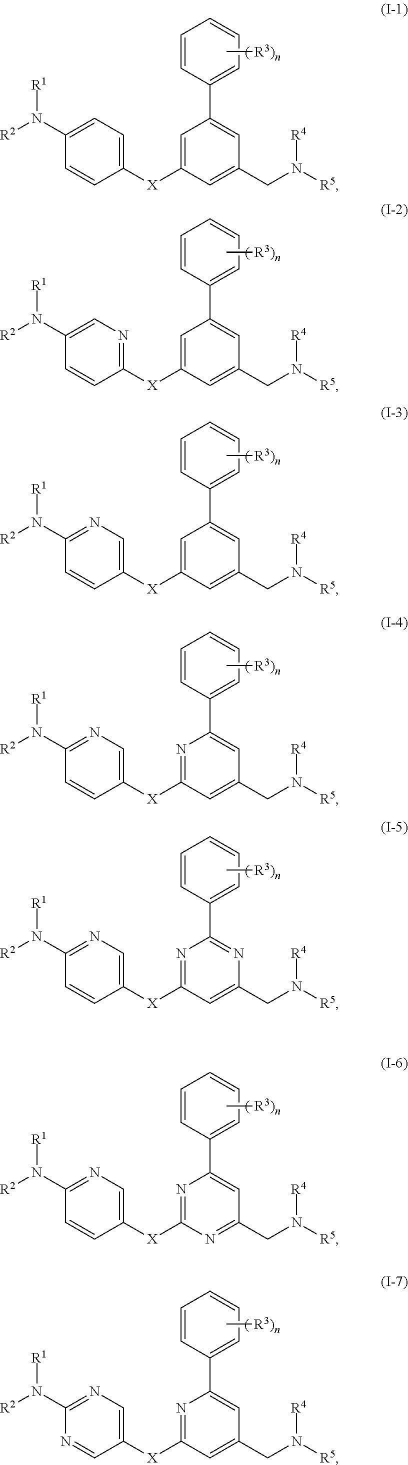

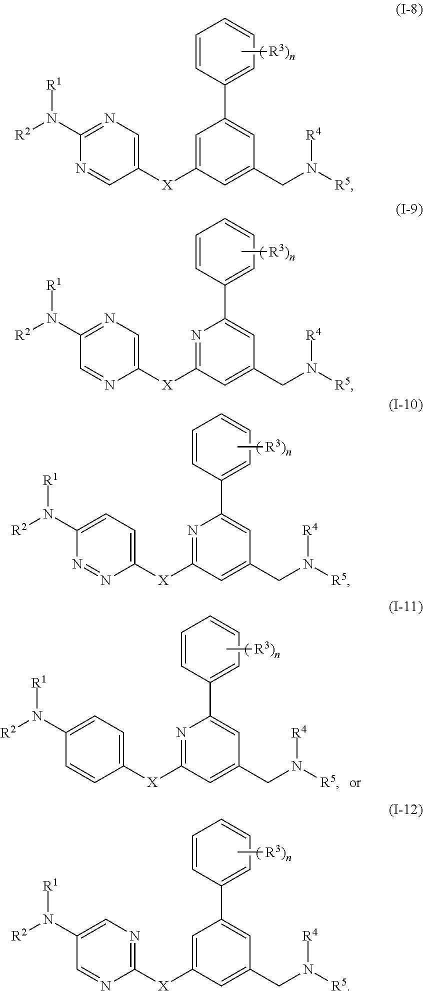

28. The method or use of claim 27, wherein the compound of Formula (I), or pharmaceutically acceptable salt thereof, is of the Formula (I-4):

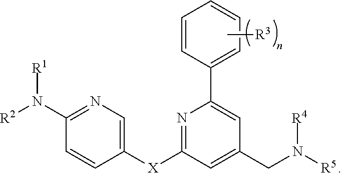

29. The method or use of claim 27, wherein the compound of Formula (I), or pharmaceutically acceptable salt thereof, is of the Formula (I-7):

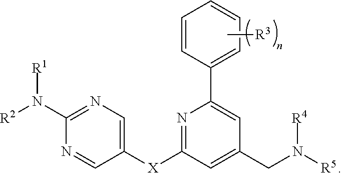

30. The method or use of any one of claims 1 to 29, wherein R³ is halogen.

31. The method or use of any one of claims 1 to 30, wherein R³ is —Cl.

32. The method or use of any one of claims 1 to 31, wherein n is 2.

33. The method or use of any one of claims 1 to 32, wherein X is —O—.

34. The method or use of any one of claims 1 to 31, wherein R¹ and R² taken together with the nitrogen atom to which they are attached form a 4-11 membered heterocyclic ring, wherein said ring is optionally substituted with one, two, or three of halogen, hydroxyl, oxo, —OCONR⁸R⁹, —CO₂R⁸, —C(O)CO₂R⁸, —R⁷, —OR⁷, —NHR⁸, —NR⁷R⁸, —C(O)R⁷, —CONHR⁸, —CONR⁷R⁸, or —SO₂R⁷.

35. The method or use of any one of claims 1 to 33, wherein R¹ and R² taken together with the nitrogen atom to which they are attached form an optionally substituted pyrrolidine, pyrazolidine, imidazolidine, piperidine, piperazine, or morpholine ring.

36. The method or use of claim 35, wherein R¹ and R² taken together with the nitrogen atom to which they are attached form a piperazine ring of the formula:

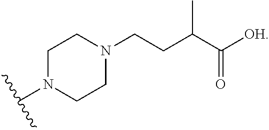 [SMILES: *N1CCN(CCC(C)C(=O)O)CC1]

37. The method or use of claim 35, wherein R¹ and R² taken together with the nitrogen atom to which they are attached form a piperazine ring of the formula:

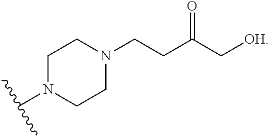 [SMILES: *N1CCN(CCC(=O)CO)CC1]

38. The method or use of any one of claims 1 to 37, wherein R⁴ and R⁵ taken together with the nitrogen atom to which they are attached form a 4-11 membered heterocyclic ring, wherein said ring is optionally substituted with one, two, or three of halogen, hydroxyl, oxo, —OCONR⁸R⁹, —CO₂R⁸, —C(O)CO₂R⁸, —SO₂(C₁ C₄)alkyl, —R⁷, —OR⁷, —NHR⁸, —NR⁷R⁸, —N(R⁸)C(O)R⁹, —N(R⁸)SO₂R⁹, —N(R⁸)CONR⁸R⁹, —N(R⁸)CON(R⁸)SO₂R⁹, —C(O)R⁷, —CONHR⁸, —CONR⁷R⁸, or —P(O)R⁸R⁹.

39. The method or use of any one of claims 1 to 38, wherein R⁴ and R⁵ taken together with the nitrogen atom to which they are attached form an optionally substituted pyrrolidine, pyrazolidine, imidazolidine, piperidine, piperazine, or morpholine ring.

40. The method or use of any one of claims 1 to 39, wherein R⁴ and R⁵ taken together with the nitrogen atom to which they are attached form a heterocyclic ring of the formula:

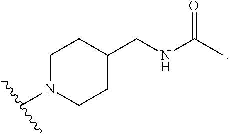 [SMILES: *N1CCC(CNC(C)=O)CC1]

41. The method or use of any one of claims 1 to 40, wherein the compound, or pharmaceutically acceptable salt thereof, is:
2-(4-((2-(3,5-dichlorophenyl)-6-((6-(piperazin-1-yl)pyridin-3-yl)oxy)pyridin-4-yl)methyl)piperazin-1-yl)-N-methylacetamide;
N-((1-((2-(3,5-dichlorophenyl)-6-((6-(piperazin-1-yl)pyridin-3-yl)oxy)pyridin-4-yl)methyl)-4-hydroxypiperidin-4-yl)methyl)acetamide;
3-(1-((2-(3,5-dichlorophenyl)-6-((6-(piperazin-1-yl)pyridin-3-yl)oxy)pyridin-4-yl)methyl)piperidin-4-yl)propanoic acid;
2-(1-((2-(3,5-dichlorophenyl)-6-((6-(piperazin-1-yl)pyridin-3-yl)oxy)pyridin-4-yl)methyl)piperidin-4-yl)acetic acid;
1-((1-((2-(3,5-dichlorophenyl)-6-((6-(piperazin-1-yl)pyridin-3-yl)oxy)pyridin-4-yl)methyl)-4-hydroxypiperidin-4-yl)methyl)-3-methylurea;
methyl ((1-((2-(3,5-dichlorophenyl)-6-((6-(piperazin-1-yl)pyridin-3-yl)oxy)pyridin-4-yl)methyl)-4-hydroxypiperidin-4-yl)methyl)carbamate;
2-(1-((2-(3,5-dichlorophenyl)-6-((6-(piperazin-1-yl)pyridin-3-yl)oxy)pyridin-4-yl)methyl)piperidin-4-yl)ethanesulfonic acid;
(1-((2-(3,5-dichlorophenyl)-6-((6-(piperazin-1-yl)pyridin-3-yl)oxy)pyridin-4-yl)methyl)piperidin-4-yl)methanesulfonic acid;
N-((1-((2-(3,5-dichlorophenyl)-6-((6-(piperazin-1-yl)pyridin-3-yl)oxy)pyridin-4-yl)methyl)piperidin-4-yl)methyl)acetamide;
2-(4-(5-((4-((4-(acetamidomethyl)piperidin-1-yl)methyl)-6-(3,5-dichlorophenyl)pyridin-2-yl)oxy)pyridin-2-yl)piperazin-1-yl)acetic acid;
3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanoic acid;
N-((1-((2-(3,5-dichlorophenyl)-6-((6-(4-(2-(methylsulfonyl)ethyl)piperazin-1-yl)pyridin-3-yl)oxy)pyridin-4-yl)methyl)piperidin-4-yl)methyl)acetamide;
3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanamide;
N-((1-((2-((6-(4-(2-(1H-tetrazol-5-yl)ethyl)piperazin-1-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-(4-(2-(methylsulfinyl)ethyl)piperazin-1-yl)pyridin-3-yl)oxy)pyridin-4-yl)methyl)piperidin-4-yl)methyl)acetamide;
N-((1-((2-(3,5-dichlorophenyl)-6-((6-(4-(4-(methylsulfonyl)butan-2-yl)piperazin-1-yl)pyridin-3-yl)oxy)pyridin-4-yl)methyl)piperidin-4-yl)methyl)acetamide;
1-((1-((2-(3,5-dichlorophenyl)-6-((2-(4-(3-(methylsulfinyl)butyl)piperazin-1-yl)pyrimidin-5-yl)oxy)pyridin-4-yl)methyl)piperidin-4-yl)methyl)-3-methylurea;
1-((1-((2-(3,5-dichlorophenyl)-6-((2-(4-(4-(methylsulfonyl)butan-2-yl)piperazin-1-yl)pyrimidin-5-yl)oxy)pyridin-4-yl)methyl)piperidin-4-yl)methyl)-3-methylurea;
N-((1-((2-(3,5-dichlorophenyl)-6-((2-(4-(4-(methylsulfonyl)butan-2-yl)piperazin-1-yl)pyrimidin-5-yl)oxy)pyridin-4-yl)methyl)piperidin-4-yl)methyl)acetamide;
1-((1-((2-(3,5-dichlorophenyl)-6-((2-(4-(4-hydroxybutan-2-yl)piperazin-1-yl)pyrimidin-5-yl)oxy)pyridin-4-yl)methyl)piperidin-4-yl)methyl)-3-methylurea;
2-(1-((2-(3,5-dichlorophenyl)-6-((2-(4-(1-hydroxypropan-2-yl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-hydroxycyclobutyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(1,3-dihydroxypropan-2-yl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-((1s,3s)-3-hydroxy-3-methylcyclobutyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-((1r,3r)-3-hydroxy-3-methylcyclobutyl)piperazin-1-yl)pyrimidin-5-yl)oxy)pyridin-4-yl)methyl)piperidin-4-yl)acetic acid;
N-((1-((2-(3,5-dichlorophenyl)-6-((6-(4-((trans)-3-(methylsulfonamido)cyclobutyl)piperazin-1-yl)pyridin-3-yl)oxy)pyridin-4-yl)methyl)piperidin-4-yl)methyl)acetamide;
N-((1-((2-(3,5-dichlorophenyl)-6-((6-(4-((cis)-3-(methylsulfonamido)cyclobutyl)piperazin-1-yl)pyridin-3-yl)oxy)pyridin-4-yl)methyl)piperidin-4-yl)methyl)acetamide;
N-((1-((2-((6-(4-(2-aminoethyl)piperazin-1-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-(4-(2,4-dihydroxybutyl)piperazin-1-yl)pyridin-3-yl)oxy)pyridin-4-yl)methyl)piperidin-4-yl)methyl)acetamide;
(2-(4-(5-((4-((4-(acetamidomethyl)piperidin-1-yl)methyl)-6-(3,5-dichlorophenyl)pyridin-2-yl)oxy)pyridin-2-yl)piperazin-1-yl)ethyl)phosphonic acid;
2-(4-(5-((4-((4-(acetamidomethyl)piperidin-1-yl)methyl)-6-(3,5-dichlorophenyl)pyridin-2-yl)oxy)pyridin-2-yl)piperazin-1-yl)ethyl carbamate;
N-((1-((2-(3,5-dichlorophenyl)-6-((6-(4-(2-(N-methylmethylsulfonamido)ethyl)piperazin-1-yl)pyridin-3-yl)oxy)pyridin-4-yl)methyl)piperidin-4-yl)methyl)acetamide;
4-(4-(5-((4-((4-(acetamidomethyl)piperidin-1-yl)methyl)-6-(3,5-dichlorophenyl)pyridin-2-yl)oxy)pyridin-2-yl)piperazin-1-yl)-4-oxobutanoic acid;
N-((1-((2-(3,5-dichlorophenyl)-6-((6-(4-((1-(hydroxymethyl)cyclopropyl)methyl)piperazin-1-yl)pyridin-3-yl)oxy)pyridin-4-yl)methyl)piperidin-4-yl)methyl)acetamide;
N-((1-((2-(3,5-dichlorophenyl)-6-((6-(4-((1-hydroxycyclopropyl)methyl)piperazin-1-yl)pyridin-3-yl)oxy)pyridin-4-yl)methyl)piperidin-4-yl)methyl)acetamide;
2-(1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)-N-ethylacetamide;
1-(2-(3,5-dichlorophenyl)-6-((2-(4-(3-(methylsulfonyl)propyl)piperazin-1-yl)pyrimidin-5-yl)oxy)pyridin-4-yl)-N-methylmethanamine;
3-(4-(5-((6-(3,5-dichlorophenyl)-4-((4-(sulfamoylmethyl)piperidin-1-yl)methyl)pyridin-2-yl)oxy)pyridin-2-yl)piperazin-1-yl)propanoic acid;
3-(4-(5-((6-(3,5-dichlorophenyl)-4-((4-((methylsulfonyl)methyl)piperidin-1-yl)methyl)pyridin-2-yl)oxy)pyridin-2-yl)piperazin-1-yl)propanoic acid;
1-((1-((2-(3,5-dichlorophenyl)-6-((6-(4-(2-hydroxyethyl)piperazin-1-yl)pyridin-3-yl)oxy)pyridin-4-yl)methyl)piperidin-4-yl)methyl)-3-methylurea;
3-(4-(5-((6-(3,5-dichlorophenyl)-4-((4-((3-methylureido)methyl)piperidin-1-yl)methyl)pyridin-2-yl)oxy)pyridin-2-yl)piperazin-1-yl)propanoic acid;
N-((1-((5-(4-aminophenoxy)-3′,5′-dichloro-[1,1′-biphenyl]-3-yl)methyl)piperidin-4-yl)methyl)acetamide;
N-((1-((5-((5-aminopyrimidin-2-yl)oxy)-3′,5′-dichloro-[1,1′-biphenyl]-3-yl)methyl)piperidin-4-yl)methyl)acetamide;
3-(4-(5-((6-(3,5-dichlorophenyl)-4-((4-(methylsulfonamidomethyl)piperidin-1-yl)methyl)pyridin-2-yl)oxy)pyridin-2-yl)piperazin-1-yl)propanoic acid;
N-((1-((5-((5-aminopyridin-2-yl)oxy)-3′,5′-dichloro-[1,1′-biphenyl]-3-yl)methyl)piperidin-4-yl)methyl)acetamide;
N-((1-((5-((6-amino-5-fluoropyridin-3-yl)oxy)-3′,5′-dichloro-[1,1′-biphenyl]-3-yl)methyl)piperidin-4-yl)methyl)acetamide;
1-((1-((2-(3,5-dichlorophenyl)-6-((2-(4-(2-hydroxyethyl)piperazin-1-yl)pyrimidin-5-yl)oxy)pyridin-4-yl)methyl)piperidin-4-yl)methyl)-3-methylurea;
3-(4-(5-((6-(3,5-dichlorophenyl)-4-((4-((3-methylureido)methyl)piperidin-1-yl)methyl)pyridin-2-yl)oxy)pyrimidin-2-yl)piperazin-1-yl)propane-1-sulfonamide;
methyl ((1-((2-(3,5-dichlorophenyl)-6-((2-(piperazin-1-yl)pyrimidin-5-yl)oxy)pyridin-4-yl)methyl)piperidin-4-yl)methyl)carbamate;
3-(4-(5-((6-(3,5-dichlorophenyl)-4-((methylamino)methyl)pyridin-2-yl)oxy)pyridin-2-yl)piperazin-1-yl)propanoic acid;
3-(4-(5-((6-(3,5-dichlorophenyl)-4-((4-fluoro-4-(((methoxycarbonyl)amino)methyl)piperidin-1-yl)methyl)pyridin-2-yl)oxy)pyrimidin-2-yl)piperazin-1-yl)propanoic acid;
2-(1-((2-(3,5-dichlorophenyl)-6-((2-(4-methylpiperazin-1-yl)pyrimidin-5-yl)oxy)pyridin-4-yl)methyl)piperidin-4-yl)ethanol;
2-((1-((2-(3,5-dichlorophenyl)-6-((2-(4-methylpiperazin-1-yl)pyrimidin-5-yl)oxy)pyridin-4-yl)methyl)piperidin-4-yl)oxy)acetic acid;
3-(4-(5-((4-((4-(2-(carbamoyloxy)ethyl)piperazin-1-yl)methyl)-6-(3,5-dichlorophenyl)pyridin-2-yl)oxy)pyrimidin-2-yl)piperazin-1-yl)propanoic acid;
3-(4-(5-((4-((4-(2-(carbamoyloxy)ethyl)piperazin-1-yl)methyl)-6-(3,5-dichlorophenyl)pyridin-2-yl)oxy)pyridin-2-yl)piperazin-1-yl)propanoic acid;
3-(3-(5-((6-(3,5-dichlorophenyl)-4-((4-(((methoxycarbonyl)amino)methyl)piperidin-1-yl)methyl)pyridin-2-yl)oxy)pyrimidin-2-yl)-3,8-diazabicyclo[3.2.1]octan-8-yl)propanoic acid;
4-(4-(5-((6-(3,5-dichlorophenyl)-4-((4-(2-hydroxyethyl)piperidin-1-yl)methyl)pyridin-2-yl)oxy)pyrimidin-2-yl)piperazin-1-yl)-2-methylbutanoic acid;
3-(4-(5-((4-((4-(cyclopropanecarboxamidomethyl)piperidin-1-yl)methyl)-6-(3,5-dichlorophenyl)pyridin-2-yl)oxy)pyridin-2-yl)piperazin-1-yl)propanoic acid;
4-(4-(5-((6-(3,5-dichlorophenyl)-4-((4-(propionamidomethyl)piperidin-1-yl)methyl)pyridin-2-yl)oxy)pyrimidin-2-yl)piperazin-1-yl)-2-methylbutanoic acid;
4-(4-(5-((6-(3,5-dichlorophenyl)-4-((4-fluoropiperidin-1-yl)methyl)pyridin-2-yl)oxy)pyrimidin-2-yl)piperazin-1-yl)butan-2-ol;
3-(4-(5-((4-((4-(acetamidomethyl)piperidin-1-yl)methyl)-6-(3-chloro-5-(difluoromethyl)phenyl)pyridin-2-yl)oxy)pyrimidin-2-yl)piperazin-1-yl)propanoic acid;
3-(4-(5-((6-(3,5-dichlorophenyl)-4-((4-(((methoxycarbonyl)amino)methyl)piperidin-1-yl)methyl)pyridin-2-yl)oxy)pyrimidin-2-yl)piperazin-1-yl)propanoic acid;
methyl ((1-((2-(3,5-dichlorophenyl)-6-((2-(4-methylpiperazin-1-yl)pyrimidin-5-yl)oxy)pyridin-4-yl)methyl)piperidin-4-yl)methyl)carbamate;
N-((1-((2-(3,5-dichlorophenyl)-6-((2-(4-methylpiperazin-1-yl)pyrimidin-5-yl)oxy)pyridin-4-yl)methyl)piperidin-4-yl)methyl)acetamide;
methyl ((1-((2-(3,5-dichlorophenyl)-6-((6-(4-methylpiperazin-1-yl)pyridin-3-yl)oxy)pyridin-4-yl)methyl)piperidin-4-yl)methyl)carbamate;
(1-((2-(3,5-dichlorophenyl)-6-((2-(4-methylpiperazin-1-yl)pyrimidin-5-yl)oxy)pyridin-4-yl)methyl)piperidin-4-yl)methyl methylcarbamate;
(1-((2-(3,5-dichlorophenyl)-6-((6-(4-methylpiperazin-1-yl)pyridin-3-yl)oxy)pyridin-4-yl)methyl)piperidin-4-yl)methyl methylcarbamate;
N-((1-((3′,5′-dichloro-5-((2-(4-methylpiperazin-1-yl)pyrimidin-5-yl)oxy)-[1,1′-biphenyl]-3-yl)methyl)piperidin-4-yl)methyl)acetamide;
1-((1-((2-(3,5-dichlorophenyl)-6-((6-(4-methylpiperazin-1-yl)pyridin-3-yl)oxy)pyridin-4-yl)methyl)piperidin-4-yl)methyl)-3-methylurea;
1-((1-((2-(3,5-dichlorophenyl)-6-((2-(4-methylpiperazin-1-yl)pyrimidin-5-yl)oxy)pyridin-4-yl)methyl)piperidin-4-yl)methyl)-3-methylurea;
N-((1-((2-(3,5-dichlorophenyl)-6-((2-(8-methyl-3,8-diazabicyclo[3.2.1]octan-3-yl)pyrimidin-5-yl)oxy)pyridin-4-yl)methyl)piperidin-4-yl)methyl)acetamide;
5-((4-((4-((1H-tetrazol-5-yl)methyl)piperidin-1-yl)methyl)-6-(3,5-dichlorophenyl)pyridin-2-yl)oxy)-2-(4-methylpiperazin-1-yl)pyrimidine;
(1-((2-(3,5-dichlorophenyl)-6-((2-(piperazin-1-yl)pyrimidin-5-yl)oxy)pyridin-4-yl)methyl)piperidin-4-yl)methyl methylcarbamate;
(1-((2-(3,5-dichlorophenyl)-6-((6-(piperazin-1-yl)pyridin-3-yl)oxy)pyridin-4-yl)methyl)piperidin-4-yl)methyl methylcarbamate;
1-((1-((2-(3,5-dichlorophenyl)-6-((2-(piperazin-1-yl)pyrimidin-5-yl)oxy)pyridin-4-yl)methyl)piperidin-4-yl)methyl)-3-methylurea;
methyl ((1-((2-(3,5-dichlorophenyl)-6-((6-(piperazin-1-yl)pyridin-3-yl)oxy)pyridin-4-yl)methyl)piperidin-4-yl)methyl)carbamate;
3-(4-(5-((6-(3,5-dichlorophenyl)-4-((4-(((methylcarbamoyl)oxy)methyl)piperidin-1-yl)methyl)pyridin-2-yl)oxy)pyridin-2-yl)piperazin-1-yl)propanoic acid;
3-(4-(5-((4-((4-(3-amino-3-oxopropyl)piperazin-1-yl)methyl)-6-(3,5-dichlorophenyl)pyridin-2-yl)oxy)pyrimidin-2-yl)piperazin-1-yl)propanoic acid;
3-(4-(5-((4-((4-(2-amino-2-oxoethyl)piperazin-1-yl)methyl)-6-(3,5-dichlorophenyl)pyridin-2-yl)oxy)pyrimidin-2-yl)piperazin-1-yl)propanoic acid;
3-(4-(5-((6-(3,5-dichlorophenyl)-4-(((1R,7S,8r)-8-(methylsulfonamido)-4-azabicyclo[5.1.0]octan-4-yl)methyl)pyridin-2-yl)oxy)pyrimidin-2-yl)piperazin-1-yl)propanoic acid;
4-(4-(5-((6-(3,5-dichlorophenyl)-4-(((1R,7S,8r)-8-(methylsulfonamido)-4-azabicyclo[5.1.0]octan-4-yl)methyl)pyridin-2-yl)oxy)pyrimidin-2-yl)piperazin-1-yl)-2-methylbutanoic acid;
4-(4-(5-((4-(((1R,7S,8r)-8-acetamido-4-azabicyclo[5.1.0]octan-4-yl)methyl)-6-(3,5-dichlorophenyl)pyridin-2-yl)oxy)pyrimidin-2-yl)piperazin-1-yl)-2-methylbutanoic acid;
4-(4-(5-((6-(3,5-dichlorophenyl)-4-(pyrrolidin-1-ylmethyl)pyridin-2-yl)oxy)pyrimidin-2-yl)piperazin-1-yl)-2-methylbutanoic acid;
3-(4-(5-((4-((4-(cyclopropanecarboxamidomethyl)piperidin-1-yl)methyl)-6-(3,5-dichlorophenyl)pyridin-2-yl)oxy)pyrimidin-2-yl)piperazin-1-yl)propanoic acid;
3-(4-(5-((6-(3,5-dichlorophenyl)-4-((4-((dimethylphosphoryl)methyl)piperidin-1-yl)methyl)pyridin-2-yl)oxy)pyridin-2-yl)piperazin-1-yl)propanoic acid;
2-(1-((2-(3,5-dichlorophenyl)-6-((2-(piperazin-1-yl)pyrimidin-5-yl)oxy)pyridin-4-yl)methyl)piperidin-4-yl)acetamide;
2-(1-((2-(3,5-dichlorophenyl)-6-((2-(4-methylpiperazin-1-yl)pyrimidin-5-yl)oxy)pyridin-4-yl)methyl)piperidin-4-yl)ethanesulfonamide;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)propanamide;
N-((1-((2-(3,5-dichlorophenyl)-6-((6-(piperazin-1-yl)pyridin-3-yl)oxy)pyrimidin-4-yl)methyl)piperidin-4-yl)methyl)acetamide;
N-((1-((6-(3,5-dichlorophenyl)-2-((6-(piperazin-1-yl)pyridin-3-yl)oxy)pyrimidin-4-yl)methyl)piperidin-4-yl)methyl)acetamide;
N-((1-((2-(3,5-dichlorophenyl)-6-((5-fluoro-6-(piperazin-1-yl)pyridin-3-yl)oxy)pyridin-4-yl)methyl)-4-hydroxypiperidin-4-yl)methyl)acetamide;
N-((1-((2-(3,5-dichlorophenyl)-6-((2-(piperazin-1-yl)pyrimidin-5-yl)oxy)pyridin-4-yl)methyl)piperidin-4-yl)methyl)acetamide;
N-((1-((2-(3,5-dichlorophenyl)-6-((2-(4-(2-hydroxyethyl)piperazin-1-yl)pyrimidin-5-yl)oxy)pyridin-4-yl)methyl)piperidin-4-yl)methyl)acetamide;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-N-methylpropanamide;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)butanamide;
4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)butanamide;
1-(5-((3′,5′-dichloro-5-(((2-methoxyethyl)amino)methyl)-[1,1′-biphenyl]-3-yl)oxy)pyridin-2-yl)-N-methylpiperidin-4-amine;
1-(3′,5′-dichloro-5-((6-(piperazin-1-yl)pyridin-3-yl)oxy)-[1,1′-biphenyl]-3-yl)-N-methylmethanamine;
N₁-(5-((3′,5′-dichloro-5-(morpholinomethyl)-[1,1′-biphenyl]-3-yl)oxy)pyridin-2-yl)ethane-1,2-diamine;
1-(5-((3′,5′-dichloro-5-((methylamino)methyl)-[1,1′-biphenyl]-3-yl)oxy)pyridin-2-yl)piperidin-4-amine;
N₁-(5-((3′,5′-dichloro-5-((methylamino)methyl)-[1,1′-biphenyl]-3-yl)oxy)pyridin-2-yl)propane-1,3-diamine;
1-(3′,5′-dichloro-5-((6-(3,3-dimethylpiperazin-1-yl)pyridin-3-yl)oxy)-[1,1′-biphenyl]-3-yl)-N-methylmethanamine;
1-(5-((6-(1,4-diazepan-1-yl)pyridin-3-yl)oxy)-3′,5′-dichloro-[1,1′-biphenyl]-3-yl)-N-methylmethanamine;
1-(3′,5′-dichloro-5-((6-(4-methylpiperazin-1-yl)pyridin-3-yl)oxy)-[1,1′-biphenyl]-3-yl)-N-methylmethanamine;
N-((1-((5-((6-((2-amino-2-methylpropyl)amino)pyridin-3-yl)oxy)-3′,5′-dichloro-[1,1′-biphenyl]-3-yl)methyl)piperidin-4-yl)methyl)acetamide;
N-((1-((5-((6-((3S,4R)-4-amino-3-fluoropiperidin-1-yl)pyridin-3-yl)oxy)-3′,5′-dichloro-[1,1′-biphenyl]-3-yl)methyl)piperidin-4-yl)methyl)acetamide;
N-((1-((3′,5′-dichloro-5-((6-(3-oxohexahydroimidazo[1,5-a]pyrazin-7(1H)-yl)pyridin-3-yl)oxy)-[1,1′-biphenyl]-3-yl)methyl)piperidin-4-yl)methyl)acetamide;
1-(5-((6-(3-chloro-5-methylphenyl)-4-((methylamino)methyl)pyridin-2-yl)oxy)pyridin-2-yl)piperidin-4-amine;
N-((1-((2-(3,5-dichlorophenyl)-6-((5-(piperazin-1-yl)pyrazin-2-yl)oxy)pyridin-4-yl)methyl)piperidin-4-yl)methyl)acetamide;
N-((1-((2-(3,5-dichlorophenyl)-6-((5-(4-methylpiperazin-1-yl)pyrazin-2-yl)oxy)pyridin-4-yl)methyl)piperidin-4-yl)methyl)acetamide;
3-(4-(5-((4-((4-(acetamidomethyl)piperidin-1-yl)methyl)-6-(3,5-dichlorophenyl)pyridin-2-yl)oxy)pyrazin-2-yl)piperazin-1-yl)propanoic acid;
2-(1-((2-(3,5-dichlorophenyl)-6-((5-(piperazin-1-yl)pyrazin-2-yl)oxy)pyridin-4-yl)methyl)piperidin-4-yl)acetic acid;
N-((1-((3′,5′-dichloro-5-((2-(4-(3-hydroxypropyl)piperazin-1-yl)pyrimidin-5-yl)oxy)-[1,1′-biphenyl]-3-yl)methyl)piperidin-4-yl)methyl)acetamide;
3-(1-((3′,5′-dichloro-5-((2-(4-(2-hydroxyethyl)piperazin-1-yl)pyrimidin-5-yl)oxy)-[1,1′-biphenyl]-3-yl)methyl)piperidin-4-yl)propanoic acid;
3-(4-(5-((4-((4-(acetamidomethyl)piperidin-1-yl)methyl)-6-(3-chloro-5-fluorophenyl)pyridin-2-yl)oxy)pyridin-2-yl)piperazin-1-yl)propanoic acid;
3-(4-(5-((4-((4-(acetamidomethyl)piperidin-1-yl)methyl)-6-(3-bromo-5-fluorophenyl)pyridin-2-yl)oxy)pyridin-2-yl)piperazin-1-yl)propanoic acid;
2-(1-((3′,5′-dichloro-5-((2-(4-methylpiperazin-1-yl)pyrimidin-5-yl)oxy)-[1,1′-biphenyl]-3-yl)methyl)piperidin-4-yl)acetic acid;
N-((1-((2-((2-(1,4-diazepan-1-yl)pyrimidin-5-yl)oxy)-6-(3,5-dichlorophenyl)pyridin-4-yl)methyl)piperidin-4-yl)methyl)acetamide;
N-((1-((2-((2-(4-aminopiperidin-1-yl)pyrimidin-5-yl)oxy)-6-(3,5-dichlorophenyl)pyridin-4-yl)methyl)piperidin-4-yl)methyl)acetamide;
2-(1-((2-(3,5-dichlorophenyl)-6-((6-(piperazin-1-yl)pyridin-3-yl)oxy)pyridin-4-yl)methyl)piperidin-4-yl)-N,N-dimethylethanamine oxide;
N-((1-((2-(3,5-dichlorophenyl)-6-((5-fluoro-6-(piperazin-1-yl)pyridin-3-yl)oxy)pyridin-4-yl)methyl)piperidin-4-yl)methyl)acetamide;
2-((1-((2-(3,5-dichlorophenyl)-6-((2-(piperazin-1-yl)pyrimidin-5-yl)oxy)pyridin-4-yl)methyl)piperidin-4-yl)oxy)acetic acid;
2-(1-((2-(3,5-dichlorophenyl)-6-((2-(6-hydroxy-1,4-diazepan-1-yl)pyrimidin-5-yl)oxy)pyridin-4-yl)methyl)piperidin-4-yl)acetic acid;
2-(1-((2-((2-(4-amino-4-(2-hydroxyethyl)piperidin-1-yl)pyrimidin-5-yl)oxy)-6-(3,5-dichlorophenyl)pyridin-4-yl)methyl)piperidin-4-yl)acetic acid;
((1-((2-(3,5-dichlorophenyl)-6-((2-(piperazin-1-yl)pyrimidin-5-yl)oxy)pyridin-4-yl)methyl)piperidin-4-yl)methyl)dimethylphosphine oxide;
2-(1-((2-(3,5-dichlorophenyl)-6-((2-(4-methyl-1,4-diazepan-1-yl)pyrimidin-5-yl)oxy)pyridin-4-yl)methyl)piperidin-4-yl)acetic acid;
3-(1-((2-(3,5-dichlorophenyl)-6-((2-(4-methylpiperazin-1-yl)pyrimidin-5-yl)oxy)pyridin-4-yl)methyl)azetidin-3-yl)butanoic acid;
2-((1-((2-(3,5-dichlorophenyl)-6-((2-(4-methylpiperazin-1-yl)pyrimidin-5-yl)oxy)pyridin-4-yl)methyl)pyrrolidin-3-yl)oxy)acetic acid;
2-(2-((2-(3,5-dichlorophenyl)-6-((2-(4-methylpiperazin-1-yl)pyrimidin-5-yl)oxy)pyridin-4-yl)methyl)octahydrocyclopenta[c]pyrrol-5-yl)acetic acid;
3-(1-((2-(3,5-dichlorophenyl)-6-((2-(4-methylpiperazin-1-yl)pyrimidin-5-yl)oxy)pyridin-4-yl)methyl)piperidin-4-yl)propanoic acid;
3-(1-((2-(3,5-dichlorophenyl)-6-((2-(4-methylpiperazin-1-yl)pyrimidin-5-yl)oxy)pyridin-4-yl)methyl)piperidin-4-yl)-2-methylpropanoic acid;
2-(1-((2-((2-(1,4-diazepan-1-yl)pyrimidin-5-yl)oxy)-6-(3,5-dichlorophenyl)pyridin-4-yl)methyl)piperidin-4-yl)acetic acid;
2-(1-((2-(3,5-dichlorophenyl)-6-((2-(5-methylhexahydropyrrolo[3,4-c]pyrrol-2(1H)-yl)pyrimidin-5-yl)oxy)pyridin-4-yl)methyl)piperidin-4-yl)acetic acid;
(S)-3-(1-((2-(3,5-dichlorophenyl)-6-((2-(4-methylpiperazin-1-yl)pyrimidin-5-yl)oxy)pyridin-4-yl)methyl)piperidin-4-yl)-2-methylpropanoic acid;
1-(7-((2-(3,5-dichlorophenyl)-6-((2-(4-methylpiperazin-1-yl)pyrimidin-5-yl)oxy)pyridin-4-yl)methyl)-2,7-diazaspiro[3.5]nonan-2-yl)-2-hydroxyethanone;
(1R,7S,8r)-4-((2-(3,5-dichlorophenyl)-6-((6-(4-methylpiperazin-1-yl)pyridin-3-yl)oxy)pyridin-4-yl)methyl)-4-azabicyclo[5.1.0]octane-8-carboxylic acid;
(R)-3-(1-((2-(3,5-dichlorophenyl)-6-((2-(4-methylpiperazin-1-yl)pyrimidin-5-yl)oxy)pyridin-4-yl)methyl)piperidin-4-yl)-2-methylpropanoic acid;
1-(1-((2-(3,5-dichlorophenyl)-6-((2-(4-methylpiperazin-1-yl)pyrimidin-5-yl)oxy)pyridin-4-yl)methyl)piperidin-4-yl)propan-2-ol;
3-(1-((2-(3,5-dichlorophenyl)-6-((2-(4-methylpiperazin-1-yl)pyrimidin-5-yl)oxy)pyridin-4-yl)methyl)piperidin-4-yl)-2-hydroxypropanoic acid;
2-((1-((2-(3,5-dichlorophenyl)-6-((2-(4-methyl-1,4-diazepan-1-yl)pyrimidin-5-yl)oxy)pyridin-4-yl)methyl)piperidin-4-yl)oxy)acetic acid;
9-((2-(3,5-dichlorophenyl)-6-((6-(4-methylpiperazin-1-yl)pyridin-3-yl)oxy)pyridin-4-yl)methyl)-2-oxa-4,9-diazaspiro[5.5]undecan-3-one;
2-(1-((2-(3,5-dichlorophenyl)-6-((2-(4-methylpiperazin-1-yl)pyrimidin-5-yl)oxy)pyridin-4-yl)methyl)piperidin-4-yl)acetamide;
1-((1-((2-(3,5-dichlorophenyl)-6-((2-(4-methylpiperazin-1-yl)pyrimidin-5-yl)oxy)pyridin-4-yl)methyl)piperidin-4-yl)oxy)cyclopropanecarboxylic acid;
2-(1-((2-(3,5-dichlorophenyl)-6-((2-(6-methoxy-4-methyl-1,4-diazepan-1-yl)pyrimidin-5-yl)oxy)pyridin-4-yl)methyl)piperidin-4-yl)acetic acid;
N-((1-((2-(3,5-dichlorophenyl)-6-((6-(6-fluoro-4-methyl-1,4-diazepan-1-yl)pyridin-3-yl)oxy)pyridin-4-yl)methyl)piperidin-4-yl)methyl)acetamide;
2-(1-((2-(3,5-dichlorophenyl)-6-((2-(6-hydroxy-4,6-dimethyl-1,4-diazepan-1-yl)pyrimidin-5-yl)oxy)pyridin-4-yl)methyl)piperidin-4-yl)acetic acid;
2-(1-((2-(3,5-dichlorophenyl)-6-((2-(6-fluoro-4-methyl-1,4-diazepan-1-yl)pyrimidin-5-yl)oxy)pyridin-4-yl)methyl)piperidin-4-yl)acetic acid;
2-(1-((2-(3,5-dichlorophenyl)-6-((4-methyl-2-(4-methylpiperazin-1-yl)pyrimidin-5-yl)oxy)pyridin-4-yl)methyl)piperidin-4-yl)acetic acid;
2-(1-((2-(3,5-dichlorophenyl)-6-((2-methyl-6-(4-methylpiperazin-1-yl)pyridin-3-yl)oxy)pyridin-4-yl)methyl)piperidin-4-yl)acetic acid;
2-(1-((2-(3,5-dichlorophenyl)-6-(4-(4-methylpiperazin-1-yl)phenoxy)pyridin-4-yl)methyl)piperidin-4-yl)acetic acid;
2-(1-((2-(3,5-dichlorophenyl)-6-((5-fluoro-6-(4-methylpiperazin-1-yl)pyridin-3-yl)oxy)pyridin-4-yl)methyl)piperidin-4-yl)acetic acid;
2-(1-((2-(3,5-dichlorophenyl)-6-((2-(3-(methylamino)pyrrolidin-1-yl)pyrimidin-5-yl)oxy)pyridin-4-yl)methyl)piperidin-4-yl)acetic acid;
(S)-2-(4-((2-(3,5-dichlorophenyl)-6-((2-(4-methylpiperazin-1-yl)pyrimidin-5-yl)oxy)pyridin-4-yl)methyl)-1,4-oxazepan-7-yl)ethanol;
N-((1R,5S,6r)-3-((2-(3,5-dichlorophenyl)-6-((2-(4-methyl-1,4-diazepan-1-yl)pyrimidin-5-yl)oxy)pyridin-4-yl)methyl)-3-azabicyclo[3.1.0]hexan-6-yl)acetamide;
1-(1-((2-(3,5-dichlorophenyl)-6-((2-(4-methylpiperazin-1-yl)pyrimidin-5-yl)oxy)pyridin-4-yl)methyl)piperidin-4-yl)propan-2-one;
2-(1-((2-(3,5-dichlorophenyl)-6-((6-(4-methyl-1,4-diazepan-1-yl)pyridin-3-yl)oxy)pyridin-4-yl)methyl)piperidin-4-yl)acetic acid;
N-((1-((2-(3,5-dichlorophenyl)-6-((6-(4-methylpiperazin-1-yl)pyridazin-3-yl)oxy)pyridin-4-yl)methyl)piperidin-4-yl)methyl)acetamide;
2-(1-((2-((2-(4-aminopiperidin-1-yl)pyrimidin-5-yl)oxy)-6-(3,5-dichlorophenyl)pyridin-4-yl)methyl)piperidin-4-yl)acetic acid;
(S)-2-(1-((2-(3,5-dichlorophenyl)-6-((2-(3-methylpiperazin-1-yl)pyrimidin-5-yl)oxy)pyridin-4-yl)methyl)piperidin-4-yl)acetic acid;
2-(1-((2-(3,5-dichlorophenyl)-6-((2-(3,3-dimethylpiperazin-1-yl)pyrimidin-5-yl)oxy)pyridin-4-yl)methyl)piperidin-4-yl)acetic acid;
2-(1-((2-(3,5-dichlorophenyl)-6-((2-(hexahydropyrrolo[3,4-c]pyrrol-2(1H)-yl)pyrimidin-5-yl)oxy)pyridin-4-yl)methyl)piperidin-4-yl)acetic acid;
2-(1-((2-((2-(3,6-diazabicyclo[3.1.1]heptan-3-yl)pyrimidin-5-yl)oxy)-6-(3,5-dichlorophenyl)pyridin-4-yl)methyl)piperidin-4-yl)acetic acid;
2-(1-((2-((2-(3,8-diazabicyclo[3.2.1]octan-3-yl)pyrimidin-5-yl)oxy)-6-(3,5-dichlorophenyl)pyridin-4-yl)methyl)piperidin-4-yl)acetic acid;
2-(1-((2-((2-(4,7-diazaspiro[2.5]octan-7-yl)pyrimidin-5-yl)oxy)-6-(3,5-dichlorophenyl)pyridin-4-yl)methyl)piperidin-4-yl)acetic acid;
2-(1-((2-((2-(4-amino-4-(hydroxymethyl)piperidin-1-yl)pyrimidin-5-yl)oxy)-6-(3,5-dichlorophenyl)pyridin-4-yl)methyl)piperidin-4-yl)acetic acid;
2-(1-((2-((6-(1,4-diazepan-1-yl)pyridin-3-yl)oxy)-6-(3,5-dichlorophenyl)pyridin-4-yl)methyl)piperidin-4-yl)acetic acid;
N-((1-((3′,5′-dichloro-5-((2-(4-(2-(methylsulfonyl)ethyl)piperazin-1-yl)pyrimidin-5-yl)oxy)-[1,1′-biphenyl]-3-yl)methyl)piperidin-4-yl)methyl)acetamide;
3-(4-(5-((5-((4-(acetamidomethyl)piperidin-1-yl)methyl)-3′,5′-dichloro-[1,1′-biphenyl]-3-yl)oxy)pyrimidin-2-yl)piperazin-1-yl)propanoic acid;
3-(4-(6-((4-((4-(acetamidomethyl)piperidin-1-yl)methyl)-6-(3,5-dichlorophenyl)pyridin-2-yl)oxy)pyridazin-3-yl)piperazin-1-yl)propanoic acid;
3-(4-(5-((5-((4-(acetamidomethyl)piperidin-1-yl)methyl)-3′,5′-dichloro-[1,1′-biphenyl]-3-yl)oxy)pyrimidin-2-yl)piperazin-1-yl)propanamide;
1-((1-((2-(3,5-dichlorophenyl)-6-((2-(4-(3-hydroxypropyl)piperazin-1-yl)pyrimidin-5-yl)oxy)pyridin-4-yl)methyl)piperidin-4-yl)methyl)-3-methylurea;
methyl (3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)propanoyl)carbamate;
1-(2-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)ethyl)cyclopropanecarboxylic acid;
4-(4-(5-((4-((4-(acetamidomethyl)piperidin-1-yl)methyl)-6-(3,5-dichlorophenyl)pyridin-2-yl)oxy)pyridin-2-yl)piperazin-1-yl)-2-methylbutanoic acid;
methyl (3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)propanoyl)carbamate;
methyl (3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanoyl)carbamate;
2-(1-((2-(3,5-dichlorophenyl)-6-((2-(4-(2,3-dihydroxypropyl)piperazin-1-yl)pyrimidin-5-yl)oxy)pyridin-4-yl)methyl)piperidin-4-yl)acetic acid;
4-(5-((4-((4-(acetamidomethyl)piperidin-1-yl)methyl)-6-(3,5-dichlorophenyl)pyridin-2-yl)oxy)pyridin-2-yl)-1-methylpiperazine 1-oxide;
4-(5-((4-((4-(acetamidomethyl)piperidin-1-yl)methyl)-6-(3,5-dichlorophenyl)pyridin-2-yl)oxy)pyridin-2-yl)-1,1-bis(2-hydroxyethyl)piperazin-1-ium;
N-((1-((2-(3,5-dichlorophenyl)-6-((6-(4-(2-hydroxyethyl)piperazin-1-yl)pyridin-3-yl)oxy)pyridin-4-yl)methyl)piperidin-4-yl)methyl)acetamide;
4-(5-((4-((4-(acetamidomethyl)piperidin-1-yl)methyl)-6-(3,5-dichlorophenyl)pyridin-2-yl)oxy)pyridin-2-yl)-1,1-dimethylpiperazin-1-ium;
N-((1-((2-((6-(4-amino-3-fluoropiperidin-1-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-((1S,4S)-5-(2-(methylsulfonyl)ethyl)-2,5-diazabicyclo[2.2.1]heptan-2-yl)pyridin-3-yl)oxy)pyridin-4-yl)methyl)piperidin-4-yl)methyl)acetamide;
N-((1-((2-((6-((3S,4R)-3-(aminomethyl)-4-hydroxypyrrolidin-1-yl)pyridin-3-yl)oxy)-6-(3,5-dichlorophenyl)pyridin-4-yl)methyl)piperidin-4-yl)methyl)acetamide;
3-((1R,5S)-3-(5-((4-((4-(acetamidomethyl)piperidin-1-yl)methyl)-6-(3,5-dichlorophenyl)pyridin-2-yl)oxy)pyridin-2-yl)-3,8-diazabicyclo[3.2.1]octan-8-yl)propanoic acid;
(S)-3-(4-(5-((4-((4-(acetamidomethyl)piperidin-1-yl)methyl)-6-(3,5-dichlorophenyl)pyridin-2-yl)oxy)pyridin-2-yl)-2-methylpiperazin-1-yl)propanoic acid;
2-(1-((2-((6-((1S,4S)-2,5-diazabicyclo[2.2.1]heptan-2-yl)pyridin-3-yl)oxy)-6-(3,5-dichlorophenyl)pyridin-4-yl)methyl)piperidin-4-yl)acetic acid;
4-(4-(5-((4-((4-(acetamidomethyl)piperidin-1-yl)methyl)-6-(3,5-dichlorophenyl)pyridin-2-yl)oxy)pyridin-2-yl)piperazin-1-yl)-2-ethylbutanoic acid;
3-(4-(5-((4-((4-(acetamidomethyl)piperidin-1-yl)methyl)-6-(3,5-dichlorophenyl)pyridin-2-yl)oxy)pyridin-2-yl)piperazin-1-yl)-2,2-dimethylpropanoic acid;
3-(4-(5-((4-((4-(acetamidomethyl)piperidin-1-yl)methyl)-6-(3,5-dichlorophenyl)pyridin-2-yl)oxy)pyridin-2-yl)-1,4-diazepan-1-yl)propanoic acid;
3-(4-(5-((4-((4-(acetamidomethyl)piperidin-1-yl)methyl)-6-(3,5-dichlorophenyl)pyridin-2-yl)oxy)pyridin-2-yl)-2,2-dimethylpiperazin-1-yl)propanoic acid;
N-((1-((2-((6-(1,4-diazepan-1-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-(4-(methylamino)piperidin-1-yl)pyridin-3-yl)oxy)pyridin-4-yl)methyl)piperidin-4-yl)methyl)acetamide;
N-((1-((2-(3,5-dichlorophenyl)-6-((6-(hexahydropyrrolo[3,4-c]pyrrol-2(1H)-yl)pyridin-3-yl)oxy)pyridin-4-yl)methyl)piperidin-4-yl)methyl)acetamide;
N-((1-((2-(3,5-dichlorophenyl)-6-((6-(3-(hydroxymethyl)piperazin-1-yl)pyridin-3-yl)oxy)pyridin-4-yl)methyl)piperidin-4-yl)methyl)acetamide;
N-((1-((2-((6-(4-aminopiperidin-1-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-(3,3-dimethylpiperazin-1-yl)pyridin-3-yl)oxy)pyridin-4-yl)methyl)piperidin-4-yl)methyl)acetamide;
N-((1-((2-((6-(4-amino-3,3-dimethylpiperidin-1-yl)pyridin-3-yl)oxy)-6-(3,5-dichlorophenyl)pyridin-4-yl)methyl)piperidin-4-yl)methyl)acetamide;
N-((1-((2-((6-(2,7-diazaspiro[4.4]nonan-2-yl)pyridin-3-yl)oxy)-6-(3,5-dichlorophenyl)pyridin-4-yl)methyl)piperidin-4-yl)methyl)acetamide;
N-((1-((2-(3,5-dichlorophenyl)-6-((6-(4-methylpiperazin-1-yl)pyridin-3-yl)oxy)pyridin-4-yl)methyl)piperidin-4-yl)methyl)acetamide;
1-(3′,5′-dichloro-5-((6-(hexahydropyrrolo[3,4-c]pyrrol-2(1H)-yl)pyridin-3-yl)oxy)-[1,1′-biphenyl]-3-yl)-N-methylmethanamine;
1-(5-((6-((1S,4S)-2,5-diazabicyclo[2.2.1]heptan-2-yl)pyridin-3-yl)oxy)-3′,5′-dichloro-[1,1′-biphenyl]-3-yl)-N-methylmethanamine;
2-(1-((2-(3,5-dichlorophenyl)-6-((6-((1S,4S)-5-methyl-2,5-diazabicyclo[2.2.1]heptan-2-yl)pyridin-3-yl)oxy)pyridin-4-yl)methyl)piperidin-4-yl)acetic acid;
2-(1-((2-(3,5-dichlorophenyl)-6-((2-(4-(3-(methylsulfinyl)butyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-(methylsulfonyl)butyl)piperazin-1-yl)pyrimidin-5-yl)oxy)pyridin-4-yl)methyl)piperidin-4-yl)acetic acid;
2-(1-((2-(3,5-dichlorophenyl)-6-((2-(piperazin-1-yl)pyrimidin-5-yl)oxy)pyridin-4-yl)methyl)piperidin-4-yl)acetic acid;
2-(1-((2-(3,5-dichlorophenyl)-6-((2-(4-methylpiperazin-1-yl)pyrimidin-5-yl)oxy)pyridin-4-yl)methyl)piperidin-4-yl)acetic acid;
4-(4-(5-((6-(3,5-dichlorophenyl)-4-((4-(((methylcarbamoyl)oxy)methyl)piperidin-1-yl)methyl)pyridin-2-yl)oxy)pyrimidin-2-yl)piperazin-1-yl)pentanoic acid;
(1-((2-(3,5-dichlorophenyl)-6-((2-(4-(4-hydroxybutan-2-yl)piperazin-1-yl)pyrimidin-5-yl)oxy)pyridin-4-yl)methyl)piperidin-4-yl)methyl methylcarbamate;
(1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)methyl methylcarbamate;
3-(4-(5-((6-(3,5-dichlorophenyl)-4-((4-(((methylcarbamoyl)oxy)methyl)piperidin-1-yl)methyl)pyridin-2-yl)oxy)pyrimidin-2-yl)piperazin-1-yl)cyclobutanecarboxylic acid;
2-(1-((2-(3,5-dichlorophenyl)-6-((6-(4-methylpiperazin-1-yl)pyridin-3-yl)oxy)pyridin-4-yl)methyl)piperidin-4-yl)acetic acid;
3-(1-((2-(3,5-dichlorophenyl)-6-((6-(4-methylpiperazin-1-yl)pyridin-3-yl)oxy)pyridin-4-yl)methyl)piperidin-4-yl)-2-methylpropanoic acid;
N-((1-((2-(3,5-dichlorophenyl)-6-((2-(4-(3-(methylsulfonyl)butyl)piperazin-1-yl)pyrimidin-5-yl)oxy)pyridin-4-yl)methyl)piperidin-4-yl)methyl)acetamide;
4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)pentanoic acid;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)cyclobutanecarboxylic acid;
methyl ((1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)methyl)carbamate;
N-((1-((2-(3,5-dichlorophenyl)-6-((6-(4-(3-(methylsulfonyl)butyl)piperazin-1-yl)pyridin-3-yl)oxy)pyridin-4-yl)methyl)piperidin-4-yl)methyl)acetamide;
1-((1-((2-(3,5-dichlorophenyl)-6-((6-(4-(3-(methylsulfonyl)butyl)piperazin-1-yl)pyridin-3-yl)oxy)pyridin-4-yl)methyl)piperidin-4-yl)methyl)-3-methylurea;
1-((1-((2-(3,5-dichlorophenyl)-6-((2-(4-(3-(methylsulfonyl)butyl)piperazin-1-yl)pyrimidin-5-yl)oxy)pyridin-4-yl)methyl)piperidin-4-yl)methyl)-3-methylurea;
3-(4-(5-((6-(3,5-dichlorophenyl)-4-((4-((3-methylureido)methyl)piperidin-1-yl)methyl)pyridin-2-yl)oxy)pyrimidin-2-yl)piperazin-1-yl)cyclobutanecarboxylic acid;
4-(4-(5-((6-(3,5-dichlorophenyl)-4-((4-(((methoxycarbonyl)amino)methyl)piperidin-1-yl)methyl)pyridin-2-yl)oxy)pyrimidin-2-yl)piperazin-1-yl)pentanoic acid;
methyl ((1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)methyl)carbamate;
2-((1R,7S,8r)-4-((2-(3,5-dichlorophenyl)-6-((2-(4-methylpiperazin-1-yl)pyrimidin-5-yl)oxy)pyridin-4-yl)methyl)-4-azabicyclo[5.1.0]octan-8-yl)acetic acid;
2-((1-((2-(3,5-dichlorophenyl)-6-((2-(4-methylpiperazin-1-yl)pyrimidin-5-yl)oxy)pyridin-4-yl)methyl)piperidin-4-yl)oxy)-2-methylpropanoic acid;
2-(1-((2-(3,5-dichlorophenyl)-6-((2-(6-hydroxy-4-methyl-1,4-diazepan-1-yl)pyrimidin-5-yl)oxy)pyridin-4-yl)methyl)piperidin-4-yl)acetic acid;
2-(1-((2-(3,5-dichlorophenyl)-6-((2-(4-(dimethylamino)piperidin-1-yl)pyrimidin-5-yl)oxy)pyridin-4-yl)methyl)piperidin-4-yl)acetic acid;
2-(1-((2-(3,5-dichlorophenyl)-6-((2-(6-methyl-3,6-diazabicyclo[3.1.1]heptan-3-yl)pyrimidin-5-yl)oxy)pyridin-4-yl)methyl)piperidin-4-yl)acetic acid;
2-(1-((2-(3,5-dichlorophenyl)-6-((2-(4-((1-hydroxycyclopropyl)methyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-ethyl-1,4-diazepan-1-yl)pyrimidin-5-yl)oxy)pyridin-4-yl)methyl)piperidin-4-yl)acetic acid;
2-(1-((2-(3,5-dichlorophenyl)-6-((2-((1S,4S)-5-methyl-2,5-diazabicyclo[2.2.1]heptan-2-yl)pyrimidin-5-yl)oxy)pyridin-4-yl)methyl)piperidin-4-yl)acetic acid;
2-(1-((2-(3,5-dichlorophenyl)-6-((2-((1S,4S)-5-ethyl-2,5-diazabicyclo[2.2.1]heptan-2-yl)pyrimidin-5-yl)oxy)pyridin-4-yl)methyl)piperidin-4-yl)acetic acid;
2-(1-((2-((2-(4-cyclopropylpiperazin-1-yl)pyrimidin-5-yl)oxy)-6-(3,5-dichlorophenyl)pyridin-4-yl)methyl)piperidin-4-yl)acetic acid;
2-(1-((2-(3,5-dichlorophenyl)-6-((2-(4-ethylpiperazin-1-yl)pyrimidin-5-yl)oxy)pyridin-4-yl)methyl)piperidin-4-yl)acetic acid;
2-(1-((2-(3,5-dichlorophenyl)-6-((2-(4-isopropylpiperazin-1-yl)pyrimidin-5-yl)oxy)pyridin-4-yl)methyl)piperidin-4-yl)acetic acid;
2-(1-((2-(3,5-dichlorophenyl)-6-((2-(4-(2-fluoroethyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-hydroxybutyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(4-hydroxybutan-2-yl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(4-(methylamino)butan-2-yl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(4-(dimethylamino)butan-2-yl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(2-((methylcarbamoyl)oxy)ethyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)acetic acid;
2-(1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)acetic acid;
2-(1-((2-(3,5-dichlorophenyl)-6-((6-(4-(2-methoxyethyl)piperazin-1-yl)pyridin-3-yl)oxy)pyridin-4-yl)methyl)piperidin-4-yl)acetic acid;
N-((1-((2-(3,5-dichlorophenyl)-6-((2-(4-(2-methoxyethyl)piperazin-1-yl)pyrimidin-5-yl)oxy)pyridin-4-yl)methyl)piperidin-4-yl)methyl)acetamide;
2-(1-((2-(3,5-dichlorophenyl)-6-((2-(4-(3-sulfamoylpropyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(2-methoxyethyl)piperazin-1-yl)pyrimidin-5-yl)oxy)pyridin-4-yl)methyl)piperidin-4-yl)acetic acid;
3-(4-(5-((6-(3,5-dichlorophenyl)-4-((4-(((methylcarbamoyl)oxy)methyl)piperidin-1-yl)methyl)pyridin-2-yl)oxy)pyrimidin-2-yl)piperazin-1-yl)propanoic acid;
4-(4-(5-((4-((4-(acetamidomethyl)piperidin-1-yl)methyl)-6-(3,5-dichlorophenyl)pyridin-2-yl)oxy)pyridin-2-yl)piperazin-1-yl)butanoic acid;
N-((1-((2-(3,5-dichlorophenyl)-6-((6-(4-(2-methoxyethyl)piperazin-1-yl)pyridin-3-yl)oxy)pyridin-4-yl)methyl)piperidin-4-yl)methyl)acetamide;
N-((1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)methyl)acetamide;
2-(1-((2-(3,5-dichlorophenyl)-6-((6-(4-(3-sulfamoylpropyl)piperazin-1-yl)pyridin-3-yl)oxy)pyridin-4-yl)methyl)piperidin-4-yl)acetic acid;
N-((1-((2-(3,5-dichlorophenyl)-6-((2-(4-(3-sulfamoylpropyl)piperazin-1-yl)pyrimidin-5-yl)oxy)pyridin-4-yl)methyl)piperidin-4-yl)methyl)acetamide;
1-((1-((2-(3,5-dichlorophenyl)-6-((6-(4-(2-methoxyethyl)piperazin-1-yl)pyridin-3-yl)oxy)pyridin-4-yl)methyl)piperidin-4-yl)methyl)-3-methylurea;
1-((1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)methyl)-3-methylurea;
3-(4-(5-((6-(3,5-dichlorophenyl)-4-((4-((3-methylureido)methyl)piperidin-1-yl)methyl)pyridin-2-yl)oxy)pyrimidin-2-yl)piperazin-1-yl)propanoic acid;
1-((1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)methyl)-3-methylurea;
3-(4-(5-((5-((4-(acetamidomethyl)piperidin-1-yl)methyl)-3′,5′-dichloro-[1,1′-biphenyl]-3-yl)oxy)pyridin-2-yl)piperazin-1-yl)propanoic acid;
N-((1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)methyl)acetamide;
3-(4-(5-((6-(3,5-dichlorophenyl)-4-((4-((3-methylureido)methyl)piperidin-1-yl)methyl)pyridin-2-yl)oxy)pyridin-2-yl)piperazin-1-yl)propane-1-sulfonamide;
3-(4-(5-((6-(3,5-dichlorophenyl)-4-((4-((3-methylureido)methyl)piperidin-1-yl)methyl)pyridin-2-yl)oxy)pyrimidin-2-yl)piperazin-1-yl)propanamide;
1-((1-((2-(3,5-dichlorophenyl)-6-((2-(4-(2-methoxyethyl)piperazin-1-yl)pyrimidin-5-yl)oxy)pyridin-4-yl)methyl)piperidin-4-yl)methyl)-3-methylurea;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)propanoic acid;
2-(1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)acetamide;
3-(4-(5-((6-(3,5-dichlorophenyl)-4-((4-hydroxy-4-((3-methylureido)methyl)piperidin-1-yl)methyl)pyridin-2-yl)oxy)pyrimidin-2-yl)piperazin-1-yl)propanamide;
3-(4-(5-((4-((4-(acetamidomethyl)piperidin-1-yl)methyl)-6-(3,5-dichlorophenyl)pyridin-2-yl)oxy)-3-fluoropyridin-2-yl)piperazin-1-yl)propanoic acid;
3-(4-(5-((6-(3,5-dichlorophenyl)-4-((4-(((ethoxycarbonyl)amino)methyl)piperidin-1-yl)methyl)pyridin-2-yl)oxy)pyrimidin-2-yl)piperazin-1-yl)propanoic acid;
3-(4-(5-((6-(3,5-dichlorophenyl)-4-((4-(2-(ethylamino)-2-oxoethyl)piperidin-1-yl)methyl)pyridin-2-yl)oxy)pyrimidin-2-yl)piperazin-1-yl)propanoic acid;
3-(1-((2-(3,5-dichlorophenyl)-6-((2-(piperazin-1-yl)pyrimidin-5-yl)oxy)pyridin-4-yl)methyl)piperidin-4-yl)-2-methylpropanoic acid;
3-(4-(5-((6-(3,5-dichlorophenyl)-4-((4-((3-methylureido)methyl)piperidin-1-yl)methyl)pyridin-2-yl)oxy)-3-fluoropyridin-2-yl)piperazin-1-yl)propanoic acid;
3-(1-((2-(3,5-dichlorophenyl)-6-((6-(4-(3-(methylsulfonyl)propyl)piperazin-1-yl)pyridin-3-yl)oxy)pyridin-4-yl)methyl)piperidin-4-yl)propanoic acid;
3-(1-((2-(3,5-dichlorophenyl)-6-((2-(4-(3-(methylsulfonyl)propyl)piperazin-1-yl)pyrimidin-5-yl)oxy)pyridin-4-yl)methyl)piperidin-4-yl)propanoic acid;
3-(4-(5-((4-((4-(acetamidomethyl)piperidin-1-yl)methyl)-6-(3-chloro-4,5-difluorophenyl)pyridin-2-yl)oxy)pyrimidin-2-yl)piperazin-1-yl)propanamide;
1-((1-((2-(3-chloro-5-fluorophenyl)-6-((2-(4-(3-(methylsulfonyl)propyl)piperazin-1-yl)pyrimidin-5-yl)oxy)pyridin-4-yl)methyl)piperidin-4-yl)methyl)-3-methylurea;
methyl ((1-((2-(3-chloro-5-fluorophenyl)-6-((2-(piperazin-1-yl)pyrimidin-5-yl)oxy)pyridin-4-yl)methyl)piperidin-4-yl)methyl)carbamate;
3-(4-(5-((6-(3,5-dichlorophenyl)-4-((4-(2-(methylsulfonyl)ethyl)piperidin-1-yl)methyl)pyridin-2-yl)oxy)pyridin-2-yl)piperazin-1-yl)propanoic acid;
3-(4-(5-((6-(3,5-dichlorophenyl)-4-((4-(2-sulfamoylethyl)piperidin-1-yl)methyl)pyridin-2-yl)oxy)pyridin-2-yl)piperazin-1-yl)propanoic acid;
2-(1-((2-(3,5-dichlorophenyl)-6-((2-(4-(2-(1-hydroxycyclopropyl)ethyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-hydroxy-3-methylbutyl)piperazin-1-yl)pyrimidin-5-yl)oxy)pyridin-4-yl)methyl)piperidin-4-yl)acetic acid;
2-(1-((2-(3,5-dichlorophenyl)-6-((2-(4-(3-hydroxy-2,2-dimethylpropyl)piperazin-1-yl)pyrimidin-5-yl)oxy)pyridin-4-yl)methyl)piperidin-4-yl)acetic acid;
3-(4-(5-((4-((4-(acetamidomethyl)piperidin-1-yl)methyl)-6-(3,5-dichlorophenyl)pyridin-2-yl)oxy)pyridin-2-yl)piperazin-1-yl)butanoic acid;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)butanoic acid;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylpropanoic acid;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylpropanamide;
N-((1-((2-(3,5-dichlorophenyl)-6-((2-(4-(2-(methylsulfonyl)ethyl)piperazin-1-yl)pyrimidin-5-yl)oxy)pyridin-4-yl)methyl)piperidin-4-yl)methyl)acetamide;
N-((1-((2-(3,5-dichlorophenyl)-6-((2-(4-(2-sulfamoylethyl)piperazin-1-yl)pyrimidin-5-yl)oxy)pyridin-4-yl)methyl)piperidin-4-yl)methyl)acetamide;
2-(4-(5-((4-((4-(acetamidomethyl)piperidin-1-yl)methyl)-6-(3,5-dichlorophenyl)pyridin-2-yl)oxy)pyridin-2-yl)piperazin-1-yl)ethanesulfonic acid;
2-((4-(5-((4-((4-(acetamidomethyl)piperidin-1-yl)methyl)-6-(3,5-dichlorophenyl)pyridin-2-yl)oxy)pyridin-2-yl)piperazin-1-yl)methyl)butanoic acid;
N-((1-((2-(3,5-dichlorophenyl)-6-((6-(4-(2-sulfamoylethyl)piperazin-1-yl)pyridin-3-yl)oxy)pyridin-4-yl)methyl)piperidin-4-yl)methyl)acetamide;
1-((1-((2-(3,5-dichlorophenyl)-6-((6-(4-(2-(methylsulfonyl)ethyl)piperazin-1-yl)pyridin-3-yl)oxy)pyridin-4-yl)methyl)piperidin-4-yl)methyl)-3-methylurea;
3-(4-(5-((6-(3,5-dichlorophenyl)-4-((4-((3-methylureido)methyl)piperidin-1-yl)methyl)pyridin-2-yl)oxy)pyrimidin-2-yl)piperazin-1-yl)-2-methylpropanamide;
3-(4-(5-((3′,5′-dichloro-5-((4-((3-methylureido)methyl)piperidin-1-yl)methyl)-[1,1′-biphenyl]-3-yl)oxy)pyrimidin-2-yl)piperazin-1-yl)-2-methylpropanoic acid;
N-((1-((3′,5′-dichloro-5-((6-(4-(2-(methylsulfonyl)ethyl)piperazin-1-yl)pyridin-3-yl)oxy)-[1,1′-biphenyl]-3-yl)methyl)piperidin-4-yl)methyl)acetamide;
(S)-3-(4-(5-((6-(3,5-dichlorophenyl)-4-((4-(((methoxycarbonyl)amino)methyl)piperidin-1-yl)methyl)pyridin-2-yl)oxy)pyrimidin-2-yl)-2-methylpiperazin-1-yl)propanoic acid;
1-((1-((2-(3,5-dichlorophenyl)-6-((2-(4-(3-hydroxybutyl)piperazin-1-yl)pyrimidin-5-yl)oxy)pyridin-4-yl)methyl)piperidin-4-yl)methyl)-3-methylurea;
3-(3-(5-((4-((4-(acetamidomethyl)piperidin-1-yl)methyl)-6-(3,5-dichlorophenyl)pyridin-2-yl)oxy)pyrimidin-2-yl)-3,8-diazabicyclo[3.2.1]octan-8-yl)propanoic acid;
methyl 3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)propanoate;
(R)-2-(1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)acetic acid;
(R)-2-(1-((2-(3,5-dichlorophenyl)-6-((6-(4-(2-hydroxypropyl)piperazin-1-yl)pyridin-3-yl)oxy)pyridin-4-yl)methyl)piperidin-4-yl)acetic acid;
2-(1-((2-(3,5-dichlorophenyl)-6-((6-(4-(2-hydroxyethyl)piperazin-1-yl)pyridin-3-yl)oxy)pyridin-4-yl)methyl)piperidin-4-yl)acetic acid;
3-(1-((2-(3,5-dichlorophenyl)-6-((2-(4-((R)-2-hydroxypropyl)piperazin-1-yl)pyrimidin-5-yl)oxy)pyridin-4-yl)methyl)piperidin-4-yl)-2-methylpropanoic acid;
(R)-N-((1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)methyl)acetamide;
N-((1-((2-(3,5-dichlorophenyl)-6-((2-(4-(2-hydroxy-2-methylpropyl)piperazin-1-yl)pyrimidin-5-yl)oxy)pyridin-4-yl)methyl)piperidin-4-yl)methyl)acetamide;
N-((1-((2-(3,5-dichlorophenyl)-6-((2-(4-(3-fluoro-2-hydroxypropyl)piperazin-1-yl)pyrimidin-5-yl)oxy)pyridin-4-yl)methyl)piperidin-4-yl)methyl)acetamide;
(R)-(1-((2-(3,5-dichlorophenyl)-6-((6-(4-(2-hydroxypropyl)piperazin-1-yl)pyridin-3-yl)oxy)pyridin-4-yl)methyl)piperidin-4-yl)methyl methylcarbamate;
2-(1-((2-(3,5-dichlorophenyl)-6-((2-(4-(2-hydroxyethyl)piperazin-1-yl)pyrimidin-5-yl)oxy)pyridin-4-yl)methyl)piperidin-4-yl)acetic acid;
(1-((2-(3,5-dichlorophenyl)-6-((2-(4-(2-hydroxyethyl)piperazin-1-yl)pyrimidin-5-yl)oxy)pyridin-4-yl)methyl)piperidin-4-yl)methyl methylcarbamate;
(R)-(1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)methyl methylcarbamate;
(R)-1-((1-((2-(3,5-dichlorophenyl)-6-((6-(4-(2-hydroxypropyl)piperazin-1-yl)pyridin-3-yl)oxy)pyridin-4-yl)methyl)piperidin-4-yl)methyl)-3-methylurea;
(R)-1-((1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)methyl)-3-methylurea;
methyl ((1-((2-(3,5-dichlorophenyl)-6-((2-(4-(2-hydroxyethyl)piperazin-1-yl)pyrimidin-5-yl)oxy)pyridin-4-yl)methyl)piperidin-4-yl)methyl)carbamate;
(R)-methyl ((1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)methyl)carbamate;
1-((1-((3′,5′-dichloro-5-((2-(4-(2-hydroxyethyl)piperazin-1-yl)pyrimidin-5-yl)oxy)-[1,1′-biphenyl]-3-yl)methyl)piperidin-4-yl)methyl)-3-methylurea;
3-(1-((2-(3,5-dichlorophenyl)-6-((6-(4-(2-hydroxyethyl)piperazin-1-yl)pyridin-3-yl)oxy)pyridin-4-yl)methyl)piperidin-4-yl)propanoic acid;
N-((1-((2-(3-chloro-5-fluorophenyl)-6-((2-(4-(2-hydroxyethyl)piperazin-1-yl)pyrimidin-5-yl)oxy)pyridin-4-yl)methyl)piperidin-4-yl)methyl)acetamide;
3-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-hydroxypropanoic acid;
(R)-2-((1-((2-(3,5-dichlorophenyl)-6-((2-(4-(2-hydroxypropyl)piperazin-1-yl)pyrimidin-5-yl)oxy)pyridin-4-yl)methyl)piperidin-4-yl)oxy)acetic acid;
((1-((2-(3,5-dichlorophenyl)-6-((2-(4-methylpiperazin-1-yl)pyrimidin-5-yl)oxy)pyridin-4-yl)methyl)piperidin-4-yl)methyl)boronic acid;
(2-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)ethyl)boronic acid;
((1-((2-(3,5-dichlorophenyl)-6-((2-(4-methylpiperazin-1-yl)pyrimidin-5-yl)oxy)pyridin-4-yl)methyl)piperidin-4-yl)methyl)dimethylphosphine oxide;
(1R,7S,8r)-4-((2-(3,5-dichlorophenyl)-6-((2-(4-methylpiperazin-1-yl)pyrimidin-5-yl)oxy)pyridin-4-yl)methyl)-4-azabicyclo[5.1.0]octane-8-carboxylic acid;
((1-((2-(3,5-dichlorophenyl)-6-((2-(4-methyl-1,4-diazepan-1-yl)pyrimidin-5-yl)oxy)pyridin-4-yl)methyl)piperidin-4-yl)methyl)boronic acid;
(2-(1-((2-(3,5-dichlorophenyl)-6-((2-(4-methylpiperazin-1-yl)pyrimidin-5-yl)oxy)pyridin-4-yl)methyl)piperidin-4-yl)ethyl)boronic acid;
(1-((2-(3,5-dichlorophenyl)-6-((6-(4-methylpiperazin-1-yl)pyridin-3-yl)oxy)pyridin-4-yl)methyl)piperidin-4-yl)methyl acetylcarbamate;
N-1-((1-((2-(3,5-dichlorophenyl)-6-((2-(4-methylpiperazin-1-yl)pyrimidin-5-yl)oxy)pyridin-4-yl)methyl)piperidin-4-yl)methyl)-N′-methoxylcarbonylurea;
N-(((1-((2-(3,5-dichlorophenyl)-6-((2-(4-methylpiperazin-1-yl)pyrimidin-5-yl)oxy)pyridin-4-yl)methyl)piperidin-4-yl)methyl)carbamoyl)methanesulfonamide;
N-(((1-((2-(3,5-dichlorophenyl)-6-((2-(4-methylpiperazin-1-yl)pyrimidin-5-yl)oxy)pyridin-4-yl)methyl)piperidin-4-yl)methyl)carbamoyl)acetamide;
N-((1-((2-(3,5-dichlorophenyl)-6-((2-(4-methylpiperazin-1-yl)pyrimidin-5-yl)oxy)pyridin-4-yl)methyl)piperidin-4-yl)carbamoyl)methanesulfonamide;
1-(1-((2-(3,5-dichlorophenyl)-6-((2-(4-methylpiperazin-1-yl)pyrimidin-5-yl)oxy)pyridin-4-yl)methyl)piperidin-4-yl)urea;
(S)-(4-((2-(3,5-dichlorophenyl)-6-((2-(4-methylpiperazin-1-yl)pyrimidin-5-yl)oxy)pyridin-4-yl)methyl)-1,4-oxazepan-7-yl)methanol;
(1-((2-(3,5-dichlorophenyl)-6-((2-(4-methylpiperazin-1-yl)pyrimidin-5-yl)oxy)pyridin-4-yl)methyl)piperidin-4-yl)dimethylphosphine oxide;
2-(1-((2-(3,5-dichlorophenyl)-6-((2-(4-methylpiperazin-1-yl)pyrimidin-5-yl)amino)pyridin-4-yl)methyl)piperidin-4-yl)acetic acid;
2-(1-((6-(3,5-dichlorophenyl)-3-methyl-2-((5-(4-methylpiperazin-1-yl)pyrazin-2-yl)oxy)pyridin-4-yl)methyl)piperidin-4-yl)acetic acid;
2-(1-((2-((2-(1,4-diazabicyclo[3.2.1]octan-4-yl)pyrimidin-5-yl)oxy)-6-(3,5-dichlorophenyl)pyridin-4-yl)methyl)piperidin-4-yl)acetic acid;
N-((1-((2-((2-(3,8-diazabicyclo[3.2.1]octan-3-yl)pyrimidin-5-yl)oxy)-6-(3,5-dichlorophenyl)pyridin-4-yl)methyl)piperidin-4-yl)methyl)acetamide;
methyl ((1-((2-((2-(1,4-diazabicyclo[3.2.1]octan-4-yl)pyrimidin-5-yl)oxy)-6-(3,5-dichlorophenyl)pyridin-4-yl)methyl)piperidin-4-yl)methyl)carbamate;
(1R,7S,8r)-4-((2-((2-(1,4-diazabicyclo[3.2.1]octan-4-yl)pyrimidin-5-yl)oxy)-6-(3,5-dichlorophenyl)pyridin-4-yl)methyl)-4-azabicyclo[5.1.0]octane-8-carboxylic acid;
4-(5-((6-(3,5-dichlorophenyl)-4-((4-fluoropiperidin-1-yl)methyl)pyridin-2-yl)oxy)pyrimidin-2-yl)-1,4-diazabicyclo[3.2.1]octane;
2-(1-((2-(3,5-dichlorophenyl)-6-((5-(4-methylpiperazin-1-yl)pyrazin-2-yl)oxy)pyridin-4-yl)methyl)piperidin-4-yl)acetic acid;
2-(1-((2-(3,5-dichlorophenyl)-6-((5-(4-methyl-1,4-diazepan-1-yl)pyrazin-2-yl)oxy)pyridin-4-yl)methyl)piperidin-4-yl)acetic acid;
4-(4-(5-((6-(3,5-dichlorophenyl)-4-((4-(2-hydroxyethyl)piperidin-1-yl)methyl)pyridin-2-yl)oxy)pyrazin-2-yl)piperazin-1-yl)-2-methylbutanoic acid;
2-(1-((2-(3,5-dichlorophenyl)-6-((5-(piperazin-1-yl)pyrazin-2-yl)oxy)pyridin-4-yl)methyl)piperidin-4-yl)ethanol;
2-(1-((2-(3,5-dichlorophenyl)-6-((5-(4-(2-hydroxy-2-methylpropyl)piperazin-1-yl)pyrazin-2-yl)oxy)pyridin-4-yl)methyl)piperidin-4-yl)acetic acid;
2-(1-((2-((6-(1,4-diazabicyclo[3.2.1]octan-4-yl)pyridazin-3-yl)oxy)-6-(3,5-dichlorophenyl)pyridin-4-yl)methyl)piperidin-4-yl)acetic acid;
2-(1-((2-(3,5-dichlorophenyl)-6-((5-(4-ethyl-1,4-diazepan-1-yl)pyrazin-2-yl)oxy)pyridin-4-yl)methyl)piperidin-4-yl)acetic acid;
2-(1-((2-(3,5-dichlorophenyl)-6-((5-((1S,4S)-5-methyl-2,5-diazabicyclo[2.2.1]heptan-2-yl)pyrazin-2-yl)oxy)pyridin-4-yl)methyl)piperidin-4-yl)acetic acid;
2-(1-((2-(3,5-dichlorophenyl)-6-((5-(4-isopropylpiperazin-1-yl)pyrazin-2-yl)oxy)pyridin-4-yl)methyl)piperidin-4-yl)acetic acid;
2-(1-((2-((5-(1,4-diazabicyclo[3.2.1]octan-4-yl)pyrazin-2-yl)oxy)-6-(3,5-dichlorophenyl)pyridin-4-yl)methyl)piperidin-4-yl)acetic acid;
2-(1-((2-(3,5-dichlorophenyl)-6-((5-(4-(3-hydroxybutyl)piperazin-1-yl)pyrazin-2-yl)oxy)pyridin-4-yl)methyl)piperidin-4-yl)acetic acid;
2-(1-((2-(3,5-dichlorophenyl)-6-((5-(4-methylpiperazin-1-yl)pyrazin-2-yl)oxy)pyridin-4-yl)methyl)piperidin-4-yl)ethanol;
3-(1-((2-(3,5-dichlorophenyl)-6-((5-(4-methyl-1,4-diazepan-1-yl)pyrazin-2-yl)oxy)pyridin-4-yl)methyl)piperidin-4-yl)-2-methylpropanoic acid;
2-(1-((2-(3,5-dichlorophenyl)-6-((5-(6-methyl-3,6-diazabicyclo[3.1.1]heptan-3-yl)pyrazin-2-yl)oxy)pyridin-4-yl)methyl)piperidin-4-yl)acetic acid;
1-((1-((2-(3,5-dichlorophenyl)-6-((5-(4-methylpiperazin-1-yl)pyrazin-2-yl)oxy)pyridin-4-yl)methyl)piperidin-4-yl)oxy)cyclopropanecarboxylic acid;
2-(1-((2-(3,5-dichlorophenyl)-6-((5-(4-(3-(methylsulfonyl)propyl)piperazin-1-yl)pyrazin-2-yl)oxy)pyridin-4-yl)methyl)piperidin-4-yl)acetic acid;
2-(1-((2-(3,5-dichlorophenyl)-6-((6-(4-ethyl-1,4-diazepan-1-yl)pyridazin-3-yl)oxy)pyridin-4-yl)methyl)piperidin-4-yl)acetic acid;
2-(1-((2-(3,5-dichlorophenyl)-6-((6-(4-methyl-1,4-diazepan-1-yl)pyridazin-3-yl)oxy)pyridin-4-yl)methyl)piperidin-4-yl)acetic acid;
2-(1-((2-(3,5-dichlorophenyl)-6-((6-((1S,4S)-5-methyl-2,5-diazabicyclo[2.2.1]heptan-2-yl)pyridazin-3-yl)oxy)pyridin-4-yl)methyl)piperidin-4-yl)acetic acid;
2-(1-((2-(3,5-dichlorophenyl)-6-((6-(4-ethylpiperazin-1-yl)pyridazin-3-yl)oxy)pyridin-4-yl)methyl)piperidin-4-yl)acetic acid;
2-(1-((2-(3,5-dichlorophenyl)-6-((6-(4-methylpiperazin-1-yl)pyridazin-3-yl)oxy)pyridin-4-yl)methyl)piperidin-4-yl)acetic acid;
2-(1-((2-(3,5-dichlorophenyl)-6-((6-(4-methylpiperazin-1-yl)pyridazin-3-yl)oxy)pyridin-4-yl)methyl)piperidin-4-yl)ethanol;
(1R,7S,8r)-4-((2-(3,5-dichlorophenyl)-6-((6-(4-methylpiperazin-1-yl)pyridazin-3-yl)oxy)pyridin-4-yl)methyl)-4-azabicyclo[5.1.0]octane-8-carboxylic acid;
(1R,7S,8r)-4-((2-(3,5-dichlorophenyl)-6-((6-(4-methyl-1,4-diazepan-1-yl)pyridazin-3-yl)oxy)pyridin-4-yl)methyl)-4-azabicyclo[5.1.0]octane-8-carboxylic acid;
3-(4-(6-((4-((4-(acetamidomethyl)piperidin-1-yl)methyl)-6-(3,5-dichlorophenyl)pyridin-2-yl)oxy)pyridazin-3-yl)piperazin-1-yl)-2-methylpropanoic acid;
(R)-3-(1-((2-(3,5-dichlorophenyl)-6-((6-(4-methylpiperazin-1-yl)pyridazin-3-yl)oxy)pyridin-4-yl)methyl)piperidin-4-yl)-2-methylpropanoic acid;
(S)-3-(1-((2-(3,5-dichlorophenyl)-6-((6-(4-methylpiperazin-1-yl)pyridazin-3-yl)oxy)pyridin-4-yl)methyl)piperidin-4-yl)-2-methylpropanoic acid;
2-(1-((2-(3,5-dichlorophenyl)-6-((6-((1S,4S)-5-ethyl-2,5-diazabicyclo[2.2.1]heptan-2-yl)pyridazin-3-yl)oxy)pyridin-4-yl)methyl)piperidin-4-yl)acetic acid;
3-(4-(5-((4-(((1R,7S,8r)-8-acetamido-4-azabicyclo[5.1.0]octan-4-yl)methyl)-6-(3,5-dichlorophenyl)pyridin-2-yl)oxy)pyrimidin-2-yl)piperazin-1-yl)propanoic acid;
methyl 4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylbutanoate;
4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2-methylbutanoic acid;
4-(4-(5-((4-((4-(acetamidomethyl)piperidin-1-yl)methyl)-6-(3,5-dichlorophenyl)pyridin-2-yl)oxy)pyrimidin-2-yl)piperazin-1-yl)-2,2-dimethylbutanoic acid;
2-(1-((6-(3,5-dichlorophenyl)-3-methyl-2-((6-(4-methylpiperazin-1-yl)pyridazin-3-yl)oxy)pyridin-4-yl)methyl)piperidin-4-yl)acetic acid;
N-((1-((2-(3-chloro-5-fluorophenyl)-6-((2-(4-(4-(methylsulfonyl)butan-2-yl)piperazin-1-yl)pyrimidin-5-yl)oxy)pyridin-4-yl)methyl)piperidin-4-yl)methyl)acetamide;
1-((1-((2-(3-chloro-5-fluorophenyl)-6-((2-(4-(4-(methylsulfonyl)butan-2-yl)piperazin-1-yl)pyrimidin-5-yl)oxy)pyridin-4-yl)methyl)piperidin-4-yl)methyl)-3-methylurea;
2-(1-((2-(3-chloro-5-fluorophenyl)-6-((2-(4-methyl-1,4-diazepan-1-yl)pyrimidin-5-yl)oxy)pyridin-4-yl)methyl)piperidin-4-yl)acetic acid;
2-(1-((2-(3,5-dichlorophenyl)-6-(isopropyl(2-(4-methylpiperazin-1-yl)pyrimidin-5-yl)amino)pyridin-4-yl)methyl)piperidin-4-yl)acetic acid;
2-(1-((3′,5′-dichloro-4-fluoro-5-((2-(4-methylpiperazin-1-yl)pyrimidin-5-yl)oxy)-[1,1′-biphenyl]-3-yl)methyl)piperidin-4-yl)acetic acid;
2-(1-((6-(3,5-dichlorophenyl)-3-fluoro-2-((6-(4-methylpiperazin-1-yl)pyridin-3-yl)oxy)pyridin-4-yl)methyl)piperidin-4-yl)acetic acid;
2-(1-((6-(3,5-dichlorophenyl)-3-fluoro-2-((2-(8-methyl-3,8-diazabicyclo[3.2.1]octan-3-yl)pyrimidin-5-yl)oxy)pyridin-4-yl)methyl)piperidin-4-yl)acetic acid;
2-(1-((6-(3,5-dichlorophenyl)-3-fluoro-2-((2-(4-methylpiperazin-1-yl)pyrimidin-5-yl)oxy)pyridin-4-yl)methyl)piperidin-4-yl)acetic acid;
2-(1-((6-(3,5-dichlorophenyl)-3-fluoro-2-((4-methyl-2-(4-methylpiperazin-1-yl)pyrimidin-5-yl)oxy)pyridin-4-yl)methyl)piperidin-4-yl)acetic acid;
4-(4-(5-((4-((4-(acetamidomethyl)piperidin-1-yl)methyl)-6-(3,5-dichlorophenyl)-3-fluoropyridin-2-yl)oxy)pyrimidin-2-yl)piperazin-1-yl)-2-methylbutanoic acid;
2-(1-((2-((2-(3,8-diazabicyclo[3.2.1]octan-3-yl)pyrimidin-5-yl)oxy)-6-(3,5-dichlorophenyl)-3-fluoropyridin-4-yl)methyl)piperidin-4-yl)acetic acid;
2-(1-((6-(3,5-dichlorophenyl)-3-fluoro-2-((2-methyl-6-(4-methylpiperazin-1-yl)pyridin-3-yl)oxy)pyridin-4-yl)methyl)piperidin-4-yl)acetic acid;
2-(1-((6-(3,5-dichlorophenyl)-3-fluoro-2-((6-(4-methylpiperazin-1-yl)pyridazin-3-yl)oxy)pyridin-4-yl)methyl)piperidin-4-yl)acetic acid;
methyl 3-(4-(5-((4-((4-(acetamidomethyl)piperidin-1-yl)methyl)-6-(3,5-dichlorophenyl)pyridin-2-yl)oxy)pyridin-2-yl)piperazin-1-yl)propanoate;
2-(1-((6-(3,5-dichlorophenyl)-3-methyl-2-((6-(4-methylpiperazin-1-yl)pyridin-3-yl)oxy)pyridin-4-yl)methyl)piperidin-4-yl)acetic acid;
2-(1-((2-((2-(4-((1H-1,2,3-triazol-5-yl)methyl)piperazin-1-yl)pyrimidin-5-yl)oxy)-6-(3,5-dichlorophenyl)pyridin-4-yl)methyl)piperidin-4-yl)acetic acid;
2-(1-((2-(3,5-dichlorophenyl)-6-(methyl(2-(4-methylpiperazin-1-yl)pyrimidin-5-yl)amino)pyridin-4-yl)methyl)piperidin-4-yl)acetic acid;
2-(1-((2-(3,5-dichlorophenyl)-6-(ethyl(2-(4-methylpiperazin-1-yl)pyrimidin-5-yl)amino)pyridin-4-yl)methyl)piperidin-4-yl)acetic acid;
2-(1-((6-(3,5-dichlorophenyl)-3-fluoro-2-((5-(4-methylpiperazin-1-yl)pyrazin-2-yl)oxy)pyridin-4-yl)methyl)piperidin-4-yl)acetic acid;
3-(4-(5-((4-((4-(acetamidomethyl)piperidin-1-yl)methyl)-6-(3,5-dichlorophenyl)pyridin-2-yl)oxy)pyrazin-2-yl)piperazin-1-yl)propanamide;
4-(4-(5-((6-(3,5-dichlorophenyl)-4-((4-(propionamidomethyl)piperidin-1-yl)methyl)pyridin-2-yl)oxy)pyrazin-2-yl)piperazin-1-yl)-2-methylbutanoic acid;
N-((1-((2-(3,5-dichlorophenyl)-6-((5-(4-(1-hydroxypropan-2-yl)piperazin-1-yl)pyrazin-2-yl)oxy)pyridin-4-yl)methyl)piperidin-4-yl)methyl)acetamide;
1-((1-((2-(3,5-dichlorophenyl)-6-((5-(4-methylpiperazin-1-yl)pyrazin-2-yl)oxy)pyridin-4-yl)methyl)piperidin-4-yl)methyl)-3-methylurea;
methyl ((1-((2-(3,5-dichlorophenyl)-6-((5-(4-methylpiperazin-1-yl)pyrazin-2-yl)oxy)pyridin-4-yl)methyl)piperidin-4-yl)methyl)carbamate;
4-(4-(5-((4-((4-(acetamidomethyl)piperidin-1-yl)methyl)-6-(3,5-dichlorophenyl)pyridin-2-yl)oxy)pyrazin-2-yl)piperazin-1-yl)-2-methylbutanoic acid;
(1R,7S,8r)-4-((2-(3,5-dichlorophenyl)-6-((2-(4-methyl-1,4-diazepan-1-yl)pyrimidin-5-yl)oxy)pyridin-4-yl)methyl)-4-azabicyclo[5.1.0]octane-8-carboxylic acid;
2-(1-((2-(3,5-dichlorophenyl)-6-((2-(8-methyl-3,8-diazabicyclo[3.2.1]octan-3-yl)pyrimidin-5-yl)oxy)pyridin-4-yl)methyl)piperidin-4-yl)acetic acid; or
2-(1-((2-(3,5-dichlorophenyl)-6-((2-(6-fluoro-1,4-diazepan-1-yl)pyrimidin-5-yl)oxy)pyridin-4-yl)methyl)piperidin-4-yl)acetic acid;
or a pharmaceutically acceptable salt thereof.

42. The method or use of any one of claims 1 to 41, wherein the compound, or pharmaceutically acceptable salt thereof, is of the formula:

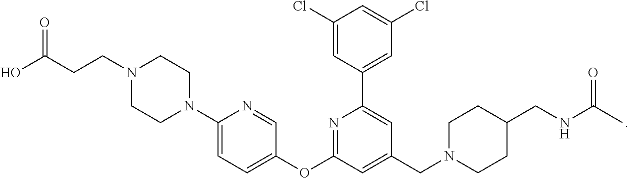 [SMILES: CC(=O)NCC1CCN(Cc2cc(Oc3ccc(N4CCN(CCC(=O)O)CC4)nc3)nc(-c3cc(Cl)cc(Cl)c3)c2)CC1]

43. The method or use of any one of claims 1 to 41, wherein the compound, or pharmaceutically acceptable salt thereof, is of the formula: 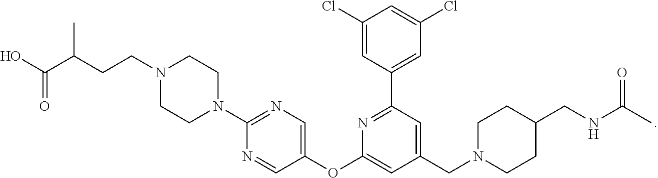 [SMILES: CC(=O)NCC1CCN(Cc2cc(Oc3cnc(N4CCN(CCC(C)C(=O)O)CC4)nc3)nc(-c3cc(Cl)cc(Cl)c3)c2)CC1]
